(19) United States
(12) Patent Application Publication
(10) Pub. No.: US 2024/0124849 A1
(43) Pub. Date: Apr. 18, 2024

(54) AAV PRODUCTION SYSTEMS FOR AAV VIRAL PARTICLES WITH IMPROVED INFECTIVITY

(71) Applicant: BioMarin Pharmaceutical Inc., Novato, CA (US)

(72) Inventors: Vishal AGRAWAL, Novato, CA (US); Teresa CHRISTIANSON, Novato, CA (US); Francisco Javier FEMENIA, Novato, CA (US); Santosh G. PANDE, Novato, CA (US)

(73) Assignee: BioMarin Pharmaceutical Inc., Novato, CA (US)

(21) Appl. No.: 18/273,430

(22) PCT Filed: Jan. 27, 2022
(86) PCT No.: PCT/US2022/014058

§ 371 Date: Jul. 20, 2023

Related U.S. Application Data
(60) Provisional application No. 63/144,104, filed on Feb. 1, 2021.

(51) Int. Cl. C12N 7/00; C07K 14/015; C12N 15/86

(52) U.S. Cl. CPC C12N 7/00; C07K 14/015; C12N 15/86; C12N 2750/14151

(57) ABSTRACT

The present invention provides modified alphaviruses and compositions, methods, and kits for preparing and using, in particular AAV viral particles pseudotyped with capsids, in particular for use in gene therapy and/or diagnostics.

### REFERENCE TO SEQUENCE LISTING

The Sequence Listing concurrently submitted herewith as a text file named “2021-02-01_Sequence-Listing_6439-0114PUS1_ST25” created on Feb. 1, 2021, and having a size of 475 kilobytes (486,939 bytes) is herein incorporated by reference pursuant to 37 C.F.R. § 1.52(e)(5).

### FIELD OF THE INVENTION

The present invention relates to methods for preparing adeno-associated virus (AAV) viral particles. The invention also relates to the herein described AAV viral particles and compositions, methods, uses, and kits related thereto.

### BACKGROUND

Adeno-associated viruses (AAV) are small, non-pathogenic satellite viruses that are believed to require a helper adenovirus for replication. AAVs are similar in structure to adenoviruses but have a smaller icosahedral nucleocapsid. AAV are non-enveloped viruses with single-stranded DNA genome with at least one inverted terminal repeat (ITR) at the termini. For example, the AAV2 serotype can have a single-stranded DNA genome of approximately 4.7-kilobases (kb), with two 145 nucleotide-long ITRs at the termini. The virus does not encode a polymerase and therefore relies on cellular polymerases for genome replication. The ITRs flank the two viral genes—rep (replication) and cap (capsid), encoding non-structural and structural proteins, respectively. The rep gene, through the use of two promoters and alternative splicing, encodes four regulatory proteins that are dubbed Rep78, Rep68, Rep52 and Rep40. These proteins are involved in AAV genome replication and packaging. The cap gene, through alternative splicing and initiation of translation, gives rise to three capsid proteins, VP1 (virion protein 1), VP2 and VP3. The molecular weight of VP1, VP2, and VP3 for AAV2 is 87, 72 and 62 kDa, respectively. These capsid proteins assemble into a near-spherical protein shell of 60 subunits. The AAV structural simplicity and non-pathogenic nature make recombinant AAV (rAAV) a useful gene therapy vector. AAV gene therapy vectors can infect both replicating and non-replicating cells and introduce transgenes without integrating into the genome of the host cell. rAAV vectors are often preferred due to their high titer, ability to infect a broad range of cells, mild immune response, and overall safety. rAAV gene therapy vectors have been found to be highly useful for a number of diseases including diabetes and other pancreatic disorders.

There remains a need for new and effective AAV viral particles, and compositions, methods, and kits related thereto.

### SUMMARY OF THE INVENTION

In various aspects, methods for preparing recombinant adeno-associated virus (rAAV) are disclosed.

In one aspect, a method for preparing rAAV is disclosed and includes the step of culturing, in a culture medium with an effective amount of a transition metal. The host cell is capable of producing an rAAV capsid. The effective amount of the transition metal increases incorporation of VP1, VP2, or VP3 protein into the rAAV capsid. Alternatively, the transition metal decreases incorporation of VP2 and increases incorporation VP1 or VP3 into the rAAV capsid. The transition metal can also decrease incorporation of VP3 and increases incorporation VP1 into the rAAV capsid.

In one aspect, a method for preparing rAAV is disclosed and includes the step of culturing a host cell in a culture medium having a nanomolar concentration of a transition metal. The host cell is capable of producing an rAAV capsid.

In one aspect, a method for preparing rAAV is disclosed and includes the step of culturing a host cell in a culture medium having a micromolar concentration of a transition metal. The host cell is capable of producing an rAAV capsid.

In one aspect, a method for preparing rAAV is disclosed and includes the step of culturing, in a culture medium with an effective amount of a transition metal. The host cell is capable of producing an rAAV capsid. The effective amount of the transition metal increases incorporation of VP1 and VP3 proteins into the rAAV capsid. The rAAV capsid has concentrations of VP1 and VP3 proteins that are greater than concentrations of VP1 and VP3 proteins of an rAAV capsid produced under the same conditions but being devoid of the effective amount of the transition metal.

In one aspect, a method for preparing rAAV is disclosed and includes the step of culturing, in a culture medium with an effective amount of a transition metal. The host cell is capable of producing an rAAV capsid. The effective amount of the transition metal increases incorporation of VP1 proteins into the rAAV capsid.

In one aspect, a method for preparing rAAV is disclosed and includes the step of culturing, in a culture medium with an effective amount of a cysteine protease inhibitor. The host cell is capable of producing an rAAV capsid. The effective amount of a cysteine protease inhibitor increases incorporation of VP1, VP2, or VP3 protein into the rAAV capsid. Alternatively, the effective amount of a cysteine protease inhibitor decreases incorporation of VP2 and increases incorporation VP1 or VP3 into the rAAV capsid. The effective amount of a cysteine protease inhibitor can also decrease incorporation of VP3 and increases incorporation VP1 into the rAAV capsid.

In one aspect, an rAAV capsid produced by any method of any aspect or embodiment is disclosed. The rAAV capsid has a concentration of VP1, VP2, or VP3 proteins that is greater than a concentration of VP1, VP2, or VP3 proteins of an rAAV capsid produced under the same conditions but being devoid of the effective amount of the transition metal.

In one aspect, an rAAV capsid produced by any method of any aspect or embodiment is disclosed. The rAAV capsid has a concentration of VP2 or VP3 proteins that is lower than a concentration of VP2 or VP3 proteins of an rAAV capsid produced under the same conditions but being devoid of the effective amount of the transition metal.

In one aspect, an rAAV capsid produced by any method of any aspect or embodiment is disclosed. The rAAV capsid exhibits enhanced infectivity.

In one aspect, an rAAV capsid produced by any method of any aspect or embodiment is a therapeutically effective rAAV.

In one embodiment, the host cell is a non-mammalian host cell. In another embodiment, the host cell is an insect cell.

In one embodiment, the transition metal is a transition metal salt. Examples of transition metals include copper acetate, cuprous oxide, cupric oxide, cupric chloride, copper oxychloride, cuprous chloride, cupric nitrate, copper cyanide, a copper soap, copper naphthenate, or copper sulfate.

In various embodiments, the transition metal is selected from one or more of copper, scandium, titanium, vanadium, chromium, manganese, iron, cobalt, nickel, copper, zinc, yttrium, zirconium, niobium, molybdenum, technetium, ruthenium, rhodium, palladium, silver, cadmium, hafnium, tantalum, tungsten, rhenium, osmium, iridium, platinum, gold, mercury, rutherfordium, dubnium, seaborgium, bohrium, hassium, meitnerium, ununnilium, unununium, and ununbium.

In various embodiments, the effective amount of the transition metal ranges from about 1 nanomolar (nM) to about 1 millimolar (mM). In various embodiments, the effective amount of the transition metal ranges from about 10 nM to about 100 micromolar (μM). In various embodiments, the effective amount of the transition metal ranges from about 20 μM to about 25 μM. In various embodiments, the nanomolar concentration of the transition metal ranges from about 1 nM to less than 1 μM.

In various embodiments, the nanomolar concentration of the transition metal ranges from about 10 nM to less than 1 μM.

In various embodiments, the micromolar concentration of the transition metal ranges from about 1 μM to less than 1 mM. In various embodiments, the micromolar concentration of the transition metal ranges from about 1 μM to about 100 μM. In various embodiments, the micromolar concentration of the transition metal ranges from about 20 μM to about 25 μM.

In various embodiments, the effective amount of the transition metal is sufficient to increase incorporation of VP1 protein into the rAAV capsid. In various embodiments, the effective amount of the transition metal is sufficient to increase or decrease incorporation of VP3 protein into the rAAV capsid. In various embodiments, the effective amount of the transition metal is sufficient to increase or decrease incorporation of VP2 protein into the rAAV capsid. In various embodiments, the effective amount of the transition metal is sufficient to increase incorporation of VP1, VP2, or VP3 protein into the rAAV capsid. In other embodiments, the effective amount of the transition metal is sufficient to decrease incorporation of VP2 or VP3 proteins into the rAAV capsid.

In various embodiments, the method of any aspect or embodiment further includes the step of isolating the rAAV capsid from the host cell.

In various embodiments, the method of any aspect or embodiment further includes host cells capable of producing rAAV capsids.

In various embodiments, the culturing step of any aspect or embodiment occurs in a volume of at least 1 liter (L), at least 10 L, at least 50 L, at least 100 L, at least 250 L, at least 500 L, at least 1000 L, at least 1500 L, at least 2000 L, or at least 2500 L.

In one aspect, the present invention provides a method for preparing an AAV viral particle. The method comprises culturing, in a culture medium comprising an effective amount of a salt, a host cell capable of producing the AAV viral particle. The AAV viral particle comprises an AAV capsid. The AAV capsid comprises a VP1 protein. The salt includes a transition metal.

In another aspect, the present invention provides a method for preparing an AAV viral particle. The method comprises culturing, in a culture medium having an effective amount of an inhibitor of a cysteine protease, a host cell capable of producing the AAV viral particle, wherein the AAV viral particle comprises an AAV capsid and the AAV capsid comprises a VP1 protein.

In other aspects, the present invention provides a therapeutically effective rAAV. The therapeutically effective rAAV is produced by any method of any aspect or any embodiment. The therapeutically effective rAAV is pseudotyped with an AAV capsid.

In some aspects, the present invention provides an rAAV particle prepared by a method comprising culturing, in a culture medium containing an effective amount of a salt, a host cell capable of producing the AAV viral particle. The AAV viral particle comprises an AAV capsid. The AAV capsid comprises a VP1 protein. The salt includes a transition metal such as a copper salt.

In one aspect, the present invention provides an rAAV particle prepared by a method comprising culturing, in a culture medium having an effective amount of an inhibitor of a cysteine protease, a host cell capable of producing the rAAV particle. The rAAV particle comprises an AAV capsid. The AAV capsid comprises a VP1 protein.

In another aspect, the present invention provides pharmaceutical formulations of rAAV particles of the invention.

In some aspects, the present invention provides uses of the rAAV particles of the invention for efficient transduction of cells, tissues, and/or organs of interest, and/or for use in gene therapy.

In other aspects, the present invention provides a kit for use with methods and compositions described herein.

### BRIEF DESCRIPTION OF THE DRAWINGS

FIG. 1 is a graph showing dose dependent effects of copper sulfate addition to Sf9 insect cell culture producing rAAV particles pseudotyped with Bba41 capsids. The Sf9 insect cell cultures were cultured with 0 micromolar (μM), 5 μM, and 50 μM concentrations of copper sulfate and infected with recombinant baculovirus (rBV) having vector(s) for Bba41 capsid production. After a predetermined time post infection, the Bba41 capsids were isolated from the insect cell cultures and the infectivity of the Bba41 capsids was assessed. FIG. 1 shows increasing transduction activity of rAAV particles pseudotyped with Bba41 capsids that are produced in Sf9 insect cells supplemented with increasing concentrations of copper sulfate.

FIG. 2 is a sodium dodecyl sulfate polyacrylamide gel electrophoresis (SDS-PAGE) showing dose dependent effects of copper sulfate addition to Sf9 insect cell culture producing rAAV particles pseudotyped with Bba41 capsids. FIG. 2 shows the VP1 from Bba41 capsids produced in copper sulfate supplemented Sf9 insect cells having a similar banding pattern to VP1 from Bba41 capsids produced in human embryonic kidney-293 (HEK293) cells. The HEK293 cultures were cultured with 0 μM, 5 μM, and 50 μM concentrations of copper sulfate and transfected with plasmids for Bba41 capsid production. After a predetermined time post transfection, the Bba41 capsids were isolated from the HEK293 cultures.

FIG. 3 is a graph showing dose dependent effects of copper sulfate addition to Sf9 insect cell and HEK293 cell cultures producing rAAV particles pseudotyped with Bba41 capsids. The insect cell cultures were shake flask productions and HEK293 cultures were shake flask and bioreactor productions. The Bba41 capsids were isolated from the cell cultures and the infectivity of the Bba41 capsids was assessed in vitro. FIG. 3 shows increasing transduction activity of rAAV particles pseudotyped with Bba41 capsids that are produced in Sf9 insect cells supplemented with increasing concentrations of copper sulfate. The increased transduction activity is comparable to rAAV particles pseudotyped with Bba41 capsids that are produced in HEK293 cells.

FIG. 4 is an agarose gel showing AAV vector genomes from rAAV produced in Sf9 insect cells and HEK293 cells.

FIG. 5 is a graph showing VP1 concentrations of rAAV capsids produced in Sf9 cells. Shake flask cultures of insect cells were cultured with 0 μM, 25 μM, and 45 μM concentrations of copper sulfate and infected with rBV having vector(s) for AAV5 capsid production. After a predetermined time post infection, the AAV5 capsids were isolated from the insect cell cultures and the VP1 concentration of the AAV5 capsids was quantified. The different concentrations of copper sulfate increased the VP1 concentrations of the produced AAV5 capsids.

FIG. 6 is a graph showing the transduction activity of rAAV capsids produced in Sf9 cells. Shake flask cultures of insect cells were cultured with 0 μM and 25 μM concentrations of copper sulfate and infected with rBV having vector(s) for AAV5 capsid production. After a predetermined time post infection, the AAV5 capsids were isolated from the insect cell cultures and the infectivity of the AAV5 capsids was assessed in vitro. 25 μNI copper sulfate improved the infectivity of the produced AAV5 capsids.

FIG. 7 is a graph showing VP1 concentrations of rAAV capsids produced in Sf9 cells that were cultured with or without copper. Bioreactor cultures of insect cells were cultured with 0 μM, and 30 μM concentrations of copper sulfate and infected with rBV having vector(s) for AAV5 capsid production. After a predetermined time post infection, the AAV5 capsids were isolated from the insect cell cultures and the VP1 concentration of the AAV5 capsids was quantified. 30 μM copper sulfate increased the VP1 concentrations of the AAV5 capsids produced at bioreactor scale.

FIG. 8 is a graph showing transgene expression in cells infected with rAAV capsids produced in Sf9 cells. Bioreactor cultures of insect cells were cultured with 0 μM and 30 μM concentrations of copper sulfate and infected with recombinant baculovirus (rBV) having vector(s) for AAV5 capsid production. After a predetermined time post infection, the AAV5 capsids were isolated from the insect cell cultures and the infectivity of the AAV5 capsids was assessed in vitro. 30 μM copper sulfate improved the infectivity of the AAV5 capsids produced at bioreactor scale.

FIG. 9 is a graph showing VP1 concentrations of rAAV capsids produced in Sf9 cells. 2000 liter (L) and 3 L bioreactor cultures and shake flask cultures of insect cells were cultured with 0 μM and 30 μM concentrations of copper sulfate and infected with recombinant baculovirus (rBV) having vector(s) for AAV5 capsid production. After a predetermined time post infection, the AAV5 capsids were isolated from the insect cell cultures and the VP1 concentration of the AAV5 capsids was quantified. The VP1 concentration of AAV5 capsids produced in the copper supplemented bioreactor and shake flask cultures were compared to the VP1 concentration of AAV5 capsids produced in shake flask cultures without copper. 30 μM copper sulfate increased the VP1 concentrations of the AAV5 capsids produced using different production types and scales.

FIG. 10 is a graph showing VP1 concentrations of rAAV capsids produced in SD cells. 100 milliliter (mL) shake flask cultures of insect cells were cultured with 0 μM and 30 μM concentrations of copper sulfate and infected with rBV having vector(s) for AAV9 capsid production. After a predetermined time post infection, the AAV9 capsids were isolated from the insect cell cultures and the VP1 concentration of the AAV9 capsids was quantified. 30 μM copper sulfate increased the VP1 concentrations of the produced AAV9 capsids.

FIG. 11 is a graph showing the transduction activity of rAAV capsids produced in Sf9 cells. Shake flask cultures of insect cells were cultured with 0 μM, 10 μM, 20 μM, and 30 μM concentrations of copper sulfate and infected with rBV having vector(s) for AAV9 capsid production at different multiplicities of infection (MOI). After a predetermined time post infection, the AAV9 capsids were isolated from the insect cell cultures and the infectivity of the AAV9 capsids was assessed in vitro. The different concentrations of copper sulfate at different MOI improved the infectivity of the produced AAV9 capsids.

### DETAILED DESCRIPTION OF THE INVENTION

As required, detailed aspects and embodiments of the present disclosure are disclosed herein; however, it is to be understood that the disclosed aspects and embodiments are merely exemplary and may be embodied in various and alternative forms.

Except in the examples, or where otherwise expressly indicated, all numerical quantities in this description indicating amounts of material or conditions of reaction and/or use are to be understood as modified by the word “about”. For example, description referring to “about X” includes description of “X.” In one example, the term “about” is understood as within a range of normal tolerance in the art, for example within 2 standard deviations of the mean. In different examples, “about” refers a variability of ±0.0001%, ±0.0005%, ±0.001%, ±0.005%, ±0.01%, ±0.05%, ±0.1%, ±0.5%, ±1%, ±5%, or ±10%. In further examples, “about” can be understood as within ±9%, ±8%, ±7%, ±6%, ±5%, ±4%, ±3%, or ±2%.

Unless otherwise clear from context, all numerical values provided herein are modified by the term about. All ranges include the endpoints of the ranges. The first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

Unless indicated otherwise, all technical and scientific terms used herein have the same meaning as commonly understood by one of ordinary skill in the art to which the present disclosure belongs.

It is also to be understood that this disclosure is not limited to the specific aspects and embodiments described below, as specific components and/or conditions may, of course, vary. Furthermore, the terminology used herein is used only for describing particular aspects and embodiments and is not intended to be limiting in any way.

Throughout this application, where publications are referenced, the disclosures of these publications in their entireties are hereby incorporated by reference into this application to more fully describe the state of the art to which this invention pertains.

It is also noted that, as used in the specification and the appended claims, the singular form “a,” “an,” and “the” comprise plural referents unless the context clearly indicates otherwise. For example, reference to a component in the singular is intended to comprise a plurality of components.

The terms “or” and “and” can be used interchangeably and can be understood to mean “and/or”.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

The terms “comprising”, “consisting of”, and “consisting essentially of” can be alternatively used. When one of these three terms is used, the presently disclosed and claimed subject matter can include the use of either of the other two terms.

In accordance with the present invention, the inventors have surprisingly discovered that by culturing a host cell capable of producing rAAV in a culture medium comprising an effective amount of a transition metal or a salt, the infectivity of a rAAV particle produced by the host cell is improved. In particular, the inventors have discovered that the effective amount of the salt in the culture medium increases the amount of at least the VP1 protein incorporated in the capsid, thereby enhancing the infectivity of the AAV viral particles produced. Thus, the inventors have discovered methods and processes of producing therapeutically effective rAAV.

Although the methods described herein may be disclosed and described as step(s), it is to be understood that the methods are not necessarily limited by the order of steps, as some steps may, in accordance with these methods, occur in different orders, and/or concurrently with other step(s) described herein and/or known in the art.

Adeno Associated Virus

In one aspect, an rAAV capsid produced by method of any aspect or embodiment is disclosed. The rAAV capsid has a concentration of VP1, VP2, or VP3 proteins that is greater than a concentration of VP1, VP2, or VP3 proteins of an rAAV capsid produced under the same conditions but being devoid of the effective amount of the transition metal.

In one aspect, an rAAV capsid produced by any method of any aspect or embodiment is disclosed. The rAAV capsid exhibits enhanced infectivity.

The rAAV and rAAV capsids include rAAV particles disclosed in or may be made according to knowing methods, for example as taught in U.S. Pat. No. 9,504,762, WO 2018/022608, WO 2019/222136, and US 2019/0376081, the disclosures of which are hereby incorporated by reference in their entirety.

“AAV” is a standard abbreviation for adeno-associated virus. Adeno-associated virus is a single-stranded DNA parvovirus having a genome encapsulated by a capsid. There are currently thirteen serotypes of AAV that have been characterized. General information and reviews of AAV can be found in, for example, Carter, 1989, Handbook of Parvoviruses, Vol. 1, pp. 169-228; and Berns, 1990, Virology, pp. 1743-1764, Raven Press, (New York). However, it is fully expected that these same principles will be applicable to additional AAV serotypes since it is well known that the various serotypes are quite closely related, both structurally and functionally, even at the genetic level. (See, e.g., Blacklowe, 1988, pp. 165-174 of Parvoviruses and Human Disease, J. R. Pattison, ed.; and Rose, Comprehensive Virology 3:1-61 (1974)). For example, all AAV serotypes apparently exhibit very similar replication properties mediated by homologous rep genes; and all bear three related capsid proteins. The degree of relatedness is further suggested by heteroduplex analysis which reveals extensive cross-hybridization between serotypes along the length of the genome; and the presence of analogous self-annealing segments at the termini that correspond to inverted terminal repeats (ITRs). The similar infectivity patterns also suggest that the replication functions in each serotype are under similar regulatory control.

An “AAV viral particle” as used herein refers to an infectious viral particle composed of at least one AAV capsid protein and an encapsidated AAV genome. “Recombinant AAV” or “rAAV”, “rAAV virion” or “rAAV viral particle” or “rAAV vector particle” or “AAV virus” refers to a viral particle composed of at least one capsid or Cap protein and an encapsidated rAAV vector genome (vg) as described herein. If the particle comprises a heterologous polynucleotide (i.e., a polynucleotide other than a wild-type AAV genome such as a transgene to be delivered to a mammalian cell), it is typically referred to as an “rAAV vector particle” or simply an “rAAV vector”. Thus, production of AAV vector particles necessarily includes production of rAAV vector, as such a vector is contained within an rAAV vector particle.

As used herein, the terms “heterologous gene”, “heterologous sequence”, “heterologous”, “heterologous regulatory sequence”, “heterologous transgene”, or “transgene” means that the referenced gene or regulatory sequence is not naturally present in the AAV vector or particle and has been artificially introduced therein. For example, these terms refer to a nucleic acid that comprises both a heterologous gene and a heterologous regulatory sequence that are operably linked to the heterologous gene that control expression of that gene in a host cell. It is contemplated that the transgene herein can encode a biomolecule (e.g., a therapeutic biomolecule), such as a protein (e.g., an enzyme), polypeptide, peptide, RNA (e.g., tRNA, dsRNA, ribosomal RNA, catalytic RNAs, siRNA, miRNA, pre-miRNA, lncRNA, snoRNA, small hairpin RNA, trans-splicing RNA, and antisense RNA), one or more components of a gene or base editing system, e.g., a CRISPR gene editing system, antisense oligonucleotides (AONs), antisense oligonucleotide (AON)-mediated exon skipping, a poison exon(s) that triggers nonsense mediated decay (NMD), or a dominant negative mutant.

The term “recombinant” refers nucleic acid molecules or proteins formed by using recombinant DNA techniques. For example, a recombinant nucleic acid molecule can be formed by combining nucleic acid sequences and sequence elements. A recombinant protein can be a protein that is produced by a cell that has received a recombinant nucleic acid molecule.

The terms “encodes,” “encoded” and “encoding” refer to the inherent property of specific sequences of nucleotides in a nucleic acid molecule, such as a gene, complementary DNA (cDNA), or messenger RNA (mRNA), to serve as templates for synthesis of other polymers and macromolecules in biological processes. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA.

“Capsid” refers to the structure in which the rAAV vector genome is packaged. The capsid includes VP1 proteins or VP3 proteins, but more typically, all three of VP1, VP2, and VP3 proteins, as found in native AAV. The sequence of the capsid proteins determines the serotype of the rAAV virions. rAAV virions include those derived from a number of AAV serotypes, including AAV1, AAV2, AAV4, AAV5, AAV6, AAV7, AAV8, AAV9, AAV10, AAV11, AAV12, AAV13, AAV-rh.10 (AAVrh10), AAV-DJ (AAVDJ), AAV-DJ8 (AAVDJ8), AAV-1, AAV-2, AAV-2G9, AAV-3, AAV3a, AAV3b, AAV3-3, AAV4, AAV4-4, AAV-5, AAV-6, AAV6.1, AAV6.2, AAV6.1.2, AAV-7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV-10, AAV-11, AAV-12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV10, or Japanese AAV10 serotypes, AAV_po.6, AAV_po., AAV_po.5, AAV_LK03, AAV_ra.1, AAV_bat_YNM, AAV_bat_Brazil, AAV_mo.1, AAV_avian_DA-1, or AAV_mouse_NY1, Bba21, Bba26, Bba27, Bba29, Bba30, Bba31, Bba32, Bba33, Bba34, Bba35, Bba36, Bba37, Bba38, Bba41, Bba42, Bba43, Bba44, Bce14, Bce15, Bce16, Bce17, Bce18, Bce20, Bce35, Bce36, Bce39, Bce40, Bce41, Bce42, Bce43, Bce44, Bce45, Bce46, Bey20, Bey22, Bey23, Bma42, Bma43, Bpo1, Bpo2, Bpo3, Bpo4, Bpo6, Bpo8, Bpo13, Bpo18, Bpo20, Bpo23, Bpo24, Bpo27, Bpo28, Bpo29, Bpo33, Bpo35, Bpo36, Bpo37, Brh26, Brh27, Brh28, Brh29, Brh30, Brh31, Brh32, Brh33, Bfm17, Bfm18, Bfm20, Bfm21, Bfm24, Bfm25, Bfm27, Bfm32, Bfm33, Bfm34, Bfm35, AAV-rh10, AAV-rh39, AAV-rh43, AAVanc80L65, or any variants thereof (see, e.g., U.S. Pat. No. 8,318,480 for its disclosure of non-natural mixed serotypes). Exemplary capsids are also provided in International Application Publication No. WO 2018/022608 and WO 2019/222136, which are incorporated herein in its entirety. The capsid proteins can also be variants of natural VP1, VP2 and VP3, including mutated, chimeric or shuffled proteins. The capsid proteins can be those of rh.10 or other subtype within the various clades of AAV; various clades and subtypes are disclosed, for example, in U.S. Pat. No. 7,906,111. In various embodiments, the capsid of the AAV viral particle has a VP1, VP2, or VP3 protein with an amino acid sequence that is at least about 50%, 55%, 60%, 65%, 70%, 75%, 80%, 85%, 90%, 95%, 96%, 97%, 98%, 99% or 100% identical to a portion of an amino acid sequence from AAV-1 (Genbank Accession No. AAD27757.1), AAV-2 (NCBI Reference Sequence No. YP_680426.1), AAV-3 (NCBI Reference Sequence No. NP_043941.1), AAV-3B (Genbank Accession No. AAB95452.1), AAV-4 (NCBI Reference Sequence No. NP_044927.1), AAV-5 (NCBI Reference Sequence No. YP_068409.1), AAV-6 (Genbank Accession No. AAB95450.1), AAV-7 (NCBI Reference Sequence No. YP_077178.1), AAV-8 (NCBI Reference Sequence No. YP_077179.1), AAV-9 (Genbank Accession No. AAS99264.1), AAV-10 (Genbank Accession No. AAT46337.1), AAV-11 (Genbank Accession No. AAT46339.1), AAV-12 (Genbank Accession No. ABI16639.1), AAV-13 (Genbank Accession No. ABZ10812.1), or any amino acid sequence disclosed in WO 2018/022608 and WO 2019/222136. Construction and use of AAV proteins of different serotypes are discussed in Chao et al., Mol. Ther. 2:619-623, 2000; Davidson et al., PNAS 97:3428-3432, 2000; Xiao et al., J. Virol. 72:2224-2232, 1998; Halbert et al., J. Virol. 74:1524-1532, 2000; Halbert et al., J. Virol. 75:6615-6624, 2001; and Auricchio et al., Hum. Molec. Genet. 10:3075-3081, 2001.

In one embodiment, the rAAV particle is pseudotyped with an AAV capsid, wherein the VP1 protein comprises the amino acid sequence of any one of SEQ ID NOs:2-76; or comprises an amino acid sequence that is at least 60%, at least 65%, at least 70%, at least 75%, at least 80%, at least 85%, at least 90%, or at least 95% identical over the full length of any one of SEQ ID NOs: 2-76.

An AAV viral particle may be a “pseudotyped” or “hybrid” AAV viral particle. The terms “hybrid” and “pseudotyped” as they relate to AAV viral particles are used interchangeably herein and are intended to indicate that the Rep proteins, inverted terminal repeat sequences (ITRs) and/or capsid proteins are of different serotypes. A large number of alternative capsid variants have been identified from, for example, humans, baboons, pigs, marmosets, chimpanzees, and rhesus, pigtailed, and/or cynomolgus macaques, for example, as disclosed by U.S. Pat. No. 9,737,618; and Gao, G. et al., Clades of Adeno-associated viruses are widely disseminated in human tissues, J. Virol., 78(12):6381-8 (2004), each of which is incorporated herein by reference in its entirety. Production of pseudotyped AAV viral particles is disclosed in, for example, WO 2018/022608 and WO 2001/83692, each of which is herein incorporated by reference in its entirety. Other types of AAV viral particle variants, for example AAV viral particles with capsid mutations, are also contemplated. See, for example, Marsic et al., Molecular Therapy, 22(11): 1900-1909 (2014), which is herein incorporated by reference its entirety. For example, without limitation, the ITRs and/or the Rep proteins may be of, for example, the capsid proteins are derived from sequences of AAV found in a mammal such as, for example, capsid sequences disclosed and designated herein as Bba21, Bba26, Bba27, Bba29, Bba30, Bba31, Bba32, Bba33, Bba34, Bba35, Bba36, Bba37, Bba38, Bba41, Bba42, Bba43, Bba44, Bce14, Bce15, Bce16, Bce17, Bce18, Bce20, Bce35, Bce36, Bce39, Bce40, Bce41, Bce42, Bce43, Bce44, Bce45, Bce46, Bey20, Bey22, Bey23, Bma42, Bma43, Bpo1, Bpo2, Bpo3, Bpo4, Bpo6, Bpo8, Bpo13, Bpo18, Bpo20, Bpo23, Bpo24, Bpo27, Bpo28, Bpo29, Bpo33, Bpo35, Bpo36, Bpo37, Brh26, Brh27, Brh28, Brh29, Brh30, Brh31, Brh32, Brh33, Bfm17, Bfm18, Bfm20, Bfm21, Bfm24, Bfm25, Bfm27, Bfm32, Bfm33, Bfm34, or Bfm35 or variants thereof.

As used herein, an “AAV vector genome”, “vector genome”, or “rAAV vector genome” refers to single-stranded nucleic acids. An rAAV viral particle has an rAAV vector genome encapsidated within a capsid. The rAAV vector genome has an AAV 5′ inverted terminal repeat (ITR) sequence and an AAV 3′ ITR flanking a protein-coding sequence (preferably a functional therapeutic protein-encoding sequence; e.g., FVIII, FIX, and PAH) operably linked to transcription regulatory elements that are heterologous to the AAV viral genome, e.g., one or more promoters and/or enhancers and, optionally, a polyadenylation sequence and/or one or more introns inserted in the regulatory elements or between the regulatory elements and the protein-coding sequence or between exons of the protein-coding sequence. rAAV vector genome refers to nucleic acids that are present in the rAAV virus particle and can be either the sense strand or the anti-sense strand of the nucleic acid sequences disclosed herein. The size of such single-stranded nucleic acids is provided in bases. The terms “inverted terminal repeat” and “ITR” as used herein refers to the art-recognized regions found at the 5′ and 3′ termini of the rAAV genome which function in cis as origins of viral DNA replication and as packaging signals for the viral genome. AAV ITRs, together with the Rep proteins, provide for efficient excision and rescue from, and integration of a nucleotide sequence interposed between two flanking ITRs into a host cell genome. Sequences of certain AAV-associated ITRs are disclosed by Yan et al., J. Virol. 79(1):364-379 (2005). ITRs are also found in a “flip” or “flop” configuration in which the sequence between the AA′ inverted repeats (that form the arms of the hairpin) are present in the reverse complement (Wilmott, Patrick, et al. Human gene therapy methods 30.6 (2019): 206-213). Construction and use of AAV vector genomes of different serotypes are discussed in Chao et al., Mol. Ther. 2:619-623, 2000; Davidson et al., PNAS 97:3428-3432, 2000; Xiao et al., J. Virol. 72:2224-2232, 1998; Halbert et al., J. Virol. 74:1524-1532, 2000; Halbert et al., J. Virol. 75:6615-6624, 2001; and Auricchio et al., Hum. Molec. Genet. 10:3075-3081, 2001. Because of wide construct availability and extensive characterization, illustrative AAV vector genomes disclosed below are derived from serotype 2.

The terms “therapeutically effective AAV”, “therapeutically effective AAV particle”, “therapeutic AAV”, “therapeutically effective rAAV”, “therapeutically effective rAAV particle”, “therapeutic rAAV”, and “therapeutically effective rAAV” refer to recombinant AAV that are capable of infecting cells such that the infected cells express (e.g., by transcription and/or by translation) an element (e.g., nucleotide sequence, protein, etc.) of interest. To this extent, the therapeutically effective rAAV particles can include AAV particles having capsids or vector genomes (vgs) with different properties. For example, the therapeutically effective rAAV particles can have capsids with different post translation modifications. In other examples, the therapeutically effective AAV particles can contain vgs with differing sizes/lengths, plus or minus strand sequences, different flip/flop ITR configurations flip/flop, flop/flip, flip/flip, flop/flop, etc.), different number of ITRs (1, 2, 3, etc.), or truncations. For example, overlapping homologous recombination occurs in rAAV infected cells between nucleic acids having 5′ end truncations and 3′ end truncations so that a “complete” nucleic acid encoding the large protein is generated, thereby reconstructing a functional, full-length gene. In other examples, complementary nucleic acid sequences having 5′ end truncations and 3′ end truncations interact with each such that a “complete” nucleic acid is formed during second strand synthesis. The “complete” nucleic acid encodes the large protein, thereby reconstructing a functional, full-length gene. Therapeutically effective rAAV particles are also referred to as heavy capsids, full capsids, or partially full capsids.

The terms “transduction” and “transduce” refers to the transfer of genetic material (e.g., vector genome) from an rAAV into a recipient cell and the expression transgene from the rAAV genetic material in the recipient cell. The transfer of the genetic material is mediated through an rAAV particle infecting a recipient cell. To this end, the term “potency” refers to the level of transgene expression in a recipient cell or recipient cells infected by rAAV particles. Thus, an rAAV having a greater potency highlights that a recipient cell infected by rAAV has greater transgene expression.

The term “therapeutically effective amount” means an amount of a therapeutic agent that is sufficient, when administered to a subject suffering from or susceptible to a disease, disorder, or condition, to treat, diagnose, prevent, or delay the onset of the symptom(s) of the disease, disorder, or condition. It will be appreciated by those of ordinary skill in the art that a therapeutically effective amount is typically administered via a dosing regimen comprising at least one unit dose. The term “therapeutically effective” refers to any element or composition of a therapeutic agent acting sufficiently such that a therapeutically effective amount of the therapeutic agent is sufficient, when administered to a subject suffering from or susceptible to a disease, disorder, and/or condition, to treat, diagnose, prevent, and/or delay the onset of the symptom(s) of the disease, disorder, and/or condition. For example as previously noted, a therapeutically effective rAAV is capable of infecting cells such that the infected cells express (e.g., by transcription and/or by translation) an element (e.g., nucleotide sequence, protein, etc.) of interest. The therapeutically effective rAAV has a vector genome that is used by cells infected by the therapeutically effective rAAV to generate therapeutically effective nucleotide sequences that are used by the infected cell to generate an element (e.g., nucleotide sequence, protein, etc.) of interest by various methods such as replication, transcription, or translation. It is also noted that a “therapeutic agent” includes therapeutically effective rAAV or a therapeutic rAAV virus.

As an example, a “therapeutic rAAV virus”, which refers to an rAAV virion, rAAV viral particle, rAAV vector particle, or rAAV virus that comprises a heterologous polynucleotide that encodes a therapeutic protein, can be used to replace or supplement the protein in vivo. The “therapeutic protein” is a polypeptide that has a biological activity that replaces or compensates for the loss or reduction of activity of a corresponding endogenous protein. For example, a functional phenylalanine hydroxylase (PAH) is a therapeutic protein for phenylketonuria (PKU). Thus, for example recombinant rAAV PAH virus can be used for a medicament for the treatment of a subject suffering from PKU. The medicament may be administered by intravenous (IV) administration and the administration of the medicament results in expression of PAH protein in the bloodstream of the subject sufficient to alter the neurotransmitter metabolite or neurotransmitter levels in the subject. Optionally, the medicament may also comprise a prophylactic and/or therapeutic corticosteroid for the prevention and/or treatment of any hepatotoxicity associated with administration of the rAAV PAH virus. The medicament comprising a prophylactic or therapeutic corticosteroid treatment may comprise at least 5, 10, 15, 20, 25, 30, 35, 40, 45, 50, 55, 60, or more mg/day of the corticosteroid. The medicament comprising a prophylactic or therapeutic corticosteroid may be administered over a continuous period of at least about 3, 4, 5, 6, 7, 8, 9, 10 weeks, or more. The PKU therapy may optionally also include tyrosine supplements.

The transgene incorporated into the AAV capsid is not limited and may be any heterologous gene of therapeutic interest. The transgene is a nucleic acid sequence, heterologous to the vector sequences flanking the transgene, which encodes a polypeptide, protein, or other product, of interest. The nucleic acid coding sequence is operatively linked to regulatory components in a manner which permits transgene transcription, translation, and/or expression in a host cell.

The composition of the transgene sequence will depend upon the use to which the resulting vector will be put. For example, one type of transgene sequence includes a reporter sequence, which upon expression produces a detectable signal. Such reporter sequences include, without limitation, DNA sequences encoding b-lactamase, b-galactosidase (LacZ), alkaline phosphatase, thymidine kinase, green fluorescent protein (GFP), chloramphenicol acetyltransferase (CAT), luciferase, membrane bound proteins including, for example, CD2, CD4, CD8, the influenza hemagglutinin protein, and others well known in the art, to which high affinity antibodies directed thereto exist or can be produced by conventional means, and fusion proteins comprising a membrane bound protein appropriately fused to an antigen tag domain from, among others, hemagglutinin or Myc.

These coding sequences, when associated with regulatory elements which drive their expression, provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA) and immunohistochemistry. For example, where the marker sequence is the LacZ gene, the presence of the vector carrying the signal is detected by assays for beta-galactosidase activity. Where the transgene is green fluorescent protein or luciferase, the vector carrying the signal may be measured visually by color or light production in a luminometer.

However, the transgene is typically a non-marker sequence encoding a product which is useful in biology and medicine, such as proteins, peptides, RNA, enzymes, dominant negative mutants, or catalytic RNAs. Desirable RNA molecules include tRNA, dsRNA, ribosomal RNA, catalytic RNAs, siRNA, small hairpin RNA, trans-splicing RNA, and antisense RNAs. One example of a useful RNA sequence is a sequence which inhibits or extinguishes expression of a targeted nucleic acid sequence in the treated animal. Typically, suitable target sequences include oncologic targets and viral diseases. See for examples of such targets the oncologic targets and viruses identified below in the section relating to immunogens.

The transgene may be used to correct or ameliorate gene deficiencies, which may include deficiencies in which normal genes are expressed at less than normal levels or deficiencies in which the functional gene product is not expressed. A preferred type of transgene sequence encodes a therapeutic protein or polypeptide which is expressed in a host cell. The vector may further include multiple transgenes, e.g., to correct or ameliorate a gene defect caused by a multi-subunit protein. In certain situations, a different transgene may be used to encode each subunit of a protein, or to encode different peptides or proteins. This is desirable when the size of the DNA encoding the protein subunit is large, e.g., for an immunoglobulin, the platelet-derived growth factor, or a dystrophin protein. In order for the cell to produce the multi-subunit protein, a cell is infected with the recombinant virus containing each of the different subunits. Alternatively, different subunits of a protein may be encoded by the same transgene. In this case, a single transgene includes the DNA encoding each of the subunits, with the DNA for each subunit separated by an internal ribozyme entry site (IRES). This is desirable when the size of the DNA encoding each of the subunits is small, e.g., the total size of the DNA encoding the subunits and the IRES is less than five kilobases (Kb). It is also noted that longer genomes (i.e., >5 (Kb)) might be feasible due to recombination of partial genomes in target cells. As an alternative to an IRES, the DNA may be separated by sequences encoding a 2A peptide, which self-cleaves in a post-translational event. See, e.g., Donnelly et al, J. Gen. Virol., 78(Pt 1): 13-21 (January 1997); Furler, et al, Gene Ther., 8(11):864-873 (June 2001); Klump et al, Gene Ther., 8(10):811-817 (May 2001). This 2A peptide is significantly smaller than an IRES, making it well suited for use when space is a limiting factor. More often, when the transgene is large, consists of multi-subunits, or two transgenes are co-delivered, rAAV carrying the desired transgene(s) or subunits are co-administered to allow them to concatamerize in vivo to form a single vector genome. In such an embodiment, a first AAV may carry an expression cassette which expresses a single transgene and a second AAV may carry an expression cassette which expresses a different transgene for co-expression in the host cell. However, the selected transgene may encode any biologically active product or other product, e.g., a product desirable for study.

Suitable transgenes may be readily selected by one of skill in the art. The selection of the transgene is not considered to be a limitation of this invention. The transgene may be a heterologous protein, and this heterologous protein may be a therapeutic protein. Exemplary therapeutic proteins include, but are not limited to, blood factors, such as b-globin, hemoglobin, tissue plasminogen activator, and coagulation factors; colony stimulating factors (CSF); interleukins, such as IL-1, IL-2, IL-3, IL-4, IL-5, IL-6, IL-7, IL-8, IL-9, etc.; growth factors, such as keratinocyte growth factor (KGF), stem cell factor (SCF), fibroblast growth factor (FGF, such as basic FGF and acidic FGF), hepatocyte growth factor (HGF), insulin-like growth factors (IGFs), bone morphogenetic protein (BMP), epidermal growth factor (EGF), growth differentiation factor-9 (GDF-9), hepatoma derived growth factor (HDGF), myostatin (GDF-8), nerve growth factor (NGF), neurotrophins, platelet-derived growth factor (PDGF), thrombopoietin (TPO), transforming growth factor alpha (TGF-a.), transforming growth factor beta (TGF-.b.), and the like; soluble receptors, such as soluble TNF-α. receptors, soluble VEGF receptors, soluble interleukin receptors (e.g., soluble IL-1 receptors and soluble type II IL-1 receptors), soluble g/d T cell receptors, ligand-binding fragments of a soluble receptor, and the like; enzymes, such as a-glucosidase, imiglucarase, b-glucocerebrosidase, and alglucerase; enzyme activators, such as tissue plasminogen activator; chemokines, such as 1P-10, monokine induced by interferon-gamma (Mig), Groa/IL-8, RANTES, MIP-1a, MIR-1b., MCP-1, PF-4, and the like; angiogenic agents, such as vascular endothelial growth factors (VEGFs, e.g., VEGF121, VEGF165, VEGF-C, VEGF-2), glioma-derived growth factor, angiogenin, angiogenin-2; and the like; anti-angiogenic agents, such as a soluble VEGF receptor; protein vaccine; neuroactive peptides, such as nerve growth factor (NGF), bradykinin, cholecystokinin, gastin, secretin, oxytocin, gonadotropin-releasing hormone, beta-endorphin, enkephalin, substance P, somatostatin, prolactin, galanin, growth hormone-releasing hormone, bombesin, dynorphin, warfarin, neurotensin, motilin, thyrotropin, neuropeptide Y, luteinizing hormone, calcitonin, insulin, glucagons, vasopressin, angiotensin II, thyrotropin-releasing hormone, vasoactive intestinal peptide, a sleep peptide, and the like; thrombolytic agents; atrial natriuretic peptide; relaxin; glial fibrillary acidic protein; follicle stimulating hormone (FSH); human alpha-1 antitrypsin; leukemia inhibitory factor (LIF); tissue factors, luteinizing hormone; macrophage activating factors; tumor necrosis factor (TNF); neutrophil chemotactic factor (NCF); tissue inhibitors of metalloproteinases; vasoactive intestinal peptide; angiogenin; angiotropin; fibrin; hirudin; IF-1 receptor antagonists; and the like. Some other non-limiting examples of protein of interest include ciliary neurotrophic factor (CNTF); brain-derived neurotrophic factor (BDNF); neurotrophins 3 and 4/5 (NT-3 and 4/5); glial cell derived neurotrophic factor (GDNF); aromatic amino acid decarboxylase (AADC); hemophilia related clotting proteins, such as Factor VIII, Factor IX, Factor X; hereditary angioedema related proteins such as Cl-inhibitor; dystrophin, mini-dystrophin, or microdystrophin; lysosomal acid lipase; phenylalanine hydroxylase (PAH); glycogen storage disease-related enzymes, such as glucose-6-phosphatase, acid maltase, glycogen debranching enzyme, muscle glycogen phosphorylase, liver glycogen phosphorylase, muscle phosphofructokinase, phosphorylase kinase (e.g., PHKA2), glucose transporter (e.g., GFUT2), aldolase A, b-enolase, and glycogen synthase; lysosomal enzymes (e.g., beta-N-acetylhexosaminidase A); and any variants thereof. Other transgenes include transgenes encoding cardiac myosin binding protein C, β-myosin heavy chain, cardiac troponin T, cardiac troponin I, myosin ventricular essential light chain 1, myosin ventricular regulatory light chain 2, cardiac α actin (ACTC), α-tropomyosin, titin, four-and-a-half LIM protein 1, and other transgenes disclosed in U.S. Pat. No. in International Application Publication No. WO 2014/170470. The AAV vector also includes conventional control elements or sequences which are operably linked to the transgene in a manner which permits its transcription, translation and/or expression in a cell transfected with the plasmid vector or infected with the virus. As used herein, “operably linked” sequences include both expression control sequences that are contiguous with the gene of interest (GOI) and expression control sequences that act in trans or at a distance to control the gene of interest. Suitable genes include those genes discussed in Anguela et al. “Entering the Modern Era of Gene Therapy”, Annual Rev. of Med. Vol. 70, pages 272-288 (2019) and Dunbar et al., “Gene Comes of Age”, Science, Vol. 359, Issue 6372, eaan4672 (2018).

Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (e.g., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) (see, e.g., Boshart el al, Cell, 41:521-530 (1985)), the SV40 promoter, the dihydrofolate reductase promoter, the b-actin promoter, the phosphoglycerol kinase (PGK) promoter, and the EF1 promoter [Invitrogen]. Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied compounds, include, the zinc-inducible sheep metallothionine (MT) promoter, the dexamethasone (Dex)-inducible mouse mammary tumor virus (MMTV) promoter, the T7 polymerase promoter system [WO 98/10088]; the ecdysone insect promoter [No et al, Proc. Natl. Acad. Sci. USA, 93:3346-3351 (1996)], the tetracyclinerepressible system [Gossen et al., Proc. Natl. Acad. Sci. USA, 89:5547-5551 (1992)], the tetracycline-inducible system [Gossen et al., Science, 268:1766-1769 (1995), see also Harvey et al., Curr. Opin. Chem. Biol., 2:512-518 (1998)], the RU486-inducible system [Wang et al., Nat. Biotech., 15:239-243 (1997) and Wang et al., Gene Ther., 4:432-441 (1997)] and the rapamycininducible system [Magari et al., J. Clin. Invest., 100:2865-2872 (1997)]. Other types of inducible promoters which may be useful in this context are those which are regulated by a specific physiological state, e.g., temperature, acute phase, a particular differentiation state of the cell, or in replicating cells only.

Optionally, the native promoter for the transgene may be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

The transgene may also include a gene operably linked to a tissue specific promoter. For instance, if expression in skeletal muscle is desired, a promoter active in muscle should be used. These include the promoters from genes encoding skeletal b-actin, myosin light chain 2A, dystrophin, muscle creatine kinase, as well as synthetic muscle promoters with activities higher than naturally-occurring promoters (see Li et al., Nat. Biotech., 17:241-245 (1999)). Examples of promoters that are tissue-specific are known for liver (albumin, Miyatake et al., J. Virol, 71:5124-32 (1997); hepatitis B virus core promoter, Sandig et al., Gene Ther., 3:1002-9 (1996); alpha-fetoprotein (AFP), Arbuthnot et al., Hum. Gene Ther., 7:1503-14 (1996)), bone osteocalcin (Stein et al., Mol. Biol. Rep., 24:185-96 (1997)); bone sialoprotein (Chen et al., J. Bone Miner. Res., 11:654-64 (1996)), lymphocytes (CD2, Hansal et al., J. Immunol., 161:1063-8 (1998); immunoglobulin heavy chain; T cell receptor chain), neuronal such as neuron-specific enolase (NSE) promoter (Andersen et al., Cell. Mol. Neurobiol., 13:503-15 (1993)), neurofilament light-chain gene (Piccioli et al., Proc. Natl. Acad. Sci. USA, 88:5611-5 (1991)), and the neuron-specific vgf gene (Piccioli et al., Neuron, 15:373-84 (1995)), among others.

The recombinant AAV can be used to produce a protein of interest in vitro, for example, in a cell culture. For example, the AAV can be used in a method for producing a protein of interest in vitro, where the method includes providing a recombinant AAV comprising a nucleotide sequence encoding the heterologous protein; and contacting the recombinant AAV with a cell in a cell culture, whereby the recombinant AAV expresses the protein of interest in the cell. The size of the nucleotide sequence encoding the protein of interest can vary. For example, the nucleotide sequence can be at least about 0.1 kilobases (kb), at least about 0.2 kb, at least about 0.3 kb, at least about 0.4 kb, at least about 0.5 kb, at least about 0.6 kb, at least about 0.7 kb, at least about 0.8 kb, at least about 0.9 kb, at least about 1 kb, at least about 1.1 kb, at least about 1.2 kb, at least about 1.3 kb, at least about 1.4 kb, at least about 1.5 kb, at least about 1.6 kb, at least about 1.7 kb, at least about 1.8 kb, at least about 2.0 kb, at least about 2.2 kb, at least about 2.4 kb, at least about 2.6 kb, at least about 2.8 kb, at least about 3.0 kb, at least about 3.2 kb, at least about 3.4 kb, at least about 3.5 kb in length, at least about 4.0 kb in length, at least about 5.0 kb in length, at least about 6.0 kb in length, at least about 7.0 kb in length, at least about 8.0 kb in length, at least about 9.0 kb in length, or at least about 10.0 kb in length. In some embodiments, the nucleotide is at least about 1.4 kb in length.

The recombinant AAV can also be used to produce a protein of interest in vivo, for example in an animal such as a mammal. Some embodiments provide a method for producing a protein of interest in vivo, where the method includes providing a recombinant AAV comprising a nucleotide sequence encoding the protein of interest; and administering the recombinant AAV to the subject, whereby the recombinant AAV expresses the protein of interest in the subject. The subject can be, in some embodiments, a non-human mammal, for example, a monkey, a dog, a cat, a mouse, or a cow. The size of the nucleotide sequence encoding the protein of interest can vary. For example, the nucleotide sequence can be at least about 0.1 kb, at least about 0.2 kb, at least about 0.3 kb, at least about 0.4 kb, at least about 0.5 kb, at least about 0.6 kb, at least about 0.7 kb, at least about 0.8 kb, at least about 0.9 kb, at least about 1 kb, at least about 1.1 kb, at least about 1.2 kb, at least about 1.3 kb, at least about 1.4 kb, at least about 1.5 kb, at least about 1.6 kb, at least about 1.7 kb, at least about 1.8 kb, at least about 2.0 kb, at least about 2.2 kb, at least about 2.4 kb, at least about 2.6 kb, at least about 2.8 kb, at least about 3.0 kb, at least about 3.2 kb, at least about 3.4 kb, at least about 3.5 kb in length, at least about 4.0 kb in length, at least about 5.0 kb in length, at least about 6.0 kb in length, at least about 7.0 kb in length, at least about 8.0 kb in length, at least about 9.0 kb in length, or at least about 10.0 kb in length. In some embodiments, the nucleotide is at least about 1.4 kb in length.

Of particular interest is the use of recombinant AAV to express one or more therapeutic proteins to treat various diseases or disorders. Non-limiting examples of the diseases include cancer such as carcinoma, sarcoma, leukemia, lymphoma; and autoimmune diseases such as multiple sclerosis. Non-limiting examples of carcinomas include esophageal carcinoma; hepatocellular carcinoma; basal cell carcinoma, squamous cell carcinoma (various tissues); bladder carcinoma, including transitional cell carcinoma; bronchogenic carcinoma; colon carcinoma; colorectal carcinoma; gastric carcinoma; lung carcinoma, including small cell carcinoma and non-small cell carcinoma of the lung; adrenocortical carcinoma; thyroid carcinoma; pancreatic carcinoma; breast carcinoma; ovarian carcinoma; prostate carcinoma; adenocarcinoma; sweat gland carcinoma; sebaceous gland carcinoma; papillary carcinoma; papillary adenocarcinoma; cystadenocarcinoma; medullary carcinoma; renal cell carcinoma; ductal carcinoma in situ or bile duct carcinoma; choriocarcinoma; seminoma; embryonal carcinoma; Wilm's tumor; cervical carcinoma; uterine carcinoma; testicular carcinoma; osteogenic carcinoma; epithelieal carcinoma; and nasopharyngeal carcinoma. Non-limiting examples of sarcomas include fibrosarcoma, myxosarcoma, liposarcoma, chondrosarcoma, chordoma, osteogenic sarcoma, osteosarcoma, angiosarcoma, endothelio sarcoma, lymphangiosarcoma, lymphangioendotheliosarcoma, synovioma, mesothelioma, Ewing's sarcoma, leiomyosarcoma, rhabdomyosarcoma, and other soft tissue sarcomas. Non-limiting examples of solid tumors include glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma. Non-limiting examples of leukemias include chronic myeloproliferative syndromes; acute myelogenous leukemias; chronic lymphocytic leukemias, including B-cell CLL, T-cell CLL prolymphocytic leukemia, and hairy cell leukemia; and acute lymphoblastic leukemias. Examples of lymphomas include, but are not limited to, B-cell lymphomas, such as Burkitt's lymphoma; Hodgkin's lymphoma; and the like.

Other non-liming examples of the diseases that can be treated using rAAV and methods disclosed herein include genetic disorders including sickle cell anemia, cystic fibrosis, lysosomal acid lipase (LAL) deficiency 1, Tay-Sachs disease, Phenylketonuria, Mucopolysaccharidoses, Glycogen storage diseases (GSD, e.g., GSD types I, II, III, IV, V, VI, VII, VIII, IX, X, XI, XII, XIII, and XIV), Galactosemia, muscular dystrophy (e.g., Duchenne muscular dystrophy), cardiomyopathies (e.g., hypertrophic cardiomyopathy, dilated cardiomyopathy, arrhythmogenic right ventricular cardiomyopathy, etc.) and hemophilia such as hemophilia A (classic hemophilia) and hemophilia B (Christmas Disease), Wilson's disease, Fabry Disease, Gaucher Disease hereditary angioedema (HAE), and alpha 1 antitrypsin deficiency. In addition, the rAAV and methods disclosed herein can be used to treat other disorders that can be treated by local expression of a transgene in the liver or by expression of a secreted protein from the liver or a hepatocyte.

The amount of the heterologous protein expressed in the subject (e.g., the serum of the subject) can vary. For example, in some embodiments the protein can be expressed in the serum of the subject in the amount of at least about 9 milligram (mg)/mL, at least about 10 mg/mL, at least about 11 mg/mL, at least about 12 mg/mL, at least about 13 mg/mL, at least about 14 mg/mL, at least about 15 mg/mL, at least about 16 mg/mL, at least about 17 mg/mL, at least about 18 mg/mL, at least about 19 mg/mL, at least about 20 mg/mL, at least about 21 mg/mL, at least about 22 mg/mL, at least about 23 mg/mL, at least about 24 mg/mL, at least about 25 mg/mL, at least about 26 mg/mL, at least about 27 mg/mL, at least about 28 mg/mL, at least about 29 mg/mL, at least about 30 mg/mL, at least about 31 mg/mL, at least about 32 mg/mL, at least about 33 mg/mL, at least about 34 mg/mL, at least about 35 mg/mL, at least about 36 mg/mL, at least about 37 mg/mL, at least about 38 mg/mL, at least about 39 mg/mL, at least about 40 mg/mL, at least about 41 mg/mL, at least about 42 mg/mL, at least about 43 mg/mL, at least about 44 mg/mL, at least about 45 mg/mL, at least about 46 mg/mL, at least about 47 mg/mL, at least about 48 mg/mL, at least about 49 mg/mL, or at least about 50 mg/mL. The protein of interest may be expressed in the serum of the subject in the amount of about 9 pg/mL, about 10 pg/mL, about 50 pg/mL, about 100 pg/mL, about 200 pg/mL, about 300 pg/mL, about 400 pg/mL, about 500 pg/mL, about 600 pg/mL, about 700 pg/mL, about 800 pg/mL, about 900 pg/mL, about 1000 pg/mL, about 1500 pg/mL, about 2000 pg/mL, about 2500 pg/mL, or a range between any two of these values. A skilled artisan will understand that the expression level in which a protein of interest is needed for therapeutic efficacy can vary depending on non-limiting factors, such as the particular protein of interest and the subject receiving the treatment, and an effective amount of the protein can be readily determined by a skilled artisan using conventional methods known in the art without undue experimentation.

Methods of Producing Adeno Associated Virus

In one aspect, a method for preparing rAAV is disclosed and includes the step of culturing, in a culture medium with an effective amount of a transition metal. The host cell is capable of producing an rAAV capsid. The effective amount of the transition metal increases incorporation of VP1, VP2, or VP3 protein into the rAAV capsid. Alternatively, the effective amount of the transition metal decreases incorporation of VP2 or VP3 protein into the rAAV capsid.

In one aspect, a method for preparing rAAV is disclosed and includes the step of culturing a host cell in a culture medium having a nanomolar concentration of a transition metal. The host cell is capable of producing an rAAV capsid.

In one aspect, a method for preparing rAAV is disclosed and includes the step of culturing a host cell in a culture medium having a micromolar concentration of a transition metal. The host cell is capable of producing an rAAV capsid.

In one aspect, a method for preparing rAAV is disclosed and includes the step of culturing, in a culture medium with an effective amount of a transition metal. The host cell is capable of producing an rAAV capsid. The effective amount of the transition metal increases incorporation of VP1 and VP3 proteins into the rAAV capsid. The rAAV capsid has concentrations of VP1 and VP3 proteins that are greater than concentrations of VP1 and VP3 proteins of an rAAV capsid produced under the same conditions but being devoid of the effective amount of the transition metal.

In one aspect, a method for preparing rAAV is disclosed and includes the step of culturing, in a culture medium with an effective amount of a transition metal. The host cell is capable of producing an rAAV capsid. The effective amount of the transition metal increases incorporation of VP1 proteins into the rAAV capsid.

In one aspect, a method for preparing rAAV is disclosed and includes the step of culturing, in a culture medium with an effective amount of a cysteine protease inhibitor. The host cell is capable of producing an rAAV capsid. The effective amount of a cysteine protease inhibitor increases incorporation of VP1, VP2, or VP3 protein into the rAAV capsid. Alternatively, the effective amount of a cysteine protease inhibitor decreases incorporation of VP2 or VP3 protein into the rAAV capsid.

In one aspect, the present invention provides a method for preparing a rAAV particle, the method comprising the step of culturing, in a culture medium comprising an effective amount of a salt, a host cell capable of producing the AAV viral particle, wherein the AAV viral particle comprises an AAV capsid comprising a VP1 protein. The effective amount of a salt increases incorporation of VP1, VP2, or VP3 protein into the rAAV capsid. Alternatively, the effective amount of a salt decreases incorporation of VP2 or VP3 protein into the rAAV capsid.

In various embodiments, the method of any aspect or embodiment includes the step culturing a host cell having one or more vectors for rAAV production. In other embodiments, the one or more vectors for rAAV production includes at least one nucleic acid molecule that provides AAV helper function, or at least one nucleic acid molecule that provides non-AAV helper function, or at least one nucleic acid molecule that generates an AAV genome vector, or any combination thereof. The method of any aspect or embodiment further includes culturing the cells under conditions that that permit production of the rAAV. The method optionally includes recovering the rAAV. For example, the AAV viral particles can be collected at about 12 hours, about 24 hours, about 36 hours, about 48 hours, about 72 hours, about 96 hours, about 120 hours, or a time between any of these two time points after the co-transfection.

In some embodiments, cultures for the production of AAV viral particle comprise one or more of the following: the host cell, a suitable helper virus function, provided by wild type or mutant adenovirus (such as temperature sensitive adenovirus), herpes virus, baculovirus, or a plasmid construct providing helper functions, an AAV rep and cap genes and gene products, a transgene (such as diagnostic and/or therapeutic transgene(s)) flanked by AAV ITR sequences, and suitable media and media components to support AAV viral particle production.

In some embodiments, the insect or mammalian cell can be transfected with the helper plasmid or helper virus, the viral construct and the plasmid encoding the AAV cap genes; and the rAAV particles can be collected at various time points after co-transfection.

In some embodiments, a novel rAAV viral particle is produced in insect cells (e.g., Sf9). In some embodiments, an AAV viral particle is prepared by providing to a host cell with an AAV genome vector comprising a transgene together with a Rep and Cap gene. In some embodiments, an AAV genome vector comprises a transgene, an AAV Rep gene and an AAV Cap gene. In some embodiments, an rAAV viral particle is prepared by providing to a host cell with two or more vectors. For example, in some embodiments, an AAV genome vector comprising a transgene is introduced (e.g., transfected or transduced) into a cell with a vector (e.g., a plasmid or baculovirus) comprising an AAV Rep gene and a AAV Cap gene. In some embodiments, a cell transfected or transduced with an AAV genome vector comprising a transgene, a vector (e.g., a plasmid or baculovirus) comprising an AAV Rep gene, and a vector (e.g., a plasmid or baculovirus) comprising an AAV Cap gene.

In various embodiments, the method of any aspect or embodiment includes the steps of infecting the host cells with rBV. The rBV includes one or more nucleic acid molecules encoding Rep proteins, one or more nucleic acid molecules encoding capsid proteins, and at least one nucleic acid molecule that generates an AAV genome vector. The method of any aspect or embodiment further includes culturing the cells under conditions that that permit production of the rAAV. The method optionally includes recovering the rAAV.

There are a number of methods for generating AAV viral particles: for example, but not limited to, transfection using vector and AAV helper sequences in conjunction with coinfection with one of the AAV helper viruses (e.g., adenovirus, herpesvirus, or vaccinia virus) or transfection with a recombinant AAV vector, an AAV helper vector, and an accessory function vector. Methods of making AAV viral particles are described in e.g., U.S. Pat. Nos. 6,204,059, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508, 5,064,764, 6,194,191, 6,566,118, 8,137,948; or International Publication Nos. WO1996039530, WO1998010088, WO1999014354, WO1999015685, WO1999047691, WO2000055342, WO2000075353, WO2001023597, WO2015191508, WO2019217513, WO2018022608, WO2019222136, WO2020232044, WO2019222132; Methods In Molecular Biology, ed. Richard, Humana Press, N J (1995); O'Reilly et al., Baculovirus Expression Vectors, A Laboratory Manual, Oxford Univ. Press (1994); Samulski et al., J. Vir. 63:3822-8 (1989); Kajigaya et al., Proc. Nat'l. Acad. Sci. USA 88: 4646-50 (1991); Ruffing et al., J. Vir. 66:6922-30 (1992); Kimbauer et al., Vir., 219:37-44 (1996); Zhao et al., Vir. 272:382-93 (2000); the contents of each of which are herein incorporated by reference in their entirety. For detailed descriptions of methods for generating AAV viral particles see, for example, U.S. Pat. Nos. 6,001,650, 6,004,797, and 9,504,762, each herein incorporated by reference in its entirety. In one embodiment, a triple transfection method (see, e.g., U.S. Pat. No. 6,001,650, herein incorporated by reference in its entirety) is used to produce AAV viral particles. This method does not require the use of an infectious helper virus, enabling AAV viral particles to be produced without any detectable helper virus present. This is accomplished by use of three vectors for AAV viral particle production, namely an AAV helper function vector, an accessory function vector, and an AAV viral particle expression vector. One of skill in the art will appreciate, however, that the nucleic acid sequences encoded by these vectors can be provided on two or more vectors in various combinations. In other embodiments, the host cell can be transfected with the helper plasmid or helper virus, the viral construct and the plasmid encoding the AAV cap genes; and the AAV viral particles can be collected at various time points after co-transfection.

For example, wild-type AAV and helper viruses may be used to provide the necessary replicative functions for producing AAV viral particles (see, e.g., U.S. Pat. No. 5,139,941, herein incorporated by reference in its entirety). Alternatively, a plasmid, containing helper function genes, in combination with infection by one of the well-known helper viruses can be used as the source of replicative functions (see e.g., U.S. Pat. Nos. 5,622,856 and 5,139,941, both herein incorporated by reference in their entireties). Similarly, a plasmid, containing accessory function genes can be used in combination with infection by wild-type AAV, to provide the necessary replicative functions. Other approaches, described herein and/or well known in the art, can also be employed by the skilled artisan to produce AAV viral particles.

In various embodiments, the culturing step of any aspect or embodiment occurs in a volume of at least 20 milliliter(s) (mL), at least 50 mL, at least 100 mL, at least 500 mL, at least 1 liter (L), at least 10 L, at least 50 L, at least 100 L, at least 250 L, at least 500 L, at least 1000 L, at least 1500 L, at least 2000 L, or at least 2500 L.

In examples, the culturing step can occur in a shake flask or shake flasks. In various embodiments, the culturing step of any aspect or embodiment occurs in a volume of 20 mL, 30 mL, 40 mL, 50 mL, 60 mL, 70 mL, 80 mL, 90 mL, 100 mL, 200 mL, 300 mL, 400 mL, 500 mL, 600 mL, 700 mL, 800 mL, 900 mL, 1 L, 2 L, 3 L, 4 L, or 5 L. In other embodiments, the volume of the culturing step is a range between any two volumes provided above.

In other examples, the culturing step can occur in a bioreactor or bioreactors. In various embodiments, the culturing step of any aspect or embodiment occurs in a volume of 1 L, 2 L, 3 L, 4 L, 5 L, 6L, 7L, 8 L, 9 L, 10 L, 11 L, 12 L, 13 L, 14 L, 15 L 16 L, 17 L, 18 L, 19 L, 20 L, 21 L, 22 L, 23 L, 24 L, 25 L, 26 L, 27 L, 28 L, 29 L, 30 L, 31 L, 32 L, 33 L, 34 L, 35 L, 36 L, 37 L, 38 L, 39 L, 40 L, 41 L, 42 L, 43 L, 44 L, 45 L, 46 L, 47 L, 48 L, 49 L, 50 L, 51 L, 52 L, 53 L, 54 L, 55 L, 56 L, 57 L, 58 L, 59 L, 60 L, 61 L, 62 L, 63 L, 64 L, 65 L, 66 L, 67 L, 68 L, 69 L, 70 L, 71 L, 72 L, 73 L, 74 L, 75 L, 76 L, 77 L, 78 L, 79 L, 80 L, 81 L, 82 L, 83 L, 84 L, 85 L, 86 L, 87 L, 88 L, 89 L, 90 L, 91 L, 92 L, 93 L, 94 L, 95 L, 96 L, 97 L, 98 L, 99 L, 100 L, 110 L, 120 L, 130 L, 140 L, 150 L, 160 L, 170 L, 180 L, 190 L, 200 L, 210 L, 220 L, 230 L, 240 L, 250 L, 260 L, 270 L, 280 L, 290 L, 300 L, 310 L, 320 L, 330 L, 340 L, 350 L, 360 L, 370 L, 380 L, 390 L, 400 L, 410 L, 420 L, 430 L, 440 L, 450 L, 460 L, 470 L, 480 L, 490 L, 500 L, 510 L, 520 L, 530 L, 540 L, 550 L, 560 L, 570 L, 580 L, 590 L, 600 L, 610 L, 620 L, 630 L, 640 L, 650 L, 660 L, 670 L, 680 L, 690 L, 700 L, 710 L, 720 L, 730 L, 740 L, 750 L, 760 L, 770 L, 780 L, 790 L, 800 L, 810 L, 820 L, 830 L, 840 L, 850 L, 860 L, 870 L, 880 L, 890 L, 900 L, 910 L, 920 L, 930 L, 940 L, 950 L, 960 L, 970 L, 980 L, 990 L, 1000 L, 1010 L, 1020 L, 1030 L, 1040 L, 1050 L, 1060 L, 1070 L, 1080 L, 1090 L, 1100 L, 1110 L, 1120 L, 1130 L, 1140 L, 1150 L, 1160 L, 1170 L, 1180 L, 1190 L, 1200 L, 1210 L, 1220 L, 1230 L, 1240 L, 1250 L, 1260 L, 1270 L, 1280 L, 1290 L, 1300 L, 1310 L, 1320 L, 1330 L, 1340 L, 1350 L, 1360 L, 1370 L, 1380 L, 1390 L, 1400 L, 1410 L, 1420 L, 1430 L, 1440 L, 1450 L, 1460 L, 1470 L, 1480 L, 1490 L, 1500 L, 1510 L, 1520 L, 1530 L, 1540 L, 1550 L, 1560 L, 1570 L, 1580 L, 1590 L, 1600 L, 1610 L, 1620 L, 1630 L, 1640 L, 1650 L, 1660 L, 1670 L, 1680 L, 1690 L, 1700 L, 1710 L, 1720 L, 1730 L, 1740 L, 1750 L, 1760 L, 1770 L, 1780 L, 1790 L, 1800 L, 1810 L, 1820 L, 1830 L, 1840 L, 1850 L, 1860 L, 1870 L, 1880 L, 1890 L, 1900 L, 1910 L, 1920 L, 1930 L, 1940 L, 1950 L, 1960 L, 1970 L, 1980 L, 1990 L, 2000 L, 2010 L, 2020 L, 2030 L, 2040 L, 2050 L, 2060 L, 2070 L, 2080 L, 2090 L, 2100 L, 2110 L, 2120 L, 2130 L, 2140 L, 2150 L, 2160 L, 2170 L, 2180 L, 2190 L, 2200 L, 2210 L, 2220 L, 2230 L, 2240 L, 2250 L, 2260 L, 2270 L, 2280 L, 2290 L, 2300 L, 2310 L, 2320 L, 2330 L, 2340 L, 2350 L, 2360 L, 2370 L, 2380 L, 2390 L, 2400 L, 2410 L, 2420 L, 2430 L, 2440 L, 2450 L, 2460 L, 2470 L, 2480 L, 2490 L, 2500 L, 2510 L, 2520 L, 2530 L, 2540 L, 2550 L, 2560 L, 2570 L, 2580 L, 2590 L, 2600 L, 2610 L, 2620 L, 2630 L, 2640 L, 2650 L, 2660 L, 2670 L, 2680 L, 2690 L, 2700 L, 2710 L, 2720 L, 2730 L, 2740 L, 2750 L, 2760 L, 2770 L, 2780 L, 2790 L, 2800 L, 2810 L, 2820 L, 2830 L, 2840 L, 2850 L, 2860 L, 2870 L, 2880 L, 2890 L, 2900 L, 2910 L, 2920 L, 2930 L, 2940 L, 2950 L, 2960 L, 2970 L, 2980 L, 2990 L, or 3000 L. In other embodiments, the volume of the culturing step is a range between any two volumes provided above.

The term “vector” is understood to refer to any genetic element, such as a plasmid, phage, transposon, cosmid, bacmid, mini-plasmid (e.g., plasmid devoid of bacterial elements), Doggybone DNA (e.g., minimal, closed-linear constructs), chromosome, virus, virion (e.g., baculovirus), etc., which is capable of replication when associated with the proper control elements and which can transfer gene sequences between cells. An “insect cell-compatible vector” or “vector” as used herein refers to a nucleic acid molecule capable of productive transformation or transfection of an insect or insect cell. Exemplary biological vectors include plasmids, linear nucleic acid molecules, and recombinant viruses. Any vector can be employed as long as it is insect cell-compatible. The vector may integrate into the insect cells genome but the presence of the vector in the insect cell need not be permanent and transient episomal vectors are also included. The vectors can be introduced by any means known, for example by chemical treatment of the cells, electroporation, or infection. Baculoviral vectors and methods for their use are described in the above cited references on molecular engineering of insect cells.

The vector from which the cell generates an rAAV vector genome may contain a promoter and a restriction site downstream of the promoter to allow insertion of a polynucleotide encoding one or more proteins of interest, wherein the promoter and the restriction site are located downstream of the 5′ AAV ITR and upstream of the 3′ AAV ITR. The vector may also contain a posttranscriptional regulatory element downstream of the restriction site and upstream of the 3′ AAV ITR. The viral construct may further comprise a polynucleotide inserted at the restriction site and operably linked with the promoter, where the polynucleotide comprises the coding region of a protein of interest. In some embodiments, the viral construct further includes a promoter and a restriction site downstream of the promoter to allow insertion of a polynucleotide encoding one or more proteins of interest, wherein the promoter and the restriction site are located downstream of the 5′ AAV ITR and upstream of the 3′ AAV ITR. In some embodiments, the viral construct further incudes a posttranscriptional regulatory element downstream of the restriction site and upstream of the 3′ AAV ITR. In some embodiments, the viral construct further includes a polynucleotide inserted at the restriction site and operably linked with the promoter, where the polynucleotide includes the coding region of a protein of interest. As a skilled artisan will appreciate, any one of the AAV vectors disclosed in the present application can be used in the method as the viral construct to produce the rAAV virions.

The term “AAV helper” refer to AAV-derived coding sequences which can be expressed to provide AAV gene products that, in turn, function in trans for productive AAV replication. Thus, AAV helper functions include both of the major AAV open reading frames (ORFs), rep and cap. The Rep expression products have been shown to possess many functions, including, among others: recognition, binding and nicking of the AAV origin of DNA replication; DNA helicase activity; and modulation of transcription from AAV (or other heterologous) promoters. The capsid (Cap) expression products supply necessary packaging functions. AAV helper functions are used herein to complement AAV functions in trans that are missing from AAV vector genomes.

For production, cells with AAV helper functions produce recombinant capsid proteins sufficient to form a capsid. This includes at least VP1 and VP3 proteins, but more typically, all three of VP1, VP2, and VP3 proteins, as found in native AAV. The sequence of the capsid proteins determines the serotype of the AAV virions produced by the host cell. Capsids useful in the invention include those derived from a number of AAV serotypes, including 1, 2, 3, 3B, 4, 5, 6, 7, 8, 9, 10, 11, 12, 13 or mixed serotypes (see, e.g., U.S. Pat. No. 8,318,480 for its disclosure of non-natural mixed serotypes). The capsid proteins can also be variants of natural VP1, VP2 and VP3, including mutated, chimeric or shuffled proteins. The capsid proteins can be those of rh.10 or other subtype within the various clades of AAV; various clades and subtypes are disclosed, for example, in U.S. Pat. No. 7,906,111. Because of wide construct availability and extensive characterization, illustrative AAV vectors disclosed below are derived from serotype 2. Construction and use of AAV vectors and AAV proteins of different serotypes are discussed in Chao et al., Mol. Ther. 2:619-623, 2000; Davidson et al., PNAS 97:3428-3432, 2000; Xiao et al., J. Virol. 72:2224-2232, 1998; Halbert et al., J. Virol. 74:1524-1532, 2000; Halbert et al., J. Virol. 75:6615-6624, 2001; and Auricchio et al., Hum. Molec. Genet. 10:3075-3081, 2001.

In various embodiments, nucleotide sequences encoding VP proteins can be operably linked to a suitable expression control sequence. In various embodiments, nucleotide sequences encoding Rep proteins can be operably linked to a suitable expression control sequence such as eukaryotic promoters. For example, the nucleotide sequences can be operably linked to eukaryotic promoters. In another example, the nucleotide sequences can be operably linked to baculoviral promoters such as the polyhedrin (Polh) promoter, ΔIE1 promoter, p5 promoter, p10 promoter, the p40 promoter, metallothionein promoter, 39K promoter, p6.9 promoter, and orf46 promoter.

For production, cells with AAV helper functions produce Rep proteins to promote production of rAAV. It has been found that infectious particles can be produced when at least one large Rep protein (Rep78 or Rep68) and at least one small Rep protein (Rep52 and Rep40) are expressed in cells. In a specific embodiment all four of Rep 78, Rep68, Rep52 and Rep 40 are expressed. Alternately, Rep78 and Rep52, Rep78 and Rep40, Rep 68 and Rep52, or Rep68 and Rep40 are expressed. Examples below demonstrate the use of the Rep78/Rep52 combination. Rep proteins can be derived from AAV-2 or other serotypes. In various embodiments, nucleotide sequences encoding Rep proteins can be operably linked to a suitable expression control sequence. In various embodiments, nucleotide sequences encoding Rep proteins can be operably linked to a suitable expression control sequence such as eukaryotic promoters. For example, the nucleotide sequences can be operably linked to eukaryotic promoters. In other examples, the nucleotide sequences can be operably linked to baculoviral promoters such as the polyhedrin (Polh) promoter, ΔIE1 promoter, p5 promoter, p10 promoter, the p40 promoter, metallothionein promoter, 39K promoter, p6.9 promoter, and orf46 promoter.

In some embodiments, the AAV cap genes are present in a plasmid or bacmid. The plasmid can further include an AAV rep gene which may or may not correspond to the same serotype as the cap genes. The cap genes and/or rep gene from any AAV serotype.

Cells with AAV helper functions can also produce assembly-activating proteins (AAP), which help assemble capsids. In various embodiments, nucleotide sequences encoding AAP can be operably linked to a suitable expression control sequence. For example, the nucleotide sequences can be operably linked to eukaryotic promoters. In other examples, the nucleotide sequences can be operably linked to baculoviral promoters such as the polyhedrin (Polh) promoter, ΔIE1 promoter, p5 promoter, p10 promoter, the p40 promoter, metallothionein promoter, 39K promoter, p6.9 promoter, and orf46 promoter.

The term “non-AAV helper function” refers to non-AAV derived viral and/or cellular functions upon which AAV is dependent for its replication. Thus, the term captures proteins and RNAs that are required in AAV replication, including those moieties involved in activation of AAV gene transcription, stage specific AAV mRNA splicing, AAV DNA replication, synthesis of Cap expression products and AAV capsid assembly. Viral-based accessory functions can be derived from any of the known helper viruses such as adenovirus, herpesvirus (other than herpes simplex virus type-1) and vaccinia virus.

The term “non-AAV helper function vector” refers generally to a nucleic acid molecule that includes nucleotide sequences providing accessory functions. An accessory function vector can be transfected into a suitable host cell, wherein the vector is then capable of supporting AAV virion production in the host cell. Expressly excluded from the term are infectious viral particles as they exist in nature, such as adenovirus, herpesvirus or vaccinia virus particles. Thus, accessory function vectors can be in the form of a plasmid, phage, transposon or cosmid. In particular, it has been demonstrated that the full-complement of adenovirus genes are not required for accessory helper functions. For example, adenovirus mutants incapable of DNA replication and late gene synthesis have been shown to be permissive for AAV replication. Ito et al., (1970) J. Gen. Virol. 9:243; Ishibashi et al, (1971) Virology 45:317. Similarly, mutants within the E2B and E3 regions have been shown to support AAV replication, indicating that the E2B and E3 regions are probably not involved in providing accessory functions. Carter et al., (1983) Virology 126:505. However, adenoviruses defective in the E1 region, or having a deleted E4 region, are unable to support AAV replication. Thus, E1A and E4 regions are likely required for AAV replication, either directly or indirectly. Laughlin et al., (1982). J. Virol. 41:868; Janik et al., (1981) Proc. Natl. Acad. Sci. USA 78:1925; Carter et al., (1983) Virology 126:505. Other characterized Ad mutants include: E1B (Laughlin et al. (1982), supra; Janik et al. (1981), supra; Ostrove et al., (1980) Virology 104:502); E2A (Handa et al., (1975) J. Gen. Virol. 29:239; Strauss et al., (1976) J. Virol. 17:140; Myers et al., (1980) J. Virol. 35:665; Jay et al., (1981) Proc. Natl. Acad. Sci. USA 78:2927; Myers et al., (1981) J. Biol. Chem. 256:567); E2B (Carter, Adeno-Associated Virus Helper Functions, in I CRC Handbook of Parvoviruses (P. Tijssen ed., 1990)); E3 (Carter et al. (1983), supra); and E4 (Carter et al. (1983), supra; Carter (1995)). Although studies of the accessory functions provided by adenoviruses having mutations in the E1B coding region have produced conflicting results, Samulski et al., (1988) J. Virol, 62:206-210, recently reported that E1B55k is required for AAV virion production, while E1B19k is not. In addition, International Publication WO 97/17458 and Matshushita et al., (1998) Gene Therapy 5:938-945, describe accessory function vectors encoding various Ad genes. Particularly preferred accessory function vectors comprise an adenovirus VA RNA coding region, an adenovirus E4 ORF6 coding region, an adenovirus E2A 72 kD coding region, an adenovirus E1A coding region, and an adenovirus E1B region lacking an intact E1B55k coding region. Such vectors are described in International Publication No. WO 01/83797.

Use of insect cells for expression of heterologous proteins is well documented, as are methods of introducing nucleic acids, such as vectors, e.g., insect-cell compatible vectors, into such cells and methods of maintaining such cells in culture. (See, e.g., METHODS IN MOLECULAR BIOLOGY, ed. Richard, Humana Press, N J (1995); O'Reilly et al., BACULOVIRUS EXPRESSION VECTORS, A LABORATORY MANUAL, Oxford Univ. Press (1994); Samulski et al., J. Vir. (1989) vol. 63, pp. 3822-3828; Kajigaya et al., Proc. Nat'l. Acad. Sci. USA (1991) vol. 88, pp. 4646-4650; Ruffing et al., J. Vir. (1992) vol. 66, pp. 6922-6930; Kirnbauer et al., Vir. (1996) vol. 219, pp. 37-44; Zhao et al., Vir. (2000) vol. 272, pp. 382-393; and U.S. Pat. No. 6,204,059). In some embodiments, the nucleic acid construct encoding AAV in insect cells is an insect cell-compatible vector. “Expression vector” refers to a vector including a recombinant polynucleotide including expression control sequences operatively linked to a nucleotide sequence to be expressed. An expression vector includes sufficient cis-acting elements for expression; other elements for expression can be supplied by the host cell or in vitro expression system. Expression vectors include all those known in the art, such as cosmids, plasmids (e.g., naked or contained in liposomes), artificial chromosomes, and viruses that incorporate the recombinant polynucleotide. An “insect cell-compatible vector” or “vector” as used herein refers to a nucleic acid molecule capable of productive transformation or transfection of an insect or insect cell. Exemplary biological vectors include plasmids, linear nucleic acid molecules, and recombinant viruses. Any vector can be employed as long as it is insect cell-compatible. The vector may integrate into the insect cells genome but the presence of the vector in the insect cell need not be permanent and transient episomal vectors are also included. The vectors can be introduced by any means known, for example by chemical treatment of the cells, electroporation, or infection. In some embodiments, the vector is a baculovirus, a viral vector, or a plasmid. In a more preferred embodiment, the vector is a baculovirus, i.e. the construct is a baculoviral vector. Baculoviral vectors and methods for their use are described in the above cited references on molecular engineering of insect cells.

The baculovirus shuttle vector or bacmids are used for generating baculoviruses. Bacmids propagate in bacteria such as Escherichia coli as a large plasmid. When transfected into insect cells, the bacmids generate baculovirus.

In some embodiments, the culture medium is an infection or transfection medium (e.g., medium in which the host cell producing the AAV viral particle is infected or transfected with genes (infection or transfection media).

In another embodiment, the culture medium is a producer medium (e.g., medium in which the host cell produces the AAV viral particle). These media include, without limitation, media produced by Life Technologies including Modified Eagle Medium (MEM), Dulbecco's Modified Eagle Medium (DMEM), custom formulations such as those described in U.S. Pat. No. 6,566,118, and Sf-900 II SFM media as described in U.S. Pat. No. 6,723,551, each of which is incorporated herein by reference in its entirety, particularly with respect to custom media formulations for use in production of AAV viral particle. Other examples of cell culture medias that can be used include Sf-900 III SFM (Life Technologies), ExpiSf CD Medium (Life Technologies), Express Five SFM (Life Technologies), IS Sf Insect (Fujifilm), TheraPEAK (Lonza), and EX-CELL® CD Insect Cell Medium (Millipore Sigma).

rAAV particles can also be produced using methods disclosed in various embodiments. In some instances, rAAV particles can be produced by using an insect or mammalian cell that stably expresses some of the necessary components for rAAV particle production. For example, a plasmid (or multiple plasmids) including AAV rep and cap genes, and a selectable marker, such as a neomycin resistance gene, can be integrated into the genome of the cell. In another example, a plasmid (or multiple plasmids) including a selectable marker, such as a neomycin resistance gene, can be integrated into the genome of the cell. The insect, fungal, or mammalian cell can then be co-infected with a helper virus (e.g., adenovirus or baculovirus providing the helper functions) and the viral vector including the 5′ and 3′ AAV ITR (and the nucleotide sequence encoding the heterologous protein, if desired). The advantages of this method are that the cells are selectable and are suitable for large-scale production of the rAAV. As another non-limiting example, adenovirus or baculovirus rather than plasmids can be used to introduce a host regulatory gene, rep gene, and cap gene into packaging cells.

Host Cells

The host cell can be any invertebrate or vertebrate cell type which allows for production of the AAV viral particle and which can be maintained in culture.

In one embodiment, the host cell is an insect cell or a mammalian cell.

In another embodiment, the host cell is an insect cell.

In some embodiments, the insect cell is from Spodoptera frugiperda, such as Sf9, Sf21, Sf900+, drosophila cell lines, mosquito cell lines, for example, Aedes albopictus derived cell lines, domestic silkworm cell lines, for example, Bombyx mori cell lines, Trichoplusia ni cell lines such as High Five cells or Lepidoptera cell lines such as Ascalapha odorata cell lines. Preferred insect cells are cells from the insect species which are susceptible to baculovirus infection, including High Five, Sf9, Se301, SeIZD2109, SeUCR1, Sf900+, Sf21, BTI-TN-5B 1-4, MG-1, Tn368, HzAml, BM-N, Ha2302, Hz2E5 and Ao38.

In other embodiments, the host cell is a Sf9 cell.

In one embodiment, the host cell is a mammalian cell.

In another embodiment, the mammalian cell is HEK293, HeLa, CHO, NSO, SP2/0, PER.C6, Vera, RD, BHK, HT 1080, A549, Cos-7, ARPE-19 or MRC-5 cells.

AAV Serotypes

There are at least thirteen serotypes of AAV that have been characterized, as shown in Table 1. The instant invention encompasses but is not limited to these specific AAV serotypes.

TABLE 1
AAV Serotypes.

| | NCBI Reference Sequence No./ |
|---|---|
| AAV | Genbank Accession No. |
| Serotype | (each herein incorporated by reference) |
| AAV-1 | NC_002077.1 |
| AAV-2 | NC_001401.2 |
| AAV-3 | NC_001729.1 |
| AAV-3B | AF028705.1 |
| AAV-4 | NC_001829.1 |
| AAV-5 | NC_006152.1 |
| AAV-6 | AF028704-1 |
| AAV-7 | NC_006260.1 |
| AAV-8 | NC_006261.1 |
| AAV-9 | AX7S3250.1 |
| AAV-10 | AY631965.1 |
| AAV-11 | AY631966.1 |
| AAV-12 | DQ813647.1 |
| AAV-13 | EU28SS62.1 |

General information and reviews of AAV can be found in, for example, Carter, 1989, Handbook of Parvoviruses, Vol. 1, pp. 169-228, and Berns, 1990, Virology, pp. 1743-1764, Raven Press, (New York). However, it is fully expected that these same principles will be applicable to additional AAV serotypes since it is well known that the various serotypes are quite closely related, both structurally and functionally, even at the genetic level. (See, for example, Blacklowe, 1988, pp. 165-174 of Parvoviruses and Human Disease, J. R. Pattison, ed.; and Rose, Comprehensive Virology 3:1-61 (1974)). For example, all AAV serotypes apparently exhibit very similar replication properties mediated by homologous rep genes; and all bear three related capsid proteins such as those expressed in AAV-6. The degree of relatedness is further suggested by heteroduplex analysis which reveals extensive cross-hybridization between serotypes along the length of the genome; and the presence of analogous self-annealing segments at the termini that correspond to ITRs. The similar infectivity patterns also suggest that the replication functions in each serotype are under similar regulatory control.

AAV “rep” and “cap” genes are genes encoding replication and encapsidation proteins, respectively. AAV rep and cap genes have been found in all AAV serotypes examined and are described herein, and in the references cited. In wild-type AAV, the rep and cap genes are generally found adjacent to each other in the viral genome (i.e., they are “coupled” together as adjoining or overlapping transcriptional units), and they are generally conserved among AAV serotypes. AAV rep and cap genes are also individually and collectively referred to as “AAV packaging genes.” The AAV cap gene in accordance with the present invention encodes a Cap protein which is capable of packaging AAV vectors in the presence of rep and adeno helper function and is capable of binding target cellular receptors. In some embodiments, the AAV cap gene encodes a capsid protein having an amino acid sequence derived from a particular AAV serotype, for example the serotypes shown in Table 1; or derived from alternative capsid variant sequences of AAV found in mammals e.g., humans, baboons, pigs, marmosets, chimpanzees, or macaques (e.g., rhesus (Macaca mulatta), cynomolgus (“long-tailed”) (M. fascicularis), or pigtailed (M. nemestrina)).

The AAV sequences employed for the production of AAV can be derived from the genome of any AAV serotype. The AAV serotypes may have genomic sequences of significant homology at the amino acid and the nucleic acid levels, provide a similar set of genetic functions, produce virions which are essentially physically and functionally equivalent, and replicate and assemble by practically identical mechanisms. See, for example, GenBank Accession number U89790; GenBank Accession number J01901; GenBank Accession number AF043303; GenBank Accession number AF085716; Chlorini et al., J. Vir. 71: 6823-33 (1997); Srivastava et al., J. Vir. 45:555-64 (1983); Chlorini et al., J. Vir. 73:1309-1319 (1999); Rutledge et al., J. Vir. 72:309-319 (1998); and Wu et al., J. Vir. 74: 8635-47 (2000), which are herein incorporated by reference for the genomic sequences of AAV serotypes and/or discussions of the genomic similarities.

The genomic organization of many of the known AAV serotypes can be very similar. The genome of AAV is a linear, single-stranded DNA molecule that is less than about 5,000 nucleotides (nt) in length. Inverted terminal repeats (ITRs) flank the unique coding nucleotide sequences for the non-structural replication (Rep) proteins and the structural (VP) proteins. The VP proteins form the capsid. The terminal 145 nt are self-complementary and are organized so that an energetically stable intramolecular duplex forming a T-shaped hairpin may be formed. These hairpin structures function as an origin for viral DNA replication, serving as primers for the cellular DNA polymerase complex. The Rep genes encode the Rep proteins, Rep78, Rep68, Rep52, and Rep40. Rep78 and Rep68 are transcribed from the p5 promoter, and Rep 52 and Rep40 are transcribed from the p19 promoter. The cap genes encode the VP proteins, VP1, VP2, and VP3. The cap genes are transcribed from the p40 promoter.

In various embodiments, a vector providing AAV helper functions includes a nucleotide sequence(s) that encode capsid proteins, Rep proteins, or AAP proteins. The cap genes, rep gene, and/or AAP gene from any AAV serotype (including, but not limited to, AAV1 (NCBI Reference Sequence No./Genbank Accession No. NC_002077.1), AAV2 (NCBI Reference Sequence No./Genbank Accession No. NC_001401.2), AAV3 (NCBI Reference Sequence No./Genbank Accession No. NC_001729.1), AAV3B (NCBI Reference Sequence No./Genbank Accession No. AF028705.1), AAV4 (NCBI Reference Sequence No./Genbank Accession No. NC_001829.1), AAV5 (NCBI Reference Sequence No./Genbank Accession No. NC_006152.1), AAV6 (NCBI Reference Sequence No./Genbank Accession No. AF028704.1), AAV7 (NCBI Reference Sequence No./Genbank Accession No. NC_006260.1), AAV8 (NCBI Reference Sequence No./Genbank Accession No. NC_006261.1), AAV9 (NCBI Reference Sequence No./Genbank Accession No. AX753250.1), AAV10 (NCBI Reference Sequence No./Genbank Accession No. AY631965.1), AAV11 (NCBI Reference Sequence No./Genbank Accession No. AY631966.1), AAV12 (NCBI Reference Sequence No./Genbank Accession No. DQ813647.1), AAV13 (NCBI Reference Sequence No./Genbank Accession No. EU285562.1), is AAV-rh.10 (AAVrh10), AAV-DJ (AAVDJ), AAV-DJ8 (AAVDJ8), AAV-1, AAV-2, AAV-2G9, AAV-3, AAV3a, AAV3b, AAV3-3, AAV4, AAV4-4, AAV-5, AAV-6, AAV6.1, AAV6.2, AAV6.1.2, AAV-7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV-10, AAV-11, AAV-12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV10, or Japanese AAV10 serotypes, AAV_po.6, AAV_po., AAV_po.5, AAV_LK03, AAV_ra.1, AAV_bat_YNM, AAV_bat_Brazil, AAV_mo.1, AAV_avian_DA-1, or AAV_mouse_NY1, Bba21, Bba26, Bba27, Bba29, Bba30, Bba31, Bba32, Bba33, Bba34, Bba35, Bba36, Bba37, Bba38, Bba41, Bba42, Bba43, Bba44, Bce14, Bce15, Bce16, Bce17, Bce18, Bce20, Bce35, Bce36, Bce39, Bce40, Bce41, Bce42, Bce43, Bce44, Bce45, Bce46, Bey20, Bey22, Bey23, Bma42, Bma43, Bpo1, Bpo2, Bpo3, Bpo4, Bpo6, Bpo8, Bpo13, Bpo18, Bpo20, Bpo23, Bpo24, Bpo27, Bpo28, Bpo29, Bpo33, Bpo35, Bpo36, Bpo37, Brh26, Brh27, Brh28, Brh29, Brh30, Brh31, Brh32, Brh33, Bfm17, Bfm18, Bfm20, Bfm21, Bfm24, Bfm25, Bfm27, Bfm32, Bfm33, Bfm34, Bfm35, AAV-rh10, AAV-rh39, AAV-rh43, AAVanc80L65, or any variants thereof) can be used herein to produce the recombinant AAV Exemplary capsids are also provided in International Application No. WO 2018/022608 and WO 2019/222136, which are incorporated herein in its entirety. Each NCBI Reference Sequence Number or Genbank Accession Numbers provided above is also incorporated by reference herein. In some embodiments, the AAV cap genes encode a capsid from serotype 1, serotype 2, serotype 3, serotype 3B, serotype 4, serotype 5, serotype 6, serotype 7, serotype 8, serotype 9, serotype 10, serotype 11, serotype 12, serotype 13, or a variant thereof.

In addition to the capsid, Rep, and AAP genes, embodiments include exogenous polynucleotides that express helper proteins. Without limitation, helper gene products that can be expressed in the host cell in various combinations include Spodoptera frugiperda FKBP46, human FKBP52, Adenovirus E1A, E1B, E2A, E4 and VA, Herpes simplex virus UL29, UL30, UL42, U15, ULB, UL52, and UL9. In an embodiment, the cell expresses at least one immunophilin analogue (i.e., an immunophilin or similar protein) and at least one helper virus gene product.

In some embodiments, the three AAV capsid proteins, namely VP1, VP2, and VP3, are produced in an overlapping fashion from the cap open reading frame (ORF) using alternative mRNA splicing of the transcript and alternative translational start codon usage. For example, VP1 can be translated from an ATG start codon (amino acid M1) on the mRNA, while VP2 and VP3 can arise from a shorter mRNA, for example, using a different start codon for VP2 production and readthrough translation to the next available start codon for the production of VP3.

The Cap proteins can be VP1 and VP3, or VP1, VP2, and VP3. The VP1, VP2 or VP3 genes can express capsid proteins of AAV serotypes AAV1, AAV2, AAV4, AAV5, AAV6, AAV7, AAV8, AAV9, AAV10, AAV11, AAV12, AAV13, AAV-rh.10 (AAVrh10), AAV-DJ (AAVDJ), AAV-DJ8 (AAVDJ8), AAV-1, AAV-2, AAV-2G9, AAV-3, AAV3a, AAV3b, AAV3-3, AAV4, AAV4-4, AAV-5, AAV-6, AAV6.1, AAV6.2, AAV6.1.2, AAV-7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV-10, AAV-11, AAV-12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh. 62, AAV2-3/rh. 61, AAV2-4/rh. 50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb 0.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu. 16, AAV52/hu.19, AAV52.1/hu.20, AAV58.2/hu.25, AAVA3.3, AAVA3.4, AAVA3.5, AAVA3.7, AAVC1, AAVC2, AAVC5, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu. 31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV10, or Japanese AAV10 serotypes, AAV_po.6, AAV_po., AAV_po.5, AAV_LK03, AAV_ra.1, AAV_bat_YNM, AAV_bat_Brazil, AAV_mo.1, AAV_avian_DA-1, or AAV_mouse_NY1, Bba21, Bba26, Bba27, Bba29, Bba30, Bba31, Bba32, Bba33, Bba34, Bba35, Bba36, Bba37, Bba38, Bba41, Bba42, Bba43, Bba44, Bce14, Bce15, Bce16, Bce17, Bce18, Bce20, Bce35, Bce36, Bce39, Bce40, Bce41, Bce42, Bce43, Bce44, Bce45, Bce46, Bey20, Bey22, Bey23, Bma42, Bma43, Bpo1, Bpo2, Bpo3, Bpo4, Bpo6, Bpo8, Bpo13, Bpo18, Bpo20, Bpo23, Bpo24, Bpo27, Bpo28, Bpo29, Bpo33, Bpo35, Bpo36, Bpo37, Brh26, Brh27, Brh28, Brh29, Brh30, Brh31, Brh32, Brh33, Bfm17, Bfm18, Bfm20, Bfm21, Bfm24, Bfm25, Bfm27, Bfm32, Bfm33, Bfm34, Bfm35, AAV-rh10, AAV-rh39, AAV-rh43, or AAVanc80L65. In another embodiment, the VP1, VP2, or VP3 genes express a capsid of a mixed serotype wherein at the VP1, VP2, and VP3 genes do not all come from the same serotype. Exemplary capsids are provided in International Application No. WO 2018/022608, incorporated herein in its entirety.

For example, the amino acid sequence of the VP1 protein of the wild-type AAV serotype 5 (AAV-5) is set forth in SEQ ID NO:1 (set forth without the initiator methionine):

(SEQ ID NO: 1)
SFVDHPPDWGLREFLGLEALEEVGPPKGEPKPNQQHQDQARGLVLPGYNYL
GPGNGLDRGEPVNRADEVAREHDISYNEQLEAGDNPYLKYNHADAEFQEKLADDTSFG
GNLGKAVFQAKKRVLEPFGLVEEGAKTAPTGKRIDDHFPKRKKARTEEDSKPSTSSDAE
AGPSGSQQLQIPAQPASSLGADTMSAGGGGPLGDNNQGADGVGNASGDWHCDSTWM
GDRVVTKSTRTWVLPSYNNHQYREIKSGSVDGSNANAYFGYSTPWGYFDFNRFHSHWS
PRDWQRLINNYWGFRPRSLRVKIFNIQVKEVTVQDSTTTIANNLTSTVQVFTDDDYQLP
YVVGNGTEGCLPAFPPQVFTLPQYGYATLNRDNTENPTERSSFFCLEYFPSKMLRTGNN
FEFTYNFEEVPFHSSFAPSQNLFKLANPLVDQYLYRFVSTNNTGGVQFNKNLAGRYANT
YKNWFPGPMGRTQGWNLGSGVNRASVSAFATTNRMELEGASYQVPPQPNGMTNNLQ
GSNTYALENTMIFNSQPANPGTTATYLEGNMLITSESETQPVNRVAYNVGGQMATNNQ
SSTTAPATGTYNLQEIVPGSVWMERDVYLQGPIWAKIPETGAHFHPSPAMGGFGLKHPP
PMMLIKNTPVPGNITSFSDVPVSSFITQYSTGQVTVEMEWELKKENSKRWNPEIQYTNN
YNDPQFVDFAPDSTGEYRTTRPIGTRYLTRPL.

In some embodiments, the VP1, VP2, and/or VP3 protein (of the capsid of the AAV viral particle) has an amino acid sequence as disclosed by WO 2018/022608 or U.S. Pat. No. 9,737,618.

In other embodiments, the VP1, VP2, and/or VP3 protein (of the capsid of the AAV viral particle) has an amino acid sequence of capsid proteins of AAV found in, for example, baboons, pigs, marmosets, chimpanzees, or macaques, wherein the AAV viral particle is pseudotyped with the VP1, VP2, and/or VP3 protein.

In one embodiment, the VP1 sequence (from baboon; denoted as Bba21) comprises the amino acid sequence of SEQ ID NO:2 (amino acids 1-742); or comprises an amino acid sequence that is at least 60%, at least 65%, at least 70%, at least 75%, at least 80%, at least 85%, at least 90%, or at least 95% identical over the full length of the amino acid sequence of SEQ ID NO:2. The VP2 capsid protein spans amino acids 138-742 of SEQ ID NO:2 and the VP3 capsid protein spans amino acids 206-742 of SEQ ID NO:2.

(SEQ ID NO: 2)
MAADGYLPDWLEDNLSEGIREWWALKPGAPQPKANQQHQDNARGLVLPGY
KYLGPGNGLDKGEPVNEADAAALEHDKAYDQQLKSGDNPYLKYNHADAEFQQRLAT
DTSFGGNLGKAVFQAKKRILEPLGLVEEGVKTAPGKKRPLEKTPNRPTNPDSGKAPAKK
KQKDGETADSARRTLDFEDSGAGDGPPEGSSSGEMSHDAEMRAAPGGNAVEAGQGAD
GVGNASGDWHCDSTWSEGRVTTTSTRTWVLPTYNNHLYLRIGTTANSNTYNGFSTPWG
YFDFNRFHCHFSPRDWQRLINNNWGLRPKSMRVKIFNIQVKEVTTSNGETTVANNLTST
VQIFADSTYELPYVMDAGQEGSLPPFPNDVFMVPQYGYCGVVTGENQNQTDRNAFYCL
EYFPSQMLRTGNNFEVSYQFEKVPFHSMYAHSQSLDRMMNPLLDQYLWHPQSTTTGNS
LNQGTATTTYGKITTGDFAYYRKNWLPGACIKQQKFSKNASQNYKIPASGGDALLKYD
THTTSNGRWSNMAPGPPMATAGAGDSDFSNSQLIFAGPNQSGNTTTSSNNLLFTSEEEIA
TTNPRDTDMFGQIADNNQNATTAPHIANLDAMGIVPGMVWQNRDIYYLGPIWAKVPHT
DGHFHPSPLMGGFGLKHPPPQIFIKNTPVPANPNTTFSAARINSFLTQYSTGQVAVQIDW
EIQKEHSKRWDPEVQFTSNYGTQNSMLWAPDNAGNYHEPRAIGSRFLTHHL.

In another embodiment, the VP1 sequence (from baboon; denoted as Bba26) comprises the amino acid sequence of SEQ ID NO:3 (amino acids 1-739); or comprises an amino acid sequence that is at least 60%, at least 65%, at least 70%, at least 75%, at least 80%, at least 85%, at least 90%, or at least 95% identical over the full length of the amino acid sequence of SEQ ID NO:3. The VP2 capsid protein spans amino acids 138-739 of SEQ ID NO:3 and the VP3 capsid protein spans amino acids 206-739 of SEQ ID NO:3.

(SEQ ID NO: 3)
MAADGYLPDWLEDNLSEGIREWWALKPGAPQPKANQQHQGNSRGLVLPGY
KYLGLFNGLDKGEPVNEADAAALEHDKAYDKQLEQGDDPYLKYNHADAEFQERLQED
TSFGGNLGRAVFQAKKRILEPLGLVEEAAKTAPGKKRPLEKTPNQPTDTQAAGQTPAKK
RPQGEQSGDSARRQLDFGPQPAAPIGQPPAAPPPVGSNTMASGGGGPMADDNQGADGV
GNASGNWHCDSTWLGDRVITTSTRTWVLPTYNNHIYKQISSESGATNDNHYFGYSTPW
GYFDFNRFHCHFSPRDWQRLINNNWGFRPKRLNFKIFNVQVKEVTQIEGGSTIANNLTST
IQVFADSEYQLPYVLGSAHQGCLPPFPADVFMIPQYGYLTLNNGSQSVGRSSFYCLEYFP
SQMLRTGNNFQFSYTFESVPFHSSYAHSQSLDRIMNPLVDQYLYYLARTQTGTGSTTSN
TRQLQFYQAGPSNMADQSRNWLPGPMYRQQRVSKTLDQNSNTNFAWTAASKYNLNG
RKSLANPGIAMATHKDDEERFFPQHGVLVFGQTNATNKTTLDNVLVTSEEEIKATNPVA
TEEYGTVSSNLQASNTNPTTETVNNQGILPGMVWQDRDVYLQGPIWAKIPHTDGHFHPS
PLMGGFGLKHPPPQILIKNTPVPANPPETFTTSKFASYITQYSTGQVSVEIEWELQKENSK
RWNPEIQYTSNYAKSNNVEFSVDAAGVYSEPRPIGTRYLTRNL.

In some embodiments, the VP1 sequence (from baboon; denoted as Bba27) comprises the amino acid sequence of SEQ ID NO:4 (amino acids 1-739); or comprises an amino acid sequence that is at least 60%, at least 65%, at least 70%, at least 75%, at least 80%, at least 85%, at least 90%, or at least 95% identical over the full length of the amino acid sequence of SEQ ID NO:4. The VP2 capsid protein spans amino acids 138-739 of SEQ ID NO:4 and the VP3 capsid protein spans amino acids 206-739 of SEQ ID NO:4.

(SEQ ID NO: 4)
VAADGYLPDWLEDNLSEGIREWWALKPGAPQPKANQQHQDNSRGLVLPGY
KYLGPFNGLDKGEPVNEADAAALEHDKAYDKQLEQGDNPYLKYNHADAELQERLQED
TSFGGNLGRAVFQAKKRILEPLGLVEEAAKTAPGKKRPLEKTPNQPTDTQAAGQTPAKK
RPQGEQSGDSARRQLDFDPQPAAPIGQPPAAPPPVGSNTMASGGGGPMADDNQGADGV
GNASGNWHRDSTWLGDRVITTSTRTWVLPTYNNHIYKQISSESGATNDNHYFGYSTPW
GYFDFNRFHCHFSPRDWQRLINNNWGFRPKRLNFKIFNIQVKEVTQIEGGSTIANNLTSTI
QVFADSEYQLPYVLGSAHQGCLPPFPADVFMIPQYGYLTLNNGSQSVGRSSFYCLEYFPS
QMLRTGNNFQFSYTFESVPFHSSYAHSQSLDRIMNPLVDQYLYYLARTQTGTGSTTSNT
RQLQFYQAGPSNMADQSRNWLPGPMYRQQRVSKTLDQNSNTNFAWTAASKYNLNGR
KSLANPGIAMATHKDDEERFFPQHGVLVFGQTNATNKTTLDNVLITSEEEIKATNPVATE
EYGTVSSNLQASNTNPTTETVNSQGILPGMVWQDRDVYLQGPIWAKIPHTDGHFHPSPL
MGGFGLKHPPPQILIKNTPVPANPPETFTTSKFASYITQYSTGQVSVEIEWELQKENGKR
WNPEIQYTSNYAKSNNVEFSVDAAGVYSEPRPIGTRYLTRNL.

In other embodiments, the VP1 sequence (from baboon; denoted as Bba29) comprises the amino acid sequence of SEQ ID NO:5 (amino acids 1-739); or comprises an amino acid sequence that is at least 60%, at least 65%, at least 70%, at least 75%, at least 80%, at least 85%, at least 90%, or at least 95% identical over the full length of the amino acid sequence of SEQ ID NO:5. The VP2 capsid protein spans amino acids 138-739 of SEQ ID NO:5 and the VP3 capsid protein spans amino acids 206-739 of SEQ ID NO:5.

(SEQ ID NO: 5)
MAADGYLPDWLEDNLSEGIREWWALKPGAPQPKANQQHQDNSRGLVLPGY
KYLGPFNGLDKGEPVNEADAAALEHDKAYDKQLEQGDNPYLKYNHADAEFQERLQED
TSFGGNLGRAVFQAKKRILEPLGLVEEAAKTAPGKRRPLEKTPNQPTDTQAAGQTPAKK
RPQGEQSGDSARRQLDFDPQPAAPIGQPPAAPSPVGSNTMASGGGGPMADDNQGADGV
GNASGNWHCDSTWLGDRVITTSTRTWVLPTYNNHIYKQISSESGATNDNHYFGYSTPW
GYFDFNRFHCHFSPRDWQRLINNNWGFRPKRLNFKIFNIQVKEVTQIEGGSTIANNLTSTI
QVFADSEYQLPYVLGSAHQGCLPPFPADVFMIPQYGYLTLNNGSQSVGRSSFYCLEYFPS
QMLRTGNNFQFSYTFESVPFHSSYAHSQSLDRIMNPLVDQCLYYLARTQTGTGSTTSNT
RQLQFYQAGPSNMADQSRNWLPGPMYRQQRVSKTLDQNSNTNFAWTAASKYNLNGR
KSLANPGIAMATHKDDEERFFPQHGVLVFGQTNATNKTTLDNVLITSEEEIKATNPVATE
EYGTVSSNLQASNTNPTTETVNNQGILPGMVWQDRDVYLQGPIWAKIPHTDGHFHPSPL
MGGFGLKHPPPQILIKNTPVPANPPETFTTSKFASYITQYSTGQVSVEIEWELQKEDSKR
WNPEIQYTSNYAKSNNVEFSVDAAGVYSEPRPIGTRYLTRNL.

In other embodiments, the VP1 sequence (from baboon; denoted as Bba30) comprises the amino acid sequence of SEQ ID NO:6 (amino acids 1-739); or comprises an amino acid sequence that is at least 60%, at least 65%, at least 70%, at least 75%, at least 80%, at least 85%, at least 90%, or at least 95% identical over the full length of the amino acid sequence of SEQ ID NO:6. The VP2 capsid protein spans amino acids 138-739 of SEQ ID NO:6 and the VP3 capsid protein spans amino acids 206-739 of SEQ ID NO:6.

(SEQ ID NO: 6)
MAADGYLPDWLEDNLSEGIREWWALKPGAPQPKANQQHQDNSRGLVLPGY
KYLGPFNGLDKGEPVNEADAAALEHDKAYDKQLEQGDNPYLKYNHADAEFQERLQED
TSFGGNLGRAVFQAKKRILEPLGLVEEAAKTAPGKKRPLEKTPNQPTDTQAAGQTPAKK
RPQGGQSGDSARRQLDFDPQPAAPIGQPPAAPSPVGSNTMASGGGGPMADDNQGADGV
GNASGNWHCDSTWLGDRVITTSTRTWVLPTYNNHIYKQISSESGATNDNHYFGYSTPW
GYFDFNRFHCHFSPRDWQRLINNNWGFRPKRLNFKIFNIQVKEVTQIEGGSTIANNLTSTI
QVFADSEYLLPYVLGSAHQGCLPPFPADVFMIPQYGYLTLNNGSQSVGRSSFYCLEYFPS
QMLRTGNNFQFSYTFESVPFHSSYAHSQSLDRIMNPLVDQYLYYLARTQTGTGSTTSNT
RQLQFYQAGPSNMADQSRNWLPGPMYRQQRVSKTLDQNSNTNFAWTAASKYNLNGR
KSLANPGIAMATHKDDEERFFPQHGVLVFGQTNATNKTTLDNVLITSEEEIKATNPVATE
EYGTVSSNLQASNTNPTTETVNNQGILPGMVWQDRDVYLQGPIWAKIPHTDGHFHPSPL
MGGFGLKHPPPQILIKNTPVPANPPETFTTSKFASYITQYSTGQVSVEIEWELQKEDSKR
WNPEIQYTSNYAKSNNVEFSVDAAGVYSEPRPIGTRYLTRNL.

In one embodiment, the VP1 sequence (from baboon; denoted as Bba31) comprises the amino acid sequence of SEQ ID NO:7 (amino acids 1-742); or comprises an amino acid sequence that is at least 60%, at least 65%, at least 70%, at least 75%, at least 80%, at least 85%, at least 90%, or at least 95% identical over the full length of the amino acid sequence of SEQ ID NO:7. The VP2 capsid protein spans amino acids 138-742 of SEQ ID NO:7 and the VP3 capsid protein spans amino acids 206-742 of SEQ ID NO:7.

(SEQ ID NO: 7)
MAADGYLPDWLEDNLSEGIREWWALKPGAPQPKANQQHQDNARGLVLPGY
KYLGPGNGLDKGEPVNEADAAALEHDKAYDQQLKSGDNPYLKYNHADAEFQQRLAT
DTSFGGNLGKAVFQAKKRILEPLGLVEEGVKTAPGKKHPLEKTPNRPTNPDSGKAPAKK
KQKDGETADSARRTLDFEDSGAGDGPPEGSSSGEMSHDAEMRAAPGGNAVEAGQGAD
GVGNASGDWHCGSTWSEGRVTTTSTRTWVLPTYNNHLYLRIGTTANSNTYNGFSTPWG
YFDFNRFHCHFSPRDWQRLINNNWGLRPKSMRVKIFNIQVKEVTTSNGETTVANNLTST
VQIFADSTYELPYVMDAGQEGSLPPFPNDVFMVPQYGYCGVVTGENQNQTDRNAFYCL
ECFPSQMLRTGNNFEISYQFEKVPFHSMYAHSQSLDRMMNPLLDQYLWHLQSTTTGNS
LNQGTATTTYGKITTGDFAYYRKNWLPGACIKQQKFSKNASQNYKIPASGGDALLKYD
THTTLNGRWSNMAPGPPMATAGAGDSDFSNSQLIFAGPNQSGNTTTSSNNLLFTSEEEIA
ATNPRDTDMFGQIADNNQNATTAPHIANLDAMGIVPGMVWQNRDIYYQGPIWAKVPH
TDGHFHPSPLMGGFGLKHPPPQIFIKNTPVPANPNTTFSAARINSFLTQYSTGQVAVQID
WEIQKEHSKRWNPEVQFTSNYGTQNSMLWAPDNAGNYHEPRAIGSRFLTHHL.

In another embodiment, the VP1 sequence (from baboon; denoted as Bba32) comprises the amino acid sequence of SEQ ID NO:8 (amino acids 1-742); or comprises an amino acid sequence that is at least 60%, at least 65%, at least 70%, at least 75%, at least 80%, at least 85%, at least 90%, or at least 95% identical over the full length of the amino acid sequence of SEQ ID NO:8. The VP2 capsid protein spans amino acids 138-742 of SEQ ID NO:8 and the VP3 capsid protein spans amino acids 206-742 of SEQ ID NO:8.

(SEQ ID NO: 8)
MAADGYLPDWLEDNLSEGIREWWALKPGAPQPKANQQHQDNARGLVLPGY
KYLGPGNGLDKGEPVNEADAAALEHDKAYDQQLKSGDNPYLKYNHADAEFQQRLAT
DTSFGGNLGKAVFQAKKRILEPLGLVEEGVKTAPGKKRPLEKTPNRPTNPDSGKAPAKK
KQKDGETADSARRTLDFEDSGAGDGPPEGSSSGEMSHDAEMRAAPGGNAVEAGQGAD
GVGNASGGWHCDSTWSEGRVTTTSTRTWVLPTYNNHLYLRIGTTANSNTYNGFSTPWG
YFDFNRFHCHFSPRDWQRLINNNWGLRPKSMRVKIFNIQVKEVTTSNGETTVANNLTST
VQIFADSTYELPYVMDAGQEGSLPPFPNDVFMVPQYGYCGVVTGENQNQTDRNAFYCL
EYFPSQMLRTGNNFEISYQFEKVPFHSMYAHSQSLDRMMNPLLDQYLWHLQSTTTGNS
LNQGTATTTYGKITTGDFAYYRKNWLPGACIKQQKFSKNASQNYKIPASGGDALLKYD
THTTLNGRWSSMAPGPPMATAGAGDSDFSNSQLIFAGPNQSGNTTTSSNNLLFTSEEEIA
TTNPRDTDMFGQIADNNQNATTAPHIANLDAMGIVPGMVWQNRDIYYQGPIWAKVPHT
DGHFHPSPLMGGFGLKHPPPQIFIKNTPVPANPNTTFSAARINSFLTQYSTGQVAVQIDW
EIQKEHSKRWNPEVQFTSNYGTQNSMLWAPGNAGNHHEPRAIGSRFLTHHL.

In some embodiment, the VP1 sequence (from baboon; denoted as Bba33) comprises the amino acid sequence of SEQ ID NO:9 (amino acids 1-742); or comprises an amino acid sequence that is at least 60%, at least 65%, at least 70%, at least 75%, at least 80%, at least 85%, at least 90%, or at least 95% identical over the full length of the amino acid sequence of SEQ ID NO:9. The VP2 capsid protein spans amino acids 138-742 of SEQ ID NO:9 and the VP3 capsid protein spans amino acids 206-742 of SEQ ID NO:9.

(SEQ ID NO: 9)
MAADGYLPDWLEDNLSEGIREWWALKPGAPQPKANQQHQDNARGLVLPGY
KYLGPGNGLDKGEPVNEADAAALEHDKAYDQQLKSGDNPYLKYNHADAEFQQRLAT
DTSFGGNLGKAVFQAKKRILEPLGLVEEGVKTAPGKKRPLEKTPNRPTNPDSGRAPAKK
KQKDGETADSARRTLDFEDSGAGDGPPEGSSSGEMSHDAEMRAAPGGNAVEAGQGAD
GVGNASGDWHCDSTWSEGRVTTTSTRTWVLPTYNNHLYLRIGTTANSNTYNGFSTPWG
YFDFNRFHCHFSPRDWQRLINNNWGLRPKSMRVKIFNIQVKEVTTSNGETTVANNLTST
VQIFADSTYELPYVMDAGQEGSLPPFPNDVFMVPQYGYCGVVTGENQNQTDRNAFYCL
EYFPSQMLRTGNNFEISYQFEKVPFHSMYAHSQSLDRMMNPLLDQYLWHLQSTTTGNS
LNQGTATTTYGKITTGDFAYYRKNWLPGACIKQQKFSKNASQNYKIPASGGDALLKYD
THTTLNGRWSNMAPGPPMATAGAGDSDFSNSQLIFAGPNQSGNTTTSSNNLLLTSEEEIA
TTNPRDTDMFGQIADNNQNATTAPHIANLDAMGIVPGMVWQNRDIYYQGPIWAKVPHT
DGHFHPSPLMGGFGLKHPPPQIFIKNTPVPANPNTTFSAARINSFLTQYSTGQVAVQIDW
EIQKEHSKRWNPEVQFTSNYGTQNSMLWAPGNAGNYHEPRAIGSRFLTHHL.

In other embodiments, the VP1 sequence (from baboon; denoted as Bba34) comprises the amino acid sequence of SEQ ID NO:10 (amino acids 1-739); or comprises an amino acid sequence that is at least 60%, at least 65%, at least 70%, at least 75%, at least 80%, at least 85%, at least 90%, or at least 95% identical over the full length of the amino acid sequence of SEQ ID NO:10. The VP2 capsid protein spans amino acids 138-739 of SEQ ID NO:10 and the VP3 capsid protein spans amino acids 206-739 of SEQ ID NO:10.

(SEQ ID NO: 10)
MAADGYLPDWLEDNLSEGIREWWALKPGAPQPKANQQHQDNSRGLVLPGY
KYLGPFNGLDKGEPVNEADAAALEHDKAYDKQLEQGDNPYLKYNHADAEFQERLQED
TSFGGILGRAVFQAKKRILEPLGLVEEAAKTAPGKKRPLEKTPNQPTDTQAAGQTPAKK
RPQGEQSGDSARRQLDFDPQPAAPIGQPPAAPSPVGSNTMASGGGGPMADDNQGADGV
GNASGNWHCDSTWLGDRVITTSTRTWVLPTYNNHIYKQISSESGATNDNHYFGYSTPW
GYFDFNRFHCHFSPRDWQRLINNNWGFRPKRLNFKIFNIQVKEVTQIEGGSTIANNLTSTI
QVFADSEYQLPYVLGSAHQGCLPPFPADVFMIPQYGYLTLNNGSQSVGRSSFYCLEYFPS
QMLRTGNNFQFSYTFESVPFHSSYAHSQSLDRIMNPLVDQYLYYLARTQTGTGSTTSNT
RQLQFYQAGPSNMADQSRNWLPGPMYRQQRVSKTLDQNSNTNFAWTAASKYNLNGR
KSLVNPGIAMATHKDDEERFFPQHGVLVFGKTNATNKTTLENVLVTDEEEVKATNPVA
TEEYGTVSSNLQSNTTNPTTETVNNQGILPGMVWQDRDVYLQGPIWAKIPHTDGHFHPS
PLMGGFGLKHPPPQILIKNTPVPANPPETFTTSKFASYITQYSTGQVSVEIEWELQKENSK
RWNPEIQYTSNYAKSNNVEFSADAAGVYSEPRPIGTRYLTRNL.

In one embodiment, the VP1 sequence (from baboon; denoted as Bba35) comprises the amino acid sequence of SEQ ID NO:11 (amino acids 1-739); or comprises an amino acid sequence that is at least 60%, at least 65%, at least 70%, at least 75%, at least 80%, at least 85%, at least 90%, or at least 95% identical over the full length of the amino acid sequence of SEQ ID NO:11. The VP2 capsid protein spans amino acids 138-739 of SEQ ID NO:11 and the VP3 capsid protein spans amino acids 206-739 of SEQ ID NO:11.

(SEQ ID NO: 11)
MAADGYLPDWLEDNLSEGIREWWALKPGAPQPKANQQHQDNSRGLVLPGY
KYLGPFNGLDKGEPVNEADAAALEHDKAYDKQLEQGDNPYLKYNHADAEFQERLQED
TSFGGNLGRAVFQAKKRILEPLGLVEEVAKTAPGKKRPLEKTPNQPTDTQAAGQTPAKK
RPQGEQSGDSARRQLDFDPQPAAPIGQPPAAPSPVGSNTMASGGGGPMADDNQGADGV
GNASGNWHCDSTWLGDRVITTSTRTWVLPTYNNHIYKQISSESGATNDNHYFGYSTPW
GYFDFNRFHCHFSPRDWQRLINNNWGFRPKRLNFKIFNIQVKEVTQIEGGSTIANNLTSTI
QVFADSEYQLPYVLGSAHQGCLPPFPADVFMIPQYGYLTLNNGSQSVGRSSFYCLEYFPS
QMLRTGNNFQFSYTFESVPFHSSYAHSQSLDRIMNPLVDQYLYYLARTQTGTGSTTSNT
RQLQFYQAGPSNMADQSRNWLPGPMYRQQRVSKTLDQNSNINFAWTAASKYNLNGR
KSLANPGIAMATHKDDEERFFPQHRVLVFGQTNATNKTTLDNVLITSEEEIKATNPVATE
EYGTVSSNLQASNTDPTTETVNNQGILPGMVWQDRDVYLQGPIWVKIPHTDGHFHPSPL
MGGFGLKHPPPQILIKNTPVPANPPETFTTSKFASYTTQYSTGQVSVEIEWGLQKENSKR
WNPEIQYTSNYAKSNNVEFSVDAAGVYSEPRPIGTRYLTRNL.

In another embodiment, the VP1 sequence (from baboon; denoted as Bba36) comprises the amino acid sequence of SEQ ID NO:12 (amino acids 1-739); or comprises an amino acid sequence that is at least 60%, at least 65%, at least 70%, at least 75%, at least 80%, at least 85%, at least 90%, or at least 95% identical over the full length of the amino acid sequence of SEQ ID NO:12. The VP2 capsid protein spans amino acids 138-739 of SEQ ID NO:12 and the VP3 capsid protein spans amino acids 206-739 of SEQ ID NO:12.

(SEQ ID NO: 12)
MAADGYLPDWLEDNLSEGIREWWALKPGAPQPKANQQHQDNSRGLVLPGY
KYLGPFNGLDKGEPVNEAYAAALEHDKAYDKQLEQGDNPYLKYNHADAEFQERLQED
TSFGGNLGRAVFQAKKRIPEPLGLVEEAAKTAPGKKRPLEKTPNQPTDTQAAGQTPAKK
RPQGEQSGDSARRQLDFDPQPAAPIGQPPAAPSPVGSNTMASGGGGPMADDNQGADGV
GNASGNWHCDSTWLGDRVITTSTRTWVLPTYNNHIYKQISSESGATNDNHYFGYSTPW
GYFDFNRFHCHFSPRDWQRLINNNWGFRPKRLNFKIFNIQVKEVTQIEGGSTIANNLTSTI
QVFADSEYQLPYVLGSAHQGCLPPFPADVFMIPQYGYLTLNNGSQSVGRPSFYCLEYFPS
QMLRTGNNFQFSYTFESVPFHSSYAHSQSLDRIMNPLVDQYLYYLARTQTGTGSTTSNT
RQLQFYQAGPSNMADQSRNWLPGPMYRQQRVSKTLDQNSNINFAWTAASKYNLNGR
KSLANPGIAMATHKDDEERFFPQHGVLVFGQTNATNKTTLDNVLITSEEEIKATNPVATE
EYGTVSSNLQASNTNPTTETVNNQGILPGMVWQDRDVYLQGPIWAKIPHTDGHFHPSPL
MGGFGLKHPPPQILIKNTPVPANPPETFTTSKFASYITQYSTGQVSVEIEWELQKENSKR
WNPEIQYTSNYAKSNNVEFSVDAAGVYSEPRPIGTRYLTRNL.

In some embodiments, the VP1 sequence (from baboon; denoted as Bba37) comprises the amino acid sequence of SEQ ID NO:13 (amino acids 1-739); or comprises an amino acid sequence that is at least 60%, at least 65%, at least 70%, at least 75%, at least 80%, at least 85%, at least 90%, or at least 95% identical over the full length of the amino acid sequence of SEQ ID NO:13. The VP2 capsid protein spans amino acids 138-739 of SEQ ID NO:13 and the VP3 capsid protein spans amino acids 206-739 of SEQ ID NO:13.

(SEQ ID NO: 13)
MAADGYLPDWLEDNLSEGIREWWALKPGAPQPKANQQHQDNSRGLVLPGY
KYLGPFNGLDKGEPVNEADAAALEHDKAYDKQLEQGDNPYLKYNHADAEFQERLQED
TSFGGNLGRAVFQAKKRILEPLGLVEEAAETAPGKKRPLEKTPNQPTDTQAAGQTPAKK
RPQGEQSGDSARRQLDFDPQPAAPIGQPPAAPSPVGSNTMASGGGGPMADDNQGADGV
GNASGNWHCDSTWLGDRVITTSTRTWVLPTYNNHIYKQISSESGATNDNHYFGYSTPW
GYFDFNRFHCHFSPRDWQRLINNNWGFRPKRLNFKIFNIQVKEVTQTEGGSTIANNLTST
IQVFADSEYQLPYVLGSAHQGCLPPFPADVFMIPQYGYLTLNNGSQSVGRSSFYCLEYFP
SQMLRTGNNFQFSYTFESVPFHSSYAHSQSLDRIINPLVDQYLYYLARTQTGTGSTTSNT
RQLQFYQAGPSNMADQSRNWLPGPMYRQQRVSKTLDQNSNTNFAWTAASKYNLNGR
KSLANPGIAMATHKDDEERFFPQHGVLVFGQTNATNKTTLDNVLITSEEEIKATNPVATE
EYGTVSSNLQASNTNPTTETVNNQGILPGMVWQDRDVYLQGPIWAKIPHTDGHFHPSPL
MGGFGLKHPPPQILIKNTPVPANPPETFTTSKFASYITQYSTGQVSVEIEWELQKENSKR
WNPEIQYTSNYAKSNNVEFSVDAAGVYSEPRPIGTRYLTRNL.

In other embodiments, the VP1 sequence (from baboon; denoted as Bba38) comprises the amino acid sequence of SEQ ID NO:14 (amino acids 1-739); or comprises an amino acid sequence that is at least 60%, at least 65%, at least 70%, at least 75%, at least 80%, at least 85%, at least 90%, or at least 95% identical over the full length of the amino acid sequence of SEQ ID NO:14. The VP2 capsid protein spans amino acids 138-739 of SEQ ID NO:14 and the VP3 capsid protein spans amino acids 206-739 of SEQ ID NO:14.

(SEQ ID NO: 14)
MAADGYLPDWLEDNLSEGIRGWWALKPGAPQPKANQQHQDNSRGLVLPGY
KYLGPFNGLDKGEPVNEADAAALEHDKAYDKQLEQGDNPCLKYNHADAEFQERLQED
TSFGGNLGRAVFQAKKRILEPLGLVEEAAKTAPGKKRPLEKTPNQPTDTQAAGQTPAKK
RPQGEQSGDSARRQLDFDPQPAAPIGQPPAAPSPVGSNTMASGGGGPMADDNQGADGV
GNASGNWHCDSTWLGDRVITTSTRTWVLPTYNNHIYKQISSESGATNDNHYFGYSTPW
GYFDFNRFHCHFSPRDWQRLINNNWGFRPKRLNFKIFNIQVKEVTQIEGGSTIANNLTSTI
QVFADSEYQLPYVLGSAHQGCLPPFPADVFMIPQYGYLTLNNGSQSVGRSSFYCLEYFPS
QMLRTGNNFQFSYTFESVPFHSSYAHSQSLDRIMNPLVDQYLYYLARTQTGTGSTTSNT
RQLQFYQAGPSNMADQSRNWLPGPMYRQQRVSKTLDQNSNINFAWTAASKYNLNGR
KSLANPGIAMATHKDDEERFFPQHGVLVFGQTNATNKTTLDNVLITSEEEIKATNPVATE
EYGTVSSNLQASNTNPTTETVNNQGILPGMVWQDRDVYLQGPTWAKIPHTDGHFHPSP
LMGGFGLKHPPPQILIKNTPVPANPPETFTTSKFASYITQYSTGQVSVEIEWELQKENSKR
WNPEIQYTSNYAKSNNVEFSVDAAGVYSEPRPIGTRYLTRNL.

In one embodiment, the VP1 sequence (from baboon; denoted as Bba41) comprises the amino acid sequence of SEQ ID NO:15 (amino acids 1-739); or comprises an amino acid sequence that is at least 60%, at least 65%, at least 70%, at least 75%, at least 80%, at least 85%, at least 90%, or at least 95% identical over the full length of the amino acid sequence of SEQ ID NO:15. The VP2 capsid protein spans amino acids 138-739 of SEQ ID NO:15 and the VP3 capsid protein spans amino acids 206-739 of SEQ ID NO:15.

(SEQ ID NO: 15)
MAADGYLPDWLEDNLSEGIREWWALKPGAPQPKANQQHQDNSRGLVLPGY
KYLGPFNGLDKGEPVNEADAAALEHDKAYDKQLEQGDNPYLKYNHADAEFQERLQED
TSFGGNLGRAVFQAKKRILEPLGLVEEAAKTAPGKKRPLEKTPNQPTDTQAAGQTPAKK
RPQGEQSGDSARRQLDFDPQPAAPIGQPPAAPSPVGSNTMASGGGGPMADDNQGADGV
GNASGNWHCDSTWLGDRVITTSTRTWVLPTYNNHIYKQISSESGATNDNHYFGYSTPW
GYFDFNRFHCHFSPRDWQRLINNNWGFRPKRLNFKIFNIQVKEVTQIEGGSTIANNLTSTI
QVFADSEYQLPYVLGSAHQGCLPPFPADVFMIPQYGYLTLNNGSQSVGRSSFYCLEYFPS
QMLRTGNSFQFSYTFESVPFHSSYAHSQSLDRIMNPLVDQYLYYLARTQTGTGSTTSNT
RQLQFYQAGPSNMADQSRNWLPGPMYRQQRVSKTLDQNSNINFAWTAASKYNLNGR
KSLANPGIAMATHKDDEERFFPQHGVLVFGQTNATNKTTLDNVLITSEEEIKATNPVATE
EYGTVSSNLQASNTNPTTETVNNQGILPGMVWQDRDVYLQGPIWAKIPHTDGHFHPSPL
MGGFGLKHPPPQILIKNTPVPANPPETFTTSKFASYITQYSTGQVSVEIEWELQKENSKR
WNPEIQYTSNYAKSNNVEFSVDAAGVYSEPRPIGTRYLTRNL.

In another embodiment, the VP1 sequence (from baboon; denoted as Bba42) comprises the amino acid sequence of SEQ ID NO:16 (amino acids 1-739); or comprises an amino acid sequence that is at least 60%, at least 65%, at least 70%, at least 75%, at least 80%, at least 85%, at least 90%, or at least 95% identical over the full length of the amino acid sequence of SEQ ID NO:16. The VP2 capsid protein spans amino acids 138-739 of SEQ ID NO:16 and the VP3 capsid protein spans amino acids 206-739 of SEQ ID NO:16.

(SEQ ID NO: 16)
MAADGYLPDWLEDNLSEGIREWWALKPGAPQPKANQQHQDNSRGLVLPGY
KYLGPFNGLDKGEPVNGADAAALEHDKAYDKQLEQGDNPYLKYNHADAEFQERLQED
TSFGGNLGRAVFQAKKRILEPLGLVEEAAKTAPGKKRPLEKTPNQPTDTQAAGQTPAKK
RPQGEQSGDSARRQLDFDPQPAAPIGQPPAAPSPVGSNTMASGGGGPMADDNQGADGV
GNASGNWHCDSTWLGDRVITTSTRTWVLPTYNNHIYKQISSESGATNDNHYFGYSTPW
GYFDFNRFHCHFSPRDWQRLINNNWGFRPKRLNFKIFNIQVKEVTQIEGGSTIANNLTSTI
QVFADSEYQLPYVLGSAHQGCLPPFPADVFMIPQYGYLTLNNGSQSVGRSSFYCLEYFPS
QMLRTGNNFQFSYTFESVPFHSSYAHSQSLDRIMNPLVDQYLYYLARTQTGTGSTTSNT
RQLQFYQAGPSNMADQSRNWLPGPMYRQQRVSKTLDQNSNINFAWTAASKYNLNGR
KSLVNPGIAMATHKDDEERFFPQHGVLVFGKTNATNKTTLENVLVTDEEEVKATNPVA
TEEYGTVSSNLQSNTTNPTTETVNNQGILPGMVWQDRDVYLQGPIWAKIPHTDGHFHPS
PLMGGFGLKHPPPQILIKNTPVPANPPETFTTSKFASYITQYSTGQVSVEIEWELQKENSK
RWNPEIQYTSNYAKSNNVEFSVDAAGVYSEPRLIGTRYLTRNL.

In some embodiments, the VP1 sequence (from baboon; denoted as Bba43) comprises the amino acid sequence of SEQ ID NO:17 (amino acids 1-739); or comprises an amino acid sequence that is at least 60%, at least 65%, at least 70%, at least 75%, at least 80%, at least 85%, at least 90%, or at least 95% identical over the full length of the amino acid sequence of SEQ ID NO:17. The VP2 capsid protein spans amino acids 138-739 of SEQ ID NO:17 and the VP3 capsid protein spans amino acids 206-739 of SEQ ID NO:17.

(SEQ ID NO: 17)
MAADGYLPDWLEDNLSEGIREWWALKPGAPQPKANQQHQDNSRGLVLPGY
KYLGPFNGLDKGEPVNEADAAALEHDKAYDKQLEQGDNPYLKYNHADAEFQERLQED
TSFGGNLGRAVFQAKKRILEPLGLVEEAAKTAPGKKRPLEKTPNQPTDTQAAGQTPAKK
RPQGEQSGDSARRQLDFDPQPAAPIGQPPAAPSPVGSNTMASGGGGPMADDNQGADGV
GNASGNWHCDSTWLGDRVITTSTRTWVLPTYNNHIYKQISSESGATNDNHYFGYSTPW
GYFDFNRFHCHFSPRDWQRLINNNWGFRPKRLNFKIFNIQVKEVTQIEGGSTIANNLTSTI
QVFADSEYQLPYVLGSAHQGCLPPFPADVFMIPQYGYLTLNNGSQSVGRSSFYCLEYFPS
QMLRTGNNFQFSYTFESVPFHSSYAHSQSLDRIMNPLVDQYLYYLARTQTGTGSTTSNT
RQLQFYQAGPSNMADQSRNWLPGPMYRQQRVSKTLDQNSNINFAWTAASKYNLNGR
KSLVNPGIAMATHKDDEERFFPQHGVLVFGKTNATNKTTLENVLVTDEEEVKATNPVA
TEEYGTVSSNLQSNTTNPTTETVNNQGILPGMVWQDRDVYLQGPIWAKIPHTDGHFHPS
PLMGGFGLKHPPPQILIKNTPVPANPPETFTTSKFASYITQYSTGQVSVEIEWELQKENSK
RWNPEIQYTSNYAKSNNVEFSVDAAGVYSEPRPIGTRYLTRNL.

In other embodiments, the VP1 sequence (from baboon; denoted as Bba44) comprises the amino acid sequence of SEQ ID NO:18 (amino acids 1-739); or comprises an amino acid sequence that is at least 60%, at least 65%, at least 70%, at least 75%, at least 80%, at least 85%, at least 90%, or at least 95% identical over the full length of the amino acid sequence of SEQ ID NO:18. The VP2 capsid protein spans amino acids 138-739 of SEQ ID NO:18 and the VP3 capsid protein spans amino acids 206-739 of SEQ ID NO:18.

(SEQ ID NO: 18)
MAADGYLPDWLEDNLSEGIREWWALKPGAPQPKANQQHQDNSRGLVLPGY
KYLGPFNGLDKGEPVNEADAAALEHDKAYDKQLEQGDNPYLKYNHADAEFQERLQED
TSFGGNLGRAVFQAKKRILEPLGLVEEAAKTAPGKKRPLEKTPNQPTDTQAAGQTPAKK
RPQGEQSGDSARRQLDFDPQPAAPIGQPPAAPSPVGSNTMASGGGGPMADDNQGADGV
GNASGNWHCDSTWLGDRVITTSTRTWVLPTYNNHIYKQISSESGATNDNHYFGYSTPW
GYFDFNRFHCHFSPRDWQRLINNNWGFRPKRLNFKIFNIQVKEVTQIEGGSTIANNLTSTI
QVFADSEYQLPYVLGSAHQGCLPPFPADVFMIPQYGYLTLNNGSQSVGRSSFYCLEYFPS
QMLRTGNNFQFSYTFESVPFHSSYAHSQSLDRIMNPLVDQYLYYLARTQTGTGSTTSNT
RQLQFYQAGPSNMADQSRNWLPGPMYRQQRVSKTLDQDSNTNFAWTAASKYNLNGR
KSLVNPGIAMATHKDDEERFFPQHGVLVFGKTNATNKTTLENVLVTDEEEVKATNPVA
TEEYGTVSSNLQSNTTNPTTETVNNQGILPGMAWQDRDVYLQGPIWAKIPHTDGHFHPS
PLMGGFGLKHPPPQILIKNTPVPANPPETFTTSKFASYITQYSTGQVSVEIEWELQKENSK
RWNPEIQYTSNYAKSNNVEFSVDAAGVYSEPRPIGTRYLTRNL.

In one embodiment, the VP1 sequence (from crab-eating macaque; denoted as Bce14) comprises the amino acid sequence of SEQ ID NO:19 (amino acids 1-736); or comprises an amino acid sequence that is at least 60%, at least 65%, at least 70%, at least 75%, at least 80%, at least 85%, at least 90%, or at least 95% identical over the full length of the amino acid sequence of SEQ ID NO:19. The VP2 capsid protein spans amino acids 138-736 of SEQ ID NO:19 and the VP3 capsid protein spans amino acids 203-736 of SEQ ID NO:19.

(SEQ ID NO: 19)
MAADGYLPDWLEDNLSEGIREWWALKPGAPQPKANQQKQDDGRGLVLPGY
KYLGPFNGLDKGEPVNEADAAALEHDKAYDQQLKAGDNPYLRYNHADAEFQERLQED
TSFGGNLGRAVFQAKKRVLEPLGLVEEAAKTAPGKKRPVEQSPQEPDSSAGIGKSGAQP
AKKRLNFGQTGDTESVPDPQPIGEPPAAPSGVGSLTMASGGGAPVADNNEGADGVGSSS
GNWHCDSQWLGDRVITTSTRTWALPTYNNHLYKQISNSTSGGSSNDNAYFGYSTPWGY
FDFNRFHCHFSPRDWQRLINNNWGFRPKRLNFKLFNIQVKEVTDNNGVKTIANNLTSTV
QVFTDSDYQLPYVLGSAHEGCLPPFPADVFMIPQYGYLTLNDGSQAVGRSSFYCLEYFP
SQMLRTGNNFTFSYEFENVPFHSSYAHSQSLDRLMNPLIDQYLYYLSKTINGSGQNQQA
LKFSVAGPSNMAVQGRNYIPGPSYRQQRVSTTVTQNNNSEFAWPGASSWALNGRNSLM
NPGPAMASHKEGEDRFFPLSGSLIFGKQGTGRDNVDADKVMITNEEEIKTTNPVATESY
GQVATNHQSAQAQAQTGWVQNQGILPGMVWQDRDVYLQGPIWAKIPHTDGNFHPSPL
VGGFGMKHPPPQILIKNTPVPADPPTAFNKDKLNSFITQYSTGQVRVEIEWELQKENSKR
WNPEIQYTSNYYKSNNVEFAVNTEGVYSEPRPIGTRYLTRNL.

In another embodiment, the VP1 sequence (from crab-eating macaque; denoted as Bce15) comprises the amino acid sequence of SEQ ID NO:20 (amino acids 1-736); or comprises an amino acid sequence that is at least 60%, at least 65%, at least 70%, at least 75%, at least 80%, at least 85%, at least 90%, or at least 95% identical over the full length of the amino acid sequence of SEQ ID NO:20. The VP2 capsid protein spans amino acids 138-736 of SEQ ID NO:20 and the VP3 capsid protein spans amino acids 203-736 of SEQ ID NO:20.

(SEQ ID NO: 20)
MAADGYLPDWLEDNLSEGIREWWALKPGAPQPKANQQKQDDGRGLVLPGY
KYLGPFNGLDKGEPVNEADAAALEHDKAYDQQLKAGDNPYLRYNHADAEFQERLQED
TSFGGNLGRAVFQAKKRVLEPLGLVEEAAKTAPGKKRPVEQSPQEPDSSAGIGKSGAQP
AKKRLNFGQTGDTESVPDPQPIGEPPAAPSGVGSLTMASGGGAPVADNNEGADGVGSSS
GNWHCDSQWLGDRVITTSTRTWALPTYNNHLYKQISNSTSGGSSNDNAYFGYSTPWGY
FDFNRFHCHFSPRDWQRLINNNWGFRPKRLNFKLFNIQVKEVTDNNGVKTIANNLTSTV
QVFTDSDYQLPYVLGSAHEGCLPPFPADVFMIPQYGYLTLNDGSQAVGRSSFYCLEYFP
SQMPRTGNNFTFSYEFENVPFHSSYAHSQSLDRLMNPLIDQYLYYLSKTINGSGQNQQTL
KFSVAGPSNMAVQGRNYIPGPSYRQQRVSTTVTQNNNSEFAWPGASSWALNGRNSLM
NPGPAMASHKEGEDRFFPLSGSLIFGKQGTGRDNVDADKVMITNEEEIKTTNPVATESY
GQVATNHQSAQAQAQTGWVQNQGILPGMVWQDRDVYLQGPIWAKIPHTDGNFHPSPL
MGGFGMKHPPPQILIKNTPVPADPPTAFNKDKLNSFITQYSTGQVSVAIEWELQKENSKR
WNPEIQYTSNYYKSNNVEFAVNTEGVYSEPRPIGTRYLTRNL.

In another embodiment, the VP1 sequence (from crab-eating macaque (denoted as Bce16)) comprises the amino acid sequence of SEQ ID NO:21 (amino acids 1-736); or comprises an amino acid sequence that is at least 60%, at least 65%, at least 70%, at least 75%, at least 80%, at least 85%, at least 90%, or at least 95% identical over the full length of the amino acid sequence of SEQ ID NO:21. The VP2 capsid protein spans amino acids 138-736 of SEQ ID NO:21 and the VP3 capsid protein spans amino acids 203-736 of SEQ ID NO:21.

(SEQ ID NO: 21)
MAADGYLPDWLEDNLSEGIREWWALKPGAPQPKANQQKQDDGRGLVLPGY
KYLGPFNGLDKGEPVNEADAAALEHDKAYDQQLKAGDNPYLRYNHADAEFQERLQED
TSFGGNLGRAVFQAKKRVLEPLGLVEEAAKTAPGKKRPVEQSPQEPDSSAGIGKSGAQP
AKKRLNFGQTGDTESVPDPQPIGEPPAAPSGVGSLTMASGGGAPVADNNEGADGVGSSS
GNWHCDSQWLGDRVITTSTRTWALPTYNNHLYKQISNSTSGGSSNDNAYFGYSTPWGY
FDFNRFHCHFSPRDWQRLINNNWGFRPKRLNFKLFNIQVKEVTDNNGVKTIANNLTSTV
QVFTDSDYQLPYVLGSAHEGCLPPFPADVFMIPQYGYLTLNDGSQAVGRSSFYCLEYFP
SQMLRTGNNFTFSYEFENVPFHSSYAHSQSLDRPMNPLIDQYLYYLSKTINGSGQNQQTL
KFSVAGPSNMAVQGRNYIPGPSYRQQRVSTTVTQNNNSEFAWPGASSWALNGRNSLM
NPGPAMASHKEGEDRFFPLSGSLIFGKQGTGRDNVDADKVMITNEEEIKTTNPVATESY
GQVATNHQSAQAQAQTGWVQNQGILPGMVWQDRDVYLQGPIWAKIPHTDGNFHPSPL
MGGFGMKHPPPQILIKNTPVPADPPTAFNKDKLNSFITQYSTGQVSVEIEWELQKENSKR
WNPEIQYTSNYYKSNNVEFAVNTEGVYSEPRPIGTRYLTRNL.

In another embodiment, the VP1 sequence (from crab-eating macaque (denoted as Bce17)) comprises the amino acid sequence of SEQ ID NO:22 (amino acids 1-736); or comprises an amino acid sequence that is at least 60%, at least 65%, at least 70%, at least 75%, at least 80%, at least 85%, at least 90%, or at least 95% identical over the full length of the amino acid sequence of SEQ ID NO:22. The VP2 capsid protein spans amino acids 138-736 of SEQ ID NO:22 and the VP3 capsid protein spans amino acids 203-736 of SEQ ID NO:22.

(SEQ ID NO: 22)
MAADGYLPDWLEDNLSEGIREWWALKPGAPQPKANQQKQDDGRGLVLPGY
KYLGPFNGLDKGEPVNEADAAALEHDKAYDQQLKAGDNPYLRYNHADAEFQERLQED
TSFGGNLGRAVFQAKKRVLEPLGLVEEAAKTAPGKKRPVEQSPQEPDSSAGIGKSGAQP
AIKRLNFGQTGDTESVPDPQPIGEPPAAPSGVGSLTMASGGGAPVADNNEGADGVGSSS
GNWHCDSQWLGDRVITTSIRTWALPTYNNHLYKQISNSTSGGSSNDNAYFGYSTPWGY
FDFSRFHCHFSPRDWQRLINNNWGFRPKRLNFKLFNIQVKEVTDNNGVKTIANNLTSTV
QVFTDSDYQLPYVLGSAHEGCLPPFPADVFMIPQYGYLTLNDGSQAVGRSSFYCLEYFP
SQMLRTGNNFTFSYEFENVPFHSSYAHSQSLDRLMNPLIDQYLYYLSKTINGSGQNQQT
LKFSVAGPSNMAVQGRNYIPGPSYRQQRVSTTVTQNNNSEFAWPGASSWALNGRNSLM
NPGPAMASHKEGEDRFFPLSGSLIFGKQGTGRDNVDADKVMITNEEEIKTTNPVATESY
GQVATNHQSAQAQAQTGWVQNQGILPGMVWQDRDVYLQGPIWAKIPHTDGNFHPSPL
MGGFGMKHPPPQILIKNTPVPADPPTAFNKDKLNSFITQYSTGQVSVEIEWELQKENSKR
WNPEIQYTSNYYKSNNVEFAVNTEGVYSEPRPIGTRYLTRNL.

In another embodiment, the VP1 sequence (from crab-eating macaque (denoted as Bce18)) comprises the amino acid sequence of SEQ ID NO:23 (amino acids 1-736); or comprises an amino acid sequence that is at least 60%, at least 65%, at least 70%, at least 75%, at least 80%, at least 85%, at least 90%, or at least 95% identical over the full length of the amino acid sequence of SEQ ID NO:23. The VP2 capsid protein spans amino acids 138-736 of SEQ ID NO:23 and the VP3 capsid protein spans amino acids 203-736 of SEQ ID NO:23.

(SEQ ID NO: 23)
MAADGYLPDWLEDNLSEGIREWWALKPGAPQPKANQQKQDDGRGLVLPGY
KYLGPFNGLDKGEPVNEADAAALEHDKAYDQQLKAGDNPYLRYNHADAEFQERLQED
TSFGGNLGRAVFQAKKRVLEPLGLVEEAAKTAPGKKRPVEQSPQEPDSSAGIGKSGAQP
AKKRLNFGQTGDTESVPDPQPIGEPPAAPSGVGSLTMASGGGAPVADNNEGADGVGSSS
GNWHCDSQWLGDRVITTSTRTWALPTYNNHLYKQISNSTSGGSSNDNAYFGYSTPWGY
FDFNRFHCHFSPRDWQRLINNNWGFRPKRLNFKLFNIQVKEVTDNNGVKTIANNLTSTV
QVFTDSDYQLPYVLGSAHEGCLPPFPADVFMIPQYGYLTLNDGSQAVGRSSFYCLEYFP
SQMLRTGNNFTFSYEFENVPFHSSYAHSQSLDRLMNPLIDQYLYYLSKTINGSGQNQQT
LKFSVAGPSNMAVQGRNYIPGPSYRQQRVSTTVTQNNNSEFAWPGASSWALNGRNSLM
NPGPAMASHKEGEDRFFPLSGSLIFGKQGTGRDNVDADKVMITNEEEIKTTNPVATESY
GQVATNHQSAQAQAQTGWVQNQGILPGMVWQDRDVYLQGPIWAKIPHTDGNFHPSPL
MGGFGMKHPPPQILIKNTPVPADPPTAFNKDKLNSFITQYSTGQVSVEIEWELQKENSKR
WNPEIQYTSNYYKSNNVEFAVNTEGVYSEPRPIGTRYLTRNL.

In another embodiment, the VP1 sequence (from crab-eating macaque (denoted as Bce20)) comprises the amino acid sequence of SEQ ID NO:24 (amino acids 1-733); or comprises an amino acid sequence that is at least 60%, at least 65%, at least 70%, at least 75%, at least 80%, at least 85%, at least 90%, or at least 95% identical over the full length of the amino acid sequence of SEQ ID NO:24. The VP2 capsid protein spans amino acids 138-733 of SEQ ID NO:24 and the VP3 capsid protein spans amino acids 203-733 of SEQ ID NO:24.

(SEQ ID NO: 24)
MAADGYLPDWLEDNLSEGIREWWALKPGAPQPKANQQKQDDGRGLVLPGY
KYLGPFNGLDKGEPVNEADAAALEHDKAYDQQLKAGDNPYLRYNHADAEFQERLQED
TSFGGNLGRAVFQAKKRVLEPLGLVEEAAKTAPGKKRPVEQSPQEPDSSAGIGKSGAQP
AKKRLNFGQTGDTESVPDPQPIGEPPAAPSGVGSLTMASGGGAPVADNNEGADGVGSSS
GNWHCDSRWLGDRVITTSTRTWALPTYNNHLYKQISNSTSGGSSNDNAYFGYSTPWGY
FDFNRFHCHFSPRDWQRLINNNWGFRPKRLNFKLFNIQVKEVTDNNGAKTIANNLTSTV
QVFTDSDYQLPYVLGSAHEGCLPPFPADVFMIPQYGYLTLNDGSQAVGRSSFYCLEYFP
SQMLRTGNNFTFSYEFENVPFHSSYAHSQSLDRLMNPLIDQYLYYLSKTINGSGQNQQT
LKFSVAGPSNMAVQGRNYIPGPSYRQQRVSTTVTQNNNSEFAWPGASSWALNGRNSLM
NPGPAMASHKEGEDRFFPLSGSLIFGKQGTGRDNVDADKVMITNEEEIKTTNPVATESY
GQVATNHQSAQAQAQTGWVQNQGILPGMVWQDRDVYLQGPIWAKIPHTDGNFHPSPL
MGGFGMKHPPPQILIKNTPVPADPPTAFNKDKLNSFITQYSTGQVSVEIEWELQKENSKR
WNPEIQYTSNYYKSNNVEFAVNTEGVYSEPRPIGTRYLT.

In another embodiment, the VP1 sequence (from crab-eating macaque (denoted as Bce35) comprises the amino acid sequence of SEQ ID NO:25 (amino acids 1-730); or comprises an amino acid sequence that is at least 60%, at least 65%, at least 70%, at least 75%, at least 80%, at least 85%, at least 90%, or at least 95% identical over the full length of the amino acid sequence of SEQ ID NO:25. The VP2 capsid protein spans amino acids 138-730 of SEQ ID NO:25 and the VP3 capsid protein spans amino acids 199-730 of SEQ ID NO:25.

(SEQ ID NO: 25)
MAADGYLPDWLEDNLSEGIREWWALKPGAPQPKANQQKQDDGRGLVLPGY
KYLGPFNGLDKGEPVNEADAAALEHDKAYDQQLKAGDNPYLRYNHADAEFQERLQED
TSFGGNLGRAVLQAKKRVLEPLGLVEEAAKTAPGKKRPVDSPDSTSGIGKKGQQPARK
RLNFGQTGDAESVPDPQPIGEPPAAPSGLGSGTMAAGGGAPMADNNEGADGVGNASG
NWHCDSTWLGNRVITTSTRTWALPTYNNHLYKQISSSSSGATNDNHYFGYSTPWGYFD
FNRFHCHFSPRDWQRLINNNWGFRPKRLRFKLFNIQVKEVTTNDGVTTIANNLTSTVQV
FSDSEYQLPYVLGSAHQGCLPPFPADVFMIPQYGYLTLNNGSQSVGRSSFYCLEYFPSQM
LRTGNNFEFSYEFEDVPFHSSYAHSQSLDRLMNPLIDQYLYYLARTQSSTGTSTRELQFH
QAGPATMAEQSKNWLPGPCFRQQRISKTTDNNNNSNFAWTGATKYHLNGRSSLTNPGV
PMATHKDDESVFFPINGVLVFGKTGASNKTTLENVLMTDEEEIKATNPVATEEYGVVSS
NIQSQNSNPTTQTVNNQGALPGMVWQNRDVYLQGPIWAKIPHTDGNFHPSPLMGGFGL
KHPPPQILIKNTPVPANPPETFTPAKFASFITQYSTGQVSVEIEWELQKENSKRWNPEIQY
TSNYDKQTGVGFAVDTQGVYSEPRPIGTRYLTRNL.

In another embodiment, the VP1 sequence (from crab-eating macaque (denoted as Bce36)) comprises the amino acid sequence of SEQ ID NO:26 (amino acids 1-730); or comprises an amino acid sequence that is at least 60%, at least 65%, at least 70%, at least 75%, at least 80%, at least 85%, at least 90%, or at least 95% identical over the full length of the amino acid sequence of SEQ ID NO:26. The VP2 capsid protein spans amino acids 138-730 of SEQ ID NO:26 and the VP3 capsid protein spans amino acids 199-730 of SEQ ID NO:26.

(SEQ ID NO: 26)
MAADGYLPDWLEDNLSEGIREWWALKPGAPQPKANQQKQDDGRGLVLPGY
KYLGPFNGLDKGEPVNEADAAALEHDKAYDQQLKAGDNPYLRYNHADAEFQERLQED
TSFGGNLGRAVFQAKKRVLEPLGLVEEAAKTAPGKKRPVDSPDSTSGIGKKGQQPARK
RLNFGQTGDAESVPDPQPIGEPPAAPSGLGSGTMAAGGGAPMADNNEGADGVGNASG
NWHCDSTWLGNRVITTSTRTWALPTYNNHLYKQISSSSSGATNDNHYFGYSTPWGYFD
FNRFHCHFSPRDWQRLINNNWGFRPKRLRFKLFNIQVKEVTTNDGVTTIANNLTSTVQV
FSDSEYQLPYVLGSAHQGCLPPFPADVFMIPQYGYLTLNNGSQSVGRSSFYCLEYFPSQM
LRTGNNFEFSYEFEDVPFHSSYAHSQSLDRLMNPLIDQYLYYLARTQSSTGTSTRELQFH
QAGPATMAEQSKNWLPGPCFRQQRISKTTDNNNNSNFAWTGATKYHLNGRNSLTNPG
VPMATHKDDESVFFPINGVLVFGKTGASNKTTLENVLMTDEEEIKATNPVATEEYGVVS
SNIQSQNSNPTTQTVNNQGALPGMVWQNRDVYLQGPIWAKIPHTDGNFHPSPLMGGFG
LKHPPPQILIKNTPVPANPPETFTPAKFASFITQYSTGQVSVEIEWELQKENSKRWNPEIQ
YTSNYDKQTGVDFAVDTQGVYSEPRPIGTRYLTRNL.

In another embodiment, the VP1 sequence (from crab-eating macaque (denoted as Bce39)) comprises the amino acid sequence of SEQ ID NO:27 (amino acids 1-736); or comprises an amino acid sequence that is at least 60%, at least 65%, at least 70%, at least 75%, at least 80%, at least 85%, at least 90%, or at least 95% identical over the full length of the amino acid sequence of SEQ ID NO:27. The VP2 capsid protein spans amino acids 138-736 of SEQ ID NO:27 and the VP3 capsid protein spans amino acids 203-736 of SEQ ID NO:27.

(SEQ ID NO: 27)
MAADGYLPDWLEDNLSEGIREWWDLKPGAPKPKANQQKQDDGRGLVLPGY
KYPGPFNGLDKGEPVNAADAAALEHDKAYDQQLKAGDNPYLRYNHADAEFQERLQED
TSFGGNLGRAVFQAKKRVFEPLGLVEEGAKTAPGKKRPVEQSPQEPDSSSGIGKTGQQP
AKKRLNFGQTGDSESVPDPQPLGEPPAAPSGLGPNTMASGGGAPMADNNEGADGVGNS
SGNWHCDSTWLGDRVITTSTRTWALPTYNNHLYKQISNSTSGGSTNDNTYFGYSTPWG
YFDFNRFHCHFSPRDWQRLINNNWGFRPKRLNFKLFNIQVKEVTTNEGTKTIANNLTST
VQVFTDSEYQLPYVLGSAHQGCLPPFPADVFMVPQYGYLTLNNGSQALGRSSFYCLEYF
PSQMLRTGNNFQFSYTFEDVPFHSSYAHSQSLDRLMNPLIDQYLYYLVRTQTTGTGGTQ
TLAFSQAGPSSMANQARNWVPGPSYRQQRVSTTKNQNNNSNFAWTGAAKFKLNGRNS
LMNPGVAMASHKDDEDRFFPSSGVLIFGKQGAGNDGVDYSQVLITDEEEIKATNPVATE
EYGEVAINDQAANTQAQTGLVHNQGVIPGMVWQNRDVYLQGPIWAKIPHTDGNFHPSP
LMGGFGLMHPPPQILIKNTPVPADPPLTFNQAKLNSFITQYSTGQVSVEIEWELQKENSK
RWNPEIQYTSNYYKSTNVDFAVNSDGVYSEPRPIGTRYLTRNL.

In another embodiment, the VP1 sequence (from crab-eating macaque (denoted as Bce40)) comprises the amino acid sequence of SEQ ID NO:28 (amino acids 1-736); or comprises an amino acid sequence that is at least 60%, at least 65%, at least 70%, at least 75%, at least 80%, at least 85%, at least 90%, or at least 95% identical over the full length of the amino acid sequence of SEQ ID NO:28. The VP2 capsid protein spans amino acids 138-736 of SEQ ID NO:28 and the VP3 capsid protein spans amino acids 203-736 of SEQ ID NO:28.

(SEQ ID NO: 28)
MAADGYLPDWLEDNLSEGIREWWDLKPGAPKPKANQQKQDDGRGLVLPGY
KYLGPFNGLDKGEPVNAADAAALEHDKAYDQQLKAGDNPYLRYNHADAEFQGRLQE
DTSFGGNLGRAVFQAKKRVLEPLGLVEEGAKTAPGKKRPVEQSPQEPDSSSGIGKTGQQ
PAKKRLNFGQTGDSESVPDPQPLGEPPAAPSGLGPNTMASGGGAPMADNNEGADGVGN
SSGNWHCDSTWLGDRVITTSTRTWALPTYNNHLYKQISNSTSGGSINDNTYFGYSTPW
GYFDFNRFHCHFSPRDWQRLINNNWGFRPKRPNFKLFNIQVKEVTTNEGTKTIANNLTS
TVQAFTDSEYQLPYVLGSAHQGCLPPFPADVFMVPQYGYLTLNNGSQALGRSSFYCLEY
FPSQMLRTGNNFQFSYTFEDVPFHSSYAHSQSLDRLMNPLIDQYLYYLVRTQTTGTGGT
QTLAFSQAGPSSMANQARNWVPGPSYRQQRVSTTKNQNNNSNFAWTGAAKFKLNGRN
SLMNPGVAMASHKDDEDRFSPSSGVLIFGKQGAGNDGVDYSQVLITDEEEIKATNPVAT
EEYGEVAINDQAANTQAQTGLVHNQGVIPGMVWQNRDVYLQGPIWAKIPHTDGNFHPS
PLMGGFGLKHPPPQILIKNTPVPADPPLTFNQAKLNSFITQYSTGQVSVEIEWELQKENSK
RWNPEIQYTSNYYKSTNVDFAVNSDGVYSEPRPIGTRYLTRNL.

In another embodiment, the VP1 sequence (from crab-eating macaque (denoted as Bce41)) comprises the amino acid sequence of SEQ ID NO:29 (amino acids 1-736); or comprises an amino acid sequence that is at least 60%, at least 65%, at least 70%, at least 75%, at least 80%, at least 85%, at least 90%, or at least 95% identical over the full length of the amino acid sequence of SEQ ID NO:29. The VP2 capsid protein spans amino acids 138-736 of SEQ ID NO:29 and the VP3 capsid protein spans amino acids 203-736 of SEQ ID NO:29.

(SEQ ID NO: 29)
MAADGYLPDWLEDNLSEGIREWWDLKPGAPKPKANQQKQDDGRGLVLPGY
KYLGPFNGLDKGEPVNAADAAALEHDKAYDQQLKAGDNPYLRYNHADAEFQGRLQE
DTSFGGNLGRAVFQAKKRVLEPLGLVEEGAKTAPGKKRPVEQSPQEPDSSSGIGKTGQQ
PAKKRLNFGQTGDSESVPDPQPLGEPPAAPSGLGPNTMASGGGAPMADNNEGADGVGN
SSGNWHCDSTWLGDRVITTSTRTWALPTYNNHLYKQISNSTSGGSTNDNTYFGYSTPW
GYFDFNRFHCHFSPRDWQRLINNNWGFRPKRLNFKLFNIQVKEVTTNEGTKTIANNLTS
TVQVFTDSEYQLPYVLGSAHQGCLPPFPADVFMVPQYGYLTLNNGSQALGRSSFYCLEY
FPSQMLRTGNNFQFSYTFEDVPFHSSYAHSQSLDRLMNPLIDQYLYYLVRTQTTGTGGT
QILAFSQAGPSSMANQARNWVPGPSYRQQRVSTTKNQNNNSNFAWTGAAKFKLNGRN
SLMNPGVAMASHKDDEDRFFPSSGVLIFGKQGAGNDGMDYSQVLITDEEEIKATNPVAT
EEYGEVAINDQAANTQAQTGLVHNQGVIPGMVWQNRDVYLQGPIWAKIPHTDGNFHPS
PLMGGFGLKHPPPQILIKNTPVPADPPLTFNQAKLNSFITQYSTGQVSVEIEWELQKENSK
RWNPEIQYTSNYYKSTNVDFAVNSDGVYSEPRPIGTRYLTRNL.

In another embodiment, the VP1 sequence (from crab-eating macaque (denoted as Bce42)) comprises the amino acid sequence of SEQ ID NO:30 (amino acids 1-736); or comprises an amino acid sequence that is at least 60%, at least 65%, at least 70%, at least 75%, at least 80%, at least 85%, at least 90%, or at least 95% identical over the full length of the amino acid sequence of SEQ ID NO:30. The VP2 capsid protein spans amino acids 138-736 of SEQ ID NO:30 and the VP3 capsid protein spans amino acids 203-736 of SEQ ID NO:30.

(SEQ ID NO: 30)
MAADGYLPDWLEDNLSEGIREWWDLKPGAPKPKANQQKQDDGRGLVLPGY
KYLGPFNGLDKGEPVNAADAAALEHDKAYDQQLKAGDNPYLRYNHADAEFQERLQED
TSFGGNLGRAVFQAKKRVLEPLGLVEEGAKTAPGKKRPVEQSPQEPDSSSGIGKTGQQP
AKKRLNFGQTGDSESVPDPQPLGEPPAAPSGLGPNTMASGGGAPMADNNEGADGVGNS
SGNWHCDSTWLGDRVITTSTRTWALPTYNNHLYKQISNSTSGGGTNDNTYFGYSTPWG
YFDFNRFHCHFSPRDWQRLINNNWGFRPKRLNFKLFNIQVKEVTTNEGTKTIANNLTST
VQVFTDSEYQLPYVLGSAHQGCLPPFPADVFMVPQYGHLTLNNGSQALGRSSFYCLEYF
PSQMLRTGNNFQFSYTFEDVPFHSSYAHSQSLDRLMNPLIDQYLYYLVRTQTTGTGGTQ
TLAFSQAGPSSMANQARNWVPGPSYRQQRVSTTKDQNNNSNFAWTGAAKFKLNGRNS
LMNPGVAMASHKDDEDRFFPSSGVLIFGKQGAGNDGVDYSQVLITDEEEIKATNPVATE
EYGEVAVNDQAANTQAQTGLVHNQGVIPGMVWQNRDVYLQGPIWAKIPHTDGNFHPS
PLMGGFGLKHPPPQILIKNTPVPADPPLTLNQAKLNSFITQYSTGQVSVEIEWELQKENSK
RWNPEIQYTSNYYKSTNVDFAVNSDGVYSEPRPIGTRYLTRNL.

In another embodiment, the VP1 sequence (from crab-eating macaque (denoted as Bce43)) comprises the amino acid sequence of SEQ ID NO:31 (amino acids 1-730); or comprises an amino acid sequence that is at least 60%, at least 65%, at least 70%, at least 75%, at least 80%, at least 85%, at least 90%, or at least 95% identical over the full length of the amino acid sequence of SEQ ID NO:31. The VP2 capsid protein spans amino acids 138-730 of SEQ ID NO:31 and the VP3 capsid protein spans amino acids 199-730 of SEQ ID NO:31.

(SEQ ID NO: 31)
MAADGYLPDWLEDNLSEGIREWWALKPGAPQPKANQQKQDDGRGLVLPGY
KYLGPFNGLDKGEPVNEADAAALEHDKAYDQQLKAGDNPYLRYNHADAEFQERLQED
TSFGGNLGRAVFQAKKRVLEPLGLVEEAAKTAPGKKRPVDSPDSTSGIGKKGQQPARK
RLNFGQTGDAESVPDPQPIGEPPAAPSGLGSGTMAAGGGAPMADNNEGADGVGNAPG
NWHCDSTWLGNRVITTSTRTWALPTYNNHLYKQISSSSSGATNDNHYFGYSTPWGYFD
FNRFHCHFSPRDWPRLINNNWGFRPKRLRFKLFNIQVKEVTTNDGVTTIANNLTSTVQV
FSDSEYQLPYVLGSAHQGCLPPFPADVFMIPQYGYLTLNNGSQSVGRSSFYCLEYFPSQM
LRTGNNFEFSYEFEDVPFHSSYAHSQSLDRLMNPLIDQYPYYLARTQSSTGTSTRELQFH
QAGPATMAEQSKNWLPGPCFRQQRISKTTDNNNNSNFAWTGATKYHLNGRNSLTNPG
VPMATHKDDESVFFPINGVLVFGKTGASNKTTLENVLMTDEEEIKATNPVATEEYGVVS
SNIQSQNSNPATQTVNNQGALPGMVWQNRDVYLQGPIWAKIPHTDGNFHPSPLMGGFG
LKHPPPQILIKNTPVPANPPETFTPAKFASFITQYSTGQVSVEIEWELQKENSKRWNPEIQ
YTSNYDKQTGVDFAVDTQGVYSEPRPIGTRYLTRNL.

In another embodiment, the VP1 sequence (from crab-eating macaque (denoted as Bce44)) comprises the amino acid sequence of SEQ ID NO:32 (amino acids 1-730); or comprises an amino acid sequence that is at least 60%, at least 65%, at least 70%, at least 75%, at least 80%, at least 85%, at least 90%, or at least 95% identical over the full length of the amino acid sequence of SEQ ID NO:32. The VP2 capsid protein spans amino acids 138-730 of SEQ ID NO:32 and the VP3 capsid protein spans amino acids 199-730 of SEQ ID NO:32.

(SEQ ID NO: 32)
MAADGYLPDWLEDNLSEGIREWWALKPGAPQPKANQQKQDDGRGLMLPG
YKYLGPFNGLDKGEPVNEADAAALEHDKAYDQQLKAGDNPYLRYNHADAEFQERLQE
DTSFGGNLGRAVFQAKKRVLEPLGLVEEAAKTAPGKKRPVDSPDSTSGIGKKGQQPAR
KRLNFGQTGDAESVPDPQPIGEPPAAPSGLGSGTMAAGGGAPMADNNEGADGVGNAS
GSWHCDSTWLGNRVITTSTRTWALPTYNNHLYKQISSSSSGATNDNHYFGYSTPWGYF
DFNRFHCHFSPRDWQRLINNNWGFRPKRLRFKLFNIQVKEVTTNDGVTTIANNLTSTVQ
VFSDSEYQLPYVLGSAHQGCLPPFPADVFMIPQYGYLTLNNGRQSVGRSSFYCLEYFPSQ
MLRTGNNFEFSYEFEDVPFHSSYAHSQSLDRLMNPLIDQYPYYLARTQSSTGTSTRELQF
HQAGPATMAEQSKNWLPGPCFRQQRISKTTDNNNNSNFAWTGATKYHLNGRNSLTNP
GVPMATHKDDESVFFPINGVLVFGKTGASNKTTLENVPMTDEEEIKATNPVATEEYGVV
SSNIQSQNSNPTTQTVNNQGALPGMVWQNRDVYLQGPIWAKIPHTDGNFHPSPLMGGF
GLKHPPPQILIKNTPVPANPPETFTPAKFASFITQYSTGQVSVEIEWELQKENNKRWNPEI
QYTSNYDKQTGVDFAVDTQGVYSEPRPIGTRYLTRNL.

In another embodiment, the VP1 sequence (from crab-eating macaque (denoted as Bce45)) comprises the amino acid sequence of SEQ ID NO:33 (amino acids 1-730); or comprises an amino acid sequence that is at least 60%, at least 65%, at least 70%, at least 75%, at least 80%, at least 85%, at least 90%, or at least 95% identical over the full length of the amino acid sequence of SEQ ID NO:33. The VP2 capsid protein spans amino acids 138-730 of SEQ ID NO:33 and the VP3 capsid protein spans amino acids 199-730 of SEQ ID NO:33.

(SEQ ID NO: 33)
MAADGYLPDWLEDNLSEGIREWWALKPGAPQPKANQQKQDDGRGLVLPGY
KYLGPFSGLDKGEPVNEADAAALEHDKAYDQQLKAGDNPYLRYNHADAEFQERLQED
TSFGGNLGRAVFQAKKRVLEPLGLVEEAAKTAPGKKRPVDSPDSTSGIGKKGQQPARK
RLNFGQTGDAESVPDPQPIGEPPAAPSGLGSGTMAAGGGAPMADNNEGADGVGNASG
NWHCDSTWLGNRVITTSTRTWALPTYNDHLYKQISSSSSGATNDNHYFGYSTPWGYFD
FNRFHCHFSPRDWQRLINNNWGFRPKRLRFKLFNIQVKEVTTNDGVTTIANNLTSTVQV
FSDSEYQLPYVLGSAHQGCLPPFPADVFMIPQYGYLTLNNGSQSVGRSSFYCLEYFPSQM
LRTGNNFEFSYEFEDVPFHSSYAHSQSLDRLMNPLIDQYLYYLARTQSSTGTSTRELQFH
QAGPATMAEQSKNWLPGPCFRQQRISKTTDNNNNSNFAWTGATKYHLNGRNSLTNPG
VPMATHKDDESVFFPINGVLVFGKTGASNKTTLENVLMTDEEEIKATNPVATEEYGVVS
SNIQSQNSNPTTQTVNNQGALPGMVWRNRDVYLQGPIWAKIPHTDGNFHPSPLMGGFG
LKHPPPQILIKNTPVPANPPETFTPAKFASFITQYSTGQVSVEIEWELQKENSKRWNPEIQ
YTSNYDKQTGVDFAVDTQGVYSEPRPIGTRYLTRNL.

In another embodiment, the VP1 sequence (from crab-eating macaque (denoted as Bce46)) comprises the amino acid sequence of SEQ ID NO:34 (amino acids 1-730); or comprises an amino acid sequence that is at least 60%, at least 65%, at least 70%, at least 75%, at least 80%, at least 85%, at least 90%, or at least 95% identical over the full length of the amino acid sequence of SEQ ID NO:34. The VP2 capsid protein spans amino acids 138-730 of SEQ ID NO:34 and the VP3 capsid protein spans amino acids 199-730 of SEQ ID NO:34.

(SEQ ID NO: 34)
MAADGYLPDWLEDNLSEGIREWWALKPGAPQPKANQQKQDDGRGLVLPGY
KYLGPFNGLDKGEPVNEADAAALEHDKAYDQQLKAGDNPYLRYNHADAEFQERLQED
TSFGGNLGRAVFQAKKRVLEPLGLVEEAAKTAPGKKRPVDSPDSTSGIGKKGQQPARK
RLNFGQTGDAESVPDPQPIGEPPAAPSGLGSGTMAAGGGAPMADNNEGADGVGNASG
NWHCDSTWLGNRVITTSTRTWALPTYNNHLYKQISSSSSGATNDNHYFGYSTPWGYFD
FNRFHCHFSPRDWQRLINNNWGFRPKRLRFKLFNIQVKEVTTNDGVTTIANNLTSTVQV
FSDSEYQLPYVLGSAHQGCLPPFPADVFMIPQYGYLTLNNGSQSVGRSSFYCLEYFPSQM
LRTGNNFEFSYEFEDVPFHSSYAHSQSLDRLMNPLIDQYLYYMARTQSSTGTSTREPQFH
QAGPATMAEQSKNWLPGPCFRQQRISKTTDNNNNSNFAWTGATKYHLNGRNSLTNPG
VPMATHKDDESVFFPINGVLVFGKTGASNKTTLENVLMTDEEEIKATNPVATEEYGVVS
SNIQSQNSNPTTQTVNNQGALPGMVWQNRDVYLQGPIWAKIPHTDGDFHPSPLMGGFG
LKHPPPQILIKNTPVPANPPETFTPAKFASFITQYSTGQVSVEIEWELQKENSKRWNPEIQ
YTSNYDKQTGVDFAVDTQGVYSEPRPIGTRYLTRNL.

In another embodiment, the VP1 sequence (from cynomolgus macaque (denoted as Bey20)) comprises the amino acid sequence of SEQ ID NO:35 (amino acids 1-730); or comprises an amino acid sequence that is at least 60%, at least 65%, at least 70%, at least 75%, at least 80%, at least 85%, at least 90%, or at least 95% identical over the full length of the amino acid sequence of SEQ ID NO:35. The VP2 capsid protein spans amino acids 138-730 of SEQ ID NO:35 and the VP3 capsid protein spans amino acids 199-730 of SEQ ID NO:35.

(SEQ ID NO: 35)
MAADGYLPDWLEDNLSEGIREWWALKPGAPQPKANQQKQDDGRGLVLPGY
KYLGPFNGLDKGEPVNEADAAALEHDKAYDQQLKAGDNPYLRYNHADAEFQERLQED
TSFGGNLGRAVFQAKKRVLEPLGLVEEAAKTAPGKKRPVDSPDSTSGIGKKGQQPARK
RLNFGQTGDAESVPDPQPIGEPPAAPSGLGSGTMAAGGGAPMADNNEGADGVGNASG
NWHCDSTWLGNRVITTSTRTWALPTYNNHLYKQISSGSSGATNDNHYFGYSTPWGYFD
FNRFHCHFSPRDWQRLINNNWGFRPKRLRFKLFNIQVKEVTTNDGVTTIANNLTSTVQV
FSDSEYQLPYVLGSAHQGCLPPFPADVFMIPQYGYLTLNNGSQSVGRSSFYCLEYFPSQV
LRTGNNFEFSYEFEDVPFHSSYAHSQSLDRLMNPLIDQYLYYLARTQSSTGTSTRELQFH
QAGPATMAEQSKNWLPGPCFRQQRISKTSDNNNNSNFAWTGATKYHLNGRNSLTNPG
VPMATHKDDESVFFPINGVLVFGKTGASNKTTLENVLMTDEEEIKATNPVATEEYGVVS
SNIQSQNSNPTTQTVNNQGALPGMVWQNRDVYLQGPIWAKIPHTDGNFHPSPLMGGFG
LKHPPPQILIKNTPVPANPPETFTPAKFASFITQYSTGQVSVEIEWELQKENSKRWNPEIQ
YTSNYDKQTGVDFAVDTQGVYSEPRPIGTRYLTRNL.

In another embodiment, the VP1 sequence (from cynomolgus macaque (denoted as Bey22)) comprises the amino acid sequence of SEQ ID NO:36 (amino acids 1-730); or comprises an amino acid sequence that is at least 60%, at least 65%, at least 70%, at least 75%, at least 80%, at least 85%, at least 90%, or at least 95% identical over the full length of the amino acid sequence of SEQ ID NO:36. The VP2 capsid protein spans amino acids 138-730 of SEQ ID NO:36 and the VP3 capsid protein spans amino acids 199-730 of SEQ ID NO:36.

(SEQ ID NO: 36)
MAADGYLPDWLEDNLSEGIREWWALKPGAPQPKANQQKQDDGRGLVLPGY
RYLGPFNGLDKGEPVNEADAAALEHDKAYDQQLKAGDNPYLRYNHADAEFQERLQED
TSFGGNLGRAVFQAKKRILEPLGLVEEAAKTAPGKERPVDSPDSTSGIGKKGQQPARKR
LNFGQTGDAESVPDPQPIGEPPAAPSGLGSGTMAAGGGAPMADNNEGADGVGNASGN
WHCDSTWLGNRVITTSTRTWALPTYDNHLYKQISSSSSGATNDNHYFGYSTPWGYFDF
NRFHCHFSPRDWQRLINNNWGFRPKRLRFKLFNIQVKEVTTNDGVTTIANNLTSTVQVF
SDSEYQLPYVLGSAHQGCLPPFPADVFMIPQYGYLTLNNGSQSVGRSSFYCLEYFPSQM
LRTGNNFEFSYEFEDVPFHSSYAHSQSLDRLMNPLIDQYLYYLARTQSSTGTSTRELQFH
QAGPATMAEQSKNWLPGPCFRQQRISKTTDNNNNSNFAWTGATKYHLNGRNSLTNPG
VPMATHKDDESVFFPINGVLVLGKTGASNKTTLENVLMTDEEEIKATNPVATEEYGVVS
SNIQSQNSNPTTQTVNNQGALPGMVWQNRDVYLQGPIWAKIPHTDGNFHPSPLMGGFG
LKHPPPQILIKNTPVPANPPETFTPAKFASFITQYSTGQVSVEIEWELQKENSKRWNPEIQ
YTSNYDKQTGVDFAVDTQGVYSEPRPIGTRYLTRNL.

In another embodiment, the VP1 sequence (from cynomolgus macaque (denoted as Bey23)) comprises the amino acid sequence of SEQ ID NO:37 (amino acids 1-730); or comprises an amino acid sequence that is at least 60%, at least 65%, at least 70%, at least 75%, at least 80%, at least 85%, at least 90%, or at least 95% identical over the full length of the amino acid sequence of SEQ ID NO:37. The VP2 capsid protein spans amino acids 138-730 of SEQ ID NO:37 and the VP3 capsid protein spans amino acids 199-730 of SEQ ID NO:37.

(SEQ ID NO: 37)
MAADGYLPDWLEDNLSEGIREWWALKPGAPRPKANQQKQDDGRGLVLPGY
KYLGPFNGLDKGEPVNEADAAALEHDKAYDQQLKAGDNPYLRYNHADAEFQERLQED
TSFGGNLGRAVFQAKKRVLEPLGLVEEAAKTAPGKKRPVDSPDSTSGIGKKGQQPARK
RLNFGQTGDAESVPDPQPIGEPPAAPSGLGSGTMAAGGGAPMADNNEGADGVGNASG
NWHCDSTWLGNRVITTSTRTWALPTYNNHLYKQISSSSSGATNDNHYFGYSTPWGYFD
FNRFHCHFSPRDWQRLINNNWGFRPKRLRFKLFNIQVKEVTTNDGVTTIANNLTSTVQV
FSDSEYQLPYVLGSAHQGCLPPFPADVFMIPQYGYLTLNNGSQSVGRSSFYRLEYFPSQM
LRTGNNFEFSYEFEDVPFHSSYAHSQSLDRLMNPLIDQYLYYLARTQSSTGTSTRELQFH
QAGPATMAEQSKNWLPGPCFRQQRISKTTDNNNNSNFAWTGATKYHLNGRNSLTNPG
VPMATHRDDESVFFPINGVLVFGKTGASNKTTLENVLMTDEEEIKATNPVATEEYGVVS
SNIQSQNSNPTTQTVNNQGALPGMVWQNRDVYLQGPIWAKIPHTDGNFHPSPLMGGFG
LKHPPPQILIKNTPVPANPPETFTPAKFASFITQYSTGQVSVEIEWELQKENSKRWNPEIQ
YTSNYDKQTGVDFAVDTQGVYSEPRPIGTRYLTRNL.

In another embodiment, the VP1 sequence (from marmoset (denoted as Bma42)) comprises the amino acid sequence of SEQ ID NO:38 (amino acids 1-736); or comprises an amino acid sequence that is at least 60%, at least 65%, at least 70%, at least 75%, at least 80%, at least 85%, at least 90%, or at least 95% identical over the full length of the amino acid sequence of SEQ ID NO:38. The VP2 capsid protein spans amino acids 138-736 of SEQ ID NO:38 and the VP3 capsid protein spans amino acids 203-736 of SEQ ID NO:38.

(SEQ ID NO: 38)
MAADGYLPDWLEDNLSEGIREWWDLKPGAPKPKANQQKQDDGRGLVLPGY
KYLGPFNGLDKGEPVNAADAAALEHDKAYDQQLKAGDNPYLRYNHADAEFQERLQED
TSFGGNLGRAVFQAKKRVLEPLGLVEEGAKTAPGKKRPVEQSPQEPDSSSGIGKTGQQP
AKKRLNFGQTGDTESVPDPQPLREPPAAPSGLGPNTMASGGGAPMADNNEGADGVGNS
SGNWHCDSTWLGDRVITTSTRTWALPTYNNHLYKQISNGTSGGSTNDNTYFGYSTPWG
YFDFNRFHCHFSPRDWQRLINNNWGFRPKRLNFKPFNIQVKEVTTNEGTKTIANNPTST
VQVFTDSEYQLPYVLGSARQGCLPPFPADVFMVPQYGYLTLNNGSQALGRSSFYCLEYF
PSQMLRTGDNFQFSYTFEDVPFHSSYAHSQSLDRLMNPLIDQYLYYLVRTQTTGTGGTQ
TLAFSQAGPSSMANQARNWVPGPCYRQQRVSTTTNQNNNSNFAWTGAAKFKLNGRDS
LMNPGVAMASHKDDEDRFFPSSGVLIFGKQGAGNDGVDYSQVLITDEEEIKATNPVATE
EYGAVAINNQAANTLAQTGLVHNQGVIPGMVWQNRDVYLQGPIWAKIPHTDGNFHPSP
LMGGFGLKHPPPQILIKNTPVPADPPLTFNQAKLNSFITQYSTGQVSVEIEWELQKENSKR
WNPEIQYTSNYYKSTNVDFAVNTEGVYSEPRPIGTRYLTRNL.

In another embodiment, the VP1 sequence (from marmoset (denoted as Bma43)) comprises the amino acid sequence of SEQ ID NO:39 (amino acids 1-736); or comprises an amino acid sequence that is at least 60%, at least 65%, at least 70%, at least 75%, at least 80%, at least 85%, at least 90%, or at least 95% identical over the full length of the amino acid sequence of SEQ ID NO:39. The VP2 capsid protein spans amino acids 138-736 of SEQ ID NO:39 and the VP3 capsid protein spans amino acids 203-736 of SEQ ID NO:39.

(SEQ ID NO: 39)
MAADGYLPDWLEDNLSEGIREWWDLKPGAPKPKANQQKQDDGRGLVLPGY
KYLGPFNGLDKGEPVNAADAAALEHDKAYDQQLKAGDNPYLRYNHADAEFQERLQED
TSFGGNLGRAVFQAKKRVLEPLGLVEEGANTAPGKKRPVEQSPQEPDSSSGIGKTGQQP
AKKRLNFGQTGDSESVPDPQPLREPPAAPSGLGPNTMASGGGAPMADNNEGADGVGNS
SGNWHCDSTWLGDRVITTSTRTWALPTYNNHLYKQISNGTSGGSTNDNTYFGYSTPWG
YFDFNRFHCHFSPRDWQRLINSNWGFRPKRLNFKLFNIQVKEVTTNEGTKTIANNLTSTV
QVFTDSEYQLPYVLGSAHQGCLPPFPADVFMVPQYGYLTLNNGSQALGRSSFYCLEYFP
SQMLRTGNNFQFSYTFEDVPFHSSYAHSQSLDRLMNPLIDQYLYYLVRTQTTGTGGTQT
LAFSQAGPSSMANQARNWVPGPCYRQQRVSTTTNQNNNSNFAWTGAAKFKLNGRDSL
MNPGVAMASHKDDEDRFFPSSGVLIFGKQGAGNDGVDYSQVLITDEEEIKATNPVATEE
YGAVAINNQAANTQAQTGLVHNQGVIPGMVWQNRDVYLQGPIWAKIPHTDGNFHPSP
LMGGFGLKHPPPQILIKNTPVPADPPLTFNQAKLNSFITQYSTGQVSVEIEWELQKENSKR
WNPEIQYTSNYYKSTNVDFAVNTEGVYSEPRPIGTRYLTRNL.

In another embodiment, the VP1 sequence (from pig (denoted as Bpo1)) comprises the amino acid sequence of SEQ ID NO:40 (amino acids 1-716); or comprises an amino acid sequence that is at least 60%, at least 65%, at least 70%, at least 75%, at least 80%, at least 85%, at least 90%, or at least 95% identical over the full length of the amino acid sequence of SEQ ID NO:40. The VP2 capsid protein spans amino acids 137-716 of SEQ ID NO:40 and the VP3 capsid protein spans amino acids 184-716 of SEQ ID NO:40.

(SEQ ID NO: 40)
MSFVDHPPDWLEEIGEGLKEFLGLEPGPPKPKPNQQKQDNARGLVLPGYNYL
GPGNGLDRGEPVNRADEVAREHDISYNEQLQAGDNPYLKYNHADAEFQEKLADDTSFG
GNLGKAVFQAKKRVLEPFGLVEEPAKTAAKGERIDDHYPKKKKARVEETEAGTSGGQQ
LQIPAQPASSLGADTMSAGGGSPLGDNNQGADGVGNASGDWHCDSTWMGDRVITKST
RTWVLPSYNNHLYKEIHSGSVDGSNANAYFGYSTPWGYFDFNRFHSHWSPRDWQRLV
NNYWGFRPRSLKVKIFNIQVKEVTVQDATTTIANNLTSTVQVFTDDDYQLPYVIGNGTE
GCLPAFPPQVFTLPQYGYATLNRNNTDDPTERSSFFCLEYFPSKMLRTGNSFEFTYSFEE
VPFHCSFAPSQNLFKLANPLVDQYLYRFVSTDTSGNIQFQKNLKARYANTYKNWFPGP
MCRTQGWYTGSGTYNRSGVTNFATSNRMDLEGASYQVNPQPNGMTNTLQDSNKYAL
ENTMIFNSQNAEPGTTSLYQENNLLITSESETQPVNRVAYDTGGQMATNAQSTNLAPTV
GTYNHQEMLPGSVWMDRDVYLQGPIWAKIPETGAHFHPSPAMGGFGLKHPPPMMLIK
NTPVPSNVTAFSEIPVKSFITQYSTGQVTVEMEWELKKEDSKRWNPEIQYTNNYNNPEF
VDFAPDTSGEYRTTRAIGTRYLTRPL.

In another embodiment, the VP1 sequence (from pig (denoted as Bpo2)) comprises the amino acid sequence of SEQ ID NO:41 (amino acids 1-716); or comprises an amino acid sequence that is at least 60%, at least 65%, at least 70%, at least 75%, at least 80%, at least 85%, at least 90%, or at least 95% identical over the full length of the amino acid sequence of SEQ ID NO:41. The VP2 capsid protein spans amino acids 137-716 of SEQ ID NO:41 and the VP3 capsid protein spans amino acids 184-716 of SEQ ID NO:41.

(SEQ ID NO: 41)
MSFVDHPPDWLEEIGEGLKEFLGLEPGPPKPKPNQQKQDNARGLVLPGYNYL
GPGNGLDRGEPVNRADEVAREHDISYNEQLQAGDNPYLKYNHADAEFQEKLADDTSFG
GNLGKAVFQAKKRALEPFGLVEEPAKTAAKGERIDDHYPKKKKARVEETEAGTSGGQQ
LQIPAQPASSLGADTMSAGGGSPLGDNNQGADGVGNASGDWHCDSTWMGDRVITKST
RTWVLPSYNNHLYKEIHSGSVDGSNANAYFGYSTPWGYFDFNRFHSHWSPRDWQRLV
NNYWGFRPRSLKVKIFNIQVKEVTVQDATTTIANNLTSTVQVFTDDDYQLPYVIGNGTE
GCLPAFPPQVFTLPQYGYATLNRNNTDDPTERSSFFCLEYFPSKMLRTGNSFEFTYSFEE
VPFHCSFAPSQNLFKLANPLVDQYLYRFVGTDTSGNIQFQKNLKARYANTYKNWFPGP
MCRTQGWYTGSGTYNRSGVTNFATSNRMDLEGASYQVNPQPNGMTNTLQDSNKYAL
ENTMIFNSQNAEPGTTSLYQENNLLITSESETQPVNRVAYDTGGQMATNAQSTNLAPTV
GTYNHQEMLPGSVWMDRDVYLQGPIWAKIPETGAHFHPSPAMGGFGLKHPPPMMLIK
NTPVPSNVTAFSEIPVKSFITQYSTGQVTVEMEWELKKENSKRWNPEIQYTNNYNNPEF
VDFAPDTSGEYRTTRAIGTRYLTRPL.

In another embodiment, the VP1 sequence (from pig (denoted as Bpo3)) comprises the amino acid sequence of SEQ ID NO:42 (amino acids 1-716); or comprises an amino acid sequence that is at least 60%, at least 65%, at least 70%, at least 75%, at least 80%, at least 85%, at least 90%, or at least 95% identical over the full length of the amino acid sequence of SEQ ID NO:42. The VP2 capsid protein spans amino acids 137-716 of SEQ ID NO:42 and the VP3 capsid protein spans amino acids 184-716 of SEQ ID NO:42.

(SEQ ID NO: 42)
MSFVDHPPDWFEEIGEGLKEFLGLEPGPPKPKPNQQKQDNARGLVLPGYNYL
GPGNGLDRGEPANRADEVAREHDISYNEQLQAGDNPYLKYNHADAEFQEKLADDTSFG
GNLGKAVFQAKKRVLEPFGLVEEPAKTAAKGERIDDHYPKKKKARVEETEAGTSGGQQ
LQIPAQPASSLGADTMSAGGGSPLGDNNQGADGVGNASGDWHCDSTWMGDRVITKST
RTWVLPSYNNHLYKEIHSGSVDGSNANAYFGYSTPWGYFDFNRFHSHWSPRDWQRLV
NNYWGFRPRSLKVKIFNIQVKEVTVQDATTTIANNLTSTVQVFTDDDYQLPYVIGNGTE
GCLPAFPPQVFTLPQYGYATLNRNNTDDPTERSSFFCLEYFPSKMLRTGNSFEFTYSFGE
VPLHCSFAPSQNLFKLANPLVDQHLYRFVSTDTSGNIQFQKNLKARYANTYKNWFPGP
MCRTQGWYTGSGTYNRPGVTNFATSNRMDLEGASYQVNPQPNGMTNTLQDSNKYAL
ENTMIFNSQNAEPGTTSLYQENNLLITSESETQPVNRVAYDTGGQMATNAQSTNLAPTV
GTYNHQEMLPGSVWMDRDVYLQGPIWAKIPETGAHFHPSPAMGGFGLKHPPPMMLTK
NTPVPSNVTAFSEIPVKSFITQYSTGQVTVEMEWELKKENSKRWNPEIQYTNNYNNPEF
VDFAPDTSGEYRTTRAIGTRYLTRPL.

In another embodiment, the VP1 sequence (from pig (denoted as Bpo4)) comprises the amino acid sequence of SEQ ID NO:43 (amino acids 1-716); or comprises an amino acid sequence that is at least 60%, at least 65%, at least 70%, at least 75%, at least 80%, at least 85%, at least 90%, or at least 95% identical over the full length of the amino acid sequence of SEQ ID NO:43. The VP2 capsid protein spans amino acids 137-716 of SEQ ID NO:43 and the VP3 capsid protein spans amino acids 184-716 of SEQ ID NO:43.

(SEQ ID NO: 43)
MSFVDHPPDWLEEIGEGLKEFLGLEPGPPKPKPNQQKQDNARGLVLPGYNYL
GPGNGLDRGEPVNRADEVAREHDISYNEQLQAGDNPYLKYNHADAEFQEKLADDTSFG
GNLGKAVFQAKKRVLEPFGLVEEPAKTAAKGERIDDHYPKKKKARVEETEAGTSGGQQ
LQIPAQPASSLGADTMSAGGGSPLGDNNQGADGVGNASGDWHCDSTWMGDRVITKST
RTWVLPSYNNHLYKEIHSGSVDGSNANAYFGYSTPWGYFDFNRFHSHWSPRDWQRLV
NNYWGFRPRSLKVKIFNIQVKEVTVQDATTTIANNLTSTVQVFTDDDYQLPYVIGNGTE
GCLPAFPPQVFTLPQYGYATLNRNNTDDPTERSSFFCLEYFPSKMLRTGNSFEFTYSFEE
VPFHCSFAPSQNLFKLANPLVDQYLYRFVSTDTSGNIQFQKNLKARYANTYKNWFPGP
MCRTQGWYTGSGTYNRSGVTNFATSNRMDLEGASYQVNPQPNGMINTLQDSNKYAL
ENTMIFNSQNAEPGTTSLYQENNLLITSESETQPVNRVAYDTGGQMATNAQSTNLAPTV
GTYNHQEMLPGSVWMDRDVYLQGPIWAKIPETGAHFHPSPAMGGFGLKHPPPMMLIK
NTPVPSNVTAFSEIPVKSFITQYSTGQVTVEMEWELKKENSKRWNPEIQYTNNYNNPEF
VDFAPDTSGEYRTTRAIGTRYLTRPL.

In another embodiment, the VP1 sequence (from pig (denoted as Bpo6)) comprises the amino acid sequence of SEQ ID NO:44 (amino acids 1-716); or comprises an amino acid sequence that is at least 60%, at least 65%, at least 70%, at least 75%, at least 80%, at least 85%, at least 90%, or at least 95% identical over the full length of the amino acid sequence of SEQ ID NO:44. The VP2 capsid protein spans amino acids 137-716 of SEQ ID NO:44 and the VP3 capsid protein spans amino acids 184-716 of SEQ ID NO:44.

(SEQ ID NO: 44)
MSFVDHPPDWLEEIGEGLKEFLGLEPGPPKPKPNQQKQDNARGLVLPGYNYL
GPGNGLDRGEPVNRADEVAREHGISYNEQLQAGDDPYLKYNHADAEFQEKLADDTSFG
GNLGKAVFQAKKRVLEPFGLVEEPAKTAAKGERIDDHYPKKKKARVEETEAGTGGGQ
QLQIPAQPASSLGADTMSAGGGSPLGDNNQGADGVGNASGDWHCDSTWMGDRVITKS
TRTWVLPSYNNHLYKEIHSGSVDGSNANAYFGYSTPWGYFDFNRFHSHWSPRDWQRL
VNNYWGFRPRSLKVKIFNIQVKEVTVQDATTTIANNLTSTVQVFTDDDYQLPYVIGNGT
EGCLPAFPPQVFTLPQYGYATLNRNNTDDPTERSSFFCLEYFPSKMLRTGNSLEFTYSFEE
VPFHCSFAPSQNLFKLANPLVDQYLYRFVSTDTSGNIQFQKNLKARYANTYKNWFPGP
MCRTQGWYTGSGTYNRSGVTNFATSNRMDLEGASYQVNPQPNGMTNTLQDSNKYAL
ENTMIFNSQNAEPGTTSLYQENNLLITSESETQPVNRVAYDTGGQMATNAQSTNLAPTV
GTYNHQEMLPGSVWMDRDVYLQGPIWAKIPETGAHFHPSPAMGGFGLKHPPPMMLIK
NTPVPSNVTAFSEIPVKSFITQYSTGQVTVEMEWELKKENSKRWNPEIQYTNNYNNPEF
VDFAPDTSGEYRTTRAIGTRYLTRPL.

In another embodiment, the VP1 sequence (from pig (denoted as Bpo8)) comprises the amino acid sequence of SEQ ID NO:45 (amino acids 1-716); or comprises an amino acid sequence that is at least 60%, at least 65%, at least 70%, at least 75%, at least 80%, at least 85%, at least 90%, or at least 95% identical over the full length of the amino acid sequence of SEQ ID NO:45. The VP2 capsid protein spans amino acids 137-716 of SEQ ID NO:45 and the VP3 capsid protein spans amino acids 184-716 of SEQ ID NO:45.

(SEQ ID NO: 45)
MSFVDHPPDWLEEIGEGLKEFLGLKPGPPKPKPNQQKQDNARGLVLPGYNYL
GPGNGLGRGEPVNRADEVAREHDISYNEQLQAGDNPYLKYNHADAEFQEKLADDTSFG
GNLGKAVFQAKKRVLEPFGLVEEPAKTAAKGERIDDHYPKKKKARVEETEAGTSGGQQ
LQIPAQPASSLGADTMSAGGGSPLGDNNQGADGVGNASGDWHCDSTWMGDRVITKST
RTWVLPSYNNHLYKEIHSGSVDGSNANAYFGYSTPWGYFDFNRFHSHWSPRDWQRLV
NNYWGFRPRSLKVKIFNIQVKEVTVQDATTTIANNLTSTVQVFTDDDYQLPYVIGNGTE
GCLPAFPPQVFTLPQYGYATLNRNNTDDPTERSSFFCLEYFPSKMLRTGNSFEFTYSFEE
VPFHCSFAPSQNLFKLANPLVDQYLYRFVSTDTSGNIQFQKNLKARYANTYKNWFPGP
MCRTQGWYTGSGTYNRSGVTNFATSNRMDLEGASYQANPQPNGMINTLQDSNKYAL
ENTMIFNSQNAEPGTTSLYQENNLLITSESETQPVNRVAYDTGGQMATNAQSTNLAPTV
GTYNHQEMLPGSVWMDRDVYLQGPIWAKIPETGAHFHPSPAMGGFGLKHPPPMMLIK
NTPVPSNVTAFSEIPVKSLITQYSTGQVTVEMEWELKKENSKRWNPEIQYTNNYNNPEF
VDFAPDTSGEYRTTRAIGTRYLTRPL.

In another embodiment, the VP1 sequence (from pig (denoted as Bpo13)) comprises the amino acid sequence of SEQ ID NO:46 (amino acids 1-716); or comprises an amino acid sequence that is at least 60%, at least 65%, at least 70%, at least 75%, at least 80%, at least 85%, at least 90%, or at least 95% identical over the full length of the amino acid sequence of SEQ ID NO:46. The VP2 capsid protein spans amino acids 137-716 of SEQ ID NO:46 and the VP3 capsid protein spans amino acids 184-716 of SEQ ID NO:46.

(SEQ ID NO: 46)
MSFVDHPPDWLEEIGEGLKEFLGLEPGPPKPKPNQQKQDNARGLVLPGYNYL
GPGNGLDRGEPVNRADEVAREHDISYNEQLQAGDNPYLKYNHADAEFQEKLADDTSFG
GNLGKAVFQAKKRVLEPFGLVEEPAKTAAKGERIDDHYPKKKKARVEETEAGTSGGQQ
LQIPAQPASSLGADTMSAGGGSPLGDNNQGADGVGNASGDWHCDSTWMGDRVITKST
RTWVLPSYNNHLYKEIHSGSVDGSNANAYFGYSTPWGYFDFNRFHSHWSPRDWQRLV
NNYWGFRPRSLKVKIFNIQVKEVTVQDATTTIANNLASTVQVFTDDDYQLPYVIGNGTE
GCLPAFPPQVFTLPQYGYATLNRNNTDDPTERSSFFCLEYFPSKMLRTGNSFEFTYSFEE
VPFHCSFAPSQNLFKLANPLVDQYLYRFVSTDTSGNIQFQKNLKARYANTYKNWFPGP
MCRTQGWYTGSGTYNRSGVTNFATSNRMDLEGASYQVNPQPNGMTNTLQDSNKYAL
ENTMIFNSQNAEPGTTSLYQENNLLITSESETQPVNRVAYDTGGQMATNAQSTNLAPTV
GTYNHQEMLPGSVWMDRDVYLQGPIWAKIPETGAHFHPSPAMGGFGLKHPPPMMLIK
NTPVPSNVTAFSEIPVKSFITQYSTGQVTVEMEWELKKENSKRWNPEIQYTNNYNNPEF
VDFAPDTSGEYRTTRAIGTRYLTRPL.

In another embodiment, the VP1 sequence (from pig (denoted as Bpo18)) comprises the amino acid sequence of SEQ ID NO:47 (amino acids 1-716); or comprises an amino acid sequence that is at least 60%, at least 65%, at least 70%, at least 75%, at least 80%, at least 85%, at least 90%, or at least 95% identical over the full length of the amino acid sequence of SEQ ID NO:47. The VP2 capsid protein spans amino acids 137-716 of SEQ ID NO:47 and the VP3 capsid protein spans amino acids 184-716 of SEQ ID NO:47.

(SEQ ID NO: 47)
MSFVDHPPDWLEEIGEGLKEFLGLEPGPPKPKPNQQKQDNARGLVLPGYNYL
GPGNGLDRGEPVNRADEVAREHDISYNEQLQAGDDPYLKYNHADAEFQEKLADDTSFG
GNLGKAVFQAKKRVLEPFGLVEEPAKTAAKGERIDDHYPKKKKARVEETEAGTSGGQQ
LQIPAQPASSLGADTMSAGGGSPLGDNNQGADGVGNASGDWHCDSTWMGDRVITKST
RTWVLPSYNNHLYKEIHSGSVDGSNANAYFGYSTPWGYFDFNRFHSHWSPRDWQRLV
NNYWGFRPRSLKVKIFNIQVKEVTVQDATTTIANNLTSTVQVFTDDDYQLPYVIGNGTE
GCLPAFPPQVFTLPQYGYATLNRNNTDDPTERSSFFCLEYFPSKMLRTGNSFEFTYSFEE
VPFHCSFAPSQNLFKLANPLVDQYLYRFVSTDTSGNIQFQKNLKARYANTYKNWFPGP
MRRTQGWYTGSGTYNRSGVTNFATSNRMDLEGASYQVNPQPNGMINTLQDSNKYAL
ENTMIFNSQNAEPGTTSLYQENNLLITSESETQPVNRVAYDTGGQMATNAQSTNLAPTV
GTYNHQEMLPGSVWMDRDVYLQGPIWAKIPETGAHFHPSPAMGGFGLKHPPPMMLIK
NTPVPSNVTAFSEIPVKSFITQYSTGQVTVEMEWELKKENSKRWNPEIQYTNNYNNPEF
VDFAPDTSGEYRTTRAIGTRYLTRPL.

In another embodiment, the VP1 sequence (from pig (denoted as Bpo20)) comprises the amino acid sequence of SEQ ID NO:48 (amino acids 1-716); or comprises an amino acid sequence that is at least 60%, at least 65%, at least 70%, at least 75%, at least 80%, at least 85%, at least 90%, or at least 95% identical over the full length of the amino acid sequence of SEQ ID NO:48. The VP2 capsid protein spans amino acids 137-716 of SEQ ID NO:48 and the VP3 capsid protein spans amino acids 184-716 of SEQ ID NO:48.

(SEQ ID NO: 48)
MSFVDHPPDWLEEIGEGLKEFLGLEPGPPKPKPNQQKQDNARGLVLPGYNYL
GPGNGLDRGEPVNRADEVAREHDISYNEQLQAGDNPYLKYNHADAEFQEKLADDTSFG
GNLGKAVFQAKKRVLEPFGLVEEPAKTAAKGERIDDHYPKKKKARVEETEAGTSGGQQ
LQIPAQPASSLGADTMSAGGGSPLGDNNQGADGVGNASGDWHCDSTWMGDRVITKST
RTWVLPSYNNHLYKEIHSGSVDGSNANAYFGYSTPWGYFDFNRFHSHWGPRDWQRLV
NNYWGFRPRSLKVKIFNIQVKEVTVQDATTTIANNLTSTVQVFTDDDYQLPYVIGNGTE
GCLPAFPPQVFTLPQYGYATLNRNNTDDPTERSSFFCLEYFPSKMLRTGNSFEFTYSFEG
VPFHCSFAPSQNLFKLANPLVDQYLYRFVSTDTSGNIQFQKNLKARYANTYKNWFPGP
MCRTQGWYTGSGTYNRSGVTNFATSNRMDLEGASYQVNPQPNGMTNTLQDSNKYAL
ENTMIFNSQNAEPGTTSLYQENNLLITSESETQPVNRVAYDTGGQMATNAQSTNLAPTV
GTYNHQEMLPGSVWMDRDVYLQGPIWAKIPETGAHFHPSPAMGGFGLKHPPPMMLIK
NTPVPSNVTAFSEIPVKSFITQYSTGQVTVEMEWELKKENSKRWNPEIQYTNNYNNPEF
VDFAPDTSGEYRTTRAIGTRYLTRPL.

In another embodiment, the VP1 sequence (from pig (denoted as Bpo23)) comprises the amino acid sequence of SEQ ID NO:49 (amino acids 1-716); or comprises an amino acid sequence that is at least 60%, at least 65%, at least 70%, at least 75%, at least 80%, at least 85%, at least 90%, or at least 95% identical over the full length of the amino acid sequence of SEQ ID NO:49. The VP2 capsid protein spans amino acids 137-716 of SEQ ID NO:49 and the VP3 capsid protein spans amino acids 184-716 of SEQ ID NO:49.

(SEQ ID NO: 49)
MSFVDHPPDWLEEIGEGLKEFLGLEPGPPKPKPNQQKRDNARGLVLPGYNYL
GPGNGLDRGEPVNRVDEVARERDISYNEQLQAGDNPYLKYNHADAEFQEKLADDTSFG
GNLGKAVFQAKKRVLEPFGLVEEPAKTAAKGERIDDHYPKKKKARVEETEAGTSGGQQ
LQIPAQPASSLGADTMSAGGGSPLGDNNQGADGVGNASGDWHCDSTWMGDRVITKST
RTWVLPSYNNHLYKEIHSGSVDGSNANAYFGYSTPWGYFDFNRFHSHWSPRDWQRLV
NNYWGFRPRSLKVKIFNIQVKEVTVQDATTTIANNLTSTVQVFTDDDYQLPYVIGNGTE
GCLPAFPLQVFTLPQYGYATLNRNNTDDPTERSSFFCLEYFPSKMLRTGNSFEFTYSFEE
VPFHCSFAPSQNLFKLANPLVDQYLYRFVSTDTSGNIQFQKNLKARYANTYKNWFPGP
MCRTQGWYTGSGTYNRSGVTNFATSNRMDLEGASYQVNPQPNGMINTLQDSNKYAL
ENTMIFNSQNAEPGTTSLYQENNLLITSESETQPVNRVAYDTGGQMATNAQSTNLAPTV
GTYNHQEMLPGSVWMDRDVYLQGPIWAKIPETGAHFHPSPAMGGFGLKHPPPMMLIK
NTPVPSNVTAFSEIPVKSFITQYSTGQVTVEMEWELKKENSKRWNPEIQYTNNYNNPEF
VDFAPDTSGEYRTTRAIGTRYLTRPL.

In another embodiment, the VP1 sequence (from pig (denoted as Bpo24)) comprises the amino acid sequence of SEQ ID NO:50 (amino acids 1-716); or comprises an amino acid sequence that is at least 60%, at least 65%, at least 70%, at least 75%, at least 80%, at least 85%, at least 90%, or at least 95% identical over the full length of the amino acid sequence of SEQ ID NO:50. The VP2 capsid protein spans amino acids 137-716 of SEQ ID NO:50 and the VP3 capsid protein spans amino acids 184-716 of SEQ ID NO:50.

(SEQ ID NO: 50)
MSFVDHPPDWLEEIGEGLKEFLGLEPGPPKPKPNQQKQDNARGLVLPGYNYL
GPGNGLDRGEPVNRADEVAREHDISYNEQLQAGDNPYLKYNHADAEFQEKLADDTSFG
GNLGKAVFQAKKRVLEPFGLVEEPAKTAAKGERIDDHYPKKKKARVEETEAGTSGGQQ
LQIPAQPASSLGADTMSAGGGSPLGDNNQGADGVGNASGDWHCDSTWMGDRVITKST
RTWVLPSYNNHLYKEIHSGSVDGSNANAYFGYSTPWGYFDFNRFHSHWSPRDWQRLV
NNYWGFRPRSLKVKIFNIQVKEVTVQDATTTIANNLTSTVQVFTDDDYQLPYVIGNGTE
GCLPAFPPQVFTLPQYGYATLNRNNTDDPTERSSFFCLEYFPSKMLRTGNSFEFTYSFEE
VPFHCSFAPSQNLFKLANPLVDQYLYRFVSTDTSGNIQFQKNLKARYANTYKNWFPGP
MCRTQGWYTGSGTYNRSGVTNFATSNRMDLEGASYQVNPQPNGMTNTLQDSNKYAL
ENTMIFNSQNAEPGTTSLYQENNLLITSESETQPVNRVAYDTGGQMATNAQSTNLAPTV
GTYNHQEMLPGSVWMDRDVYLQGPIWAKIPETGAHFHPSPAMGGFGLKHPPPMMLIK
NTPVPSNVTAFSEIPVKSFITQYSTGQVTVEMEWELKKENSKRWNPEIQYTNNYNNPEF
VDFAPDTSGECRTTRAIGTRYLTRPL.

In another embodiment, the VP1 sequence (from pig (denoted as Bpo27)) comprises the amino acid sequence of SEQ ID NO:51 (amino acids 1-716); or comprises an amino acid sequence that is at least 60%, at least 65%, at least 70%, at least 75%, at least 80%, at least 85%, at least 90%, or at least 95% identical over the full length of the amino acid sequence of SEQ ID NO:51. The VP2 capsid protein spans amino acids 137-716 of SEQ ID NO:51 and the VP3 capsid protein spans amino acids 184-716 of SEQ ID NO:51.

(SEQ ID NO: 51)
MSFVDHPPDWLEEIGEGLKEFLGLEPGPPKPKPNQQKQDNARGLVLPGYNYL
GPGNGLDRGEPVNRADEVAREHDISYNEQLQAGDNPYLKYNHADAEFQEKLADDTSFG
GNLGKAVFQAKKRVLEPFGLVEEPAKTAAKGERIDDHYPKKKKARVEETEAGTSGGQQ
LQIPAQPASSLGADTMSAGGGSPLGDNNQGADGVGNASGDWHCDSTWMGDRVITKST
RTWVLPSYNNHLYKEIHSGSVDGSNANAYFGYSTPWGYFDFNRFHSHWSPRDWQRLV
DNYWGFRPRSLKVKIFNIQVKEVTVQDATTTIANNLTSTVQVFTDDDYQLPYVIGNGTE
GCLPAFPPQVFTLPQYGYATLNRNNTDDPTERSSFFCLEYFPSKMLRTGNSFEFTYSFEE
VPFHCSFAPSQNLFKLANPLVDQYLYRFVSTDTSGNIQFQKNLKARYANTYKNWFPGP
MCRTQGWYTGSGTYNRSGVTNFATSNRMDLEGASYQVNPQPNGMTNTLQDSNKYAL
ENTMVFNSQNAEPGTTSLYQENNLLITSESETQPVNRVAYNTGGQMATNAQSTNLAPTV
GTYNHQEMLPGSVWMDRDVYLQGPIWAKIPETGAHFHPSPAMGGFGLKHPPPMMLIK
NTPVPSNVTAFSEIPVKSFITQYSTGQVTVEMEWELKKENSKRWNPEIQYTNNYNNPEF
VDFAPDTSGEYRTTRAIGTRYLTRPL.

In another embodiment, the VP1 sequence (from pig (denoted as Bpo28)) comprises the amino acid sequence of SEQ ID NO:52 (amino acids 1-716); or comprises an amino acid sequence that is at least 60%, at least 65%, at least 70%, at least 75%, at least 80%, at least 85%, at least 90%, or at least 95% identical over the full length of the amino acid sequence of SEQ ID NO:52. The VP2 capsid protein spans amino acids 137-716 of SEQ ID NO:52 and the VP3 capsid protein spans amino acids 184-716 of SEQ ID NO:52.

(SEQ ID NO: 52)
MSFVDHPPDWLEEIGEGLKEFLGLEPGPPKPKPNQQKRDNARGLVLPGYNYL
GPGNGLGRGEPVNRADEVAREHDISYNEQLQAGDNPYLKYNHADAEFQEKLADDTSFG
GNLGKAVFQAKKRVLEPFGLVEEPAKTAAKGERIDDHYPKKKKARVEETEAGTSGGQQ
LQIPAQPASSLGADTMSAGGGSPLGDNNQGADGVGNASGDWHCDSTWMGDRVITKST
RTWVLPSYNNHLYKEIHSGSVDGSNANAYFGYSTPWGYFDFNRFHSHWSPRDWQRLV
NNYWGLRPRSLKVKIFNIQVKEVTVQDATTTIANNLTSTVQVFTDDDYQLPYVIGNGTE
GCLPAFPPQVFTLPQYGYATLNRNNTDDPTERSSFFCLEYFPSKMLRTGNSFEFTYSFEE
VPFHCSFAPSQNLFKLANPLADQYLYRFVSTDTSGNIQFQKNLKARYANTYKNWFPGP
MCRTQGWYTGSGTYNRSGVTNFATSNRMDLEGASYQVNPQPNGMTNTLQDSNKYAL
ENTMISNSQNAEPGTTSLYRENNLLITSESETQPVNRVAYDTGGQMATNAQSTNLAPTV
GTYNHQEMLPGSVWMDRDVYLQGPIWAKIPETGAHFHPSPAMGGFGLKHPPPMMLIK
NTPVPSNVTAFSEIPVKSFITQYSTGQVTVEMEWELKKENSKRWNPEIQYTNNYNNPEF
VDFAPDTSGEYRTTRAIGTRYLTRPL.

In another embodiment, the VP1 sequence (from pig (denoted as Bpo29)) comprises the amino acid sequence of SEQ ID NO:53 (amino acids 1-716); or comprises an amino acid sequence that is at least 60%, at least 65%, at least 70%, at least 75%, at least 80%, at least 85%, at least 90%, or at least 95% identical over the full length of the amino acid sequence of SEQ ID NO:53. The VP2 capsid protein spans amino acids 137-716 of SEQ ID NO:53 and the VP3 capsid protein spans amino acids 184-716 of SEQ ID NO:53.

(SEQ ID NO: 53)
MSFVDHPPDWLEEIGEGLKEFLGLEPGPPKPKPNQQKQDNARGLVLPGYNYL
GPGNGLDRGEPVNRADEVAREHDISYNEQLQAGDNPYLKYNHADAEFQEKLADDTSFG
GNLGKAVFQAKKRVLEPFGLVEEPAKTAAKGERIDDHYPKKKKARVEETEAGTSGGQQ
LQIPAQPASSLGADTMSAGGGSPLGDNNQGADGVGNASGDWHCDSTWMGDRVITKST
RTWVLPSYNNHLYKEIHSGSVDGSNANAYFGYSTPWGYFDFNRFHSHWSPRDWQRLV
NNYWGFRPRSLKVKIFNIQVKEVTVQDATTTIANNLTSTVQVFTDDDYQLPYVIGNGTE
GCLPAFPPQVFTLPQYGYATLNRNNTDDPTERSSFFCLEYFPSKMLRTGNSFEFTYSFEE
VPFHCSFAPSQNLFKLANPLVDQYLYRFVSTDTSGNIQFQKNLKARYANTYKNWFPGP
MCRTQGWYTGSGTYNRSGVTNFATSNRMDLEGASYQVNPQPNGMTNTLQDSNKYAL
ENTMIFNSQNAEPGTTSLYQENNLLITSESGTQPVNRVAYDTGGQMATNAQSTNLAPTV
GTYNHQEMLPGSVWMDRGVYLQGPIWAKIPETGAHFHPSPAMGGFGLKHPPPMMLIK
NTPVPSNVTAFSEIPVKSFVTQYSTGQVTVEMEWELKKENSKRWNPEIQYTNNYNNPEF
VDFAPDTSGEYRTTRAIGTRYLTRPL.

In another embodiment, the VP1 sequence (from pig (denoted as Bpo33)) comprises the amino acid sequence of SEQ ID NO:54 (amino acids 1-716); or comprises an amino acid sequence that is at least 60%, at least 65%, at least 70%, at least 75%, at least 80%, at least 85%, at least 90%, or at least 95% identical over the full length of the amino acid sequence of SEQ ID NO:54. The VP2 capsid protein spans amino acids 137-716 of SEQ ID NO:54 and the VP3 capsid protein spans amino acids 184-716 of SEQ ID NO:54.

(SEQ ID NO: 54)
MSFVDHPPDWLEEIGEGLKEFLGLEPGPPKPKPNQQKQDNARGLVLPGYNYL
GPGNGLDRGEPVNRADEVAREHDISYNEQLQAGDNPYLKYNHADAEFQEKLADDTSFG
GNLGKAVFQAKKRVLEPFGLVEEPAKTAAKGERIDDHYPKKKKARVEETEAGTSGGQQ
LQIPAQPASSLGADTMSAGGGSPLGDNNQGADGVGNASGDWHCDSTWMGDRVITKST
RTWVLPSYNNHLYKEIHSGSVDGSNAHAYFGYSTPWGYFDFNRFHSHWSPRDWQRLV
NNYWGFRPRSLKVKIFNIQVKEVTVQDATTTIANNLTSTVQVFTDDDYQLPYVIGNGTE
GCLPAFPPQVFTLPQYGYATLNRNNTDDPTERSSFFCLEYFPSKMLRTGNSFEFTYSFEE
VPFHCSFAPSQNLFKLANPLVDQYLYRFVSTDTSGNIQFQKNLKARYANTYKNWFPGP
MCRTQGWYTGSGTYNRSGVTNFATSNRMDLEGASYQVNPQPNGMTNTLQDSNKYAL
ENTMIFNSQNAEPGTTSLYQENNLLITSESETQPVNRVAYDTGGQMATNAQSTNLAPTV
GTYNHQEMLPGSVWMDRDVYLQGPIWAKIPETGAHFHPSPAMGGFGLKHPPPMMLIK
NTPVPSNVTAFSEIPVKSFITQYSTGQVTVEMEWELKKENSKRWNPEIQYTNNYNNPEF
VDFAPDTSGEYRTTRAIGTRYLTRPL.

In another embodiment, the VP1 sequence (from pig (denoted as Bpo35)) comprises the amino acid sequence of SEQ ID NO:55 (amino acids 1-716); or comprises an amino acid sequence that is at least 60%, at least 65%, at least 70%, at least 75%, at least 80%, at least 85%, at least 90%, or at least 95% identical over the full length of the amino acid sequence of SEQ ID NO:55. The VP2 capsid protein spans amino acids 137-716 of SEQ ID NO:55 and the VP3 capsid protein spans amino acids 184-716 of SEQ ID NO:55.

(SEQ ID NO: 55)
MSFVDHPPDWLEEIGEGLKEFLGLEPGPPKPKPNQQKRDNARGLVLPGYNYL
GPGNGLDRGEPVNRADEVAREHDISYNEQLQAGDNPYLKYNHADAEFQEKLADDTSFG
GNLGKAVFQAKKRVLEPFGLVEEPAKTAAKGERIDDHYPKKKKARVEETEAGTSGGQQ
LQIPAQPASSLGADTMSAGGGSPLGDNNQGADGVGNASGDWHCDSTWMGDRVITKST
RTWVLPSYNNHLYKEIHSGSVDGSNANAYFGYSTPWGYFDFNRFHSHWSPRDWQRLV
NNYWGFRPRSLKVKIFNIQVKEVTVQDATTTIANNLTSTVQVFTDDDYQLPYVIGNGTE
GCLPAFPPQVFTLPQYGYATLNRNNTDDPTERSSFFCLEYFPSKMLRTGNSFEFTYSFEE
VPFHCSFAPSQNLFKLANPLVDQYLYRFVSTDTSGNIQFQKNLKARYANTYKNWFPGP
MCRTQGWYTGSGTYNRSGVTNFATSNRMDLEGASYQVNPQPNGMTNTLQDSNKYAL
ENTMIFNSQNAEPGTTSLYQENNLLITSESETQPVNRVAYDTGGQMATNAQSTNLAPTV
GTYNHQEMLPGSVWMDRDVYLQGPIWAKIPETGAHFHPSPAMGGFGLKHPPPMMLIK
NTPVPSNVTAFSEIPVKSFITQYSTGQVTVEMEWELKKENSKRWNPEIQYTNNYNNPEF
VGFAPDTSGEYRTTRAIGTRYLTRPL.

In another embodiment, the VP1 sequence (from pig (denoted as Bpo36)) comprises the amino acid sequence of SEQ ID NO:56 (amino acids 1-716); or comprises an amino acid sequence that is at least 60%, at least 65%, at least 70%, at least 75%, at least 80%, at least 85%, at least 90%, or at least 95% identical over the full length of the amino acid sequence of SEQ ID NO:56. The VP2 capsid protein spans amino acids 137-716 of SEQ ID NO:56 and the VP3 capsid protein spans amino acids 184-716 of SEQ ID NO:56.

(SEQ ID NO: 56)
MSFVDHPPDWLEEIGEGLKEFLGLEPGPPKPKPNQQKQDNARGLVLPGYNYL
GPGNGLDRGEPVNRADEVAREHDISYNEQLQAGDNPYLKYNHADAEFQEKLADDTSFG
GNLGKAVFQAKKRVLEPFGLVEEPAKTAAKGERIGDHYPKKKKARVEETEAGTSGGQQ
PQIPAQPASSLGADTMSAGGGSPLGDNNQGADGVGNASGDWHCDSTWMGDRVITKST
RTWVLPSYNNHLYKEIHSGSVDGSNANAYFGYSTPWGYFDFNRFHSHWSPRDWQRLV
NNYWGFRPRSLKVKIFNIQVKEVTVQDATTTIANNLTSTVQVFTDDDYQLPYVIGNGTE
GCLPAFPPQVFTLPQYGYATLNRNNTDDPTERSSFFCLEYLPSKMLRTGNSFEFTYSFEE
VPFHCSFAPSQNLFKLANPLVDQYLYRFVSTDTSGNIQFQKNLKARYANTYKNWFPGP
MCRTQGWYTGSGTYNRSGVTNFATSNRMDLEGASYQVNPQPNGMINTLQDSNKYAL
ENTMIFNSQNAEPGTTSLYQENNLLITSESETQPVNRVAYNTGGQMATNAQSTNLAPTV
GTYNHQEMLPGSVWMDRDVYLQGPIWAKIPETGAHFHPSPAMGGFGLKHPPPMMLIK
NTPVPSNVTAFSEIPVKSFITQYSTGQVTVEMEWELKKENSKRWNPEIQYTNNYNNPEF
VDFAPDTSGEYRTTRAIGTRYLTRPL.

In another embodiment, the VP1 sequence (from pig (denoted as Bpo37)) comprises the amino acid sequence of SEQ ID NO:57 and (amino acids 1-716); or comprises an amino acid sequence that is at least 60%, at least 65%, at least 70%, at least 75%, at least 80%, at least 85%, at least 90%, or at least 95% identical over the full length of the amino acid sequence of SEQ ID NO:57. The VP2 capsid protein spans amino acids 137-716 of SEQ ID NO:57 and the VP3 capsid protein spans amino acids 184-716 of SEQ ID NO:57.

(SEQ ID NO: 57)
MSFVDHPPDWLEEIGEGLKEFLGLKPGPLKPKPNQQKQDNARGLVLPGYNYL
GPGNGLDRGEPVNRADEVAREHDISYNEQLQAGDNPYLKYNHADAEFQEKLADDTSFG
GNLGKAVFQAKKRVLEPFGLVEEPAKTAAKGERIDDHYPKKKKARVEETEAGTSGGQQ
LQIPAQPASSLGADTMSAGGGSPLGDNNQGADGVGNASGDWHCDSTWMGDRVITKST
RTWVLPSYNNHLYKEIHSGSVDGSNANAYFGYSTPWGYFDFNRFHSHWSPRDWQRLV
NNYWGFRPRSLKVKIFNIQVKEVTVQDATTTIANNLTSTVQVFTDDDYQLPYVIGNGTE
GCLPAFPPQVFTLPQYGYATLNRNNTDDPTERSSFFCLEYFPSKMLRTGNSFEFTYSFEE
VPFHCSFAPSQNLFKLANPLVDQYLYRFVSTDTPGNIQFQKNLKARYANTYKNWFPGP
MCRTQGWYTGSGTYNRSGVTNFATSNRMDLEGASYQANPQPNGMTNTLQDSNKYAL
ENTMIFNSQNAEPGTTSLYQENNLLITSESETQPVNRVAYDTGGQMATNAQSTNLAPTV
GTYNHQEMLPGSVWMDRDVYLQGPIWAKIPETGAHFHPSPAMGGFGLKHPPPMMLIK
NTPVPSNVTAFSEIPVKSLITQYSTGQVTVEMEWELKKENSKRWNPEIQYTNNYNNPEF
VDFAPDTSGEYRTTRAIGTRYLTRPL.

In another embodiment, the VP1 sequence (from rhesus macaque (denoted as Brh26)) comprises the amino acid sequence of SEQ ID NO:58 and (amino acids 1-736); or comprises an amino acid sequence that is at least 60%, at least 65%, at least 70%, at least 75%, at least 80%, at least 85%, at least 90%, or at least 95% identical over the full length of the amino acid sequence of SEQ ID NO:58. The VP2 capsid protein spans amino acids 138-736 of SEQ ID NO:58 and the VP3 capsid protein spans amino acids 203-736 of SEQ ID NO:58.

(SEQ ID NO: 58)
MAADGYLPDWLEDNLSEGIREWWDLKPGAPKPKANQQKQDDGRGLVLPGY
KYLGPFNGLDKGEPANAADAAALEHDKAYDQQLKAGDNPYLRYNHADAEFQERLQED
TSFGGNLGRAIFQAKKRVLEPLGLVEEGAKTAPGKKGPVEQSPQEPDSSSGIGKTGQQPA
KKRLNFGQTGDSESVPDPQPLGEPPAAPSGLGPNTMASGGGAPMADNNEGADGVGNSS
GNWHCDSTWLGDRVITTSTRTWALPTYNNHLYKQISNGTSGGSTNDNTYFGYSTPWGY
FDFNRFHCHFSPRDWQRLINNNWGFRPKRLNFKLFNIQVKEVTTNEGTKTIANNLTSTV
QVFTDSEYQLPYVLGSAHQGCLPPFPADVFMVPQYGYLTLNNGSQALGRSSFYCLEYFP
SQMLRTGNNFQFSYTFEDVPFHSSYAHSQSLDRLMNPLIDQYLYYLVRTQTTGTGGTQT
LAFSQAGPSSMANQARNWVPGPCYRQQRVSTTTNQNNNSNFAWTGAAKFKLNGRDSL
MNPGVAMASHKDDDDRFFPSSGVLIFGKQGTGNDGVDYSQVLITDEEEIKATNPVATEE
YGAVAINNQAANTQAQTGLVHNQGVIPGMVWQNRDVYLQGPIWAKIPHTDGNFHPSP
LMGGFGLKHPPPQILIKNTPVPADPPLTFNQAKLNSFITQYSTGQVSVEIEWELQKENSKR
WNPEIQYTSNYYKSTNVDFAVNTEGVYSEPRPIGTRYLTRNL.

In another embodiment, the VP1 sequence (from rhesus macaque (denoted as Brh27)) comprises the amino acid sequence of SEQ ID NO:59 and (amino acids 1-736); or comprises an amino acid sequence that is at least 60%, at least 65%, at least 70%, at least 75%, at least 80%, at least 85%, at least 90%, or at least 95% identical over the full length of the amino acid sequence of SEQ ID NO:59. The VP2 capsid protein spans amino acids 138-736 of SEQ ID NO:59 and the VP3 capsid protein spans amino acids 203-736 of SEQ ID NO:59.

(SEQ ID NO: 59)
MAADGYLPDWLEDNLSEGIREWWDLKPGAPKPKANQQKQDDGRGLVLPGY
KYLGPFNGLDKGEPVNAADAAALEHDKAYDQQLKAGDNPYLRYNHADAEFQERLQED
TSFGGNLGRAIFQAKKRVLEPLGLVEEGAKTAPGKKRPVEQSPQEPDSSSGIGKTGQQPA
KKRLNFGQTGDSESVPDPQPLGEPPAAPSGLGPNTMASGGGAPMADNNEGADGVGNPS
GNWHCDSTRLGDRVITTSTRTWALPTYNNHLYKQISNGTSGGSTNDNTYFGYSTPWGY
FDFNRFHCHFSPRDWQRLINNNWGFRPKRLNFKLFNIQVKEVTTNEGTKTIANNLTSTV
QVFTDSEYQLPYVLGSAHQGCLPPFPADVFMVPQYGYLTLNNGSQALGRSSFYCLEYFP
SQMLRTSNNFQFSYTFEDVPFHSSYAHSQSLDRLMNPLIDQYLYYLVRTQTTGTGGTQT
LAFSQAGPSSMANQARNWVPGPCYRQQRVSTTTNQNNNSNFAWTGAAKFKLNGRDSL
MNPGVAMASHKDDDDRFFPSSGVLIFGKQGTGNDGVDYSQVLITDEEEIKATNPVATEE
YGAVAINNQAANTQAQTGLVHNQGVIPGMVWQNRDVYLQGPIWAKIPHTDGNFHPSP
LMGGFGLKLPPPQILIKNTPVPADPPLTFNQAKLNSFITQYSTGQVSVEIEWELQKENSKR
WNPEIQYTSNYYKSTNVDFAVNTEGVYSEPRPIGTRYLTRNL.

In another embodiment, the VP1 sequence (from rhesus macaque (denoted as Brh28)) comprises the amino acid sequence of SEQ ID NO:60 and (amino acids 1-736); or comprises an amino acid sequence that is at least 60%, at least 65%, at least 70%, at least 75%, at least 80%, at least 85%, at least 90%, or at least 95% identical over the full length of the amino acid sequence of SEQ ID NO:60. The VP2 capsid protein spans amino acids 138-736 of SEQ ID NO:60 and the VP3 capsid protein spans amino acids 203-736 of SEQ ID NO:60.

(SEQ ID NO: 60)
MAADGYLPDWLEDNLSEGIREWWDLKPGAPKPKANQQKQDDGRGLVLPGY
KYLGPFNGLDKGEPVNAADAAALEHDKAYDQQLKAGDNPYLRYNHADAEFQERLQED
TSFGGNLGRAIFQAKKRVLEPLGLVEEGAKTAPGKKRPVEQSPQEPDSPSGIGKTGQQPA
KKRLNFGQTGDSESVPDPQPLGEPPAAPSGLGPNTMASGGGAPMADNNEGADGVGNSS
GNWHCDSTWLGDRVITTSTRTWALPTYNNHLYKQISNGTSGGSTNDNTYFGYSTPWGY
FDFNRFHCHFSPRDWQRLINNNWGFRPKRLNFKLFNIQVKEVTTNEGTKTIANNLTSTV
QVFTDSEYQLPYVLGSAHQGCLPPFPADVFMVPQYGYLTLNNGSQALGRSSFYCLEYFP
SQMLRTGNNFQFSYTFEDVPFHSSYAHSQSLDRLMNPLIDQYLYYLVRTQTTGTGGTQT
LAFSQAGPSSMANQARNWVPGPCYRQQRVSTTTNQNNNSNFAWTGAAKFKLNGRDSL
MNPGVAMASHKDDDDRFFPSSGVLIFGKQGTGNDGVDYSQVLITDEEEIKATNPVATEE
YGAVAINNQAANTQAQTGLVHNQGVIPGMVWQNRDVYLQGPIWAKIPHTDGNFHPSP
LMGGFGLKHPPPQILIKNTPVPADPPLTFNQAKLNSFITQYSTGQVSVEIEWELQKENSKR
WNPEIQYTSNYYKSTNVDFAVNTEGVYSEPRPIGTRYLTRNL.

In another embodiment, the VP1 sequence (from rhesus macaque (denoted as Brh29)) comprises the amino acid sequence of SEQ ID NO:61 and (amino acids 1-736); or comprises an amino acid sequence that is at least 60%, at least 65%, at least 70%, at least 75%, at least 80%, at least 85%, at least 90%, or at least 95% identical over the full length of the amino acid sequence of SEQ ID NO:61. The VP2 capsid protein spans amino acids 138-736 of SEQ ID NO:61 and the VP3 capsid protein spans amino acids 203-736 of SEQ ID NO:61.

(SEQ ID NO: 61)
MAADGYLPDWLEDNLSEGIREWWDLKPGAPKPKANQQKQDDGRGLVLPGY
KYLGPFNGLDKGEPVNAADAAALEHDKAYDQQLKAGDNPYLRYNHADAEFQERLQED
TSFGGNLGRAIFQAKRRVLEPLGLVEEGAKTAPGKKRPVEQSPQEPDSSSGIGKTGQQPA
KKRLNFGQTGDSESVPDPQPLGEPPAAPSGLGPNTMASGGGAPMADNNEGADGVGNSS
GNWHCDSTWLGDRVITTSTRTWALPTYNNHLYKQISNGTSGGSTNDNTYFGYSTPWGY
FDFNRFHCHFSPRDWQRLINNNWGFRPKRLNFKLFNIQVKEVTTNEGTKTIANNLTSTV
QVFTDSEYQLPYVLGSAHQGCLPPFPADVFMVPQYGYLTLNNGSQALGRSSFYCLEYFP
SQMLRTSNNFQFSYTFEDVPFHSSCAHSQSLDRLMNPLIDQYLYYLVRTQTTGTGGTQT
LAFSQAGPGSMANQARNWVPGPCYRQQRVSTTTNQNNNSNFAWTGAAKFKLNGRDSL
MNPGVAMASHKDDDDRFFPSSGVLIFGKQGTGNDGVDYSQVLITDEEEIKATNPVATEE
YGAVAINNQAANTQAQTGLVHNQGVIPGMVWQNRDVYLQGPIWAKIPHTDGNFHPPP
LMGGFGLKHPPPQILIKNTPVPADPPLTFNQAKLNSFITQYSTGQVSVEIEWELQKENSKR
WNPEIQYTSNYYKSTNVDFAVNTEGVYSEPRPIGTRYLTRNL.

In another embodiment, the VP1 sequence (from rhesus macaque (denoted as Brh30)) comprises the amino acid sequence of SEQ ID NO:62 and (amino acids 1-736); or comprises an amino acid sequence that is at least 60%, at least 65%, at least 70%, at least 75%, at least 80%, at least 85%, at least 90%, or at least 95% identical over the full length of the amino acid sequence of SEQ ID NO:62. The VP2 capsid protein spans amino acids 138-736 of SEQ ID NO:62 and the VP3 capsid protein spans amino acids 203-736 of SEQ ID NO:62.

(SEQ ID NO: 62)
MAADGYLPDWLEDNLSEGIREWWDLKPGAPKPKANQQKQD
DGRGLVFPGYKYLGPFNGLDKGEPVNAADAAALEHDKAYD
QQLKAGDNPYLRYNHADAEFQERLQEDTSFGGNLGRAIFQ
AKKRVLEPLGLVEEGAKTAPGKKRPVEQSPQEPDSSSGIG
KTGRQPAKKRLNFGQTGDSESVPDPQPLGEPPAAPSGLGP
NTMASGGGAPMADNNEGADGVGNSSGNWHCDSTWLGDRVI
TTSTRTWALPTYNNHLYKQISNGTPGGSTNDNTYFGYSAP
WGYFDFNRFHCHFSPRDWQRLINNNWGFRPKRLNFKLFNI
QVKEVTTNEGTKTIANNLTSTVQVFTDSEYQLPYVLGSAH
QGCLPPFPADVFMVPQYGYLTLNNGSQALGRSSFYCLEYF
PSQMLRTGNNFQFSYTFEDVPFHSSYAHSQSLDRLMNPLI
DQYLYYLVRTQTTGTGGTQTLAFSQAGPSSMANQARNWVP
GPCYRQQRVSTTTNQNNNSNFAWTGAAKFKLNGRDSLMNP
GVAMASHKDDDDRFFPSSGVLIFGKQGTGNDGVDYSQVLI
TDEEEIKATNPVATEEYGAVAINNQAANTQAQTGLVHNQG
VIPGMVWQNRDVYQQGPIWAKIPHTDGNFHPSPLMGGFGL
KHPPPQILIKNTPVPADPPLTFNQAKLNSFITQYSTGQVS
VEIEWELQKENSKRWNPEIQYTSNYYKSTNVDFAVNTEGV
YSEPRPIGTRYLTRNL.

In another embodiment, the VP1 sequence (from rhesus macaque (denoted as Brh31)) comprises the amino acid sequence of SEQ ID NO:63 and (amino acids 1-736); or comprises an amino acid sequence that is at least 60%, at least 65%, at least 70%, at least 75%, at least 80%, at least 85%, at least 90%, or at least 95% identical over the full length of the amino acid sequence of SEQ ID NO:63. The VP2 capsid protein spans amino acids 138-736 of SEQ ID NO:63 and the VP3 capsid protein spans amino acids 203-736 of SEQ ID NO:63.

(SEQ ID NO: 63)
MAADGYLPDWLEDNLSEGIREWWDLKPGAPKPKANQQKQD
DGRGLVLPGYKYLGPFNGLDKGEPVNAADAAALEHDKAYD
QQLKAGDNPYLRYNHADAEFQERLQEDTSFGGNLGRAIFQ
AKKRVLEPLGLVEEGAKTAPGKKRPVEQSPQEPDSSSGIG
KTGQQPAKKRLNSGQTGDSESVPDPQPLGEPPAAPSGLGP
NTMASGGGAPMADNNEGADGVGNSSGNWHCDSTWLGDRVI
TTSTRTWALPTYNNHLYKQISNGTSGGSTNDNTYFGYSTP
WGYFDFNRFHCHFSPRDWQRLINNNWGFRPKRLNFKLFNI
QVKEVTTNEGTKTIANNLTSTVQVFTDSEYQLPYVLGSAY
QGCLPPFPADVFMVPQYGYLTLNNGSQALGRSSFYCLEYF
PSQMLRTGNNFQSSYTFEDVPFHSSYAHSQSLDRLMNPLI
DQYLYYLVRTQTTGTGGTQTLAFSQAGPSSMANQARNWVP
GPCYRQQRVSTTTNQNNNSNFAWTGAAKFKLNGRDSLMNP
GVAMASHKDDDDRFFPSSGVLIFGKQGTGNDGVDYSQVLI
TDEEEIKATNPVATEEYGAVAINNQAANTQAQTGLVHNQG
VIPGMVWQNRDVYLQGPIWAKIPHTDGNFHPSPLMGGFGL
KHPPPQILIKNTPVPADPPLTFNQAKLNSFITQYSTGQVS
VEIEWELQKENSKRWNPEIQYTSNYYKSTNVDFAVNTEGV
YSEPRPIGTRYLTRNL.

In another embodiment, the VP1 sequence (from rhesus macaque (denoted as Brh32)) comprises the amino acid sequence of SEQ ID NO:64 and (amino acids 1-736); or comprises an amino acid sequence that is at least 60%, at least 65%, at least 70%, at least 75%, at least 80%, at least 85%, at least 90%, or at least 95% identical over the full length of the amino acid sequence of SEQ ID NO:64. The VP2 capsid protein spans amino acids 138-736 of SEQ ID NO:64 and the VP3 capsid protein spans amino acids 203-736 of SEQ ID NO:64.

(SEQ ID NO: 64)
MAADGYLPDWLEDNLSEGIREWWDLKPGASKPKANQQKQD
DGRGLVLPGYKYLGPFNGLDKGEPVNAADAAALEHDKAYD
QQLKAGDNPYLRYNHADAEFQERLQEDTSFGGNLGRAVFQ
AKKRVLEPLGLVEEGAKTVPGKKRPVEQSPQEPDSSSGIG
KTGQQPAKKRLNFGQTGDSESVPDPQPLGEPPAAPSGLGP
NTMASGGGAPMADNNEGADGVGNSSGNWHCDSTWLGDRVI
TTSTRTWALPTYNNHLYKQISNGTSGGSTNDNTYFGYSTP
WGYFDFNRFHCHFSPRDWQRLINNNWGFRPKRLNFKLFNI
QVKEVTTNEGTKTIANNLTSTVQVFTDSEYQLPYVLGSAY
QGCLPPFPADVFMVPQYGYLTLNNGSQALGRSSFYCLEYF
PSQMLRTGNNFQFSYTFEDVPFHSSYAHSQSLDRLMNPLI
DQYLYYLVRTQTTGTGGTQTLAFSQAGPSSMANQARNWVP
GPCYRQQRVSTTTNQNNNSNFAWTGAAKFKLNGRDSLMNP
GVAMASHKDDDDRFFPSSGVLIFGKQGTGNDGVDYSQVLI
TDEEEIKATNPVATEEYGAVAINNQAANTQAQTGLVHNQG
VIPGMVWQNRDVYLQGPIWAKIPHTDGNFHPSPLMGGFGL
KHPPPQILIKNTPVPADPPLTFNQAKLNSFITQYSTGQVS
VEIEWELQKENSKRWNPEIQYTSNYYKSTNVDFAVNTEGV
YSEPRPIGTRYLTRNL.

In another embodiment, the VP1 sequence (from rhesus macaque (denoted as Brh33)) comprises the amino acid sequence of SEQ ID NO:65 and (amino acids 1-736); or comprises an amino acid sequence that is at least 60%, at least 65%, at least 70%, at least 75%, at least 80%, at least 85%, at least 90%, or at least 95% identical over the full length of the amino acid sequence of SEQ ID NO:65. The VP2 capsid protein spans amino acids 138-736 of SEQ ID NO:65 and the VP3 capsid protein spans amino acids 203-736 of SEQ ID NO:65.

(SEQ ID NO: 65)
MAADGYLPDWLEDNLSEGIREWWDLKPGASKPKANQQKQD
DGRGLVLPGYKYLGPFNGLDKGEPVNAADAAALEHDKAYD
QQLKAGDNPYLRYNHADAEFQERLQEDTSFGGNLGRAVFQ
AKKRVLEPLGLVEEGAKTVPGKKRPVEQSPQEPDSSSGIG
KTGQQPAKKRLNFGQTGDSESVPDPQPLGEPPAAPSGLGP
NTMASGGGAPMADNNEGADGVGNSSGNWHCDSTWLGDRVI
TTSTRTWALPTYNNHLYKQISNGTSGGSTNDNTYFGYSTP
WGYFDFNRFHCHFSPRDWQRLINNNWGFRPKRLNFKLFNI
QVKEVTTNEGTKTIANNLTSTVQVFTDSEYQLPYVLGSAY
QGCLPPFPADVFMVPQYGYLTLNNGSQALGRSSFYCLEYF
PSQMLRTGNNFQFSYTFEDVPFHSSYAHSQSLDRLMNPLI
DQYLYYLVRTQTTGTGGTQTLAFSQAGPSSMANQARNWVP
GPCYRQQRVSTTTNQNNNSNFAWTGAAKFKLNGRDSLMNP
GVAMASHKDDDDRFFPSSGVLIFGKQGTGNDGVDYSQVLI
TDEEEIKATNPVATEEYGAVAINNQAANTQAQTGLVHNQG
VIPGMVWQNRDVYLQGPIWAKIPHTDGNFHPSPLMGGFGL
KHPPPQILIKNTPVPADPPLTFNQAKLNSFITQYSTGQVS
VEIEWELQKENSKRWNPEIQYTSNYYKSTNVDFAVNTEGV
YSEPRPIGTRYLTRNL.

In another embodiment, the VP1 sequence (from formosan macaque (denoted as Bfm17)) comprises the amino acid sequence of SEQ ID NO:66 and (amino acids 1-737); or comprises an amino acid sequence that is at least 60%, at least 65%, at least 70%, at least 75%, at least 80%, at least 85%, at least 90%, or at least 95% identical over the full length of the amino acid sequence of SEQ ID NO:66. The VP2 capsid protein spans amino acids 138-736 of SEQ ID NO:66 and the VP3 capsid protein spans amino acids 203-736 of SEQ ID NO:66.

(SEQ ID NO: 66)
MAADGYLPDWLEDNLSEGIREWWALKPGAPKPKANQQKQD
DGRGLVLPGYKYLGPFNGLDKGEPVNAADAAALEHDKAYD
QQLQAGDNPYLRYNHADAEFQERLQEDTSFGGNLGRAVFQ
AKKRVLEPLGLVEEGAKTAPGKKRPVEQSPQEPDSSSGIG
KKGQQPAKKRLNFGQTGDSESVPDPQPLGEPPAAPSSLGL
NTMAAGGGAPMADNNEGADGVGSSSGNWHCDSTRLGDRVI
TTSTRTWALPTYNNHLYKQISNGTSGGSTNDNTYFGYSTP
WGYFDFNRFHCHFSPRDWQRLINNNWGFRPKKLSFKLFNI
QVKEVTQNEGTKTIANNLTSTIQVFTDSEYQLSYVLGSAH
QGCLPPFPADVFMIPQYGYLTLNNGSQAVGRSSFYCLEYF
PSQMLRTGNNFSFSYTFEDVPFHSSYAHSQSLDRLMNPLI
DQYLYYLSRTQSTGGTAGTQQLLFSQAGPNNMSAQAKNWL
PGPMYRQQRVSTTLSQNNNSNFAWTGGTKYHLNGRDSLVN
PGVAMATNKDDEDRFFPSSGVLMFGKQGAGKDNVDYSSVM
LTSEEEIKTTNPVATEQYGVVADNLQQQNTAPIVGAVNSQ
EALPGMVWQNRDVYLQGPIWAKIPHTDGNFHPSPLMGGFG
LKHPPPQILIKNTPVPADPPTAFNQAKLNSFITQYSTGQV
SVEIEWELQKENSKRWNPEIQYTSNYYKSTNVDFAVDTEG
VYSEHRPIGTRYLTRNL.

In another embodiment, the VP1 sequence (from formosan macaque (denoted as Bfm18)) comprises the amino acid sequence of SEQ ID NO:67 and (amino acids 1-737); or comprises an amino acid sequence that is at least 60%, at least 65%, at least 70%, at least 75%, at least 80%, at least 85%, at least 90%, or at least 95% identical over the full length of the amino acid sequence of SEQ ID NO:67. The VP2 capsid protein spans amino acids 138-736 of SEQ ID NO:67 and the VP3 capsid protein spans amino acids 203-736 of SEQ ID NO:67.

(SEQ ID NO: 67)
MAADGYLPDWLEDNLSEGIREWWALKPGAPKPKANQQKQD
DGRGLVLPGYKYLGPFNGLDKGEPVNAADAAALEHDKAYD
QQLQAGDNPYLRYNHADAEFQERLQEDTSFGGNLGRAVFQ
AKKRVLEPLGLVEEGAKTAPGKKRPVEQSPQEPDSSSGIG
KKGQQPAKKRLNFGQTGDSESVPDPQPLGEPPAAPSSLGL
NTMAAGGGAPMADNNEGADGVGSSSGNWHCDSTWLGDRVI
TTSTRTWALPTYNNHLYKQISNGTSGGSTNDNTYFGYSTP
WGYFDFNRFHCHFSPRDWQRLINNNWGFRPKKLSFKLFNI
QVKEVTQNEGTKTIANNLTSTIQVFTDSEYQLPYVPGSAH
QGCLPPFPADVFMIPQYGYLTLNNGSQAVGRSSFYCLEYF
PSQMLRTGNNFSFSYTFEDVPFHSSYAHSQSLDRLMNPLI
DQYLYYLSRTQSTGGTAGTQQLLFSQAGPNNMSAQAKNWL
PGPMYRQQRVSTTLSQNNNSNFAWTGGTKYHLNGRDSLVN
PGVAMATNKDDEDRFFPSSGVLMFGKQGAGKDNVDYSSVM
LTSEEEIKTTNPVATEQYGVVADNLQQQNTAPIVGAVNSQ
GALPGMVWQNRDVYPQGPIWAKIPHTDGNFHPSPLMGGFG
LKHPPPQILIKNTPVPADPPTAFNQAKLNSFITQYSTGQV
SVEIEWELQKENSKRWNPEIQYTSNYYKSTNVDFAVNTEG
VYSEPRPIGTRYLTRNL.

In another embodiment, the VP1 sequence (from formosan macaque (denoted as Bfm.20)) comprises the amino acid sequence of SEQ ID NO:68 and (amino acids 1-737); or comprises an amino acid sequence that is at least 60%, at least 65%, at least 70%, at least 75%, at least 80%, at least 85%, at least 90%, or at least 95% identical over the full length of the amino acid sequence of SEQ ID NO:68. The VP2 capsid protein spans amino acids 138-736 of SEQ ID NO:68 and the VP3 capsid protein spans amino acids 203-736 of SEQ ID NO:68.

(SEQ ID NO: 68)
MAADGYLPDWLEDNLSEGIREWWALKPGAPKPKANQQKQD
DGRGLVLPGYKYLGPFNGLDKGEPVNAADAAALEHDKAYD
QQLQAGDNPYLRYNHADAEFQERLQEDTSFGGNLGRAVFQ
AKKRVLEPPGLVEEGAKTAPGKKRPVEQSPQEPDSSSGIG
KKGQQPAKKRLNFGQTGDSESVPDPQPLGEPPAAPSSLGL
NTMAAGGGAPMADNNEGADGVGSSSGNWHCDSTWLGDRVI
TTSTRTWALPTYNNHLYKQISNGTSGGSTNDNTYFGYSTP
WGYFDFNRFHCHFSPRDWQRLINNNWGFRPKKLSFKLFNI
QVKEVTQNEGTKTIANNLTSTIQVFTDSEYQLPYVLGSAH
QGCLPPFPADVFMIPQYGYLTLNNGSQAVGRSFFYCLEYF
PSQMLRTGNNFSFSYTFEDVPFHSSYAHSQSLDRLMNPLI
DQYLYYLSRTQSTGGTAGTQQLLFSQAGPNNMSAQAKNWL
PGPMYRQQRVSTTLSQNNNSNFAWTGGTKYHLNGRDSLVN
PGVAMATNKDDEDRFFPSSGVLMFGKQGAGKDNVDYSSVM
LTSEEEIKTTNPVATEQYGVVADNLQQQNTAPIVGAVNSQ
GALPGMVWQNRDVYLQGPIWAKIPHTDGNFHPSPLMGGFG
LKHPPPQILIKNTPVPADPPTAFNQAKLNSFITQYSTGQV
SVEIERELQKENSKRWNPEIQYTSNYYKSTNVDFAVNTEG
VYSEPRPIGTRYLTRNL.

In another embodiment, the VP1 sequence (from formosan macaque (denoted as Bfm21)) comprises the amino acid sequence of SEQ ID NO:69 and (amino acids 1-737); or comprises an amino acid sequence that is at least 60%, at least 65%, at least 70%, at least 75%, at least 80%, at least 85%, at least 90%, or at least 95% identical over the full length of the amino acid sequence of SEQ ID NO:69. The VP2 capsid protein spans amino acids 138-736 of SEQ ID NO:69 and the VP3 capsid protein spans amino acids 203-736 of SEQ ID NO:69.

(SEQ ID NO: 69)
MAADGYLPDWLEDNLSEGIREWWALKPGAPKPKANQQKQD
DGRGLVLPGYKYLGPFNGLDKGEPVNAADAAALEHDKAYD
QQLQAGDNPYLRYNHADAEFQERLQEDTSFGGNLGRAVFQ
AKKRVLEPLGLVEEGAKTAPGKERPVEQSPQEPDSSSGIG
KKGQQPAKKRLNFGQTGDSESVPDPQPLGEPPAAPSSLGL
NTMAAGGGAPMADNNEGADGVGSSSGNWHCDSTWLGDRVI
TTSTRTWALPTYNNHLYKQISNGTSGGSTNDNTYFGYSTP
WGYFDFNRFHCHFSPRDWQRLINNNWGFRPKKLSFKLFNI
QVKEVTQNEGTKTIANNLTSTIQVFTDSEYQLPYVLGSAH
QGCLPPFPADVFMIPQYGYLTLNNGSQAVGRSSFYCLEYF
PSQMLRTGNNFSFSYTFEDVPFHSSYAHSQSLDRLMNPLI
DQYLYYLSRTQSTGGTAGTQQLLFSQAGPNNMSAQAKNWL
PGPMYRQQRVSTTLSQNNNSNFAWTGGTKYHLNGRDSLVN
PGVAMATNKDDEDRFFPSSAVLMFGKQGAGKDNVDYSSVM
LTSEEEIKTTNPVTTEQYGVVADNLQQQNTAPIVGAVNSQ
GALPGMVWQNRDVYLQGPIWAKIPHTDGNFHPSPLMGGFG
LKHPPPQILIKNTPVPADPPTAFNQAKLNSFITQYSTGQV
SVEIEWELQKENSKRWNPEIQYTSNYYKSTNVDFAVNTES
VYSEPRPIGTRYLTRNL.

In another embodiment, the VP1 sequence (from formosan macaque (denoted as Bfm24)) comprises the amino acid sequence of SEQ ID NO:70 and (amino acids 1-737); or comprises an amino acid sequence that is at least 60%, at least 65%, at least 70%, at least 75%, at least 80%, at least 85%, at least 90%, or at least 95% identical over the full length of the amino acid sequence of SEQ ID NO:70. The VP2 capsid protein spans amino acids 138-736 of SEQ ID NO:70 and the VP3 capsid protein spans amino acids 203-736 of SEQ ID NO:70.

(SEQ ID NO: 70)
MAADGYLPDWLEDNLSEGIREWWALKPGAPKPKANQQKQD
DGRGLVLPGYKYLGPFNGPDKGEPVNAADAAALEHDKAYD
QQLQAGDNPYLRYNHADAEFQERLQEDTSFGGNLGRAVFQ
AKKRVLEPLGLVEEGAKTAPGKKRPVEQSPQEPDSSSGIG
KKGQQPAKKRLNFGQTGDSESVPDPQPLGEPPAAPSSLGL
NTMAAGGGAPMADNNEGADGVGSSSGNWHCDSTWLGDRVI
TTSTRTWALPTYNNHLYKQISNGTSGGSTNDNTYFGYSTP
WGYFDFNRSHCHFSPRDWQRLINNNWGFRPKKLSFKLFNI
QVKEVTQNEGTKTIANNLTSTIQVFTDSEYQLPYVLGSAH
QGCLPPFPADVFMIPQYGYLTLNNGSQAVGRSSFYCLEYF
PSQMLRTGNNFSFSYTFEDVPFHSSYAHSQSLDRLMNPLI
DQYLYYLSRTQSTGGTAGTQQLLFSQAGPNNMSAQAKNWL
PGPMYRQQRVSTTLSQNNNSNFAWTGGTKYHLNGRDSLVN
PGVAMATNKDDEDRFFPSSGVLMFGKQGAGKDNVDYSSVM
LTSEEEIRTTNPVATEQYGVVADNLQQQNTAPIVGAVNSQ
GALPGMVWQNRDVYLQGPIWAKIPHTDGNFHPSPLMGGFG
LKHPPPQILIKNTPVPADPPTAFNQAKLNSFITQYSTGQV
SVEIEWELQKENSKRWNPEIQYTSNYYKSTNVDFAVNTEG
VYSEPRPIGTRYLTRNL.

In another embodiment, the VP1 sequence (from formosan macaque (denoted as Bfm25)) comprises the amino acid sequence of SEQ ID NO:71 and (amino acids 1-737); or comprises an amino acid sequence that is at least 60%, at least 65%, at least 70%, at least 75%, at least 80%, at least 85%, at least 90%, or at least 95% identical over the full length of the amino acid sequence of SEQ ID NO:71. The VP2 capsid protein spans amino acids 138-736 of SEQ ID NO:71 and the VP3 capsid protein spans amino acids 203-736 of SEQ ID NO:71.

(SEQ ID NO: 71)
MAADGYLPDWLEDNLSEGIREWWALKPGAPKPKANQQKQD
DGRGLVLPGYKYLGPFNGLDKGEPVNAADAAALEHDKAYD
QQLQAGDNPYLRYNHADAEFQERLQEDTSFGGNLGRAVFQ
AKKRVLEPLGLVEEGAKTAPGKKRPVEQSPQEPDSSSGIG
KKGQQPAKKRLNFGQTGDSESVPDPQPLGEPPAAPSSLGL
NTMAAGGGAPMADNNEGADGVGSSSGNWHCDSTWLGDRVI
TTSTRTWALPTYNNHLYKQISNGTSGGSTNDNTYFGYSTP
WGYFDFNRFHCHFSPRDWQRLINNNWGFRPKKLSFKLFNI
QVKEVTQNEGTKTIANNLTSTIQVFTDSEYQLPYVLGSAH
QGCLPPFPADVFMIPQYGYLTLNNGSQAVGRSSFYCLEYF
PSQMLRTGNNFSFSYTFEDVPFHSSYAHSQSLDRLMNPLI
DQYLYYLSRTQSTGGTAGTQQLLFSQAGPNNMSAQAKNWL
PGPMYRQQRVSTTLSQNNNSNFAWTGGTKYHLNGRDSLVN
PGVAMATNKDDEDRLFPSSGVLMFGKQGAGKDNVDYSSVM
LTSEEEVKTTNPVATEQYGVVADNLQQQNTAPIVGAVNSQ
GALPGMVWQNRDVYLQGPIWAKIPHTDGNFHPSPLMGGFG
LKHPPPQILIKNTPVPADPPTAFNQAKLNSFITQYSTGQV
SVEIEWELQKENSKRWNPEIQYTSNYYKSTNVDFAVNTEG
VYSEPRPIGTRYLTRNL.

In another embodiment, the VP1 sequence (from formosan macaque (denoted as Bfm27)) comprises the amino acid sequence of SEQ ID NO:72 and (amino acids 1-737); or comprises an amino acid sequence that is at least 60%, at least 65%, at least 70%, at least 75%, at least 80%, at least 85%, at least 90%, or at least 95% identical over the full length of the amino acid sequence of SEQ ID NO:72. The VP2 capsid protein spans amino acids 138-736 of SEQ ID NO:72 and the VP3 capsid protein spans amino acids 203-736 of SEQ ID NO:72.

(SEQ ID NO: 72)
MAADGYLPDWLEDNLSEGIREWWALKPGAPKPKANQQKQD
DGRGLVLPGYKYLGPFNGLDKGEPVNAADAAALEHDKAYD
QQLQAGDNPYLRYNHADAEFQERLQEDTSFGGNLGRAVFQ
AKRRVLEPLGLVEEGAKTAPGKKRPVEQSPQEPDSSSGIG
KKGQQPAKKRLNFGQTGDSESVPDPQPLGEPPAAPSSLGL
NTMAAGGGAPMADNNEGADGVGSSSGNWHCDSTWLGDRVI
TTSTRTWALPTYNNHLYKQISNGTSGGSTNDNTYFGYSTP
WGYFDFNRFHCHFSPRDWQRLINNNWGFRPKKLSFKLFNI
QVKEVTQNEGTKTIANNLTSTIQVFTDSEYQLPYVLGSAR
QGCLPPFPADVFMIPQYGYLTLNNGSQAVGRSSFYCLEYF
PSQMLRTGNNFSFSYTFEDVPFHSSYAHSQSLDRLMNPLI
DQYLYYLSRTQSTGGTAGTQQLLFSQAGPNNMSAQAKNWL
PGPMYRQQRVSTTLSQNNNSNFAWTGGTKYHLNGRDSLVN
PGVAMATNKDDEDRFFPSSGVLMFGKQGAGKDNVDYSSVM
LTSEEEIKTTNPVATEQYGVVADNLQQQNTAPIVGAVNSQ
GALPGMVWQNRDVYLQGPIWAKIPHTDGNFHPSPLMGGFG
LKHPPPQILIKNTPVPADPPTAFNQAKLNSFITQYSTGQV
SVEIEWELQKENSKRWNPEIQYTSNYYKSTNVDFAVDTEG
VYSEPRPIGTRYLTRNL.

In another embodiment, the VP1 sequence (from formosan macaque (denoted as Bfm32)) comprises the amino acid sequence of SEQ ID NO:73 and (amino acids 1-737); or comprises an amino acid sequence that is at least 60%, at least 65%, at least 70%, at least 75%, at least 80%, at least 85%, at least 90%, or at least 95% identical over the full length of the amino acid sequence of SEQ ID NO:73. The VP2 capsid protein spans amino acids 138-736 of SEQ ID NO:73 and the VP3 capsid protein spans amino acids 203-736 of SEQ ID NO:73.

(SEQ ID NO: 73)
MAADGYLPDWLEDNLSEGIREWWALKPGAPKPKANQQKQD
DGRGLVLPGYKYLGPFNGLDKGEPVNAADAAALEHDKAYD
QQLQAGDNPYLRYNHADAEFQERLQEDTSFGGNLGRAVFQ
AKKRVLEPLGLVEEGAKTAPGKKRPVEQSPQEPDSSSGIG
KKGQQPAKKRLNFGQTGDSESVPDPQPLGEPPAAPSSLGL
NTMAAGGGAPMADNNEGADGVGSSSGNWHCDSTWLGDRVI
TTSTRTWALPTYNNHLYKQISNGTSGGSTNDNTYFGYSTP
WGYFDFNRFHCHFSPRDWQRLINNNWGFRPKKLSFKLFDI
QVKEVTQNEGTKTIANNLTSTIQVFTDSEYQLPYVLGSAH
QGCLPPFPADVFMIPQYGYLTLNNGSQAVGRSSFYCLEYF
PSQMLRTGNNFSFSYTFEDVPFHSSYAHSQSLDRLMNPLI
DQYLYYLSRTQSTGGTAGTQQLLFSQAGPNNMSAQAKNWL
PGPMYRQQRVSTTLSQNNNSNFAWTGGTKYHLNGRDSLVN
PGVAMATNKDDEDRFFPSSGVLMFGKQGAGKDNVDYSSVM
LTSEEEIKTTNPVATEQYGVVADNLQQQNTAPIVGAVNSQ
GALPGMVWQNRDVYLQGPIWAKIPHTDGNFHPSPLMGGFG
LKHPPPQILIKNTPVPADPPTAFNQAKLNSFITQYSTGQV
SVEIEWELQKENSKRWNPEIQYTSNYYKSTNVDFAVNTEG
VYSEPRPIGTRYLTRNL.

In another embodiment, the VP1 sequence (from formosan macaque (denoted as Bfm33)) comprises the amino acid sequence of SEQ ID NO:74 and (amino acids 1-737); or comprises an amino acid sequence that is at least 60%, at least 65%, at least 70%, at least 75%, at least 80%, at least 85%, at least 90%, or at least 95% identical over the full length of the amino acid sequence of SEQ ID NO:74. The VP2 capsid protein spans amino acids 138-736 of SEQ ID NO:74 and the VP3 capsid protein spans amino acids 203-736 of SEQ ID NO:74.

(SEQ ID NO: 74)
MAADGYLPDWLEDNLSEGIREWWALKPGAPKPKANQQKQD
DGRGLVLPGYKYLGPFNGLDKGEPVNAADAAALEHDKAYD
QQLQAGDNPYLRYNHADAEFQERLQEDTSFGGNLGRAVFQ
AKKRVLEPLGLVEEGAKTAPGKKRPVEQSPQEPDSSSGIG
KKGQQPAKKRLNFGQTGDSESVPDPQPLGEPPAAPSSLGL
NTMAAGGGAPMADNNEGADGVGSSSGNWHCDSTWLGDRVI
TTSTRTWALPTYNNHLYKQISNGTSGGSTNDNTYFGYSTP
WGYFDFNRFHCHFSPRDWQRLINNNWGFRPKKLSFKLFNI
QVKEVTQNEGTKTIANNLTSTIQVFTDSEYQLPYVLGSAH
QGCLPPFPADVFMIPQYGYLTLNNGSQAVGRSSFYCLEYF
PSQMLRTGNNFSFSYTFEDVPFHSSYAHSQSLDRLMNPLI
DQYLYYLSRTQSTGGTAGTQQLLFSQAGPNNMSAQAKNWL
PGPMYRQQRVSTTLSQNNNSNFAWTGGTKYHLNGRDSLVN
PGVAMATNKDDEDRFFPSSAVLMFGKQGAGKDNVDYSSVM
LTSEEEIKTTNPVATEQYGVVADNLQQQNTAPIVGAVNSQ
GALPGMVWQNRDVYLQGPIWAKIPHTDGNFHPSPLMGGFG
LKHPPPQILIKNTPVPADPPTAFNQAKLNSFITQYSTGQV
SVEIEWELQKENSKRWNPEIQYTSNYYKSTNVDFAVNTES
VYSEPRPIGTRYLTRNL.

In another embodiment, the VP1 sequence (from formosan macaque (denoted as Bfm34)) comprises the amino acid sequence of SEQ ID NO:75 and (amino acids 1-737); or comprises an amino acid sequence that is at least 60%, at least 65%, at least 70%, at least 75%, at least 80%, at least 85%, at least 90%, or at least 95% identical over the full length of the amino acid sequence of SEQ ID NO:75. The VP2 capsid protein spans amino acids 138-736 of SEQ ID NO:75 and the VP3 capsid protein spans amino acids 203-736 of SEQ ID NO:75.

(SEQ ID NO: 75)
MAADGYLPDWLEDNLSEGIREWWALKPGAPKPKANQQKQD
DGRGLVLPGYKYLGPFNGLDKGEPVNAADAAALEHDKAYD
QQLQAGDNPYLRYNHADAEFQERLQEDTSFGGNLGRAVFQ
AKKRVLEPLGLVEEGAKTAPGKKRPVEQSPQEPDSSSGIG
KKGQQPAKKRLNFGQTGDSESVPDPQPLGEPPAAPSSLGL
NTMAAGGGAPMADNNEGADGVGSSSGNWHCDSTWLGDRVI
TTSTRTWALPTYNNHLYKQISNGTSGGSTNDNTYFGYSTP
WGYFDFNRFHCHFSPRDWQRLINNNWGFRPKKLSFKLFNI
QVKEVTQNEGTKTIANNLTSTIQVFTDSEYQLPYVLGSAH
QGCLPPFPADVFMIPQYGYLTLNNGSQAVGRSSFYCLEYF
PSQMLRTGNNFSFSYTFEDVPFHSSYAHSQSLDRLMNPLI
DQYLYYLSRTQSTGGTAGTQQLLFSQAGPNNMSAQAKNWL
PGPMYRQQRVSTTLSQNNNSNFAWTGGTKYHLNGRDSLVY
PGVAMATNKDDEDRFFPSSGVLMFGKQGAGKDNVDYSSVM
LTSEEEIKTTNPVATEQYGVVADNLQQQNTAPIVGAVNSQ
GALPGMVWQNRDVYLQGPIWAKIPHTDGNFHPSPLMGGFG
LKHPPPQILIKNTPVPADPPTAFNQAKLNSFITQYSTGQV
SVEIEWELQKENSKRWNPEIQYTSNYYKSTNVDFAVNTEG
VYSEPRPIGTRYLTRNL.

In another embodiment, the VP1 sequence (from formosan macaque (denoted as Bfm35)) comprises the amino acid sequence of SEQ ID NO:76 and (amino acids 1-737); or comprises an amino acid sequence that is at least 60%, at least 65%, at least 70%, at least 75%, at least 80%, at least 85%, at least 90%, or at least 95% identical over the full length of the amino acid sequence of SEQ ID NO:76. The VP2 capsid protein spans amino acids 138-736 of SEQ ID NO:76 and the VP3 capsid protein spans amino acids 203-736 of SEQ ID NO:76.

(SEQ ID NO: 76)
MAADGYLPDWLEDNLSEGIREWWALKPGAPKPKANQQKQD
DGRGLVLPGYKYLGPFNGLDKGGPVNAADAAALEHDKAYD
QQLQAGDNPYLRYNHADAEFRERLQEDTSFGGNLGRAVFQ
AKKRVLEPLGLVEEGAKTAPGKKRPVEQSPQEPDSSSGIG
KKGQQPAKKRLNFGQTGDSESVPDPQPLGEPPAAPSSLGL
NTMAAGGGAPMADNNEGADGVGSSSGNWHCDSTWLGDRVI
TTSTRTWALPTYNNHLYKQISNGTSGGSTNDSTYFGYSTP
WGYFDFNRSHCHFSPRDWQRLINNNWGFRPKKLSFKLFNI
QVKEVTQNEGTKTIANNLTSTIQVFTDSEYQLPYVLGSAH
QGCLPPFPADVFMIPQYGYLTLNNGGQAVGRSSFYCLEYF
PSQMLRTGNNFSFSYIFEDVPFHSSYAHSQSLDRLMNPLI
DQYLYYLSRTQSTGGTAGTQQLLFSQAGPNNMSAQAKNWL
PGPMYRQQRVSTTLSQNNNSNFAWTGGTKYHLNGRDSLVN
PGVAMATNKDDEDRFFPSSGVLMFGKQGAGKDNVDYSSVM
LTSEEEIKTTNPVATEQYGVVADNLQQQNTAPIVGAVNSQ
GALPGMVWQNRDVYLQGPIWAKIPHTDGNFHPSPLMGGFG
LKHPPPQILIKNTPVPADPPTAFNQAKLNSFITQYSTGQV
SVEIEWELLKESSKRWNPEIQYTSNYYKSTNVDFAVNTEG
VYSEPRPIGTRYLTRNL.

In one embodiment, the AAV viral particle is serotype 1 (AAV-1), serotype 2 (AAV-2), serotype 3 (AAV-3), serotype 3B (AAV-3B), serotype 4 (AAV-4), serotype 5 (AAV-5), serotype 6 (AAV-6), serotype 7 (AAV-7), serotype 8 (AAV-8), serotype 9 (AAV-9), serotype 10 (AAV-10), serotype 11 (AAV-11), serotype 12 (AAV-12), or serotype 13 (AAV-13).

In another embodiment, the AAV viral particle is pseudotyped with a capsid protein derived from AAV particles of a human, a baboon, a marmoset, a pig, a chimpanzee, or a macaque.

In some embodiments, the AAV viral particle is pseudotyped with capsid proteins derived from AAV particles from a baboon.

In other embodiments, the AAV viral particle is pseudotyped with Bba.21, Bba.26, Bba.27, Bba.29, Bba.30, Bba.31, Bba.32, Bba.33, Bba.34, Bba.35, Bba.36, Bba.37, Bba.38, Bba.41, Bba.42, Bba.43, Bba.44, Bce.14, Bce.15, Bce.16, Bce.17, Bce.18, Bce.20, Bce.35, Bce.36, Bce.39, Bce.40, Bce.41, Bce.42, Bce.43, Bce.44, Bce.45, Bce.46, Bey.20, Bey.22, Bey.23, Bma.42, Bma.43, Bpo.1, Bpo.2, Bpo.3, Bpo.4, Bpo.6, Bpo.8, Bpo.13, Bpo.18, Bpo.20, Bpo.23, Bpo.24, Bpo.27, Bpo.28, Bpo.29, Bpo.33, Bpo.35, Bpo.36, Bpo.37, Brh.26, Brh.27, Brh.28, Brh.29, Brh.30, Brh.31, Brh.32, Brh.33, Bfm.17, Bfm.18, Bfm.20, Bfm.21, Bfm.24, Bfm.25, Bfm.27, Bfm.32, Bfm.33, Bfm.34, or Bfm.35 capsid.

In one embodiment, the AAV viral particle is serotype 1 (AAV-1), serotype 2 (AAV-2), serotype 3 (AAV-3), serotype 3B (AAV-3B), serotype 4 (AAV-4), serotype 5 (AAV-5), serotype 6 (AAV-6), serotype 7 (AAV-7), serotype 8 (AAV-8), serotype 9 (AAV-9), serotype 10 (AAV-10), serotype 11 (AAV-11), serotype 12 (AAV-12), or serotype 13 (AAV-13), wherein the AAV particle is pseudotyped with a capsid protein derived from a human, a marmoset, a baboon, a chimpanzee, or a macaque.

In another embodiment, the AAV viral particle is serotype 1 (AAV-1), serotype 2 (AAV-2), serotype 3 (AAV-3), serotype 3B (AAV-3B), serotype 4 (AAV-4), serotype 5 (AAV-5), serotype 6 (AAV-6), serotype 7 (AAV-7), serotype 8 (AAV-8), serotype 9 (AAV-9), serotype 10 (AAV-10), serotype 11 (AAV-11), serotype 12 (AAV-12), or serotype 13 (AAV-13), wherein the AAV particle is pseudotyped with Bba.21, Bba.26, Bba.27, Bba.29, Bba.30, Bba.31, Bba.32, Bba.33, Bba.34, Bba.35, Bba.36, Bba.37, Bba.38, Bba.41, Bba.42, Bba.43, Bba.44, Bce.14, Bce.15, Bce.16, Bce.17, Bce.18, Bce.20, Bce.35, Bce.36, Bce.39, Bce.40, Bce.41, Bce.42, Bce.43, Bce.44, Bce.45, Bce.46, Bey.20, Bey.22, Bey.23, Bma.42, Bma.43, Bpo.1, Bpo.2, Bpo.3, Bpo.4, Bpo.6, Bpo.8, Bpo.13, Bpo.18, Bpo.20, Bpo.23, Bpo.24, Bpo.27, Bpo.28, Bpo.29, Bpo.33, Bpo.35, Bpo.36, Bpo.37, Brh.26, Brh.27, Brh.28, Brh.29, Brh.30, Brh.31, Brh.32, Brh.33, Bfm.17, Bfm.18, Bfm.20, Bfm.21, Bfm.24, Bfm.25, Bfm.27, Bfm.32, Bfm.33, Bfm.34, or Bfm.35 capsid protein, or a variant thereof.

In other embodiments, the AAV viral particle is pseudotyped with a capsid having a VP1 protein comprising the amino acid sequence of any one of SEQ ID NOs:2-76; or comprising an amino acid sequence that is at least 60%, at least 65%, at least 70%, at least 75%, at least 80%, at least 85%, at least 90%, or at least 95% identical over the full length of any one of SEQ ID NOs:2-76.

In any aspect or embodiment, any sequence of VP1, VP2, or VP3 is post-translationally modified before incorporation into the capsid. For example, any sequence VP1, VP2, or VP3 is acetylated, where the methionine in position 1 of the sequence is removed and replaced with an acetyl group.

Transition Metal or Salt

The salt can be an organic or inorganic salt. In some embodiments, the salt is a metal salt.

In other embodiments, the salt comprises a transition metal. The salt includes any ion of the transition metal (e.g., copper (I), copper (II), iron (II), iron (III)).

In various embodiments, the transition metal is copper, scandium, titanium, vanadium, chromium, manganese, iron, cobalt, nickel, copper, zinc, yttrium, zirconium, niobium, molybdenum, technetium, ruthenium, rhodium, palladium, silver, cadmium, hafnium, tantalum, tungsten, rhenium, osmium, iridium, platinum, gold, mercury, rutherfordium, dubnium, seaborgium, bohrium, hassium, meitnerium, ununnilium, unununium, or ununbium.

In another embodiment, the transition metal is copper.

In various embodiments, the transition metal or salt is copper sulfate, copper nitrate, copper selenide, copper hydroxide, copper oxide, copper phosphate, copper silicate, copper borate, copper carbonate aluminum chloride, magnesium chloride, lithium selenide, sodium carbonate, lithium chloride, sodium hydrogen phosphate, sodium metasilicate, strontium hydroxide, trisodium phosphate, potassium fluoride, magnesium sulfate, calcium chloride, sodium sulfate, aluminum sulfate, sodium tetraborate, magnesium sulfate, magnesium bromide, rubidium aluminum sulfate, barium hydroxide, potassium aluminum sulfate, magnesium nitrate, sodium hydrogen phosphate, nickel sulfate, zinc sulfate, beryllium sulfate, lithium nitrate, strontium chloride, zinc nitrate, sodium pyrophosphate, calcium bromide, copper nitrate, aluminum nitrate, sodium tetraborate, silver fluoride, calcium iodide, lithium bromide, lithium iodide, strontium bromide, calcium nitrate, strontium iodide, sodium bromide and strontium nitrate, sodium aluminum lactate, sodium acetate, sodium dehydroacetate, sodium butoxy ethoxy acetate, sodium caprylate, sodium citrate, sodium lactate, sodium dihydroxy glycinate, sodium gluconate, sodium glutamate, sodium hydroxymethane sulfonate, or sodium oxalate. The salt includes hydrates of the salts and anhydrous salts.

In other embodiments, the salt is a copper salt.

In some embodiments, copper can be added to the culture medium in the form of a copper salt, a copper chelate, or a combination thereof.

In one embodiment, the copper salt is a copper sulfate, copper nitrate, copper selenide, copper hydroxide, copper oxide, copper phosphate, copper silicate, copper borate, or a copper carbonate.

In another embodiment, the copper salt is copper sulfate.

Effective Amount

In some embodiments, the effective amount of the salt in the culture medium is sufficient to increase incorporation of the VP1 in the capsid of the AAV viral particle produced by the host cell as compared to the amount of VP1 that is incorporated in a capsid of a AAV viral particle produced by a host cell cultured under similar or substantially similar culture conditions except without the effective amount of copper in the medium.

The amount of VP1 incorporated in the capsid can be detected and quantified by one or more methods known to one of ordinary skill in the art. These may include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immune-diffusion, immuno-electrophoresis, radioimmunoassay (RIA), enzyme-linked immunosorbent assays (ELISAs), immunofluorescent assays, western blotting, and the like.

In one embodiment, a total concentration of the salt in the culture medium is about 1 nM to about 1 mM, illustratively, about 5 nM to about 900 μM, about 10 nM to about 800 μM, about 20 nM to about 700 μM, about 30 nM to about 600 μM, about 40 nM to about 500 μM, about 50 nM to about 400 μM, about 60 nM to about 300 μM, about 70 nM to about 200 μM, about 80 nM to about 100 μM, and about 90 nM to about 95 nM.

In various embodiments, the total concentration of the transition metal or salt in the culture medium is 1 nM, 2 nM, 3 nM, 4 nM, 5 nM, 6 nM, 7 nM, 8 nM, 9 nM, 10 nM, 11 nM, 12 nM, 13 nM, 14 nM, 15 nM, 16 nM, 17 nM, 18 nM, 19 nM, 20 nM, 21 nM, 22 nM, 23 nM, 24 nM, 25 nM, 26 nM, 27 nM, 28 nM, 29 nM, 30 nM, 31 nM, 32 nM, 33 nM, 34 nM, 35 nM, 36 nM, 37 nM, 38 nM, 39 nM, 40 nM, 41 nM, 42 nM, 43 nM, 44 nM, 45 nM, 46 nM, 47 nM, 48 nM, 49 nM, 50 nM, 51 nM, 52 nM, 53 nM, 54 nM, 55 nM, 56 nM, 57 nM, 58 nM, 59 nM, 60 nM, 61 nM, 62 nM, 63 nM, 64 nM, 65 nM, 66 nM, 67 nM, 68 nM, 69 nM, 70 nM, 71 nM, 72 nM, 73 nM, 74 nM, 75 nM, 76 nM, 77 nM, 78 nM, 79 nM, 80 nM, 81 nM, 82 nM, 83 nM, 84 nM, 85 nM, 86 nM, 87 nM, 88 nM, 89 nM, 90 nM, 91 nM, 92 nM, 93 nM, 94 nM, 95 nM, 96 nM, 97 nM, 98 nM, 99 nM, 100 nM, 101 nM, 102 nM, 103 nM, 104 nM, 105 nM, 106 nM, 107 nM, 108 nM, 109 nM, 110 nM, 111 nM, 112 nM, 113 nM, 114 nM, 115 nM, 116 nM, 117 nM, 118 nM, 119 nM, 120 nM, 121 nM, 122 nM, 123 nM, 124 nM, 125 nM, 126 nM, 127 nM, 128 nM, 129 nM, 130 nM, 131 nM, 132 nM, 133 nM, 134 nM, 135 nM, 136 nM, 137 nM, 138 nM, 139 nM, 140 nM, 141 nM, 142 nM, 143 nM, 144 nM, 145 nM, 146 nM, 147 nM, 148 nM, 149 nM, 150 nM, 151 nM, 152 nM, 153 nM, 154 nM, 155 nM, 156 nM, 157 nM, 158 nM, 159 nM, 160 nM, 161 nM, 162 nM, 163 nM, 164 nM, 165 nM, 166 nM, 167 nM, 168 nM, 169 nM, 170 nM, 171 nM, 172 nM, 173 nM, 174 nM, 175 nM, 176 nM, 177 nM, 178 nM, 179 nM, 180 nM, 181 nM, 182 nM, 183 nM, 184 nM, 185 nM, 186 nM, 187 nM, 188 nM, 189 nM, 190 nM, 191 nM, 192 nM, 193 nM, 194 nM, 195 nM, 196 nM, 197 nM, 198 nM, 199 nM, 200 nM, 201 nM, 202 nM, 203 nM, 204 nM, 205 nM, 206 nM, 207 nM, 208 nM, 209 nM, 210 nM, 211 nM, 212 nM, 213 nM, 214 nM, 215 nM, 216 nM, 217 nM, 218 nM, 219 nM, 220 nM, 221 nM, 222 nM, 223 nM, 224 nM, 225 nM, 226 nM, 227 nM, 228 nM, 229 nM, 230 nM, 231 nM, 232 nM, 233 nM, 234 nM, 235 nM, 236 nM, 237 nM, 238 nM, 239 nM, 240 nM, 241 nM, 242 nM, 243 nM, 244 nM, 245 nM, 246 nM, 247 nM, 248 nM, 249 nM, 250 nM, 251 nM, 252 nM, 253 nM, 254 nM, 255 nM, 256 nM, 257 nM, 258 nM, 259 nM, 260 nM, 261 nM, 262 nM, 263 nM, 264 nM, 265 nM, 266 nM, 267 nM, 268 nM, 269 nM, 270 nM, 271 nM, 272 nM, 273 nM, 274 nM, 275 nM, 276 nM, 277 nM, 278 nM, 279 nM, 280 nM, 281 nM, 282 nM, 283 nM, 284 nM, 285 nM, 286 nM, 287 nM, 288 nM, 289 nM, 290 nM, 291 nM, 292 nM, 293 nM, 294 nM, 295 nM, 296 nM, 297 nM, 298 nM, 299 nM, 300 nM, 301 nM, 302 nM, 303 nM, 304 nM, 305 nM, 306 nM, 307 nM, 308 nM, 309 nM, 310 nM, 311 nM, 312 nM, 313 nM, 314 nM, 315 nM, 316 nM, 317 nM, 318 nM, 319 nM, 320 nM, 321 nM, 322 nM, 323 nM, 324 nM, 325 nM, 326 nM, 327 nM, 328 nM, 329 nM, 330 nM, 331 nM, 332 nM, 333 nM, 334 nM, 335 nM, 336 nM, 337 nM, 338 nM, 339 nM, 340 nM, 341 nM, 342 nM, 343 nM, 344 nM, 345 nM, 346 nM, 347 nM, 348 nM, 349 nM, 350 nM, 351 nM, 352 nM, 353 nM, 354 nM, 355 nM, 356 nM, 357 nM, 358 nM, 359 nM, 360 nM, 361 nM, 362 nM, 363 nM, 364 nM, 365 nM, 366 nM, 367 nM, 368 nM, 369 nM, 370 nM, 371 nM, 372 nM, 373 nM, 374 nM, 375 nM, 376 nM, 377 nM, 378 nM, 379 nM, 380 nM, 381 nM, 382 nM, 383 nM, 384 nM, 385 nM, 386 nM, 387 nM, 388 nM, 389 nM, 390 nM, 391 nM, 392 nM, 393 nM, 394 nM, 395 nM, 396 nM, 397 nM, 398 nM, 399 nM, 400 nM, 401 nM, 402 nM, 403 nM, 404 nM, 405 nM, 406 nM, 407 nM, 408 nM, 409 nM, 410 nM, 411 nM, 412 nM, 413 nM, 414 nM, 415 nM, 416 nM, 417 nM, 418 nM, 419 nM, 420 nM, 421 nM, 422 nM, 423 nM, 424 nM, 425 nM, 426 nM, 427 nM, 428 nM, 429 nM, 430 nM, 431 nM, 432 nM, 433 nM, 434 nM, 435 nM, 436 nM, 437 nM, 438 nM, 439 nM, 440 nM, 441 nM, 442 nM, 443 nM, 444 nM, 445 nM, 446 nM, 447 nM, 448 nM, 449 nM, 450 nM, 451 nM, 452 nM, 453 nM, 454 nM, 455 nM, 456 nM, 457 nM, 458 nM, 459 nM, 460 nM, 461 nM, 462 nM, 463 nM, 464 nM, 465 nM, 466 nM, 467 nM, 468 nM, 469 nM, 470 nM, 471 nM, 472 nM, 473 nM, 474 nM, 475 nM, 476 nM, 477 nM, 478 nM, 479 nM, 480 nM, 481 nM, 482 nM, 483 nM, 484 nM, 485 nM, 486 nM, 487 nM, 488 nM, 489 nM, 490 nM, 491 nM, 492 nM, 493 nM, 494 nM, 495 nM, 496 nM, 497 nM, 498 nM, 499 nM, 500 nM, 501 nM, 502 nM, 503 nM, 504 nM, 505 nM, 506 nM, 507 nM, 508 nM, 509 nM, 510 nM, 511 nM, 512 nM, 513 nM, 514 nM, 515 nM, 516 nM, 517 nM, 518 nM, 519 nM, 520 nM, 521 nM, 522 nM, 523 nM, 524 nM, 525 nM, 526 nM, 527 nM, 528 nM, 529 nM, 530 nM, 531 nM, 532 nM, 533 nM, 534 nM, 535 nM, 536 nM, 537 nM, 538 nM, 539 nM, 540 nM, 541 nM, 542 nM, 543 nM, 544 nM, 545 nM, 546 nM, 547 nM, 548 nM, 549 nM, 550 nM, 551 nM, 552 nM, 553 nM, 554 nM, 555 nM, 556 nM, 557 nM, 558 nM, 559 nM, 560 nM, 561 nM, 562 nM, 563 nM, 564 nM, 565 nM, 566 nM, 567 nM, 568 nM, 569 nM, 570 nM, 571 nM, 572 nM, 573 nM, 574 nM, 575 nM, 576 nM, 577 nM, 578 nM, 579 nM, 580 nM, 581 nM, 582 nM, 583 nM, 584 nM, 585 nM, 586 nM, 587 nM, 588 nM, 589 nM, 590 nM, 591 nM, 592 nM, 593 nM, 594 nM, 595 nM, 596 nM, 597 nM, 598 nM, 599 nM, 600 nM, 601 nM, 602 nM, 603 nM, 604 nM, 605 nM, 606 nM, 607 nM, 608 nM, 609 nM, 610 nM, 611 nM, 612 nM, 613 nM, 614 nM, 615 nM, 616 nM, 617 nM, 618 nM, 619 nM, 620 nM, 621 nM, 622 nM, 623 nM, 624 nM, 625 nM, 626 nM, 627 nM, 628 nM, 629 nM, 630 nM, 631 nM, 632 nM, 633 nM, 634 nM, 635 nM, 636 nM, 637 nM, 638 nM, 639 nM, 640 nM, 641 nM, 642 nM, 643 nM, 644 nM, 645 nM, 646 nM, 647 nM, 648 nM, 649 nM, 650 nM, 651 nM, 652 nM, 653 nM, 654 nM, 655 nM, 656 nM, 657 nM, 658 nM, 659 nM, 660 nM, 661 nM, 662 nM, 663 nM, 664 nM, 665 nM, 666 nM, 667 nM, 668 nM, 669 nM, 670 nM, 671 nM, 672 nM, 673 nM, 674 nM, 675 nM, 676 nM, 677 nM, 678 nM, 679 nM, 680 nM, 681 nM, 682 nM, 683 nM, 684 nM, 685 nM, 686 nM, 687 nM, 688 nM, 689 nM, 690 nM, 691 nM, 692 nM, 693 nM, 694 nM, 695 nM, 696 nM, 697 nM, 698 nM, 699 nM, 700 nM, 701 nM, 702 nM, 703 nM, 704 nM, 705 nM, 706 nM, 707 nM, 708 nM, 709 nM, 710 nM, 711 nM, 712 nM, 713 nM, 714 nM, 715 nM, 716 nM, 717 nM, 718 nM, 719 nM, 720 nM, 721 nM, 722 nM, 723 nM, 724 nM, 725 nM, 726 nM, 727 nM, 728 nM, 729 nM, 730 nM, 731 nM, 732 nM, 733 nM, 734 nM, 735 nM, 736 nM, 737 nM, 738 nM, 739 nM, 740 nM, 741 nM, 742 nM, 743 nM, 744 nM, 745 nM, 746 nM, 747 nM, 748 nM, 749 nM, 750 nM, 751 nM, 752 nM, 753 nM, 754 nM, 755 nM, 756 nM, 757 nM, 758 nM, 759 nM, 760 nM, 761 nM, 762 nM, 763 nM, 764 nM, 765 nM, 766 nM, 767 nM, 768 nM, 769 nM, 770 nM, 771 nM, 772 nM, 773 nM, 774 nM, 775 nM, 776 nM, 777 nM, 778 nM, 779 nM, 780 nM, 781 nM, 782 nM, 783 nM, 784 nM, 785 nM, 786 nM, 787 nM, 788 nM, 789 nM, 790 nM, 791 nM, 792 nM, 793 nM, 794 nM, 795 nM, 796 nM, 797 nM, 798 nM, 799 nM, 800 nM, 801 nM, 802 nM, 803 nM, 804 nM, 805 nM, 806 nM, 807 nM, 808 nM, 809 nM, 810 nM, 811 nM, 812 nM, 813 nM, 814 nM, 815 nM, 816 nM, 817 nM, 818 nM, 819 nM, 820 nM, 821 nM, 822 nM, 823 nM, 824 nM, 825 nM, 826 nM, 827 nM, 828 nM, 829 nM, 830 nM, 831 nM, 832 nM, 833 nM, 834 nM, 835 nM, 836 nM, 837 nM, 838 nM, 839 nM, 840 nM, 841 nM, 842 nM, 843 nM, 844 nM, 845 nM, 846 nM, 847 nM, 848 nM, 849 nM, 850 nM, 851 nM, 852 nM, 853 nM, 854 nM, 855 nM, 856 nM, 857 nM, 858 nM, 859 nM, 860 nM, 861 nM, 862 nM, 863 nM, 864 nM, 865 nM, 866 nM, 867 nM, 868 nM, 869 nM, 870 nM, 871 nM, 872 nM, 873 nM, 874 nM, 875 nM, 876 nM, 877 nM, 878 nM, 879 nM, 880 nM, 881 nM, 882 nM, 883 nM, 884 nM, 885 nM, 886 nM, 887 nM, 888 nM, 889 nM, 890 nM, 891 nM, 892 nM, 893 nM, 894 nM, 895 nM, 896 nM, 897 nM, 898 nM, 899 nM, 900 nM, 901 nM, 902 nM, 903 nM, 904 nM, 905 nM, 906 nM, 907 nM, 908 nM, 909 nM, 910 nM, 911 nM, 912 nM, 913 nM, 914 nM, 915 nM, 916 nM, 917 nM, 918 nM, 919 nM, 920 nM, 921 nM, 922 nM, 923 nM, 924 nM, 925 nM, 926 nM, 927 nM, 928 nM, 929 nM, 930 nM, 931 nM, 932 nM, 933 nM, 934 nM, 935 nM, 936 nM, 937 nM, 938 nM, 939 nM, 940 nM, 941 nM, 942 nM, 943 nM, 944 nM, 945 nM, 946 nM, 947 nM, 948 nM, 949 nM, 950 nM, 951 nM, 952 nM, 953 nM, 954 nM, 955 nM, 956 nM, 957 nM, 958 nM, 959 nM, 960 nM, 961 nM, 962 nM, 963 nM, 964 nM, 965 nM, 966 nM, 967 nM, 968 nM, 969 nM, 970 nM, 971 nM, 972 nM, 973 nM, 974 nM, 975 nM, 976 nM, 977 nM, 978 nM, 979 nM, 980 nM, 981 nM, 982 nM, 983 nM, 984 nM, 985 nM, 986 nM, 987 nM, 988 nM, 989 nM, 990 nM, 991 nM, 992 nM, 993 nM, 994 nM, 995 nM, 996 nM, 997 nM, 998 nM, 999 nM, 1000 nM/1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 21 μM, 22 μM, 23 μM, 24 μM, 25 μM, 26 μM, 27 μM, 28 μM, 29 μM, 30 μM, 31 μM, 32 μM, 33 μM, 34 μM, 35 μM, 36 μM, 37 μM, 38 μM, 39 μM, 40 μM, 41 μM, 42 μM, 43 μM, 44 μM, 45 μM, 46 μM, 47 μM, 48 μM, 49 μM, 50 μM, 51 μM, 52 μM, 53 μM, 54 μM, 55 μM, 56 μM, 57 μM, 58 μM, 59 μM, 60 μM, 61 μM, 62 μM, 63 μM, 64 μM, 65 μM, 66 μM, 67 μM, 68 μM, 69 μM, 70 μM, 71 μM, 72 μM, 73 μM, 74 μM, 75 μM, 76 μM, 77 μM, 78 μM, 79 μM, 80 μM, 81 μM, 82 μM, 83 μM, 84 μM, 85 μM, 86 μM, 87 μM, 88 μM, 89 μM, 90 μM, 91 μM, 92 μM, 93 μM, 94 μM, 95 μM, 96 μM, 97 μM, 98 μM, 99 μM, 100 μM, 101 μM, 102 μM, 103 μM, 104 μM, 105 μM, 106 μM, 107 μM, 108 μM, 109 μM, 110 μM, 111 μM, 112 μM, 113 μM, 114 μM, 115 μM, 116 μM, 117 μM, 118 μM, 119 μM, 120 μM, 121 μM, 122 μM, 123 μM, 124 μM, 125 μM, 126 μM, 127 μM, 128 μM, 129 μM, 130 μM, 131 μM, 132 μM, 133 μM, 134 μM, 135 μM, 136 μM, 137 μM, 138 μM, 139 μM, 140 μM, 141 μM, 142 μM, 143 μM, 144 μM, 145 μM, 146 μM, 147 μM, 148 μM, 149 μM, 150 μM, 151 μM, 152 μM, 153 μM, 154 μM, 155 μM, 156 μM, 157 μM, 158 μM, 159 μM, 160 μM, 161 μM, 162 μM, 163 μM, 164 μM, 165 μM, 166 μM, 167 μM, 168 μM, 169 μM, 170 μM, 171 μM, 172 μM, 173 μM, 174 μM, 175 μM, 176 μM, 177 μM, 178 μM, 179 μM, 180 μM, 181 μM, 182 μM, 183 μM, 184 μM, 185 μM, 186 μM, 187 μM, 188 μM, 189 μM, 190 μM, 191 μM, 192 μM, 193 μM, 194 μM, 195 μM, 196 μM, 197 μM, 198 μM, 199 μM, 200 μM, 201 μM, 202 μM, 203 μM, 204 μM, 205 μM, 206 μM, 207 μM, 208 μM, 209 μM, 210 μM, 211 μM, 212 μM, 213 μM, 214 μM, 215 μM, 216 μM, 217 μM, 218 μM, 219 μM, 220 μM, 221 μM, 222 μM, 223 μM, 224 μM, 225 μM, 226 μM, 227 μM, 228 μM, 229 μM, 230 μM, 231 μM, 232 μM, 233 μM, 234 μM, 235 μM, 236 μM, 237 μM, 238 μM, 239 μM, 240 μM, 241 μM, 242 μM, 243 μM, 244 μM, 245 μM, 246 μM, 247 μM, 248 μM, 249 μM, 250 μM, 251 μM, 252 μM, 253 μM, 254 μM, 255 μM, 256 μM, 257 μM, 258 μM, 259 μM, 260 μM, 261 μM, 262 μM, 263 μM, 264 μM, 265 μM, 266 μM, 267 μM, 268 μM, 269 μM, 270 μM, 271 μM, 272 μM, 273 μM, 274 μM, 275 μM, 276 μM, 277 μM, 278 μM, 279 μM, 280 μM, 281 μM, 282 μM, 283 μM, 284 μM, 285 μM, 286 μM, 287 μM, 288 μM, 289 μM, 290 μM, 291 μM, 292 μM, 293 μM, 294 μM, 295 μM, 296 μM, 297 μM, 298 μM, 299 μM, 300 μM, 301 μM, 302 μM, 303 μM, 304 μM, 305 μM, 306 μM, 307 μM, 308 μM, 309 μM, 310 μM, 311 μM, 312 μM, 313 μM, 314 μM, 315 μM, 316 μM, 317 μM, 318 μM, 319 μM, 320 μM, 321 μM, 322 μM, 323 μM, 324 μM, 325 μM, 326 μM, 327 μM, 328 μM, 329 μM, 330 μM, 331 μM, 332 μM, 333 μM, 334 μM, 335 μM, 336 μM, 337 μM, 338 μM, 339 μM, 340 μM, 341 μM, 342 μM, 343 μM, 344 μM, 345 μM, 346 μM, 347 μM, 348 μM, 349 μM, 350 μM, 351 μM, 352 μM, 353 μM, 354 μM, 355 μM, 356 μM, 357 μM, 358 μM, 359 μM, 360 μM, 361 μM, 362 μM, 363 μM, 364 μM, 365 μM, 366 μM, 367 μM, 368 μM, 369 μM, 370 μM, 371 μM, 372 μM, 373 μM, 374 μM, 375 μM, 376 μM, 377 μM, 378 μM, 379 μM, 380 μM, 381 μM, 382 μM, 383 μM, 384 μM, 385 μM, 386 μM, 387 μM, 388 μM, 389 μM, 390 μM, 391 μM, 392 μM, 393 μM, 394 μM, 395 μM, 396 μM, 397 μM, 398 μM, 399 μM, 400 μM, 401 μM, 402 μM, 403 μM, 404 μM, 405 μM, 406 μM, 407 μM, 408 μM, 409 μM, 410 μM, 411 μM, 412 μM, 413 μM, 414 μM, 415 μM, 416 μM, 417 μM, 418 μM, 419 μM, 420 μM, 421 μM, 422 μM, 423 μM, 424 μM, 425 μM, 426 μM, 427 μM, 428 μM, 429 μM, 430 μM, 431 μM, 432 μM, 433 μM, 434 μM, 435 μM, 436 μM, 437 μM, 438 μM, 439 μM, 440 μM, 441 μM, 442 μM, 443 μM, 444 μM, 445 μM, 446 μM, 447 μM, 448 μM, 449 μM, 450 μM, 451 μM, 452 μM, 453 μM, 454 μM, 455 μM, 456 μM, 457 μM, 458 μM, 459 μM, 460 μM, 461 μM, 462 μM, 463 μM, 464 μM, 465 μM, 466 μM, 467 μM, 468 μM, 469 μM, 470 μM, 471 μM, 472 μM, 473 μM, 474 μM, 475 μM, 476 μM, 477 μM, 478 μM, 479 μM, 480 μM, 481 μM, 482 μM, 483 μM, 484 μM, 485 μM, 486 μM, 487 μM, 488 μM, 489 μM, 490 μM, 491 μM, 492 μM, 493 μM, 494 μM, 495 μM, 496 μM, 497 μM, 498 μM, 499 μM, 500 μM, 501 μM, 502 μM, 503 μM, 504 μM, 505 μM, 506 μM, 507 μM, 508 μM, 509 μM, 510 μM, 511 μM, 512 μM, 513 μM, 514 μM, 515 μM, 516 μM, 517 μM, 518 μM, 519 μM, 520 μM, 521 μM, 522 μM, 523 μM, 524 μM, 525 μM, 526 μM, 527 μM, 528 μM, 529 μM, 530 μM, 531 μM, 532 μM, 533 μM, 534 μM, 535 μM, 536 μM, 537 μM, 538 μM, 539 μM, 540 μM, 541 μM, 542 μM, 543 μM, 544 μM, 545 μM, 546 μM, 547 μM, 548 μM, 549 μM, 550 μM, 551 μM, 552 μM, 553 μM, 554 μM, 555 μM, 556 μM, 557 μM, 558 μM, 559 μM, 560 μM, 561 μM, 562 μM, 563 μM, 564 μM, 565 μM, 566 μM, 567 μM, 568 μM, 569 μM, 570 μM, 571 μM, 572 μM, 573 μM, 574 μM, 575 μM, 576 μM, 577 μM, 578 μM, 579 μM, 580 μM, 581 μM, 582 μM, 583 μM, 584 μM, 585 μM, 586 μM, 587 μM, 588 μM, 589 μM, 590 μM, 591 μM, 592 μM, 593 μM, 594 μM, 595 μM, 596 μM, 597 μM, 598 μM, 599 μM, 600 μM, 601 μM, 602 μM, 603 μM, 604 μM, 605 μM, 606 μM, 607 μM, 608 μM, 609 μM, 610 μM, 611 μM, 612 μM, 613 μM, 614 μM, 615 μM, 616 μM, 617 μM, 618 μM, 619 μM, 620 μM, 621 μM, 622 μM, 623 μM, 624 μM, 625 μM, 626 μM, 627 μM, 628 μM, 629 μM, 630 μM, 631 μM, 632 μM, 633 μM, 634 μM, 635 μM, 636 μM, 637 μM, 638 μM, 639 μM, 640 μM, 641 μM, 642 μM, 643 μM, 644 μM, 645 μM, 646 μM, 647 μM, 648 μM, 649 μM, 650 μM, 651 μM, 652 μM, 653 μM, 654 μM, 655 μM, 656 μM, 657 μM, 658 μM, 659 μM, 660 μM, 661 μM, 662 μM, 663 μM, 664 μM, 665 μM, 666 μM, 667 μM, 668 μM, 669 μM, 670 μM, 671 μM, 672 μM, 673 μM, 674 μM, 675 μM, 676 μM, 677 μM, 678 μM, 679 μM, 680 μM, 681 μM, 682 μM, 683 μM, 684 μM, 685 μM, 686 μM, 687 μM, 688 μM, 689 μM, 690 μM, 691 μM, 692 μM, 693 μM, 694 μM, 695 μM, 696 μM, 697 μM, 698 μM, 699 μM, 700 μM, 701 μM, 702 μM, 703 μM, 704 μM, 705 μM, 706 μM, 707 μM, 708 μM, 709 μM, 710 μM, 711 μM, 712 μM, 713 μM, 714 μM, 715 μM, 716 μM, 717 μM, 718 μM, 719 μM, 720 μM, 721 μM, 722 μM, 723 μM, 724 μM, 725 μM, 726 μM, 727 μM, 728 μM, 729 μM, 730 μM, 731 μM, 732 μM, 733 μM, 734 μM, 735 μM, 736 μM, 737 μM, 738 μM, 739 μM, 740 μM, 741 μM, 742 μM, 743 μM, 744 μM, 745 μM, 746 μM, 747 μM, 748 μM, 749 μM, 750 μM, 751 μM, 752 μM, 753 μM, 754 μM, 755 μM, 756 μM, 757 μM, 758 μM, 759 μM, 760 μM, 761 μM, 762 μM, 763 μM, 764 μM, 765 μM, 766 μM, 767 μM, 768 μM, 769 μM, 770 μM, 771 μM, 772 μM, 773 μM, 774 μM, 775 μM, 776 μM, 777 μM, 778 μM, 779 μM, 780 μM, 781 μM, 782 μM, 783 μM, 784 μM, 785 μM, 786 μM, 787 μM, 788 μM, 789 μM, 790 μM, 791 μM, 792 μM, 793 μM, 794 μM, 795 μM, 796 μM, 797 μM, 798 μM, 799 μM, 800 μM, 801 μM, 802 μM, 803 μM, 804 μM, 805 μM, 806 μM, 807 μM, 808 μM, 809 μM, 810 μM, 811 μM, 812 μM, 813 μM, 814 μM, 815 μM, 816 μM, 817 μM, 818 μM, 819 μM, 820 μM, 821 μM, 822 μM, 823 μM, 824 μM, 825 μM, 826 μM, 827 μM, 828 μM, 829 μM, 830 μM, 831 μM, 832 μM, 833 μM, 834 μM, 835 μM, 836 μM, 837 μM, 838 μM, 839 μM, 840 μM, 841 μM, 842 μM, 843 μM, 844 μM, 845 μM, 846 μM, 847 μM, 848 μM, 849 μM, 850 μM, 851 μM, 852 μM, 853 μM, 854 μM, 855 μM, 856 μM, 857 μM, 858 μM, 859 μM, 860 μM, 861 μM, 862 μM, 863 μM, 864 μM, 865 μM, 866 μM, 867 μM, 868 μM, 869 μM, 870 μM, 871 μM, 872 μM, 873 μM, 874 μM, 875 μM, 876 μM, 877 μM, 878 μM, 879 μM, 880 μM, 881 μM, 882 μM, 883 μM, 884 μM, 885 μM, 886 μM, 887 μM, 888 μM, 889 μM, 890 μM, 891 μM, 892 μM, 893 μM, 894 μM, 895 μM, 896 μM, 897 μM, 898 μM, 899 μM, 900 μM, 901 μM, 902 μM, 903 μM, 904 μM, 905 μM, 906 μM, 907 μM, 908 μM, 909 μM, 910 μM, 911 μM, 912 μM, 913 μM, 914 μM, 915 μM, 916 μM, 917 μM, 918 μM, 919 μM, 920 μM, 921 μM, 922 μM, 923 μM, 924 μM, 925 μM, 926 μM, 927 μM, 928 μM, 929 μM, 930 μM, 931 μM, 932 μM, 933 μM, 934 μM, 935 μM, 936 μM, 937 μM, 938 μM, 939 μM, 940 μM, 941 μM, 942 μM, 943 μM, 944 μM, 945 μM, 946 μM, 947 μM, 948 μM, 949 μM, 950 μM, 951 μM, 952 μM, 953 μM, 954 μM, 955 μM, 956 μM, 957 μM, 958 μM, 959 μM, 960 μM, 961 μM, 962 μM, 963 μM, 964 μM, 965 μM, 966 μM, 967 μM, 968 μM, 969 μM, 970 μM, 971 μM, 972 μM, 973 μM, 974 μM, 975 μM, 976 μM, 977 μM, 978 μM, 979 μM, 980 μM, 981 μM, 982 μM, 983 μM, 984 μM, 985 μM, 986 μM, 987 μM, 988 μM, 989 μM, 990 μM, 991 μM, 992 μM, 993 μM, 994 μM, 995 μM, 996 μM, 997 μM, 998 μM, 999 μM, or <1000 μM. In various embodiments, the total concentration of the transition or salt in the culture medium is a range between any two concentrations provided above.

In various embodiments, the total concentration of the salt in the culture medium is about 10 nM to about 100 μM.

In various embodiments, the total concentration of the salt in the culture medium is about 5 nM to about 50 μM.

In various embodiments, the total concentration of the salt in the culture medium is about 20 μM to about 25 μM.

In other embodiments, the culture medium is supplemented with an amount of the salt sufficient to provide the total concentration of the salt in the culture medium.

In one embodiment, the salt is copper sulfate and the host cell is an insect cell, wherein the total concentration of the salt in the culture medium is about 10 nM to about 100 μM.

In various embodiments, the effective amount of the transition metal or salt increases the incorporation of VP1 proteins, where the VP1 proteins are 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% of capsid proteins. In other embodiments, the VP1 protein percentage is a range between any two percentages provided above.

In various embodiments, the effective amount of the transition metal or salt increases the incorporation, where the average number of VP1 proteins in the capsids is 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, or 40. In other embodiments, the average number of VP1 proteins in the capsids is a range between two values provided above. In other embodiments, the VP1 protein per rAAV capsid is at least 5, at least 6, at least 7, at least 8, at least 9, at least 10, 5 to 10, 6 to 9, 5, 6, 7, 8, 9, or 10.

In other embodiments, the method further comprises isolating the AAV viral particle produced by the host cell, for example, from the culture medium.

Inhibition of Cysteine Protease and Cysteine Protease Inhibitor

Although not wishing to be bound by theory, it is believed that inhibition of a cysteine protease increases incorporation of the VP1 in the capsid of the AAV viral particle produced by a host cultured in a culture medium.

Accordingly, in other aspects, the present invention provides a method for preparing an adeno-associated virus (AAV) viral particle, the method comprising the step of culturing, in a culture medium comprising an effective amount of an inhibitor of a cysteine protease, a host cell capable of producing the AAV viral particle, wherein the AAV viral particle comprises an AAV capsid comprising a VP1 protein.

In some embodiments, the host cell and the AAV viral particle are as disclosed herein.

In one embodiment, the method comprises modulating the expression or activity of the inhibitor in the host cell either directly or indirectly through modulation of a protein or factor that modulates the expression or activity of the inhibitor, thereby providing the effective amount of the inhibitor sufficient to inhibit or reduce the activity of the cysteine protease, thereby increasing incorporation of the VP1 in the capsid of the AAV viral particle produced by the host cell as compared to the amount of VP1 that is incorporated in a capsid of a AAV viral particle produced by a host cell cultured under similar or substantially similar culture conditions except without the effective amount of the inhibitor.

In another embodiment, the modulating the expression or activity of the inhibitor in the host cell comprises adding the inhibitor to the culture medium.

In some embodiments, the inhibitor can inhibit a cysteine protease activity of bromelain, calpain, caspase, cathepsin (e.g., B, H, and/or L), chymopapain, ficin, or papain.

In other embodiments, the inhibitor comprises leupeptin (Ac-Leu-Leu-Arg-CHO), E-64 (L-trans-3-carboxyoxiran-2-carbonyl-L-leucylagmatin), L-trans epoxysuccinyl-L-leucylamido-3-methyl-butaine, antipain dihydrochloride, chymostatin microbial, N-ethylmaleimide, α2-macroglobulin, or phenylmethanesulfonyl fluoride.

In various embodiments, the effective amount of the cysteine protease inhibitor increases the incorporation of VP1 proteins, where the VP1 proteins are 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% of capsid proteins. In other embodiments, the VP1 protein percentage is a range between any two percentages provided above.

In various embodiments, the effective amount of the cysteine protease inhibitor increases the incorporation, where the average number of VP1 proteins in the capsids is 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, or 40. In other embodiments, the average number of VP1 proteins in the capsids is a range between two values provided above. In other embodiments, the VP1 protein per rAAV capsid is at least 5, at least 6, at least 7, at least 8, at least 9, at least 10, 5 to 10, 6 to 9, 5, 6, 7, 8, 9, or 10.

Baculovirus Virions

In some embodiments, a baculoviral system is employed.

Baculoviruses are enveloped DNA viruses of arthropods, two members of which are well known for producing recombinant proteins in cell cultures. Baculoviruses have circular double-stranded genomes (80-200 kbp) which can be engineered to allow the delivery of large genomic content to specific cells. The viruses used as a vector are generally Autographa californica multicapsid nucleopolyhedro virus (AcMNPV) or Bombyx mori (Bm)NPV).

Baculoviruses are commonly used for the infection of insect cells for the expression of recombinant proteins. In particular, expression of heterologous genes in insects can be accomplished as described in for instance U.S. Pat. No. 4,745,051; Friesen et al (1986); EP 127,839; and EP 155,476. Numerous baculovirus strains and variants and corresponding permissive insect host cells that can be used for protein production are known in the art.

For example, the commercially available Bac-to-Bac® system (Thermo Fisher Scientific, Rockford, IL) (Catalog No. 10359016) includes expression vectors for recombinant protein expression. The pFastBac™ 1 vector (Thermo Fisher Scientific, Rockford, IL) has the strong polyhedrin promoter for high-level protein expression and a large multiple cloning site for simplified cloning. The pFastBac™ Dual (Thermo Fisher Scientific, Rockford, IL) is a single vector featuring two strong promoters, the polyhedrin promoter and the p10 promoter in a single vector for simultaneous expression of two proteins in insect cells.

Briefly, by way of illustration, a baculoviral system such as Bac-to-Bac® relies on the generation of recombinant baculovirus by site-specific transposition in E. coli rather than homologous recombination in insect cells. For example, a gene of interest can be cloned into a pFastBac™ vector and transformed into DH10Bac™ competent E. coli (Thermo Fisher Scientific, Rockford, IL). DH10Bac™ contains a parent bacmid with a lacZ-mini-attTn7 fusion. Transposition occurs between the elements of the pFastBac™ vector and the parent bacmid in the presence of the transposition proteins provided by a helper plasmid. When the transposition is successful, the expression cassette disrupts the lacZ gene and the new expression bacmid can be visualized as white bacterial colonies. The new expression bacmid can be isolated and used to transfect, for example, Sf9 or Sf21 cells using a suitable transfection reagent. Examples of transfection reagents include liposomes, cationic polymers (e.g., poly(ethyleneimine)), cationic peptides (e.g., poly-L-lysine), Lipofectin (Thermo Fisher Scientific), Cellfectin (Thermo Fisher Scientific), Cellfectin II (Thermo Fisher Scientific), Expifectamine Sf (Thermo Fisher Scientific), TransIT® (Mirus Bio), Insect GeneJuice® (Biontex), and transfection reagents disclosed in U.S. Pat. Nos. 5,674,908, 5,834,439, and 6,110,916, all of which are incorporated herein by reference in their entirety. After an appropriate amount of time in culture, recombinant baculovirus can be isolated. The recombinant baculovirus can be used to infect the cell to produce AAV viral particles and/or to express gene(s) of interest.

In one embodiment, a baculovirus system of the present invention comprises baculovirus-transfected cells maintained in conditions such that baculovirus virions (BVs) are produced. These produced baculovirus virions are then collected for their subsequent use for infecting the host cell.

In some embodiments, the method for preparing the adeno-associated virus (AAV) viral particle comprises
culturing, in the culture medium comprising the effective amount of the salt, the host cell capable of producing the AAV viral particle, wherein the AAV viral particle comprises the AAV capsid comprising the VP1 protein,
wherein the host cell comprises a first nucleic acid vector comprising 5′ and 3′ AAV inverted terminal repeat sequences flanking one or more transgenes comprising one or more heterologous genes operably linked to regulatory sequences that control expression of the one or more heterologous genes in the host cell, and a second nucleic acid vector comprising AAV rep and cap nucleic acids sequences,
wherein said cap nucleic acid sequence encodes an AAV capsid,
wherein the AAV particle is pseudotyped with the AAV capsid, and
wherein the first nucleic acid vector is introduced into the host cell by infection of the host cell by a baculovirus comprising the first nucleic acid vector.

In other embodiments, the first and second nucleic acid vectors are introduced into the host cell by infection of the host cell by a first baculovirus comprising the first nucleic acid vector and a second baculovirus comprising the second nucleic acid vector.

In some embodiments, concentrations of salt in transfection or infection medium and in the producer medium is increased such that the concentrations of salt in the transfection or infection medium and in the producer medium is higher than the concentration of salt in the pre-production culture medium.

In another embodiment, the method further comprises isolating, purifying or otherwise recovering the AAV viral particle from the host cell and/or supernatant of the host cell.

Purification of AAV Viral Particles

In other embodiments, AAV viral particles can be purified from the host cell using a variety of conventional purification methods, such as column chromatography, CsCl gradients, and the like. For example, a plurality of column purification steps can be used, such as purification over an anion exchange column, an affinity column, and/or a cation exchange column. See, for example, International Publication No. WO 02/12455. Further, if infection is employed to express the accessory functions, residual helper virus can be inactivated, using known methods. For example, adenovirus can be inactivated by heating to temperatures of approximately 60° C. for, for example, 20 minutes or more. This treatment effectively inactivates only the helper virus since AAV is extremely heat stable while the helper adenovirus is heat labile.

In one embodiment, the AAV viral particle stock is then treated to remove empty capsids, for example, using column chromatography techniques.

In another embodiment, AAV viral particle preparations are obtained by lysing transfected cells to obtain a crude cell lysate. The crude cell lysate can then be clarified to remove cell debris by techniques well known in the art, such as filtering, centrifuging, and the like, to render a clarified cell lysate. The crude cell lysate or clarified cell lysate, which may contain both AAV viral particles and AAV empty capsids, can then be applied to a first cation exchange matrix under non-separating conditions, wherein the first cation exchange column functions to further separate the AAV viral particles and the AAV empty capsids from cellular and other components present in the cell lysate preparation. Methods for performing the initial purification of the cell lysate are known. One representative method is described in U.S. Pat. No. 6,593,123, herein incorporated by reference in its entirety.

Pharmaceutical Formulations

In other aspects, the present invention is directed to pharmaceutical formulations of AAV viral particles of the present invention useful for administration to a subject.

In one embodiment, the pharmaceutical formulations of the present invention are liquid formulations that comprise AAV viral particles disclosed herein, wherein the concentration of AAV viral particles in the formulation may vary widely. AAV viral particles and compositions may be formulated in dosage unit form for ease of administration and uniformity of dosage. Dosage unit form as used herein refers to physically discrete units suited as unitary dosages for an individual to be treated; each unit containing a predetermined quantity of active compound calculated to produce the desired therapeutic effect in association with the required pharmaceutical carrier. The dosage unit forms are dependent upon the amount of AAV viral particles necessary to produce the desired effect(s). The amount necessary can be formulated in a single dose or can be formulated in multiple dosage units. The dose may be adjusted to a suitable AAV viral particle concentration, optionally combined with one or more other agents, and packaged for use.

In another embodiment, pharmaceutical compositions will include sufficient genetic material to provide a prophylactically or therapeutically effective amount, i.e., an amount sufficient to prevent, reduce or ameliorate symptoms of a disease state in question or an amount sufficient to confer the desired benefit.

In other embodiments, the AAV viral particle containing pharmaceutical formulation of the invention comprises one or more pharmaceutically acceptable excipients to provide the formulation with advantageous properties for storage and/or administration to subjects. In certain embodiments, the pharmaceutical formulations of the present invention are capable of being stored at <65° C. for a period of at least 2 weeks, preferably at least 4 weeks, more preferably at least 6 weeks and yet more preferably at least about 8 weeks, without detectable change in stability. In this regard, the term “stable” means that the AAV viral particles present in the formulation essentially retains its physical stability, chemical stability and/or biological activity during storage. In other embodiments of the present invention, the AAV viral particle present in the pharmaceutical formulation retains at least about 80% of its biological activity in a subject during storage for a determined period of time at −65° C., more preferably at least about 85%, 90%, 95%, 98% or 99% of its biological activity in a subject.

In some embodiments, sodium phosphate dibasic at a concentration of about 0.1 mg/ml to about 3 mg/ml, about 0.5 mg/ml to about 2.5 mg/ml, about 1 mg/ml to about 2 mg/ml, or about 1.4 mg/ml to about 1.6 mg/ml. In a particularly preferred embodiment, the AAV viral particle formulation of the present invention comprises about 1.42 mg/ml of sodium phosphate, dibasic (dried).

In other embodiments, another buffering agent that may find use in the AAV viral particle formulations of the present invention is sodium phosphate, monobasic monohydrate which, in some embodiments, finds use at a concentration of from about 0.1 mg/ml to about 3 mg/ml, about 0.5 mg/ml to about 2.5 mg/ml, about 1 mg/ml to about 2 mg/ml, or about 1.3 mg/ml to about 1.5 mg/ml. In one embodiment, the AAV viral particle formulation of the present invention comprises about 1.38 mg/ml of sodium phosphate, monobasic monohydrate. In another embodiment, the AAV viral particle formulation of the present invention comprises about 1.42 mg/ml of sodium phosphate, dibasic and about 1.38 mg/ml of sodium phosphate, monobasic monohydrate.

In one embodiment, the AAV viral particle formulation of the present invention may comprise one or more isotonicity agents, such as sodium chloride, preferably at a concentration of about 1 mg/ml to about 20 mg/ml, for example, about 1 mg/ml to about 10 mg/ml, about 5 mg/ml to about 15 mg/ml, or about 8 mg/ml to about 20 mg/ml. In another embodiment, the formulation of the present invention comprises about 8.18 mg/ml sodium chloride. Other buffering agents and isotonicity agents known in the art are suitable and may be routinely employed for use in the formulations of the present disclosure.

In another embodiment, the AAV viral particle formulations of the present invention may comprise one or more bulking agents. Exemplary bulking agents include without limitation mannitol, sucrose, dextran, lactose, trehalose, and povidone (PVP K24). In certain preferred embodiments, the formulations of the present invention comprise mannitol, which may be present in an amount from about 5 mg/ml to about 40 mg/ml, or from about 10 mg/ml to about 30 mg/ml, or from about 15 mg/ml to about 25 mg/ml. In a particularly preferred embodiment, mannitol is present at a concentration of about 20 mg/ml.

In some embodiment, the AAV viral particle formulations of the present invention may comprise one or more surfactants, which may be non-ionic surfactants.

Exemplary surfactants include, but are not limited to, ionic surfactants, non-ionic surfactants, and combinations thereof. For example, the surfactant can be, without limitation, TWEEN 80 (also known as polysorbate 80, or its chemical name polyoxyethylene sorbitan monooleate), sodium dodecylsulfate, sodium stearate, ammonium lauryl sulfate, TRITON AG 98 (Rhone-Poulenc), poloxamer 407, poloxamer 188 and the like, and combinations thereof.

In some embodiments, the formulation of the present invention comprises poloxamer 188, which may be present at a concentration of from about 0.1 mg/ml to about 4 mg/ml, or from about 0.5 mg/ml to about 3 mg/ml, from about 1 mg/ml to about 3 mg/ml, about 1.5 mg/ml to about 2.5 mg/ml, or from about 1.8 mg/ml to about 2.2 mg/ml. In a particularly preferred embodiment, poloxamer 188 is present at a concentration of about 2.0 mg/ml.

In other embodiments, the pharmaceutical formulation of the present invention comprises AAV viral particle formulated in a liquid solution that comprises about 1.42 mg/ml of sodium phosphate, dibasic, about 1.38 mg/ml of sodium phosphate, monobasic monohydrate, about 8.18 mg/ml sodium chloride, about 20 mg/ml mannitol and about 2 mg/ml poloxamer 188.

In some embodiments, the AAV viral particle-containing formulations of the present disclosure are stable and can be stored for extended periods of time without an unacceptable change in quality, potency, or purity.

In one embodiment, the formulation is stable at a temperature of about 5° C. (e.g., 2° C. to 8° C.) for at least 1 month, for example, at least 1 month, at least 3 months, at least 6 months, at least 12 months, at least 18 months, at least 24 months, or more.

In another embodiment, the formulation is stable at a temperature of less than or equal to about −20° C. for at least 6 months, for example, at least 6 months, at least 12 months, at least 18 months, at least 24 months, at least 36 months, or more.

In some embodiments, the formulation is stable at a temperature of less than or equal to about −40° C. for at least 6 months, for example, at least 6 months, at least 12 months, at least 18 months, at least 24 months, at least 36 months, or more.

In other embodiments, the formulation is stable at a temperature of less than or equal to about −60° C. for at least 6 months, for example, at least 6 months, at least 12 months, at least 18 months, at least 24 months, at least 36 months, or more.

Transduction/Treatment

In one aspect, the present invention provides uses of the AAV viral particles of the invention for efficient transduction of cells, tissues, and/or organs of interest, and/or for use in gene therapy.

In one embodiment, the present invention provides a method for transduction of cells, tissues, and/or organs of interest, comprising introducing into a cell, a composition comprising an effective amount of the AAV viral particles of the present invention.

In some embodiments, AAV viral particles of the invention are used for transduction of cells, tissues, and/or organs of interest of a subject.

In other embodiments, a method for transduction of cells, tissues, and/or organs of interest, comprising introducing into a cell is provided, the method comprising a composition comprising an effective amount of AAV viral particles of the present invention.

In one embodiment, methods for prophylactic or therapeutic treatment of a subject are provided.

In another embodiment, the subject is need thereof of the prophylactic or therapeutic treatment.

In some embodiments, the subject comprises a condition or disease, wherein the subject is need of treatment for said condition or disease.

In other embodiments, the subject is a mammal.

In one embodiment, the subject is a non-rodent mammal.

In another embodiment, the subject is a primate.

In some embodiments, the subject is a human.

In other embodiments, the subject is a livestock.

In one embodiment, the subject is a horse, sheep, goat, pig, dog, or cat.

In another embodiment, AAV viral particles of the present invention may be administered to the subject through a variety of known administration techniques.

In some embodiments, the AAV viral particle is administered by intravenous injection either as a single bolus or over a prolonged time period, which may be at least about 1, 5, 10, 15, 30, 45, 60, 75, 90, 120, 150, 180, 210 or 240 minutes, or more.

In one embodiment, cells (e.g., ependymal cells) comprising the cerebrospinal fluid (CSF) of the subject are transduced by said AAV viral particles. In some embodiments, cells (e.g., ependymal cells) transduced with the AAV viral particles express and secrete the transgene(s) into the CSF of said mammal.

In another embodiment, administration of the AAV viral particles comprises administration to the cisterna magna, intraventricular space, brain ventricle, subarachnoid space, intrathecal space and/or ependyma of the subject.

In other embodiments, administration of the AAV viral particles comprises administration to the cerebral spinal fluid (CSF) of said subject.

In some embodiments, administration of the AAV viral particles comprises contacting ependymal cells of said subject with the AAV viral particles.

In one embodiment, administration of the AAV viral particles comprises contacting a pial cell, endothelial cell, or meningeal cell of said subject with said AAV viral particles.

In another embodiment, administration of the AAV viral particles comprises injection of the AAV viral particles into a tissue or fluid of the brain or spinal cord of said subject.

In some embodiments, administration of the AAV viral particles comprises injection of the AAV viral particles into cerebral spinal fluid of said subject.

Kits

In another aspect, the present invention provides a kit for use with methods and compositions described herein. Compositions and virus formulations may be provided in the kit. The kits can also include a suitable container and optionally one or more additional agents. In some embodiments, the container is a vial, test tube, flask, bottle, syringe and/or other container. In other embodiments, the kit comprises the AAV viral particle, a pharmaceutically acceptable carrier, and instructional material for the use thereof, for example, for directing the administration of the AAV viral particle.

The invention will be further described in the following examples, which do not limit the scope of the invention described in the claims.

### EXAMPLES

### Example 1

### Supplementing Media with Copper Sulfate Increased VP1 Expression of Bba41 Capsids and Improved Infectivity of AAV Particles Pseudotyped with Bba41 Capsids

### Summary

An insect cell based rAAV production platform was used to produce rAAV capsids. Product quality and potency analysis of this capsid showed a much lower VP1% of capsid protein as compared to the VP1% of rAAV produced in a HEK293 system. Consequently, rAAV capsids produced in insect cells showed significantly lower potency compared to rAAV capsids produced in HEK293 cells, as measured by a transduction assay quantifying luciferase expression by packaged AAV transgene.

In an effort to improve the potency of the capsids produced in an insect cell based rAAV production platform, we evaluated the effect of copper sulfate (CuSO₄) supplementation to the insect cell culture. A range of copper concentrations (0 μM-50 μM) was tested to study the effects on AAV production. rAAV batches produced in the presence of or in absence of copper were purified and analyzed. We identified that copper supplementation in insect cells producing AAV capsids significantly improved the capsid potency when compared to AAV capsids produced without copper supplementation. For example, there was about a 10-fold increase in the AAV capsid potency when compared with the control AAV capsids produced in the insect cell system without any copper supplementation. Analysis of the AAV capsid protein showed a significant increase in the VP1% of AAV produced in the copper supplemented AAV production batches as compared to VP1% of control production batches without copper. In one example, almost two-fold increase in the VP1% of AAV capsids was observed as compared to control AAV capsids produced without copper supplementation. A comparison of AAV capsids produced with the copper supplemented insect cell production platform showed similar potencies to AAV capsids produced in the HEK293 production platform.

Materials and Methods

Cell Culture: Sf9 insect cells (Thermo Fisher Scientific) were cultured at 28 degrees Celsius (° C.).

Suspension Expi293 cells (Thermo Fisher Scientific) were grown in a shaker incubator set at 37° C.

Adherent HEK293T cells used for transduction assays were grown at 37° C. in a static incubator.

A Vi-Cell counter (Beckman Coulter) was used to measure cell density, viability, and average cell diameter. A Nova Flex automated cell counter (Nova Medical) was used to measure the cell culture concentration of metabolites and gases in the cell culture media during AAV production in insect and Expi293 cells.

AAV Production and Purification: To produce rAAV pseudotyped with Bba41 capsids (SEQ ID NO:15), an insect cell-based production process was used to produce green fluorescent protein-luciferase (GFP-Luc) rAAV in a Sf9 derived cell line. GFP-Luc rAAV particles were produced by co-infecting the Sf9 derived cells with recombinant baculoviruses (rBVs) carrying the vg carrying green fluorescent protein (GFP) and luciferase (Luc) transgenes and providing AAV Rep and Cap (Bba41) genes. The rBVs were produced from bacmids that are shuttle vectors that can propagate rBVs when transfected into the insect cells.

For AAV production in insect cells, the rBVs derived from the Autographa californica nuclear polyhydrosis virus (AcNPV) were produced using the Bac-to-Bac baculovirus expression system (Thermo Fisher Scientific). The AAV production process included batch cell cultures and harvest of insect cells infected with the rBVs.

For AAV production in HEK293 cells, plasmids containing Rep, Cap, Ad-helper, and a Gene of interest (GOI) elements needed for rAAV production were transfected using expifectamine transfection reagent. The rAAV were then purified from the harvests.

VP1% analysis of recombinant AAV Capsids: VP1% of recombinant AAV capsids were analyzed on SDS-PAGE by loading a two-step purified AAV samples at amounts equal to 2E+11 capsids per lane. The simply blue Coomassie (Thermo Fisher Scientific) stained gel was visualized via the Chemidoc imager and the densitometric analysis of the resolved bands was performed using the lab image software suite.

In Vitro Transduction Assay: Adherent HEK293T Cells (ATCC) plated in 96-well plates were infected with purified AAV at a target multiplicity of infection. Purified rAAV were diluted in the plating media to reach the final volume. The viral suspension was added to the plated HEK293T cells plated in 96-well plates and incubated. The One-Glo luciferase assay reagent (Promega) was added to the cells and plates were read in the GloMax reader (Promgea). The transduction efficiency was measured as a function of relative luminescence unit (RLU) calculated by output data from the GloMax plate reader.

Analysis of DNA package by AAV capsids: 0.8% alkaline agarose gel was used to resolve the packaged DNA as per standard protocol. 1E+11 vgs as measured by a droplet digital polymerase chain reaction method was used for every sample. SYBR Gold dye (Thermo Fisher Scientific) was used to stain the gel. The stained gel was imaged on Chemidoc's Epiblue transilluminator setting.

### Results and Discussion

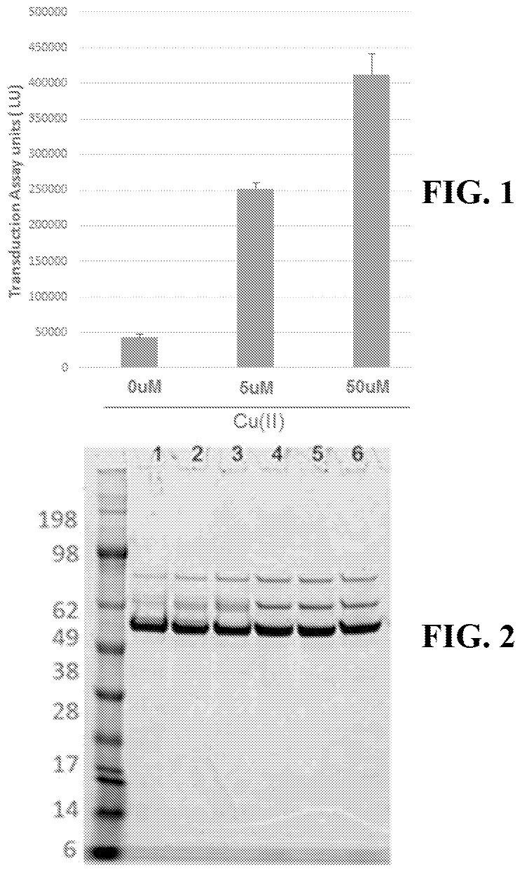

Copper supplemented insect cell cultures producing AAV resulted in significant improvement in the transduction activity of AAV capsids: Copper was added at either 0 μM, 5 μM, or 50 μM concentrations to the insect cell cultures producing AAV capsids. rAAV produced from various copper supplemented insect cultures were isolated, purified, and analyzed. The potency of the purified AAV was measured using the transduction assay. 0 μM copper sulfate supplemented insect cell culture produced rAAV at a titer of 3.4×10E11 vg/ml. 5 μM copper sulfate supplemented insect cell culture produced rAAV at a titer of 2.5×10E11 vg/ml. 50 μM copper sulfate supplemented insect cell culture produced rAAV at a titer of 1.6×10E11 vg/ml. It was noted that increasing concentrations of copper produced acceptable rAAV titers, even though the titers did not increase. As shown in FIG. 1, the transduction assay shows a dose dependent effect of copper on the potency of the novel AAV capsids. AAV capsids produced in 50 μM copper supplemented insect cell culture showed almost ten-fold higher potency than AAV produced without any copper as measured by the luminescence units (LU) in the transduction assay. Similarly, AAV capsids produced in 5 μM copper supplemented insect cell culture showed almost four-fold higher potency than the AAV produced without any copper.

AAV capsids produced by copper supplemented insect cell culture significantly improves VP1%: rAAV produced using the insect cell production process or HEK293 production process in the presence of or absence of copper were isolated, purified, and analyzed. FIG. 2 is a sodium dodecyl sulfate polyacrylamide gel electrophoresis (SDS-PAGE) of purified capsids at final amount of 2×10E+11 capsids per lane for various copper supplemented conditions were resolved on the SDS-PAGE gel (4-12% bis Tris gel, MOPS running buffer). The molecular weight standard used in the SDS-PAGE gel was Seeblue plus. Lane 1 of the gel was from capsids produced in Sf9 cells supplemented with 0 μM copper, where VP1 protein made up 2.3% of the total VP1-3 protein concentration of the capsids. Lane 2 of the gel was from capsids produced in Sf9 cells supplemented with 5 μM copper, where VP1 protein made up 2.9% of the total VP1-3 protein concentration of the capsids. Lane 3 of the gel was from capsids produced in Sf9 cells supplemented with 50 μM copper, where VP1 protein made up 4.5% of the total VP1-3 protein concentration of the capsids. Lane 4 of the gel was from capsids produced in HEK293 cells supplemented with 0 μM copper, where VP1 protein made up 6% of the total VP1-3 protein concentration of the capsids. Lane 5 of the gel was from capsids produced in HEK293 cells supplemented with 5 μM copper, where VP1 protein made up 5.9% of the total VP1-3 protein concentration of the capsids. Lane 6 of the gel was from capsids produced in HEK293 cells supplemented with 50 μM copper, where VP1 protein made up 5.7% of the total VP1-3 protein concentration of the capsids. VP1, VP2 and VP3 capsid proteins were visualized by staining the gel by Coomassie blue stain. As measured by densitometry analysis, VP1% of capsids produced by supplementing 50 μM of copper in Sf9 cell cultures is almost two-fold higher than capsids produced without any copper supplementation. In a separate experiment, rAAV produced by supplementing copper in the 293 production process were also resolved on the SDS PAGE gel. However, no significant improvement in the VP1% is observed in rAAV produced in HEK293 cells supplemented with 50 μM copper. It was unexpected that copper supplementation would increase the VP1% of rAAV produced in insect cells and maintain titers similar to those of rAAV producing insect cells without copper supplementation. VP1% is known to play a direct role in the transduction efficiency of AAV capsids and hence higher potency of AAV capsids is attributed to the improved VP1% in these capsids as a result of copper supplementation in insect cell culture. It was noted that lanes 2 and 3 had a similar banding pattern to lanes 4-6. which indicates that copper supplementation did not appear to affect banding pattern of the VP1 protein. Not wishing to be bound by theory, it is believed that copper supplementation alters the composition VP proteins of capsids produced in insect cells such that the VP protein profile is similar to capsids produced in HEK293 cells.

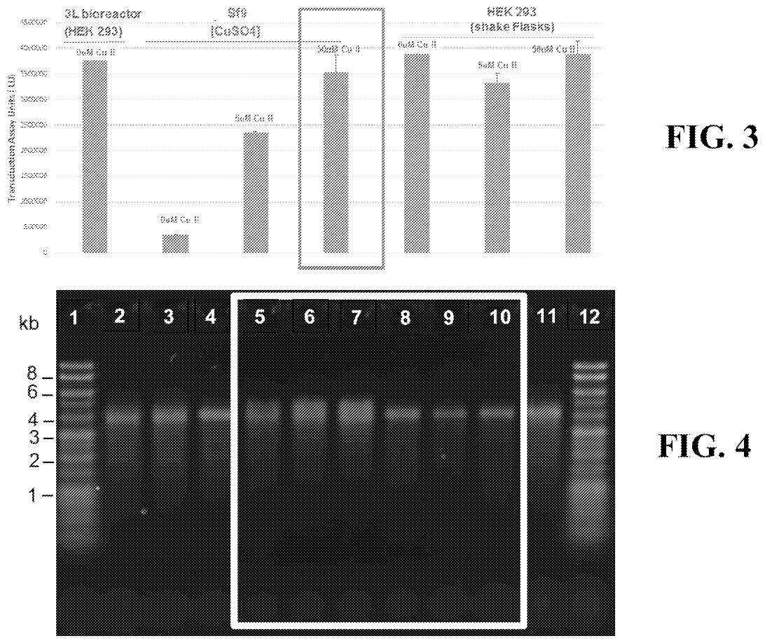

AAV capsids produced by copper supplemented insect cell cultures show similar potency when compared with AAV capsids produced by the HEK293 production platform: Transduction assays were performed on the purified AAV capsids produced in insect cells or HEK293 cells. As shown in FIG. 3, AAV capsids produced by 50 μM copper supplemented insect cell culture has potency similar to the AAV capsids produced by HEK293 cells in either shaker flasks or bioreactors. While capsids produced in insect cell culture with copper supplementation had poor potency, copper supplementation of insect cell cultures generated AAV capsids with similar potency to AAV capsids produced in HEK293 cells. It was unexpected that copper supplementation would increase the potency of rAAV produced in insect cells and maintain titers similar to those of rAAV producing insect cells without copper supplementation. Thus, copper supplementation increases the therapeutic effectiveness of the rAAV particles.

Packaged transgene DNA profile in the AAV capsids with copper supplemented insect cell production platform and 293 production platform as shown by alkaline agarose gel: Purified AAV capsids produced either by insect cell production platform or 293 production platform were resolved on alkaline agarose gel as described previously. FIG. 4 is a 0.8% alkaline agarose gel with SYBR Gold, where 1×10E11 vg were loaded onto the gel. Lanes 1 and 12 of FIG. 4 are Ladder Promega G5711 molecular weight standards. Lane 2 is from 1.74×10E14 rAAV titer produced from 2×1 L shake flasks of Sf cells, the rAAV had a vg size of >4 kilo base pairs (Kb). Lane 3 is from 4.2×10E14 rAAV titer produced from 2×1 L shake flasks of Sf cells, the rAAV had a vg size of >4 Kb. Lane 4 is from 6.5×10E13 rAAV titer produced from 3 L bioreactor of HEK293 cells, the rAAV had a vg size of >4 Kb. Lane 5 is from 7.8×10E13 rAAV titer produced from Sf9 cells supplemented with 0 μM copper sulfate, the rAAV had a vg size of >4 Kb. Lane 6 is from 2.2×10E13 rAAV titer produced from Sf9 cells supplemented with 5 copper sulfate, the rAAV had a vg size of >4 Kb. Lane 7 is from 1.5×10E13 rAAV titer produced from Sf9 cells supplemented with 50 μM copper sulfate, the rAAV had a vg size of >4 Kb. Lane 8 is from 1.0×10E13 rAAV titer produced from HEK293 cells supplemented with 0 μM copper sulfate, the rAAV had a vg size of >4 Kb. Lane 9 is from 1.0×10E13 rAAV titer produced from HEK293 cells supplemented with 50 μM copper sulfate, the rAAV had a vg size of >4 Kb. Lane 10 is from 6.6×10E12 rAAV titer produced from HEK293 cells supplemented with 50 μM copper sulfate, the rAAV had a vg size of >4 Kb. Lane 10 is from 6.5×10E14 rAAV titer, the rAAV had a vg size of >4 Kb. As shown in FIG. 4, the DNA profile of the packaged DNA remains very similar in both with copper (lanes 6 and 7) and without copper (lane 5) supplemented insect cell produced AAV capsids.

### Example 2

### Supplementing Media with Copper Sulfate Increased VP1 Expression of AAV5 Capsids and Improved Infectivity of AAV Particles Pseudotyped with AAV5 Capsids

AAV5 Production in Shake Flasks: For AAV production in insect cells, recombinant baculoviruses (rBVs) derived from the Autographa californica nuclear polyhydrosis virus (AcNPV) were produced using the Bac-to-Bac baculovirus expression system as per the manufacturer's protocol (Thermo Fisher Scientific). The AAV production process includes batch cell culture in shake flasks and harvest of insect cells that have been co-infected with rBVs with a transgene polynucleotide sequence (GUI) and rBVs with sequences encoding Rep and Cap (Viral vector replication and capsid proteins), followed by purification using AVB sepharose affinity capture. In this example, the sequence encoding Cap, particularly encodes the AAV5 serotype capsid (NCBI Reference Sequence No. YP_068409.1). During the production process, copper sulfate (CuSO₄) was added to the cell culture. Different copper sulfate concentrations (5 μM, 25 μM, and 45 μM) were tested. Two flasks were included as control (0 μM copper sulfate).

Reverse phase high-performance liquid chromatography (RP-HPLC) was used to assess the VP1 levels of capsids of the rAAV particles produced with different copper sulfate concentrations. RP-HPLC method employs a C3 column and an acetonitrile gradient in the presence of the ion-pairing agent, trifluoroacetic acid. The rAAV particles were injected to the column, where the rAAV particles are dissociated and the three types of capsid proteins (VP1, VP2 and VP3) are separated. The relative peak area percentages of the three viral coat proteins were quantitated using areas under the UV 214 nm peaks and VP1% is reported.

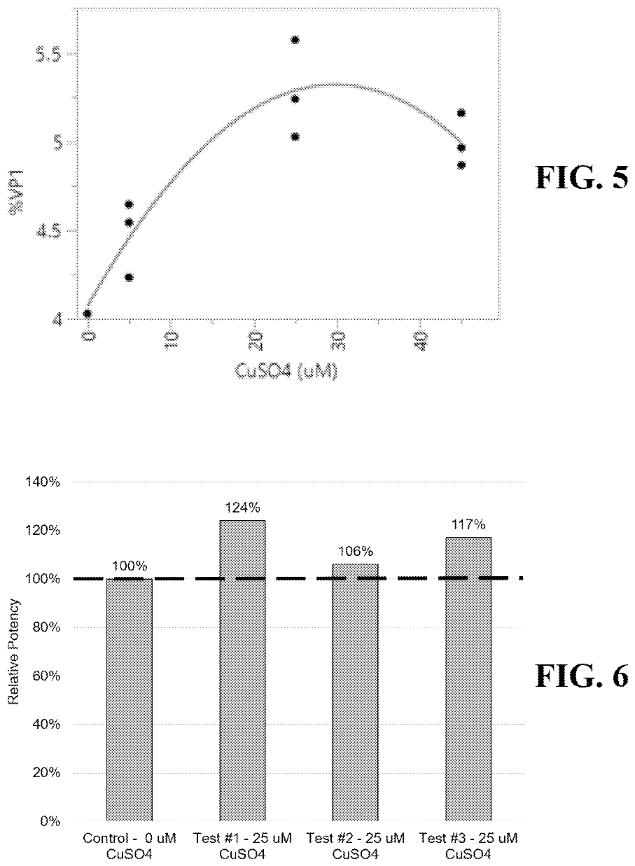

As shown in FIG. 5, the addition of copper to the cell culture increased the incorporation of VP1 into the capsids of the rAAV particles. Particularly, the increase of VP1 in the capsids were seen when 5 μM, 25 μM, and 45 μM of copper sulfate was added to the cell culture. The VP1% of AAV5 capsids produced with 5 μM copper sulfate ranged from ˜4.25% to >4.5%. The VP1% of AAV5 capsids produced with 25 μM copper sulfate was ≥5%. The VP1% of AAV5 capsids produced with 45 μM copper sulfate was ≥4.5%. The VP1% of AAV5 capsids produced without copper sulfate was 4%. Accordingly, different copper concentration increased the VP1 content of the AAV % capsids. FIG. 5 also shows a concentration of ˜30 μM of copper sulfate being able to improve VP1 content in the capsids as compared to a control production of rAAV particles.

To test the effect on the infectivity of the rAAV particles, HepG2 cells (ATCC) were infected with the rAAV particles. To quantify protein expression of the gene of interest in the infected HepG2 cells, the supernatant from the cell culture was harvested and infected HepG2 cells were lysed. Protein samples were analyzed via enzyme-linked immunosorbent assay (ELISA). The protein samples were incubated with a capture antibody immobilized onto a surface of a well. The capture antibody specifically binds the protein encoded by the GOI. After binding of the protein encoded by the GOI to the capture antibody, a detection antibody targeting the protein encoded by the GOI to the capture antibody was added to the well. The capture antibody was conjugated with a detection element such as horse-radish peroxidase for quantification of the capture protein. Using a protein standard curve, the protein encoded by the GOI was quantified.

As shown in FIG. 6, the addition of copper to the cell culture enhanced the infectivity of the rAAV particles. Particularly, AAV5 capsids produced with 25 μM copper sulfate had infectivities/relative potencies of 124%, 106%, and 117% relative to the infectivity of AAV5 capsids produced without copper. Thus, copper supplementation increases the therapeutic effectiveness of the rAAV particles.

AAV5 Produced in Bioreactors: To show the effect of copper on larger scale rAAV productions, rAAV production was undertaken in bioreactors. Particularly, the AAV production process includes batch cell culture in 3 L and 2000 L bioreactors and harvest of insect cells that have been co-infected with rBVs with a transgene polynucleotide sequence (Gene of interest (GOI)) and rBV with sequences encoding Rep and Cap (Viral vector replication and capsid proteins), followed by purification using AVB sepharose affinity capture and/or ion exchange chromatography. In this example, the sequence encoding Cap also encodes the AAV5 serotype capsid. During the production process, 30 μM copper sulfate was added to the cell culture. RP-HPLC was used to assess the VP1 levels of capsids of the rAAV particles.

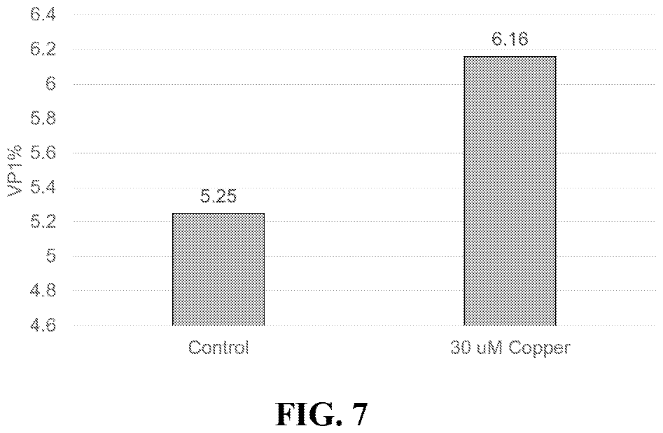

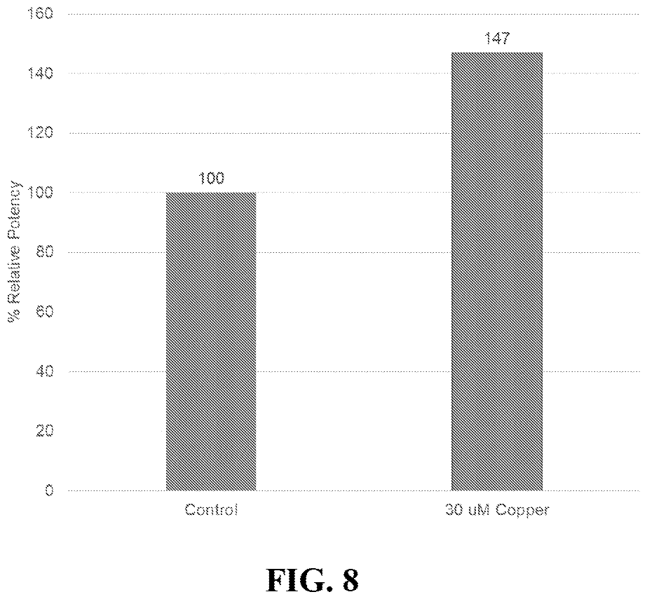

FIG. 7 shows the RP-HPLC analysis of the rAAV particles, where the addition of copper sulfate in a scaled up rAAV production increased the VP1 levels of capsids of the rAAV particles. The VP1% of AAV5 capsids produced with 30 μM copper sulfate was 6.16%, whereas the VP1% of AAV5 capsids produced without copper sulfate was 5.25%. FIG. 8 also shows the addition of copper sulfate in the scaled up rAAV production increased the potency of the rAAV particles. Particularly, AAV5 capsids produced with 30 μM copper sulfate had an infectivity/relative potency of 147% relative to the infectivity of AAV5 capsids produced without copper. For FIGS. 7 and 8, the rAAV particles were purified by AVB sepharose affinity capture and ion exchange chromatography.

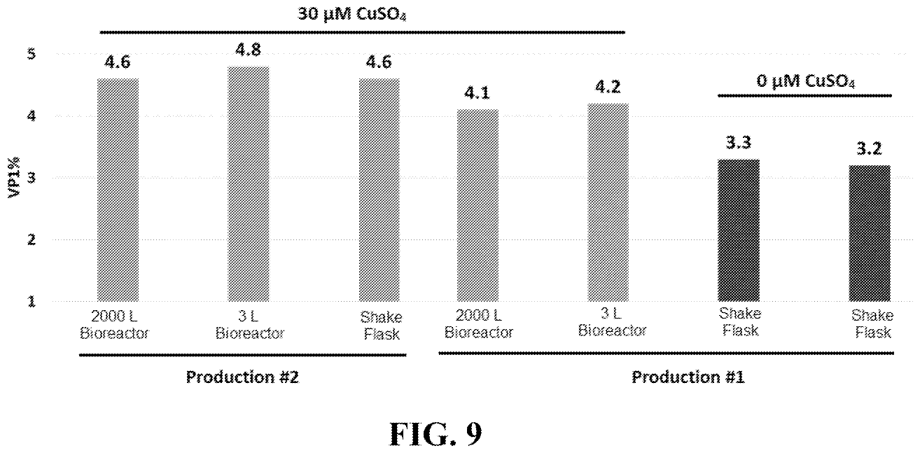

FIG. 9 shows the RP-HPLC analysis of rAAV particles produced in shake flasks, 3 L bioreactors, and 2000 L bioreactors. In production #1, rAAV particles produced in 3 L and 2000 L bioreactors supplemented with 30 μM copper sulfate had increased VP1 levels as compared to rAAV particles produced in shake flask cultures without copper. In production #2, 30 μM copper sulfate increased VP1 levels in shake flask, 3 L bioreactor, and 2000 L bioreactor productions. The rAAV particles in productions #1 and #2 were purified by AVB sepharose affinity capture. Particularly as shown in FIG. 9, the addition 30 μM copper sulfate increased the VP1% to 4.2% for a 3 L bioreactor production, 4.1% for a 2000 L bioreactor production, 4.6% for a shake flask production, 4.8% for a second 3 L bioreactor production, and 4.6% for a second 2000 L bioreactor production. These VP1% were greater than the VP1% of AAV5 capsids produced without copper (3.3% and 3.2% for shake flask productions).

As shown in FIGS. 7-9, copper supplementation improved VP1 content in the capsids and increased the therapeutic effectiveness of the rAAV particles.

### Example 3

### Supplementing Media with Copper Sulfate Increased VP1 Expression of AAV9 Capsids and Improved Infectivity of AAV Particles Pseudotyped with AAV9 Capsids

For AAV production in insect cells, rBVs derived from AcNPV were produced using the Bac-to-Bac baculovirus expression system as per the manufacturer's protocol (Thermo Fisher Scientific). The AAV production process includes batch cell culture in shake flasks and harvest of insect cells that have been co-infected with rBVs with a transgene polynucleotide sequence (GOI) and rBVs with sequences encoding Rep and Cap (Viral vector replication and capsid proteins), followed by purification using immunochromotography capture. In this example, the sequence encoding Cap, particularly encodes the AAV9 serotype capsid (Genbank Accession No. AAS99264.1). During the production process, copper sulfate (CuSO₄) was added to the cell culture. Different copper sulfate concentrations (0 μM, 10 μM, 20 μM, and 30 μM) were tested.

Capillary electrophoresis (UV 214 nm) was used to assess the VP1 levels of capsids of the rAAV particles produced with different copper sulfate concentrations.

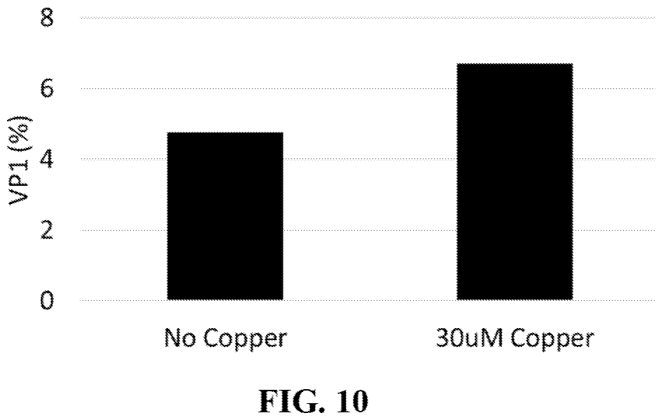

FIG. 10 shows the concentration of 30 μM of copper sulfate improving VP1 content in the capsids as compared to a control production of rAAV particles. Particularly, 30 μM of copper sulfate increased the VP1% to >6% of AAV9 capsids, whereas AAV9 capsids produced without copper had a VP1% of less than 5%.

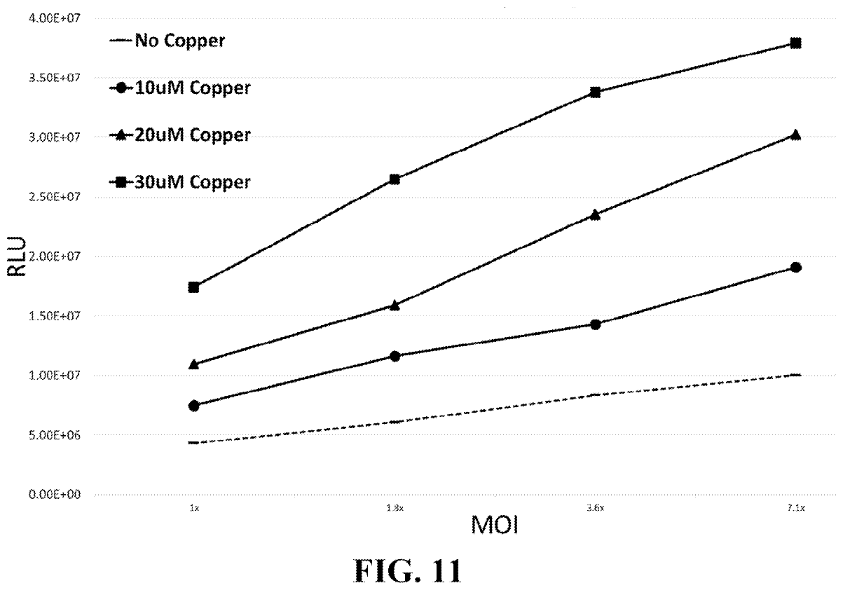

To test the infectivity of the rAAV particles, HEK293 cells were infected with the rAAV particles at different multiplicities of infection (MOIs; the ratio of virus to cells)). To quantify protein expression of the gene of interest (e.g., luciferase) in the infected HEK293 cells, the supernatant from the cell culture was harvested and infected HEK293 cells were lysed. Luciferin was added to the protein samples and the relative light units (RLU) were quantified. As shown in FIG. 11, the addition of 10 μM, 20 μM, and 30 μM copper sulfate to the cell culture enhanced the infectivity of the rAAV particles as compared to control production cultures without copper. The HEK293 cells infected with AAV9 capsids produced using copper supplementation exhibited higher RLUs (>7E06 to >3.5E07 RLUs) as compared to AAV9 capsids produced without copper (<5E06 to ˜1E07 RLUs). Thus, 30 μM of copper sulfate improved infectivity over 10 μM and 20 μM copper concentration.

As shown in FIGS. 10 and 11, copper supplementation increases the VP1 content of the produced AAV9 capsids and improved the therapeutic effectiveness of the AAV9 capsids.

### Example 4

### In Vivo Analysis of Transduction/Infectivity of AAV5 Capsids Produced in Copper Sulfate Supplemented Media

rAAV pseudotyped with AAV5 capsids was produced using a baculovirus/Sf9 expression system supplemented with copper sulfate (30 μM copper sulfate; productions #1 and #2 from Example 2). The vector genome of the rAAV included a gene of interest (GOI) encoding a protein (GOI protein). The purified vectors were quantified by quantitative polymerase chain reaction (qPCR) and intravenously dosed at different doses ranging from 2e14 to 2e12 vector genomes per kilogram (vg/kg) into 30 Rag2^{-/-} mice alongside a vehicle control group (5 Rag2^{-/-} mice).

At 52 weeks post dosing, liver tissue samples were extracted for subsequent quantification of vector-derived DNA and RNA. DNA and RNA were extracted from the liver tissue samples using the DNeasy Blood and Tissue Kit and RNeasy Plus Mini Kit as per the manufacturer's protocol (Qiagen). Concentrations of the extracted DNA and RNA and diluted prior to digital droplet polymerase chain reaction (ddPCR) analysis. Examples of ddPCR are described in Pasi, K. John, et al. “Multiyear Follow-Up of AAV5-FVIII-SQ Gene Therapy for Hemophilia. A,” New England Journal of Medicine 382.1 (2020): 29-40; Regan, John F., et al, “A Rapid Molecular Approach for Chromosomal Phasing.” PloS one 10.3 (2015): e0118270; and Furuta-Hanawa, Birei, Teruhide Yamaguchi, and Eriko Uchida. “Two-Dimensional Droplet Digital PCR as a Tool for Titration and Integrity Evaluation of Recombinant Adeno-Associated Viral Vectors” Human gene therapy methods 30.4 (2019): 127-136. Measurement of vector-derived DNA and mRNA extracted from liver tissue collected at 52 weeks post administration showed dose-dependent increases in both vector DNA and the resulting transcript, which ranged from ˜1e7 to ˜2e4 copies per microgram (μg) of DNA and ˜2.3e9 to 8e6 copies per μg of RNA. The vehicle control showed levels of DNA below the assay's lower limit of quantitation and undetectable levels of mRNA.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

1. A method for preparing recombinant adeno-associated virus (rAAV), the method comprising the step of:
culturing, in a culture medium with an effective amount of a transition metal, host cells capable of producing an rAAV capsid;
wherein the effective amount of the transition metal increases incorporation of VP1, VP2, or VP3 protein into the rAAV capsid.

2. (canceled)

3. (canceled)

4. A method for preparing a recombinant adeno-associated virus (rAAV), the method comprising the step of:
culturing, in a culture medium with an effective amount of a transition metal, a host cell capable of producing an rAAV capsid;
wherein the effective amount of the transition metal increases incorporation of VP1 and VP3 proteins into the rAAV capsid.
wherein the rAAV capsid has concentrations of VP1 and VP3 proteins that are greater than concentrations of VP1 and VP3 proteins of an rAAV capsid produced under the same conditions but being devoid of the effective amount of the transition metal.

5. (canceled)

6. A method for preparing recombinant adeno-associated virus (rAAV), the method comprising the step of:
culturing a host cell in a culture medium having an effective amount of a cysteine protease inhibitor, the host cell being capable of producing an rAAV capsid;
wherein the effective amount of a cysteine protease inhibitor increases incorporation of VP1, VP2, or VP3 protein into the rAAV capsid.

7. (canceled)

8. (canceled)

9. The method as in claim 1 wherein the host cell is a non-mammalian host cell.

10. The method as in claim 1, wherein the host cell is an insect cell.

11. The method as in claim 1, wherein the transition metal is selected from one or more of copper, scandium, titanium, vanadium, chromium, manganese, iron, cobalt, nickel, copper, zinc, yttrium, zirconium, niobium, molybdenum, technetium, ruthenium, rhodium, palladium, silver, cadmium, hafnium, tantalum, tungsten, rhenium, osmium, iridium, platinum, gold, mercury, rutherfordium, dubnium, seaborgium, bohrium, hassium, meitnerium, ununnilium, unununium, and ununbium.

12. (canceled)

13. The method as in claim 1, wherein the transition metal is selected from one or more of a copper sulfate, copper nitrate, copper selenide, copper hydroxide, copper oxide, copper phosphate, copper silicate, copper borate, copper carbonate aluminum chloride, magnesium chloride, lithium selenide, sodium carbonate, lithium chloride, sodium hydrogen phosphate, sodium metasilicate, strontium hydroxide, trisodium phosphate, potassium fluoride, magnesium sulfate, calcium chloride, sodium sulfate, aluminum sulfate, sodium tetraborate, magnesium sulfate, magnesium bromide, rubidium aluminum sulfate, barium hydroxide, potassium aluminum sulfate, magnesium nitrate, sodium hydrogen phosphate, nickel sulfate, zinc sulfate, beryllium sulfate, lithium nitrate, strontium chloride, zinc nitrate, sodium pyrophosphate, calcium bromide, copper nitrate, aluminum nitrate, sodium tetraborate, silver fluoride, calcium iodide, lithium bromide, lithium iodide, strontium bromide, calcium nitrate, strontium iodide, sodium bromide and strontium nitrate, sodium aluminum lactate, sodium acetate, sodium dehydroacetate, sodium butoxy ethoxy acetate, sodium caprylate, sodium citrate, sodium lactate, sodium dihydroxy glycinate, sodium gluconate, sodium glutamate, sodium hydroxymethane sulfonate, and sodium oxalate.

14. The method of claim 1, wherein the effective amount ranges from about 1 nM to about 1 mM.

15. The method of claim 1, wherein the effective amount ranges from about 10 nM to about 100 μM.

16. The method of claim 1, wherein the effective amount ranges from about 20 μM to about 25 μM.

17.-26. (canceled)

27. The method as in claim 1 further comprising the step of isolating the rAAV capsid by the host cell.

28. The method as in claim 1 further comprising host cells capable of producing rAAV capsids.

29. The method as in claim 1 further comprising host cells capable of producing a concentration of rAAV capsids, wherein the effective amount reduces the concentration of rAAV capsids.

30. The method as in claim 1 further comprising host cells capable of producing a concentration of rAAV capsids, wherein the concentration of rAAV capsids is less than a concentration of rAAV capsids produced under the same conditions but being devoid of the effective amount.

31.-34. (canceled)

35. The method as in claim 1, wherein the culturing step occurs in a volume of 25 milliliters or more.

36. The method as in claim 1, wherein the culturing step occurs in a volume of 100 milliliters or more.

37. The method as in claim 1, wherein the culturing step occurs in a volume of 1 liter or more.

38. The method as in claim 1, wherein the culturing step occurs in a volume of 10 liters or more.

39. The method as in claim 1, wherein the culturing step occurs in a volume of 100 liters or more.

40. The method as in claim 1, wherein the culturing step occurs in a volume of 500 liters or more.


SEQUENCE LISTING (76 sequences)

SEQ ID NO: 1 - protein, 723 aa, Adeno-associated virus
  misc_feature (1)..(723): VP1 protein of the wild-type AAV serotype 5 (AAV-5)
SFVDHPPDWGLREFLGLEALEEVGPPKGEPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 2 - protein, 742 aa, Papio hamadryas
  misc_feature (1)..(742): VP1 sequence (from baboon; denoted as Bba21)
MAADGYLPDWLEDNLSEGIREWWALKPGAPQPKANQQHQDNARGLVLPGYKYLGPGNGLDKGEPVNEADAAALEHDKAYDQQLKSGDNPYLKYNHADAEFQQRLATDTSFGGNLGKAVFQAKKRILEPLGLVEEGVKTAPGKKRPLEKTPNRPTNPDSGKAPAKKKQKDGETADSARRTLDFEDSGAGDGPPEGSSSGEMSHDAEMRAAPGGNAVEAGQGADGVGNASGDWHCDSTWSEGRVTTTSTRTWVLPTYNNHLYLRIGTTANSNTYNGFSTPWGYFDFNRFHCHFSPRDWQRLINNNWGLRPKSMRVKIFNIQVKEVTTSNGETTVANNLTSTVQIFADSTYELPYVMDAGQEGSLPPFPNDVFMVPQYGYCGVVTGENQNQTDRNAFYCLEYFPSQMLRTGNNFEVSYQFEKVPFHSMYAHSQSLDRMMNPLLDQYLWHPQSTTTGNSLNQGTATTTYGKITTGDFAYYRKNWLPGACIKQQKFSKNASQNYKIPASGGDALLKYDTHTTSNGRWSNMAPGPPMATAGAGDSDFSNSQLIFAGPNQSGNTTTSSNNLLFTSEEEIATTNPRDTDMFGQIADNNQNATTAPHIANLDAMGIVPGMVWQNRDIYYLGPIWAKVPHTDGHFHPSPLMGGFGLKHPPPQIFIKNTPVPANPNTTFSAARINSFLTQYSTGQVAVQIDWEIQKEHSKRWDPEVQFTSNYGTQNSMLWAPDNAGNYHEPRAIGSRFLTHHL

SEQ ID NO: 3 - protein, 739 aa, Papio hamadryas
  misc_feature (1)..(739): VP1 sequence (from baboon; denoted as Bba26)
MAADGYLPDWLEDNLSEGIREWWALKPGAPQPKANQQHQGNSRGLVLPGYKYLGLFNGLDKGEPVNEADAAALEHDKAYDKQLEQGDDPYLKYNHADAEFQERLQEDTSFGGNLGRAVFQAKKRILEPLGLVEEAAKTAPGKKRPLEKTPNQPTDTQAAGQTPAKKRPQGEQSGDSARRQLDFGPQPAAPIGQPPAAPPPVGSNTMASGGGGPMADDNQGADGVGNASGNWHCDSTWLGDRVITTSTRTWVLPTYNNHIYKQISSESGATNDNHYFGYSTPWGYFDFNRFHCHFSPRDWQRLINNNWGFRPKRLNFKIFNVQVKEVTQIEGGSTIANNLTSTIQVFADSEYQLPYVLGSAHQGCLPPFPADVFMIPQYGYLTLNNGSQSVGRSSFYCLEYFPSQMLRTGNNFQFSYTFESVPFHSSYAHSQSLDRIMNPLVDQYLYYLARTQTGTGSTTSNTRQLQFYQAGPSNMADQSRNWLPGPMYRQQRVSKTLDQNSNTNFAWTAASKYNLNGRKSLANPGIAMATHKDDEERFFPQHGVLVFGQTNATNKTTLDNVLVTSEEEIKATNPVATEEYGTVSSNLQASNTNPTTETVNNQGILPGMVWQDRDVYLQGPIWAKIPHTDGHFHPSPLMGGFGLKHPPPQILIKNTPVPANPPETFTTSKFASYITQYSTGQVSVEIEWELQKENSKRWNPEIQYTSNYAKSNNVEFSVDAAGVYSEPRPIGTRYLTRNL

SEQ ID NO: 4 - protein, 739 aa, Papio hamadryas
  misc_feature (1)..(739): VP1 sequence (from baboon; denoted as Bba27)
VAADGYLPDWLEDNLSEGIREWWALKPGAPQPKANQQHQDNSRGLVLPGYKYLGPFNGLDKGEPVNEADAAALEHDKAYDKQLEQGDNPYLKYNHADAELQERLQEDTSFGGNLGRAVFQAKKRILEPLGLVEEAAKTAPGKKRPLEKTPNQPTDTQAAGQTPAKKRPQGEQSGDSARRQLDFDPQPAAPIGQPPAAPPPVGSNTMASGGGGPMADDNQGADGVGNASGNWHRDSTWLGDRVITTSTRTWVLPTYNNHIYKQISSESGATNDNHYFGYSTPWGYFDFNRFHCHFSPRDWQRLINNNWGFRPKRLNFKIFNIQVKEVTQIEGGSTIANNLTSTIQVFADSEYQLPYVLGSAHQGCLPPFPADVFMIPQYGYLTLNNGSQSVGRSSFYCLEYFPSQMLRTGNNFQFSYTFESVPFHSSYAHSQSLDRIMNPLVDQYLYYLARTQTGTGSTTSNTRQLQFYQAGPSNMADQSRNWLPGPMYRQQRVSKTLDQNSNTNFAWTAASKYNLNGRKSLANPGIAMATHKDDEERFFPQHGVLVFGQTNATNKTTLDNVLITSEEEIKATNPVATEEYGTVSSNLQASNTNPTTETVNSQGILPGMVWQDRDVYLQGPIWAKIPHTDGHFHPSPLMGGFGLKHPPPQILIKNTPVPANPPETFTTSKFASYITQYSTGQVSVEIEWELQKENGKRWNPEIQYTSNYAKSNNVEFSVDAAGVYSEPRPIGTRYLTRNL

SEQ ID NO: 5 - protein, 739 aa, Papio hamadryas
  misc_feature (1)..(739): VP1 sequence (from baboon; denoted as Bba29)
MAADGYLPDWLEDNLSEGIREWWALKPGAPQPKANQQHQDNSRGLVLPGYKYLGPFNGLDKGEPVNEADAAALEHDKAYDKQLEQGDNPYLKYNHADAEFQERLQEDTSFGGNLGRAVFQAKKRILEPLGLVEEAAKTAPGKRRPLEKTPNQPTDTQAAGQTPAKKRPQGEQSGDSARRQLDFDPQPAAPIGQPPAAPSPVGSNTMASGGGGPMADDNQGADGVGNASGNWHCDSTWLGDRVITTSTRTWVLPTYNNHIYKQISSESGATNDNHYFGYSTPWGYFDFNRFHCHFSPRDWQRLINNNWGFRPKRLNFKIFNIQVKEVTQIEGGSTIANNLTSTIQVFADSEYQLPYVLGSAHQGCLPPFPADVFMIPQYGYLTLNNGSQSVGRSSFYCLEYFPSQMLRTGNNFQFSYTFESVPFHSSYAHSQSLDRIMNPLVDQCLYYLARTQTGTGSTTSNTRQLQFYQAGPSNMADQSRNWLPGPMYRQQRVSKTLDQNSNTNFAWTAASKYNLNGRKSLANPGIAMATHKDDEERFFPQHGVLVFGQTNATNKTTLDNVLITSEEEIKATNPVATEEYGTVSSNLQASNTNPTTETVNNQGILPGMVWQDRDVYLQGPIWAKIPHTDGHFHPSPLMGGFGLKHPPPQILIKNTPVPANPPETFTTSKFASYITQYSTGQVSVEIEWELQKEDSKRWNPEIQYTSNYAKSNNVEFSVDAAGVYSEPRPIGTRYLTRNL

SEQ ID NO: 6 - protein, 739 aa, Papio hamadryas
  misc_feature (1)..(739): VP1 sequence (from baboon; denoted as Bba30)
MAADGYLPDWLEDNLSEGIREWWALKPGAPQPKANQQHQDNSRGLVLPGYKYLGPFNGLDKGEPVNEADAAALEHDKAYDKQLEQGDNPYLKYNHADAEFQERLQEDTSFGGNLGRAVFQAKKRILEPLGLVEEAAKTAPGKKRPLEKTPNQPTDTQAAGQTPAKKRPQGGQSGDSARRQLDFDPQPAAPIGQPPAAPSPVGSNTMASGGGGPMADDNQGADGVGNASGNWHCDSTWLGDRVITTSTRTWVLPTYNNHIYKQISSESGATNDNHYFGYSTPWGYFDFNRFHCHFSPRDWQRLINNNWGFRPKRLNFKIFNIQVKEVTQIEGGSTIANNLTSTIQVFADSEYLLPYVLGSAHQGCLPPFPADVFMIPQYGYLTLNNGSQSVGRSSFYCLEYFPSQMLRTGNNFQFSYTFESVPFHSSYAHSQSLDRIMNPLVDQYLYYLARTQTGTGSTTSNTRQLQFYQAGPSNMADQSRNWLPGPMYRQQRVSKTLDQNSNTNFAWTAASKYNLNGRKSLANPGIAMATHKDDEERFFPQHGVLVFGQTNATNKTTLDNVLITSEEEIKATNPVATEEYGTVSSNLQASNTNPTTETVNNQGILPGMVWQDRDVYLQGPIWAKIPHTDGHFHPSPLMGGFGLKHPPPQILIKNTPVPANPPETFTTSKFASYITQYSTGQVSVEIEWELQKEDSKRWNPEIQYTSNYAKSNNVEFSVDAAGVYSEPRPIGTRYLTRNL

SEQ ID NO: 7 - protein, 742 aa, Papio hamadryas
  misc_feature (1)..(742): VP1 sequence (from baboon; denoted as Bba31)
MAADGYLPDWLEDNLSEGIREWWALKPGAPQPKANQQHQDNARGLVLPGYKYLGPGNGLDKGEPVNEADAAALEHDKAYDQQLKSGDNPYLKYNHADAEFQQRLATDTSFGGNLGKAVFQAKKRILEPLGLVEEGVKTAPGKKHPLEKTPNRPTNPDSGKAPAKKKQKDGETADSARRTLDFEDSGAGDGPPEGSSSGEMSHDAEMRAAPGGNAVEAGQGADGVGNASGDWHCGSTWSEGRVTTTSTRTWVLPTYNNHLYLRIGTTANSNTYNGFSTPWGYFDFNRFHCHFSPRDWQRLINNNWGLRPKSMRVKIFNIQVKEVTTSNGETTVANNLTSTVQIFADSTYELPYVMDAGQEGSLPPFPNDVFMVPQYGYCGVVTGENQNQTDRNAFYCLECFPSQMLRTGNNFEISYQFEKVPFHSMYAHSQSLDRMMNPLLDQYLWHLQSTTTGNSLNQGTATTTYGKITTGDFAYYRKNWLPGACIKQQKFSKNASQNYKIPASGGDALLKYDTHTTLNGRWSNMAPGPPMATAGAGDSDFSNSQLIFAGPNQSGNTTTSSNNLLFTSEEEIAATNPRDTDMFGQIADNNQNATTAPHIANLDAMGIVPGMVWQNRDIYYQGPIWAKVPHTDGHFHPSPLMGGFGLKHPPPQIFIKNTPVPANPNTTFSAARINSFLTQYSTGQVAVQIDWEIQKEHSKRWNPEVQFTSNYGTQNSMLWAPDNAGNYHEPRAIGSRFLTHHL

SEQ ID NO: 8 - protein, 742 aa, Papio hamadryas
  misc_feature (1)..(742): VP1 sequence (from baboon; denoted as Bba32)
MAADGYLPDWLEDNLSEGIREWWALKPGAPQPKANQQHQDNARGLVLPGYKYLGPGNGLDKGEPVNEADAAALEHDKAYDQQLKSGDNPYLKYNHADAEFQQRLATDTSFGGNLGKAVFQAKKRILEPLGLVEEGVKTAPGKKRPLEKTPNRPTNPDSGKAPAKKKQKDGETADSARRTLDFEDSGAGDGPPEGSSSGEMSHDAEMRAAPGGNAVEAGQGADGVGNASGGWHCDSTWSEGRVTTTSTRTWVLPTYNNHLYLRIGTTANSNTYNGFSTPWGYFDFNRFHCHFSPRDWQRLINNNWGLRPKSMRVKIFNIQVKEVTTSNGETTVANNLTSTVQIFADSTYELPYVMDAGQEGSLPPFPNDVFMVPQYGYCGVVTGENQNQTDRNAFYCLEYFPSQMLRTGNNFEISYQFEKVPFHSMYAHSQSLDRMMNPLLDQYLWHLQSTTTGNSLNQGTATTTYGKITTGDFAYYRKNWLPGACIKQQKFSKNASQNYKIPASGGDALLKYDTHTTLNGRWSSMAPGPPMATAGAGDSDFSNSQLIFAGPNQSGNTTTSSNNLLFTSEEEIATTNPRDTDMFGQIADNNQNATTAPHIANLDAMGIVPGMVWQNRDIYYQGPIWAKVPHTDGHFHPSPLMGGFGLKHPPPQIFIKNTPVPANPNTTFSAARINSFLTQYSTGQVAVQIDWEIQKEHSKRWNPEVQFTSNYGTQNSMLWAPGNAGNHHEPRAIGSRFLTHHL

SEQ ID NO: 9 - protein, 742 aa, Papio hamadryas
  misc_feature (1)..(742): VP1 sequence (from baboon; denoted as Bba33)
MAADGYLPDWLEDNLSEGIREWWALKPGAPQPKANQQHQDNARGLVLPGYKYLGPGNGLDKGEPVNEADAAALEHDKAYDQQLKSGDNPYLKYNHADAEFQQRLATDTSFGGNLGKAVFQAKKRILEPLGLVEEGVKTAPGKKRPLEKTPNRPTNPDSGRAPAKKKQKDGETADSARRTLDFEDSGAGDGPPEGSSSGEMSHDAEMRAAPGGNAVEAGQGADGVGNASGDWHCDSTWSEGRVTTTSTRTWVLPTYNNHLYLRIGTTANSNTYNGFSTPWGYFDFNRFHCHFSPRDWQRLINNNWGLRPKSMRVKIFNIQVKEVTTSNGETTVANNLTSTVQIFADSTYELPYVMDAGQEGSLPPFPNDVFMVPQYGYCGVVTGENQNQTDRNAFYCLEYFPSQMLRTGNNFEISYQFEKVPFHSMYAHSQSLDRMMNPLLDQYLWHLQSTTTGNSLNQGTATTTYGKITTGDFAYYRKNWLPGACIKQQKFSKNASQNYKIPASGGDALLKYDTHTTLNGRWSNMAPGPPMATAGAGDSDFSNSQLIFAGPNQSGNTTTSSNNLLLTSEEEIATTNPRDTDMFGQIADNNQNATTAPHIANLDAMGIVPGMVWQNRDIYYQGPIWAKVPHTDGHFHPSPLMGGFGLKHPPPQIFIKNTPVPANPNTTFSAARINSFLTQYSTGQVAVQIDWEIQKEHSKRWNPEVQFTSNYGTQNSMLWAPGNAGNYHEPRAIGSRFLTHHL

SEQ ID NO: 10 - protein, 739 aa, Papio hamadryas
  misc_feature (1)..(739): VP1 sequence (from baboon; denoted as Bba34)
MAADGYLPDWLEDNLSEGIREWWALKPGAPQPKANQQHQDNSRGLVLPGYKYLGPFNGLDKGEPVNEADAAALEHDKAYDKQLEQGDNPYLKYNHADAEFQERLQEDTSFGGILGRAVFQAKKRILEPLGLVEEAAKTAPGKKRPLEKTPNQPTDTQAAGQTPAKKRPQGEQSGDSARRQLDFDPQPAAPIGQPPAAPSPVGSNTMASGGGGPMADDNQGADGVGNASGNWHCDSTWLGDRVITTSTRTWVLPTYNNHIYKQISSESGATNDNHYFGYSTPWGYFDFNRFHCHFSPRDWQRLINNNWGFRPKRLNFKIFNIQVKEVTQIEGGSTIANNLTSTIQVFADSEYQLPYVLGSAHQGCLPPFPADVFMIPQYGYLTLNNGSQSVGRSSFYCLEYFPSQMLRTGNNFQFSYTFESVPFHSSYAHSQSLDRIMNPLVDQYLYYLARTQTGTGSTTSNTRQLQFYQAGPSNMADQSRNWLPGPMYRQQRVSKTLDQNSNTNFAWTAASKYNLNGRKSLVNPGIAMATHKDDEERFFPQHGVLVFGKTNATNKTTLENVLVTDEEEVKATNPVATEEYGTVSSNLQSNTTNPTTETVNNQGILPGMVWQDRDVYLQGPIWAKIPHTDGHFHPSPLMGGFGLKHPPPQILIKNTPVPANPPETFTTSKFASYITQYSTGQVSVEIEWELQKENSKRWNPEIQYTSNYAKSNNVEFSADAAGVYSEPRPIGTRYLTRNL

SEQ ID NO: 11 - protein, 739 aa, Papio hamadryas
  misc_feature (1)..(739): VP1 sequence (from baboon; denoted as Bba35)
MAADGYLPDWLEDNLSEGIREWWALKPGAPQPKANQQHQDNSRGLVLPGYKYLGPFNGLDKGEPVNEADAAALEHDKAYDKQLEQGDNPYLKYNHADAEFQERLQEDTSFGGNLGRAVFQAKKRILEPLGLVEEVAKTAPGKKRPLEKTPNQPTDTQAAGQTPAKKRPQGEQSGDSARRQLDFDPQPAAPIGQPPAAPSPVGSNTMASGGGGPMADDNQGADGVGNASGNWHCDSTWLGDRVITTSTRTWVLPTYNNHIYKQISSESGATNDNHYFGYSTPWGYFDFNRFHCHFSPRDWQRLINNNWGFRPKRLNFKIFNIQVKEVTQIEGGSTIANNLTSTIQVFADSEYQLPYVLGSAHQGCLPPFPADVFMIPQYGYLTLNNGSQSVGRSSFYCLEYFPSQMLRTGNNFQFSYTFESVPFHSSYAHSQSLDRIMNPLVDQYLYYLARTQTGTGSTTSNTRQLQFYQAGPSNMADQSRNWLPGPMYRQQRVSKTLDQNSNTNFAWTAASKYNLNGRKSLANPGIAMATHKDDEERFFPQHRVLVFGQTNATNKTTLDNVLITSEEEIKATNPVATEEYGTVSSNLQASNTDPTTETVNNQGILPGMVWQDRDVYLQGPIWVKIPHTDGHFHPSPLMGGFGLKHPPPQILIKNTPVPANPPETFTTSKFASYTTQYSTGQVSVEIEWGLQKENSKRWNPEIQYTSNYAKSNNVEFSVDAAGVYSEPRPIGTRYLTRNL

SEQ ID NO: 12 - protein, 739 aa, Papio hamadryas
  misc_feature (1)..(739): VP1 sequence (from baboon; denoted as Bba36)
MAADGYLPDWLEDNLSEGIREWWALKPGAPQPKANQQHQDNSRGLVLPGYKYLGPFNGLDKGEPVNEAYAAALEHDKAYDKQLEQGDNPYLKYNHADAEFQERLQEDTSFGGNLGRAVFQAKKRIPEPLGLVEEAAKTAPGKKRPLEKTPNQPTDTQAAGQTPAKKRPQGEQSGDSARRQLDFDPQPAAPIGQPPAAPSPVGSNTMASGGGGPMADDNQGADGVGNASGNWHCDSTWLGDRVITTSTRTWVLPTYNNHIYKQISSESGATNDNHYFGYSTPWGYFDFNRFHCHFSPRDWQRLINNNWGFRPKRLNFKIFNIQVKEVTQIEGGSTIANNLTSTIQVFADSEYQLPYVLGSAHQGCLPPFPADVFMIPQYGYLTLNNGSQSVGRPSFYCLEYFPSQMLRTGNNFQFSYTFESVPFHSSYAHSQSLDRIMNPLVDQYLYYLARTQTGTGSTTSNTRQLQFYQAGPSNMADQSRNWLPGPMYRQQRVSKTLDQNSNTNFAWTAASKYNLNGRKSLANPGIAMATHKDDEERFFPQHGVLVFGQTNATNKTTLDNVLITSEEEIKATNPVATEEYGTVSSNLQASNTNPTTETVNNQGILPGMVWQDRDVYLQGPIWAKIPHTDGHFHPSPLMGGFGLKHPPPQILIKNTPVPANPPETFTTSKFASYITQYSTGQVSVEIEWELQKENSKRWNPEIQYTSNYAKSNNVEFSVDAAGVYSEPRPIGTRYLTRNL

SEQ ID NO: 13 - protein, 739 aa, Papio hamadryas
  misc_feature (1)..(739): VP1 sequence (from baboon; denoted as Bba37)
MAADGYLPDWLEDNLSEGIREWWALKPGAPQPKANQQHQDNSRGLVLPGYKYLGPFNGLDKGEPVNEADAAALEHDKAYDKQLEQGDNPYLKYNHADAEFQERLQEDTSFGGNLGRAVFQAKKRILEPLGLVEEAAETAPGKKRPLEKTPNQPTDTQAAGQTPAKKRPQGEQSGDSARRQLDFDPQPAAPIGQPPAAPSPVGSNTMASGGGGPMADDNQGADGVGNASGNWHCDSTWLGDRVITTSTRTWVLPTYNNHIYKQISSESGATNDNHYFGYSTPWGYFDFNRFHCHFSPRDWQRLINNNWGFRPKRLNFKIFNIQVKEVTQTEGGSTIANNLTSTIQVFADSEYQLPYVLGSAHQGCLPPFPADVFMIPQYGYLTLNNGSQSVGRSSFYCLEYFPSQMLRTGNNFQFSYTFESVPFHSSYAHSQSLDRIINPLVDQYLYYLARTQTGTGSTTSNTRQLQFYQAGPSNMADQSRNWLPGPMYRQQRVSKTLDQNSNTNFAWTAASKYNLNGRKSLANPGIAMATHKDDEERFFPQHGVLVFGQTNATNKTTLDNVLITSEEEIKATNPVATEEYGTVSSNLQASNTNPTTETVNNQGILPGMVWQDRDVYLQGPIWAKIPHTDGHFHPSPLMGGFGLKHPPPQILIKNTPVPANPPETFTTSKFASYITQYSTGQVSVEIEWELQKENSKRWNPEIQYTSNYAKSNNVEFSVDAAGVYSEPRPIGTRYLTRNL

SEQ ID NO: 14 - protein, 739 aa, Papio hamadryas
  misc_feature (1)..(739): VP1 sequence (from baboon; denoted as Bba38)
MAADGYLPDWLEDNLSEGIRGWWALKPGAPQPKANQQHQDNSRGLVLPGYKYLGPFNGLDKGEPVNEADAAALEHDKAYDKQLEQGDNPCLKYNHADAEFQERLQEDTSFGGNLGRAVFQAKKRILEPLGLVEEAAKTAPGKKRPLEKTPNQPTDTQAAGQTPAKKRPQGEQSGDSARRQLDFDPQPAAPIGQPPAAPSPVGSNTMASGGGGPMADDNQGADGVGNASGNWHCDSTWLGDRVITTSTRTWVLPTYNNHIYKQISSESGATNDNHYFGYSTPWGYFDFNRFHCHFSPRDWQRLINNNWGFRPKRLNFKIFNIQVKEVTQIEGGSTIANNLTSTIQVFADSEYQLPYVLGSAHQGCLPPFPADVFMIPQYGYLTLNNGSQSVGRSSFYCLEYFPSQMLRTGNNFQFSYTFESVPFHSSYAHSQSLDRIMNPLVDQYLYYLARTQTGTGSTTSNTRQLQFYQAGPSNMADQSRNWLPGPMYRQQRVSKTLDQNSNTNFAWTAASKYNLNGRKSLANPGIAMATHKDDEERFFPQHGVLVFGQTNATNKTTLDNVLITSEEEIKATNPVATEEYGTVSSNLQASNTNPTTETVNNQGILPGMVWQDRDVYLQGPTWAKIPHTDGHFHPSPLMGGFGLKHPPPQILIKNTPVPANPPETFTTSKFASYITQYSTGQVSVEIEWELQKENSKRWNPEIQYTSNYAKSNNVEFSVDAAGVYSEPRPIGTRYLTRNL

SEQ ID NO: 15 - protein, 739 aa, Papio hamadryas
  misc_feature (1)..(739): VP1 sequence (from baboon; denoted as Bba41)
MAADGYLPDWLEDNLSEGIREWWALKPGAPQPKANQQHQDNSRGLVLPGYKYLGPFNGLDKGEPVNEADAAALEHDKAYDKQLEQGDNPYLKYNHADAEFQERLQEDTSFGGNLGRAVFQAKKRILEPLGLVEEAAKTAPGKKRPLEKTPNQPTDTQAAGQTPAKKRPQGEQSGDSARRQLDFDPQPAAPIGQPPAAPSPVGSNTMASGGGGPMADDNQGADGVGNASGNWHCDSTWLGDRVITTSTRTWVLPTYNNHIYKQISSESGATNDNHYFGYSTPWGYFDFNRFHCHFSPRDWQRLINNNWGFRPKRLNFKIFNIQVKEVTQIEGGSTIANNLTSTIQVFADSEYQLPYVLGSAHQGCLPPFPADVFMIPQYGYLTLNNGSQSVGRSSFYCLEYFPSQMLRTGNSFQFSYTFESVPFHSSYAHSQSLDRIMNPLVDQYLYYLARTQTGTGSTTSNTRQLQFYQAGPSNMADQSRNWLPGPMYRQQRVSKTLDQNSNTNFAWTAASKYNLNGRKSLANPGIAMATHKDDEERFFPQHGVLVFGQTNATNKTTLDNVLITSEEEIKATNPVATEEYGTVSSNLQASNTNPTTETVNNQGILPGMVWQDRDVYLQGPIWAKIPHTDGHFHPSPLMGGFGLKHPPPQILIKNTPVPANPPETFTTSKFASYITQYSTGQVSVEIEWELQKENSKRWNPEIQYTSNYAKSNNVEFSVDAAGVYSEPRPIGTRYLTRNL

SEQ ID NO: 16 - protein, 739 aa, Papio hamadryas
  misc_feature (1)..(739): VP1 sequence (from baboon; denoted as Bba42)
MAADGYLPDWLEDNLSEGIREWWALKPGAPQPKANQQHQDNSRGLVLPGYKYLGPFNGLDKGEPVNGADAAALEHDKAYDKQLEQGDNPYLKYNHADAEFQERLQEDTSFGGNLGRAVFQAKKRILEPLGLVEEAAKTAPGKKRPLEKTPNQPTDTQAAGQTPAKKRPQGEQSGDSARRQLDFDPQPAAPIGQPPAAPSPVGSNTMASGGGGPMADDNQGADGVGNASGNWHCDSTWLGDRVITTSTRTWVLPTYNNHIYKQISSESGATNDNHYFGYSTPWGYFDFNRFHCHFSPRDWQRLINNNWGFRPKRLNFKIFNIQVKEVTQIEGGSTIANNLTSTIQVFADSEYQLPYVLGSAHQGCLPPFPADVFMIPQYGYLTLNNGSQSVGRSSFYCLEYFPSQMLRTGNNFQFSYTFESVPFHSSYAHSQSLDRIMNPLVDQYLYYLARTQTGTGSTTSNTRQLQFYQAGPSNMADQSRNWLPGPMYRQQRVSKTLDQNSNTNFAWTAASKYNLNGRKSLVNPGIAMATHKDDEERFFPQHGVLVFGKTNATNKTTLENVLVTDEEEVKATNPVATEEYGTVSSNLQSNTTNPTTETVNNQGILPGMVWQDRDVYLQGPIWAKIPHTDGHFHPSPLMGGFGLKHPPPQILIKNTPVPANPPETFTTSKFASYITQYSTGQVSVEIEWELQKENSKRWNPEIQYTSNYAKSNNVEFSVDAAGVYSEPRLIGTRYLTRNL

SEQ ID NO: 17 - protein, 739 aa, Papio hamadryas
  misc_feature (1)..(739): VP1 sequence (from baboon; denoted as Bba43)
MAADGYLPDWLEDNLSEGIREWWALKPGAPQPKANQQHQDNSRGLVLPGYKYLGPFNGLDKGEPVNEADAAALEHDKAYDKQLEQGDNPYLKYNHADAEFQERLQEDTSFGGNLGRAVFQAKKRILEPLGLVEEAAKTAPGKKRPLEKTPNQPTDTQAAGQTPAKKRPQGEQSGDSARRQLDFDPQPAAPIGQPPAAPSPVGSNTMASGGGGPMADDNQGADGVGNASGNWHCDSTWLGDRVITTSTRTWVLPTYNNHIYKQISSESGATNDNHYFGYSTPWGYFDFNRFHCHFSPRDWQRLINNNWGFRPKRLNFKIFNIQVKEVTQIEGGSTIANNLTSTIQVFADSEYQLPYVLGSAHQGCLPPFPADVFMIPQYGYLTLNNGSQSVGRSSFYCLEYFPSQMLRTGNNFQFSYTFESVPFHSSYAHSQSLDRIMNPLVDQYLYYLARTQTGTGSTTSNTRQLQFYQAGPSNMADQSRNWLPGPMYRQQRVSKTLDQNSNTNFAWTAASKYNLNGRKSLVNPGIAMATHKDDEERFFPQHGVLVFGKTNATNKTTLENVLVTDEEEVKATNPVATEEYGTVSSNLQSNTTNPTTETVNNQGILPGMVWQDRDVYLQGPIWAKIPHTDGHFHPSPLMGGFGLKHPPPQILIKNTPVPANPPETFTTSKFASYITQYSTGQVSVEIEWELQKENSKRWNPEIQYTSNYAKSNNVEFSVDAAGVYSEPRPIGTRYLTRNL

SEQ ID NO: 18 - protein, 739 aa, Papio hamadryas
  misc_feature (1)..(739): VP1 sequence (from baboon; denoted as Bba44)
MAADGYLPDWLEDNLSEGIREWWALKPGAPQPKANQQHQDNSRGLVLPGYKYLGPFNGLDKGEPVNEADAAALEHDKAYDKQLEQGDNPYLKYNHADAEFQERLQEDTSFGGNLGRAVFQAKKRILEPLGLVEEAAKTAPGKKRPLEKTPNQPTDTQAAGQTPAKKRPQGEQSGDSARRQLDFDPQPAAPIGQPPAAPSPVGSNTMASGGGGPMADDNQGADGVGNASGNWHCDSTWLGDRVITTSTRTWVLPTYNNHIYKQISSESGATNDNHYFGYSTPWGYFDFNRFHCHFSPRDWQRLINNNWGFRPKRLNFKIFNIQVKEVTQIEGGSTIANNLTSTIQVFADSEYQLPYVLGSAHQGCLPPFPADVFMIPQYGYLTLNNGSQSVGRSSFYCLEYFPSQMLRTGNNFQFSYTFESVPFHSSYAHSQSLDRIMNPLVDQYLYYLARTQTGTGSTTSNTRQLQFYQAGPSNMADQSRNWLPGPMYRQQRVSKTLDQDSNTNFAWTAASKYNLNGRKSLVNPGIAMATHKDDEERFFPQHGVLVFGKTNATNKTTLENVLVTDEEEVKATNPVATEEYGTVSSNLQSNTTNPTTETVNNQGILPGMAWQDRDVYLQGPIWAKIPHTDGHFHPSPLMGGFGLKHPPPQILIKNTPVPANPPETFTTSKFASYITQYSTGQVSVEIEWELQKENSKRWNPEIQYTSNYAKSNNVEFSVDAAGVYSEPRPIGTRYLTRNL

SEQ ID NO: 19 - protein, 736 aa, Macaca fascicularis
  misc_feature (1)..(736): VP1 sequence (from crab-eating macaque; denoted as Bce14)
MAADGYLPDWLEDNLSEGIREWWALKPGAPQPKANQQKQDDGRGLVLPGYKYLGPFNGLDKGEPVNEADAAALEHDKAYDQQLKAGDNPYLRYNHADAEFQERLQEDTSFGGNLGRAVFQAKKRVLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFTFSYEFENVPFHSSYAHSQSLDRLMNPLIDQYLYYLSKTINGSGQNQQALKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLVGGFGMKHPPPQILIKNTPVPADPPTAFNKDKLNSFITQYSTGQVRVEIEWELQKENSKRWNPEIQYTSNYYKSNNVEFAVNTEGVYSEPRPIGTRYLTRNL

SEQ ID NO: 20 - protein, 736 aa, Macaca fascicularis
  misc_feature (1)..(736): VP1 sequence (from crab-eating macaque; denoted as Bce15)
MAADGYLPDWLEDNLSEGIREWWALKPGAPQPKANQQKQDDGRGLVLPGYKYLGPFNGLDKGEPVNEADAAALEHDKAYDQQLKAGDNPYLRYNHADAEFQERLQEDTSFGGNLGRAVFQAKKRVLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMPRTGNNFTFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVAIEWELQKENSKRWNPEIQYTSNYYKSNNVEFAVNTEGVYSEPRPIGTRYLTRNL

SEQ ID NO: 21 - protein, 736 aa, Macaca fascicularis
  misc_feature (1)..(736): VP1 sequence (from crab-eating macaque (denoted as Bce16))
MAADGYLPDWLEDNLSEGIREWWALKPGAPQPKANQQKQDDGRGLVLPGYKYLGPFNGLDKGEPVNEADAAALEHDKAYDQQLKAGDNPYLRYNHADAEFQERLQEDTSFGGNLGRAVFQAKKRVLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFTFSYEFENVPFHSSYAHSQSLDRPMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 22 - protein, 736 aa, Macaca fascicularis
  misc_feature (1)..(736): VP1 sequence (from crab-eating macaque (denoted as Bce17))
MAADGYLPDWLEDNLSEGIREWWALKPGAPQPKANQQKQDDGRGLVLPGYKYLGPFNGLDKGEPVNEADAAALEHDKAYDQQLKAGDNPYLRYNHADAEFQERLQEDTSFGGNLGRAVFQAKKRVLEPLGLVEEAAKTAPGKKRPVEQSPQEPDSSAGIGKSGAQPAIKRLNFGQTGDTESVPDPQPIGEPPAAPSGVGSLTMASGGGAPVADNNEGADGVGSSSGNWHCDSQWLGDRVITTSIRTWALPTYNNHLYKQISNSTSGGSSNDNAYFGYSTPWGYFDFSRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFTFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 23 - protein, 736 aa, Macaca fascicularis
  misc_feature (1)..(736): VP1 sequence (from crab-eating macaque (denoted as Bce18))
MAADGYLPDWLEDNLSEGIREWWALKPGAPQPKANQQKQDDGRGLVLPGYKYLGPFNGLDKGEPVNEADAAALEHDKAYDQQLKAGDNPYLRYNHADAEFQERLQEDTSFGGNLGRAVFQAKKRVLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFTFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 24 - protein, 733 aa, Macaca fascicularis
  misc_feature (1)..(733): VP1 sequence (from crab-eating macaque (denoted as Bce20))
MAADGYLPDWLEDNLSEGIREWWALKPGAPQPKANQQKQDDGRGLVLPGYKYLGPFNGLDKGEPVNEADAAALEHDKAYDQQLKAGDNPYLRYNHADAEFQERLQEDTSFGGNLGRAVFQAKKRVLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSRWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGAKTIANNLTSTVQVFTDSDYQLPYVLGSAHEGCLPPFPADVFMIPQYGYLTLNDGSQAVGRSSFYCLEYFPSQMLRTGNNFTFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLT

SEQ ID NO: 25 - protein, 730 aa, Macaca fascicularis
  misc_feature (1)..(730): VP1 sequence (from crab-eating macaque (denoted as Bce35)
MAADGYLPDWLEDNLSEGIREWWALKPGAPQPKANQQKQDDGRGLVLPGYKYLGPFNGLDKGEPVNEADAAALEHDKAYDQQLKAGDNPYLRYNHADAEFQERLQEDTSFGGNLGRAVLQAKKRVLEPLGLVEEAAKTAPGKKRPVDSPDSTSGIGKKGQQPARKRLNFGQTGDAESVPDPQPIGEPPAAPSGLGSGTMAAGGGAPMADNNEGADGVGNASGNWHCDSTWLGNRVITTSTRTWALPTYNNHLYKQISSSSSGATNDNHYFGYSTPWGYFDFNRFHCHFSPRDWQRLINNNWGFRPKRLRFKLFNIQVKEVTTNDGVTTIANNLTSTVQVFSDSEYQLPYVLGSAHQGCLPPFPADVFMIPQYGYLTLNNGSQSVGRSSFYCLEYFPSQMLRTGNNFEFSYEFEDVPFHSSYAHSQSLDRLMNPLIDQYLYYLARTQSSTGTSTRELQFHQAGPATMAEQSKNWLPGPCFRQQRISKTTDNNNNSNFAWTGATKYHLNGRSSLTNPGVPMATHKDDESVFFPINGVLVFGKTGASNKTTLENVLMTDEEEIKATNPVATEEYGVVSSNIQSQNSNPTTQTVNNQGALPGMVWQNRDVYLQGPIWAKIPHTDGNFHPSPLMGGFGLKHPPPQILIKNTPVPANPPETFTPAKFASFITQYSTGQVSVEIEWELQKENSKRWNPEIQYTSNYDKQTGVGFAVDTQGVYSEPRPIGTRYLTRNL

SEQ ID NO: 26 - protein, 730 aa, Macaca fascicularis
  misc_feature (1)..(730): VP1 sequence (from crab-eating macaque (denoted as Bce36))
MAADGYLPDWLEDNLSEGIREWWALKPGAPQPKANQQKQDDGRGLVLPGYKYLGPFNGLDKGEPVNEADAAALEHDKAYDQQLKAGDNPYLRYNHADAEFQERLQEDTSFGGNLGRAVFQAKKRVLEPLGLVEEAAKTAPGKKRPVDSPDSTSGIGKKGQQPARKRLNFGQTGDAESVPDPQPIGEPPAAPSGLGSGTMAAGGGAPMADNNEGADGVGNASGNWHCDSTWLGNRVITTSTRTWALPTYNNHLYKQISSSSSGATNDNHYFGYSTPWGYFDFNRFHCHFSPRDWQRLINNNWGFRPKRLRFKLFNIQVKEVTTNDGVTTIANNLTSTVQVFSDSEYQLPYVLGSAHQGCLPPFPADVFMIPQYGYLTLNNGSQSVGRSSFYCLEYFPSQMLRTGNNFEFSYEFEDVPFHSSYAHSQSLDRLMNPLIDQYLYYLARTQSSTGTSTRELQFHQAGPATMAEQSKNWLPGPCFRQQRISKTTDNNNNSNFAWTGATKYHLNGRNSLTNPGVPMATHKDDESVFFPINGVLVFGKTGASNKTTLENVLMTDEEEIKATNPVATEEYGVVSSNIQSQNSNPTTQTVNNQGALPGMVWQNRDVYLQGPIWAKIPHTDGNFHPSPLMGGFGLKHPPPQILIKNTPVPANPPETFTPAKFASFITQYSTGQVSVEIEWELQKENSKRWNPEIQYTSNYDKQTGVDFAVDTQGVYSEPRPIGTRYLTRNL

SEQ ID NO: 27 - protein, 736 aa, Macaca fascicularis
  misc_feature (1)..(736): VP1 sequence (from crab-eating macaque (denoted as Bce39))
MAADGYLPDWLEDNLSEGIREWWDLKPGAPKPKANQQKQDDGRGLVLPGYKYPGPFNGLDKGEPVNAADAAALEHDKAYDQQLKAGDNPYLRYNHADAEFQERLQEDTSFGGNLGRAVFQAKKRVFEPLGLVEEGAKTAPGKKRPVEQSPQEPDSSSGIGKTGQQPAKKRLNFGQTGDSESVPDPQPLGEPPAAPSGLGPNTMASGGGAPMADNNEGADGVGNSSGNWHCDSTWLGDRVITTSTRTWALPTYNNHLYKQISNSTSGGSTNDNTYFGYSTPWGYFDFNRFHCHFSPRDWQRLINNNWGFRPKRLNFKLFNIQVKEVTTNEGTKTIANNLTSTVQVFTDSEYQLPYVLGSAHQGCLPPFPADVFMVPQYGYLTLNNGSQALGRSSFYCLEYFPSQMLRTGNNFQFSYTFEDVPFHSSYAHSQSLDRLMNPLIDQYLYYLVRTQTTGTGGTQTLAFSQAGPSSMANQARNWVPGPSYRQQRVSTTKNQNNNSNFAWTGAAKFKLNGRNSLMNPGVAMASHKDDEDRFFPSSGVLIFGKQGAGNDGVDYSQVLITDEEEIKATNPVATEEYGEVAINDQAANTQAQTGLVHNQGVIPGMVWQNRDVYLQGPIWAKIPHTDGNFHPSPLMGGFGLMHPPPQILIKNTPVPADPPLTFNQAKLNSFITQYSTGQVSVEIEWELQKENSKRWNPEIQYTSNYYKSTNVDFAVNSDGVYSEPRPIGTRYLTRNL

SEQ ID NO: 28 - protein, 736 aa, Macaca fascicularis
  misc_feature (1)..(736): VP1 sequence (from crab-eating macaque (denoted as Bce40))
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQGRLQEDTSFGGNLGRAVFQAKKRVLEPLGLVEEGAKTAPGKKRPVEQSPQEPDSSSGIGKTGQQPAKKRLNFGQTGDSESVPDPQPLGEPPAAPSGLGPNTMASGGGAPMADNNEGADGVGNSSGNWHCDSTWLGDRVITTSTRTWALPTYNNHLYKQISNSTSGGSTNDNTYFGYSTPWGYFDFNRFHCHFSPRDWQRLINNNWGFRPKRPNFKLFNIQVKEVTTNEGTKTIANNLTSTVQAFTDSEYQLPYVLGSAHQGCLPPFPADVFMVPQYGYLTLNNGSQALGRSSFYCLEYFPSQMLRTGNNFQFSYTFEDVPFHSSYAHSQSLDRLMNPLIDQYLYYLVRTQTTGTGGTQTLAFSQAGPSSMANQARNWVPGPSYRQQRVSTTKNQNNNSNFAWTGAAKFKLNGRNSLMNPGVAMASHKDDEDRFSPSSGVLIFGKQGAGNDGVDYSQVLITDEEEIKATNPVATEEYGEVAINDQAANTQAQTGLVHNQGVIPGMVWQNRDVYLQGPIWAKIPHTDGNFHPSPLMGGFGLKHPPPQILIKNTPVPADPPLTFNQAKLNSFITQYSTGQVSVEIEWELQKENSKRWNPEIQYTSNYYKSTNVDFAVNSDGVYSEPRPIGTRYLTRNL

SEQ ID NO: 29 - protein, 736 aa, Macaca fascicularis
  misc_feature (1)..(736): VP1 sequence (from crab-eating macaque (denoted as Bce41))
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQGRLQEDTSFGGNLGRAVFQAKKRVLEPLGLVEEGAKTAPGKKRPVEQSPQEPDSSSGIGKTGQQPAKKRLNFGQTGDSESVPDPQPLGEPPAAPSGLGPNTMASGGGAPMADNNEGADGVGNSSGNWHCDSTWLGDRVITTSTRTWALPTYNNHLYKQISNSTSGGSTNDNTYFGYSTPWGYFDFNRFHCHFSPRDWQRLINNNWGFRPKRLNFKLFNIQVKEVTTNEGTKTIANNLTSTVQVFTDSEYQLPYVLGSAHQGCLPPFPADVFMVPQYGYLTLNNGSQALGRSSFYCLEYFPSQMLRTGNNFQFSYTFEDVPFHSSYAHSQSLDRLMNPLIDQYLYYLVRTQTTGTGGTQILAFSQAGPSSMANQARNWVPGPSYRQQRVSTTKNQNNNSNFAWTGAAKFKLNGRNSLMNPGVAMASHKDDEDRFFPSSGVLIFGKQGAGNDGMDYSQVLITDEEEIKATNPVATEEYGEVAINDQAANTQAQTGLVHNQGVIPGMVWQNRDVYLQGPIWAKIPHTDGNFHPSPLMGGFGLKHPPPQILIKNTPVPADPPLTFNQAKLNSFITQYSTGQVSVEIEWELQKENSKRWNPEIQYTSNYYKSTNVDFAVNSDGVYSEPRPIGTRYLTRNL

SEQ ID NO: 30 - protein, 736 aa, Macaca fascicularis
  misc_feature (1)..(736): VP1 sequence (from crab-eating macaque (denoted as Bce42))
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPAAPSGLGPNTMASGGGAPMADNNEGADGVGNSSGNWHCDSTWLGDRVITTSTRTWALPTYNNHLYKQISNSTSGGGTNDNTYFGYSTPWGYFDFNRFHCHFSPRDWQRLINNNWGFRPKRLNFKLFNIQVKEVTTNEGTKTIANNLTSTVQVFTDSEYQLPYVLGSAHQGCLPPFPADVFMVPQYGHLTLNNGSQALGRSSFYCLEYFPSQMLRTGNNFQFSYTFEDVPFHSSYAHSQSLDRLMNPLIDQYLYYLVRTQTTGTGGTQTLAFSQAGPSSMANQARNWVPGPSYRQQRVSTTKDQNNNSNFAWTGAAKFKLNGRNSLMNPGVAMASHKDDEDRFFPSSGVLIFGKQGAGNDGVDYSQVLITDEEEIKATNPVATEEYGEVAVNDQAANTQAQTGLVHNQGVIPGMVWQNRDVYLQGPIWAKIPHTDGNFHPSPLMGGFGLKHPPPQILIKNTPVPADPPLTLNQAKLNSFITQYSTGQVSVEIEWELQKENSKRWNPEIQYTSNYYKSTNVDFAVNSDGVYSEPRPIGTRYLTRNL

SEQ ID NO: 31 - protein, 730 aa, Macaca fascicularis
  misc_feature (1)..(730): VP1 sequence (from crab-eating macaque (denoted as Bce43))
MAADGYLPDWLEDNLSEGIREWWALKPGAPQPKANQQKQDDGRGLVLPGYKYLGPFNGLDKGEPVNEADAAALEHDKAYDQQLKAGDNPYLRYNHADAEFQERLQEDTSFGGNLGRAVFQAKKRVLEPLGLVEEAAKTAPGKKRPVDSPDSTSGIGKKGQQPARKRLNFGQTGDAESVPDPQPIGEPPAAPSGLGSGTMAAGGGAPMADNNEGADGVGNAPGNWHCDSTWLGNRVITTSTRTWALPTYNNHLYKQISSSSSGATNDNHYFGYSTPWGYFDFNRFHCHFSPRDWPRLINNNWGFRPKRLRFKLFNIQVKEVTTNDGVTTIANNLTSTVQVFSDSEYQLPYVLGSAHQGCLPPFPADVFMIPQYGYLTLNNGSQSVGRSSFYCLEYFPSQMLRTGNNFEFSYEFEDVPFHSSYAHSQSLDRLMNPLIDQYPYYLARTQSSTGTSTRELQFHQAGPATMAEQSKNWLPGPCFRQQRISKTTDNNNNSNFAWTGATKYHLNGRNSLTNPGVPMATHKDDESVFFPINGVLVFGKTGASNKTTLENVLMTDEEEIKATNPVATEEYGVVSSNIQSQNSNPATQTVNNQGALPGMVWQNRDVYLQGPIWAKIPHTDGNFHPSPLMGGFGLKHPPPQILIKNTPVPANPPETFTPAKFASFITQYSTGQVSVEIEWELQKENSKRWNPEIQYTSNYDKQTGVDFAVDTQGVYSEPRPIGTRYLTRNL

SEQ ID NO: 32 - protein, 730 aa, Macaca fascicularis
  misc_feature (1)..(730): VP1 sequence (from crab-eating macaque (denoted as Bce44))
MAADGYLPDWLEDNLSEGIREWWALKPGAPQPKANQQKQDDGRGLMLPGYKYLGPFNGLDKGEPVNEADAAALEHDKAYDQQLKAGDNPYLRYNHADAEFQERLQEDTSFGGNLGRAVFQAKKRVLEPLGLVEEAAKTAPGKKRPVDSPDSTSGIGKKGQQPARKRLNFGQTGDAESVPDPQPIGEPPAAPSGLGSGTMAAGGGAPMADNNEGADGVGNASGSWHCDSTWLGNRVITTSTRTWALPTYNNHLYKQISSSSSGATNDNHYFGYSTPWGYFDFNRFHCHFSPRDWQRLINNNWGFRPKRLRFKLFNIQVKEVTTNDGVTTIANNLTSTVQVFSDSEYQLPYVLGSAHQGCLPPFPADVFMIPQYGYLTLNNGRQSVGRSSFYCLEYFPSQMLRTGNNFEFSYEFEDVPFHSSYAHSQSLDRLMNPLIDQYPYYLARTQSSTGTSTRELQFHQAGPATMAEQSKNWLPGPCFRQQRISKTTDNNNNSNFAWTGATKYHLNGRNSLTNPGVPMATHKDDESVFFPINGVLVFGKTGASNKTTLENVPMTDEEEIKATNPVATEEYGVVSSNIQSQNSNPTTQTVNNQGALPGMVWQNRDVYLQGPIWAKIPHTDGNFHPSPLMGGFGLKHPPPQILIKNTPVPANPPETFTPAKFASFITQYSTGQVSVEIEWELQKENNKRWNPEIQYTSNYDKQTGVDFAVDTQGVYSEPRPIGTRYLTRNL

SEQ ID NO: 33 - protein, 730 aa, Macaca fascicularis
  misc_feature (1)..(730): VP1 sequence (from crab-eating macaque (denoted as Bce45))
MAADGYLPDWLEDNLSEGIREWWALKPGAPQPKANQQKQDDGRGLVLPGYKYLGPFSGLDKGEPVNEADAAALEHDKAYDQQLKAGDNPYLRYNHADAEFQERLQEDTSFGGNLGRAVFQAKKRVLEPLGLVEEAAKTAPGKKRPVDSPDSTSGIGKKGQQPARKRLNFGQTGDAESVPDPQPIGEPPAAPSGLGSGTMAAGGGAPMADNNEGADGVGNASGNWHCDSTWLGNRVITTSTRTWALPTYNDHLYKQISSSSSGATNDNHYFGYSTPWGYFDFNRFHCHFSPRDWQRLINNNWGFRPKRLRFKLFNIQVKEVTTNDGVTTIANNLTSTVQVFSDSEYQLPYVLGSAHQGCLPPFPADVFMIPQYGYLTLNNGSQSVGRSSFYCLEYFPSQMLRTGNNFEFSYEFEDVPFHSSYAHSQSLDRLMNPLIDQYLYYLARTQSSTGTSTRELQFHQAGPATMAEQSKNWLPGPCFRQQRISKTTDNNNNSNFAWTGATKYHLNGRNSLTNPGVPMATHKDDESVFFPINGVLVFGKTGASNKTTLENVLMTDEEEIKATNPVATEEYGVVSSNIQSQNSNPTTQTVNNQGALPGMVWRNRDVYLQGPIWAKIPHTDGNFHPSPLMGGFGLKHPPPQILIKNTPVPANPPETFTPAKFASFITQYSTGQVSVEIEWELQKENSKRWNPEIQYTSNYDKQTGVDFAVDTQGVYSEPRPIGTRYLTRNL

SEQ ID NO: 34 - protein, 730 aa, Macaca fascicularis
  misc_feature (1)..(730): VP1 sequence (from crab-eating macaque (denoted as Bce46))
MAADGYLPDWLEDNLSEGIREWWALKPGAPQPKANQQKQDDGRGLVLPGYKYLGPFNGLDKGEPVNEADAAALEHDKAYDQQLKAGDNPYLRYNHADAEFQERLQEDTSFGGNLGRAVFQAKKRVLEPLGLVEEAAKTAPGKKRPVDSPDSTSGIGKKGQQPARKRLNFGQTGDAESVPDPQPIGEPPAAPSGLGSGTMAAGGGAPMADNNEGADGVGNASGNWHCDSTWLGNRVITTSTRTWALPTYNNHLYKQISSSSSGATNDNHYFGYSTPWGYFDFNRFHCHFSPRDWQRLINNNWGFRPKRLRFKLFNIQVKEVTTNDGVTTIANNLTSTVQVFSDSEYQLPYVLGSAHQGCLPPFPADVFMIPQYGYLTLNNGSQSVGRSSFYCLEYFPSQMLRTGNNFEFSYEFEDVPFHSSYAHSQSLDRLMNPLIDQYLYYMARTQSSTGTSTREPQFHQAGPATMAEQSKNWLPGPCFRQQRISKTTDNNNNSNFAWTGATKYHLNGRNSLTNPGVPMATHKDDESVFFPINGVLVFGKTGASNKTTLENVLMTDEEEIKATNPVATEEYGVVSSNIQSQNSNPTTQTVNNQGALPGMVWQNRDVYLQGPIWAKIPHTDGDFHPSPLMGGFGLKHPPPQILIKNTPVPANPPETFTPAKFASFITQYSTGQVSVEIEWELQKENSKRWNPEIQYTSNYDKQTGVDFAVDTQGVYSEPRPIGTRYLTRNL

SEQ ID NO: 35 - protein, 730 aa, Macaca fascicularis
  misc_feature (1)..(730): VP1 sequence (from cynomolgus macaque (denoted as Bey20))
MAADGYLPDWLEDNLSEGIREWWALKPGAPQPKANQQKQDDGRGLVLPGYKYLGPFNGLDKGEPVNEADAAALEHDKAYDQQLKAGDNPYLRYNHADAEFQERLQEDTSFGGNLGRAVFQAKKRVLEPLGLVEEAAKTAPGKKRPVDSPDSTSGIGKKGQQPARKRLNFGQTGDAESVPDPQPIGEPPAAPSGLGSGTMAAGGGAPMADNNEGADGVGNASGNWHCDSTWLGNRVITTSTRTWALPTYNNHLYKQISSGSSGATNDNHYFGYSTPWGYFDFNRFHCHFSPRDWQRLINNNWGFRPKRLRFKLFNIQVKEVTTNDGVTTIANNLTSTVQVFSDSEYQLPYVLGSAHQGCLPPFPADVFMIPQYGYLTLNNGSQSVGRSSFYCLEYFPSQVLRTGNNFEFSYEFEDVPFHSSYAHSQSLDRLMNPLIDQYLYYLARTQSSTGTSTRELQFHQAGPATMAEQSKNWLPGPCFRQQRISKTSDNNNNSNFAWTGATKYHLNGRNSLTNPGVPMATHKDDESVFFPINGVLVFGKTGASNKTTLENVLMTDEEEIKATNPVATEEYGVVSSNIQSQNSNPTTQTVNNQGALPGMVWQNRDVYLQGPIWAKIPHTDGNFHPSPLMGGFGLKHPPPQILIKNTPVPANPPETFTPAKFASFITQYSTGQVSVEIEWELQKENSKRWNPEIQYTSNYDKQTGVDFAVDTQGVYSEPRPIGTRYLTRNL

SEQ ID NO: 36 - protein, 730 aa, Macaca fascicularis
  misc_feature (1)..(730): VP1 sequence (from cynomolgus macaque (denoted as Bey22))
MAADGYLPDWLEDNLSEGIREWWALKPGAPQPKANQQKQDDGRGLVLPGYRYLGPFNGLDKGEPVNEADAAALEHDKAYDQQLKAGDNPYLRYNHADAEFQERLQEDTSFGGNLGRAVFQAKKRILEPLGLVEEAAKTAPGKERPVDSPDSTSGIGKKGQQPARKRLNFGQTGDAESVPDPQPIGEPPAAPSGLGSGTMAAGGGAPMADNNEGADGVGNASGNWHCDSTWLGNRVITTSTRTWALPTYDNHLYKQISSSSSGATNDNHYFGYSTPWGYFDFNRFHCHFSPRDWQRLINNNWGFRPKRLRFKLFNIQVKEVTTNDGVTTIANNLTSTVQVFSDSEYQLPYVLGSAHQGCLPPFPADVFMIPQYGYLTLNNGSQSVGRSSFYCLEYFPSQMLRTGNNFEFSYEFEDVPFHSSYAHSQSLDRLMNPLIDQYLYYLARTQSSTGTSTRELQFHQAGPATMAEQSKNWLPGPCFRQQRISKTTDNNNNSNFAWTGATKYHLNGRNSLTNPGVPMATHKDDESVFFPINGVLVLGKTGASNKTTLENVLMTDEEEIKATNPVATEEYGVVSSNIQSQNSNPTTQTVNNQGALPGMVWQNRDVYLQGPIWAKIPHTDGNFHPSPLMGGFGLKHPPPQILIKNTPVPANPPETFTPAKFASFITQYSTGQVSVEIEWELQKENSKRWNPEIQYTSNYDKQTGVDFAVDTQGVYSEPRPIGTRYLTRNL

SEQ ID NO: 37 - protein, 730 aa, Macaca fascicularis
  misc_feature (1)..(730): VP1 sequence (from cynomolgus macaque (denoted as Bey23))
MAADGYLPDWLEDNLSEGIREWWALKPGAPRPKANQQKQDDGRGLVLPGYKYLGPFNGLDKGEPVNEADAAALEHDKAYDQQLKAGDNPYLRYNHADAEFQERLQEDTSFGGNLGRAVFQAKKRVLEPLGLVEEAAKTAPGKKRPVDSPDSTSGIGKKGQQPARKRLNFGQTGDAESVPDPQPIGEPPAAPSGLGSGTMAAGGGAPMADNNEGADGVGNASGNWHCDSTWLGNRVITTSTRTWALPTYNNHLYKQISSSSSGATNDNHYFGYSTPWGYFDFNRFHCHFSPRDWQRLINNNWGFRPKRLRFKLFNIQVKEVTTNDGVTTIANNLTSTVQVFSDSEYQLPYVLGSAHQGCLPPFPADVFMIPQYGYLTLNNGSQSVGRSSFYRLEYFPSQMLRTGNNFEFSYEFEDVPFHSSYAHSQSLDRLMNPLIDQYLYYLARTQSSTGTSTRELQFHQAGPATMAEQSKNWLPGPCFRQQRISKTTDNNNNSNFAWTGATKYHLNGRNSLTNPGVPMATHRDDESVFFPINGVLVFGKTGASNKTTLENVLMTDEEEIKATNPVATEEYGVVSSNIQSQNSNPTTQTVNNQGALPGMVWQNRDVYLQGPIWAKIPHTDGNFHPSPLMGGFGLKHPPPQILIKNTPVPANPPETFTPAKFASFITQYSTGQVSVEIEWELQKENSKRWNPEIQYTSNYDKQTGVDFAVDTQGVYSEPRPIGTRYLTRNL

SEQ ID NO: 38 - protein, 736 aa, Callithrix jacchus
  misc_feature (1)..(736): VP1 sequence (from marmoset (denoted as Bma42))
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDTESVPDPQPLREPPAAPSGLGPNTMASGGGAPMADNNEGADGVGNSSGNWHCDSTWLGDRVITTSTRTWALPTYNNHLYKQISNGTSGGSTNDNTYFGYSTPWGYFDFNRFHCHFSPRDWQRLINNNWGFRPKRLNFKPFNIQVKEVTTNEGTKTIANNPTSTVQVFTDSEYQLPYVLGSARQGCLPPFPADVFMVPQYGYLTLNNGSQALGRSSFYCLEYFPSQMLRTGDNFQFSYTFEDVPFHSSYAHSQSLDRLMNPLIDQYLYYLVRTQTTGTGGTQTLAFSQAGPSSMANQARNWVPGPCYRQQRVSTTTNQNNNSNFAWTGAAKFKLNGRDSLMNPGVAMASHKDDEDRFFPSSGVLIFGKQGAGNDGVDYSQVLITDEEEIKATNPVATEEYGAVAINNQAANTLAQTGLVHNQGVIPGMVWQNRDVYLQGPIWAKIPHTDGNFHPSPLMGGFGLKHPPPQILIKNTPVPADPPLTFNQAKLNSFITQYSTGQVSVEIEWELQKENSKRWNPEIQYTSNYYKSTNVDFAVNTEGVYSEPRPIGTRYLTRNL

SEQ ID NO: 39 - protein, 736 aa, Callithrix jacchus
  misc_feature (1)..(736): VP1 sequence (from marmoset (denoted as Bma43))
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGANTAPGKKRPVEQSPQEPDSSSGIGKTGQQPAKKRLNFGQTGDSESVPDPQPLREPPAAPSGLGPNTMASGGGAPMADNNEGADGVGNSSGNWHCDSTWLGDRVITTSTRTWALPTYNNHLYKQISNGTSGGSTNDNTYFGYSTPWGYFDFNRFHCHFSPRDWQRLINSNWGFRPKRLNFKLFNIQVKEVTTNEGTKTIANNLTSTVQVFTDSEYQLPYVLGSAHQGCLPPFPADVFMVPQYGYLTLNNGSQALGRSSFYCLEYFPSQMLRTGNNFQFSYTFEDVPFHSSYAHSQSLDRLMNPLIDQYLYYLVRTQTTGTGGTQTLAFSQAGPSSMANQARNWVPGPCYRQQRVSTTTNQNNNSNFAWTGAAKFKLNGRDSLMNPGVAMASHKDDEDRFFPSSGVLIFGKQGAGNDGVDYSQVLITDEEEIKATNPVATEEYGAVAINNQAANTQAQTGLVHNQGVIPGMVWQNRDVYLQGPIWAKIPHTDGNFHPSPLMGGFGLKHPPPQILIKNTPVPADPPLTFNQAKLNSFITQYSTGQVSVEIEWELQKENSKRWNPEIQYTSNYYKSTNVDFAVNTEGVYSEPRPIGTRYLTRNL

SEQ ID NO: 40 - protein, 716 aa, Sus scrofa
  misc_feature (1)..(716): VP1 sequence (from pig (denoted as Bpol))
MSFVDHPPDWLEEIGEGLKEFLGLEPGPPKPKPNQQKQDNARGLVLPGYNYLGPGNGLDRGEPVNRADEVAREHDISYNEQLQAGDNPYLKYNHADAEFQEKLADDTSFGGNLGKAVFQAKKRVLEPFGLVEEPAKTAAKGERIDDHYPKKKKARVEETEAGTSGGQQLQIPAQPASSLGADTMSAGGGSPLGDNNQGADGVGNASGDWHCDSTWMGDRVITKSTRTWVLPSYNNHLYKEIHSGSVDGSNANAYFGYSTPWGYFDFNRFHSHWSPRDWQRLVNNYWGFRPRSLKVKIFNIQVKEVTVQDATTTIANNLTSTVQVFTDDDYQLPYVIGNGTEGCLPAFPPQVFTLPQYGYATLNRNNTDDPTERSSFFCLEYFPSKMLRTGNSFEFTYSFEEVPFHCSFAPSQNLFKLANPLVDQYLYRFVSTDTSGNIQFQKNLKARYANTYKNWFPGPMCRTQGWYTGSGTYNRSGVTNFATSNRMDLEGASYQVNPQPNGMTNTLQDSNKYALENTMIFNSQNAEPGTTSLYQENNLLITSESETQPVNRVAYDTGGQMATNAQSTNLAPTVGTYNHQEMLPGSVWMDRDVYLQGPIWAKIPETGAHFHPSPAMGGFGLKHPPPMMLIKNTPVPSNVTAFSEIPVKSFITQYSTGQVTVEMEWELKKEDSKRWNPEIQYTNNYNNPEFVDFAPDTSGEYRTTRAIGTRYLTRPL

SEQ ID NO: 41 - protein, 716 aa, Sus scrofa
  misc_feature (1)..(716): VP1 sequence (from pig (denoted as Bpo2))
MSFVDHPPDWLEEIGEGLKEFLGLEPGPPKPKPNQQKQDNARGLVLPGYNYLGPGNGLDRGEPVNRADEVAREHDISYNEQLQAGDNPYLKYNHADAEFQEKLADDTSFGGNLGKAVFQAKKRALEPFGLVEEPAKTAAKGERIDDHYPKKKKARVEETEAGTSGGQQLQIPAQPASSLGADTMSAGGGSPLGDNNQGADGVGNASGDWHCDSTWMGDRVITKSTRTWVLPSYNNHLYKEIHSGSVDGSNANAYFGYSTPWGYFDFNRFHSHWSPRDWQRLVNNYWGFRPRSLKVKIFNIQVKEVTVQDATTTIANNLTSTVQVFTDDDYQLPYVIGNGTEGCLPAFPPQVFTLPQYGYATLNRNNTDDPTERSSFFCLEYFPSKMLRTGNSFEFTYSFEEVPFHCSFAPSQNLFKLANPLVDQYLYRFVGTDTSGNIQFQKNLKARYANTYKNWFPGPMCRTQGWYTGSGTYNRSGVTNFATSNRMDLEGASYQVNPQPNGMTNTLQDSNKYALENTMIFNSQNAEPGTTSLYQENNLLITSESETQPVNRVAYDTGGQMATNAQSTNLAPTVGTYNHQEMLPGSVWMDRDVYLQGPIWAKIPETGAHFHPSPAMGGFGLKHPPPMMLIKNTPVPSNVTAFSEIPVKSFITQYSTGQVTVEMEWELKKENSKRWNPEIQYTNNYNNPEFVDFAPDTSGEYRTTRAIGTRYLTRPL

SEQ ID NO: 42 - protein, 716 aa, Sus scrofa
  misc_feature (1)..(716): VP1 sequence (from pig (denoted as Bpo3))
MSFVDHPPDWFEEIGEGLKEFLGLEPGPPKPKPNQQKQDNARGLVLPGYNYLGPGNGLDRGEPANRADEVAREHDISYNEQLQAGDNPYLKYNHADAEFQEKLADDTSFGGNLGKAVFQAKKRVLEPFGLVEEPAKTAAKGERIDDHYPKKKKARVEETEAGTSGGQQLQIPAQPASSLGADTMSAGGGSPLGDNNQGADGVGNASGDWHCDSTWMGDRVITKSTRTWVLPSYNNHLYKEIHSGSVDGSNANAYFGYSTPWGYFDFNRFHSHWSPRDWQRLVNNYWGFRPRSLKVKIFNIQVKEVTVQDATTTIANNLTSTVQVFTDDDYQLPYVIGNGTEGCLPAFPPQVFTLPQYGYATLNRNNTDDPTERSSFFCLEYFPSKMLRTGNSFEFTYSFGEVPLHCSFAPSQNLFKLANPLVDQHLYRFVSTDTSGNIQFQKNLKARYANTYKNWFPGPMCRTQGWYTGSGTYNRPGVTNFATSNRMDLEGASYQVNPQPNGMTNTLQDSNKYALENTMIFNSQNAEPGTTSLYQENNLLITSESETQPVNRVAYDTGGQMATNAQSTNLAPTVGTYNHQEMLPGSVWMDRDVYLQGPIWAKIPETGAHFHPSPAMGGFGLKHPPPMMLTKNTPVPSNVTAFSEIPVKSFITQYSTGQVTVEMEWELKKENSKRWNPEIQYTNNYNNPEFVDFAPDTSGEYRTTRAIGTRYLTRPL

SEQ ID NO: 43 - protein, 716 aa, Sus scrofa
  misc_feature (1)..(716): VP1 sequence (from pig (denoted as Bpo4))
MSFVDHPPDWLEEIGEGLKEFLGLEPGPPKPKPNQQKQDNARGLVLPGYNYLGPGNGLDRGEPVNRADEVAREHDISYNEQLQAGDNPYLKYNHADAEFQEKLADDTSFGGNLGKAVFQAKKRVLEPFGLVEEPAKTAAKGERIDDHYPKKKKARVEETEAGTSGGQQLQIPAQPASSLGADTMSAGGGSPLGDNNQGADGVGNASGDWHCDSTWMGDRVITKSTRTWVLPSYNNHLYKEIHSGSVDGSNANAYFGYSTPWGYFDFNRFHSHWSPRDWQRLVNNYWGFRPRSLKVKIFNIQVKEVTVQDATTTIANNLTSTVQVFTDDDYQLPYVIGNGTEGCLPAFPPQVFTLPQYGYATLNRNNTDDPTERSSFFCLEYFPSKMLRTGNSFEFTYSFEEVPFHCSFAPSQNLFKLANPLVDQYLYRFVSTDTSGNIQFQKNLKARYANTYKNWFPGPMCRTQGWYTGSGTYNRSGVTNFATSNRMDLEGASYQVNPQPNGMTNTLQDSNKYALENTMIFNSQNAEPGTTSLYQENNLLITSESETQPVNRVAYDTGGQMATNAQSTNLAPTVGTYNHQEMLPGSVWMDRDVYLQGPIWAKIPETGAHFHPSPAMGGFGLKHPPPMMLIKNTPVPSNVTAFSEIPVKSFITQYSTGQVTVEMEWELKKENSKRWNPEIQYTNNYNNPEFVDFAPDTSGEYRTTRAIGTRYLTRPL

SEQ ID NO: 44 - protein, 716 aa, Sus scrofa
  misc_feature (1)..(716): VP1 sequence (from pig (denoted as Bpo6))
MSFVDHPPDWLEEIGEGLKEFLGLEPGPPKPKPNQQKQDNARGLVLPGYNYLGPGNGLDRGEPVNRADEVAREHGISYNEQLQAGDDPYLKYNHADAEFQEKLADDTSFGGNLGKAVFQAKKRVLEPFGLVEEPAKTAAKGERIDDHYPKKKKARVEETEAGTGGGQQLQIPAQPASSLGADTMSAGGGSPLGDNNQGADGVGNASGDWHCDSTWMGDRVITKSTRTWVLPSYNNHLYKEIHSGSVDGSNANAYFGYSTPWGYFDFNRFHSHWSPRDWQRLVNNYWGFRPRSLKVKIFNIQVKEVTVQDATTTIANNLTSTVQVFTDDDYQLPYVIGNGTEGCLPAFPPQVFTLPQYGYATLNRNNTDDPTERSSFFCLEYFPSKMLRTGNSLEFTYSFEEVPFHCSFAPSQNLFKLANPLVDQYLYRFVSTDTSGNIQFQKNLKARYANTYKNWFPGPMCRTQGWYTGSGTYNRSGVTNFATSNRMDLEGASYQVNPQPNGMTNTLQDSNKYALENTMIFNSQNAEPGTTSLYQENNLLITSESETQPVNRVAYDTGGQMATNAQSTNLAPTVGTYNHQEMLPGSVWMDRDVYLQGPIWAKIPETGAHFHPSPAMGGFGLKHPPPMMLIKNTPVPSNVTAFSEIPVKSFITQYSTGQVTVEMEWELKKENSKRWNPEIQYTNNYNNPEFVDFAPDTSGEYRTTRAIGTRYLTRPL

SEQ ID NO: 45 - protein, 716 aa, Sus scrofa
  misc_feature (1)..(716): VP1 sequence (from pig (denoted as Bpo8))
MSFVDHPPDWLEEIGEGLKEFLGLKPGPPKPKPNQQKQDNARGLVLPGYNYLGPGNGLGRGEPVNRADEVAREHDISYNEQLQAGDNPYLKYNHADAEFQEKLADDTSFGGNLGKAVFQAKKRVLEPFGLVEEPAKTAAKGERIDDHYPKKKKARVEETEAGTSGGQQLQIPAQPASSLGADTMSAGGGSPLGDNNQGADGVGNASGDWHCDSTWMGDRVITKSTRTWVLPSYNNHLYKEIHSGSVDGSNANAYFGYSTPWGYFDFNRFHSHWSPRDWQRLVNNYWGFRPRSLKVKIFNIQVKEVTVQDATTTIANNLTSTVQVFTDDDYQLPYVIGNGTEGCLPAFPPQVFTLPQYGYATLNRNNTDDPTERSSFFCLEYFPSKMLRTGNSFEFTYSFEEVPFHCSFAPSQNLFKLANPLVDQYLYRFVSTDTSGNIQFQKNLKARYANTYKNWFPGPMCRTQGWYTGSGTYNRSGVTNFATSNRMDLEGASYQANPQPNGMTNTLQDSNKYALENTMIFNSQNAEPGTTSLYQENNLLITSESETQPVNRVAYDTGGQMATNAQSTNLAPTVGTYNHQEMLPGSVWMDRDVYLQGPIWAKIPETGAHFHPSPAMGGFGLKHPPPMMLIKNTPVPSNVTAFSEIPVKSLITQYSTGQVTVEMEWELKKENSKRWNPEIQYTNNYNNPEFVDFAPDTSGEYRTTRAIGTRYLTRPL

SEQ ID NO: 46 - protein, 716 aa, Sus scrofa
  misc_feature (1)..(716): VP1 sequence (from pig (denoted as Bpo13))
MSFVDHPPDWLEEIGEGLKEFLGLEPGPPKPKPNQQKQDNARGLVLPGYNYLGPGNGLDRGEPVNRADEVAREHDISYNEQLQAGDNPYLKYNHADAEFQEKLADDTSFGGNLGKAVFQAKKRVLEPFGLVEEPAKTAAKGERIDDHYPKKKKARVEETEAGTSGGQQLQIPAQPASSLGADTMSAGGGSPLGDNNQGADGVGNASGDWHCDSTWMGDRVITKSTRTWVLPSYNNHLYKEIHSGSVDGSNANAYFGYSTPWGYFDFNRFHSHWSPRDWQRLVNNYWGFRPRSLKVKIFNIQVKEVTVQDATTTIANNLASTVQVFTDDDYQLPYVIGNGTEGCLPAFPPQVFTLPQYGYATLNRNNTDDPTERSSFFCLEYFPSKMLRTGNSFEFTYSFEEVPFHCSFAPSQNLFKLANPLVDQYLYRFVSTDTSGNIQFQKNLKARYANTYKNWFPGPMCRTQGWYTGSGTYNRSGVTNFATSNRMDLEGASYQVNPQPNGMTNTLQDSNKYALENTMIFNSQNAEPGTTSLYQENNLLITSESETQPVNRVAYDTGGQMATNAQSTNLAPTVGTYNHQEMLPGSVWMDRDVYLQGPIWAKIPETGAHFHPSPAMGGFGLKHPPPMMLIKNTPVPSNVTAFSEIPVKSFITQYSTGQVTVEMEWELKKENSKRWNPEIQYTNNYNNPEFVDFAPDTSGEYRTTRAIGTRYLTRPL

SEQ ID NO: 47 - protein, 716 aa, Sus scrofa
  misc_feature (1)..(716): VP1 sequence (from pig (denoted as Bpo18))
MSFVDHPPDWLEEIGEGLKEFLGLEPGPPKPKPNQQKQDNARGLVLPGYNYLGPGNGLDRGEPVNRADEVAREHDISYNEQLQAGDDPYLKYNHADAEFQEKLADDTSFGGNLGKAVFQAKKRVLEPFGLVEEPAKTAAKGERIDDHYPKKKKARVEETEAGTSGGQQLQIPAQPASSLGADTMSAGGGSPLGDNNQGADGVGNASGDWHCDSTWMGDRVITKSTRTWVLPSYNNHLYKEIHSGSVDGSNANAYFGYSTPWGYFDFNRFHSHWSPRDWQRLVNNYWGFRPRSLKVKIFNIQVKEVTVQDATTTIANNLTSTVQVFTDDDYQLPYVIGNGTEGCLPAFPPQVFTLPQYGYATLNRNNTDDPTERSSFFCLEYFPSKMLRTGNSFEFTYSFEEVPFHCSFAPSQNLFKLANPLVDQYLYRFVSTDTSGNIQFQKNLKARYANTYKNWFPGPMRRTQGWYTGSGTYNRSGVTNFATSNRMDLEGASYQVNPQPNGMTNTLQDSNKYALENTMIFNSQNAEPGTTSLYQENNLLITSESETQPVNRVAYDTGGQMATNAQSTNLAPTVGTYNHQEMLPGSVWMDRDVYLQGPIWAKIPETGAHFHPSPAMGGFGLKHPPPMMLIKNTPVPSNVTAFSEIPVKSFITQYSTGQVTVEMEWELKKENSKRWNPEIQYTNNYNNPEFVDFAPDTSGEYRTTRAIGTRYLTRPL

SEQ ID NO: 48 - protein, 716 aa, Sus scrofa
  misc_feature (1)..(716): VP1 sequence (from pig (denoted as Bpo20))
MSFVDHPPDWLEEIGEGLKEFLGLEPGPPKPKPNQQKQDNARGLVLPGYNYLGPGNGLDRGEPVNRADEVAREHDISYNEQLQAGDNPYLKYNHADAEFQEKLADDTSFGGNLGKAVFQAKKRVLEPFGLVEEPAKTAAKGERIDDHYPKKKKARVEETEAGTSGGQQLQIPAQPASSLGADTMSAGGGSPLGDNNQGADGVGNASGDWHCDSTWMGDRVITKSTRTWVLPSYNNHLYKEIHSGSVDGSNANAYFGYSTPWGYFDFNRFHSHWGPRDWQRLVNNYWGFRPRSLKVKIFNIQVKEVTVQDATTTIANNLTSTVQVFTDDDYQLPYVIGNGTEGCLPAFPPQVFTLPQYGYATLNRNNTDDPTERSSFFCLEYFPSKMLRTGNSFEFTYSFEGVPFHCSFAPSQNLFKLANPLVDQYLYRFVSTDTSGNIQFQKNLKARYANTYKNWFPGPMCRTQGWYTGSGTYNRSGVTNFATSNRMDLEGASYQVNPQPNGMTNTLQDSNKYALENTMIFNSQNAEPGTTSLYQENNLLITSESETQPVNRVAYDTGGQMATNAQSTNLAPTVGTYNHQEMLPGSVWMDRDVYLQGPIWAKIPETGAHFHPSPAMGGFGLKHPPPMMLIKNTPVPSNVTAFSEIPVKSFITQYSTGQVTVEMEWELKKENSKRWNPEIQYTNNYNNPEFVDFAPDTSGEYRTTRAIGTRYLTRPL

SEQ ID NO: 49 - protein, 716 aa, Sus scrofa
  misc_feature (1)..(716): VP1 sequence (from pig (denoted as Bpo23))
MSFVDHPPDWLEEIGEGLKEFLGLEPGPPKPKPNQQKRDNARGLVLPGYNYLGPGNGLDRGEPVNRVDEVARERDISYNEQLQAGDNPYLKYNHADAEFQEKLADDTSFGGNLGKAVFQAKKRVLEPFGLVEEPAKTAAKGERIDDHYPKKKKARVEETEAGTSGGQQLQIPAQPASSLGADTMSAGGGSPLGDNNQGADGVGNASGDWHCDSTWMGDRVITKSTRTWVLPSYNNHLYKEIHSGSVDGSNANAYFGYSTPWGYFDFNRFHSHWSPRDWQRLVNNYWGFRPRSLKVKIFNIQVKEVTVQDATTTIANNLTSTVQVFTDDDYQLPYVIGNGTEGCLPAFPLQVFTLPQYGYATLNRNNTDDPTERSSFFCLEYFPSKMLRTGNSFEFTYSFEEVPFHCSFAPSQNLFKLANPLVDQYLYRFVSTDTSGNIQFQKNLKARYANTYKNWFPGPMCRTQGWYTGSGTYNRSGVTNFATSNRMDLEGASYQVNPQPNGMTNTLQDSNKYALENTMIFNSQNAEPGTTSLYQENNLLITSESETQPVNRVAYDTGGQMATNAQSTNLAPTVGTYNHQEMLPGSVWMDRDVYLQGPIWAKIPETGAHFHPSPAMGGFGLKHPPPMMLIKNTPVPSNVTAFSEIPVKSFITQYSTGQVTVEMEWELKKENSKRWNPEIQYTNNYNNPEFVDFAPDTSGEYRTTRAIGTRYLTRPL

SEQ ID NO: 50 - protein, 716 aa, Sus scrofa
  misc_feature (1)..(716): VP1 sequence (from pig (denoted as Bpo24))
MSFVDHPPDWLEEIGEGLKEFLGLEPGPPKPKPNQQKQDNARGLVLPGYNYLGPGNGLDRGEPVNRADEVAREHDISYNEQLQAGDNPYLKYNHADAEFQEKLADDTSFGGNLGKAVFQAKKRVLEPFGLVEEPAKTAAKGERIDDHYPKKKKARVEETEAGTSGGQQLQIPAQPASSLGADTMSAGGGSPLGDNNQGADGVGNASGDWHCDSTWMGDRVITKSTRTWVLPSYNNHLYKEIHSGSVDGSNANAYFGYSTPWGYFDFNRFHSHWSPRDWQRLVNNYWGFRPRSLKVKIFNIQVKEVTVQDATTTIANNLTSTVQVFTDDDYQLPYVIGNGTEGCLPAFPPQVFTLPQYGYATLNRNNTDDPTERSSFFCLEYFPSKMLRTGNSFEFTYSFEEVPFHCSFAPSQNLFKLANPLVDQYLYRFVSTDTSGNIQFQKNLKARYANTYKNWFPGPMCRTQGWYTGSGTYNRSGVTNFATSNRMDLEGASYQVNPQPNGMTNTLQDSNKYALENTMIFNSQNAEPGTTSLYQENNLLITSESETQPVNRVAYDTGGQMATNAQSTNLAPTVGTYNHQEMLPGSVWMDRDVYLQGPIWAKIPETGAHFHPSPAMGGFGLKHPPPMMLIKNTPVPSNVTAFSEIPVKSFITQYSTGQVTVEMEWELKKENSKRWNPEIQYTNNYNNPEFVDFAPDTSGECRTTRAIGTRYLTRPL

SEQ ID NO: 51 - protein, 716 aa, Sus scrofa
  misc_feature (1)..(716): VP1 sequence (from pig (denoted as Bpo27))
MSFVDHPPDWLEEIGEGLKEFLGLEPGPPKPKPNQQKQDNARGLVLPGYNYLGPGNGLDRGEPVNRADEVAREHDISYNEQLQAGDNPYLKYNHADAEFQEKLADDTSFGGNLGKAVFQAKKRVLEPFGLVEEPAKTAAKGERIDDHYPKKKKARVEETEAGTSGGQQLQIPAQPASSLGADTMSAGGGSPLGDNNQGADGVGNASGDWHCDSTWMGDRVITKSTRTWVLPSYNNHLYKEIHSGSVDGSNANAYFGYSTPWGYFDFNRFHSHWSPRDWQRLVDNYWGFRPRSLKVKIFNIQVKEVTVQDATTTIANNLTSTVQVFTDDDYQLPYVIGNGTEGCLPAFPPQVFTLPQYGYATLNRNNTDDPTERSSFFCLEYFPSKMLRTGNSFEFTYSFEEVPFHCSFAPSQNLFKLANPLVDQYLYRFVSTDTSGNIQFQKNLKARYANTYKNWFPGPMCRTQGWYTGSGTYNRSGVTNFATSNRMDLEGASYQVNPQPNGMTNTLQDSNKYALENTMVFNSQNAEPGTTSLYQENNLLITSESETQPVNRVAYNTGGQMATNAQSTNLAPTVGTYNHQEMLPGSVWMDRDVYLQGPIWAKIPETGAHFHPSPAMGGFGLKHPPPMMLIKNTPVPSNVTAFSEIPVKSFITQYSTGQVTVEMEWELKKENSKRWNPEIQYTNNYNNPEFVDFAPDTSGEYRTTRAIGTRYLTRPL

SEQ ID NO: 52 - protein, 716 aa, Sus scrofa
  misc_feature (1)..(716): VP1 sequence (from pig (denoted as Bpo28))
MSFVDHPPDWLEEIGEGLKEFLGLEPGPPKPKPNQQKRDNARGLVLPGYNYLGPGNGLGRGEPVNRADEVAREHDISYNEQLQAGDNPYLKYNHADAEFQEKLADDTSFGGNLGKAVFQAKKRVLEPFGLVEEPAKTAAKGERIDDHYPKKKKARVEETEAGTSGGQQLQIPAQPASSLGADTMSAGGGSPLGDNNQGADGVGNASGDWHCDSTWMGDRVITKSTRTWVLPSYNNHLYKEIHSGSVDGSNANAYFGYSTPWGYFDFNRFHSHWSPRDWQRLVNNYWGLRPRSLKVKIFNIQVKEVTVQDATTTIANNLTSTVQVFTDDDYQLPYVIGNGTEGCLPAFPPQVFTLPQYGYATLNRNNTDDPTERSSFFCLEYFPSKMLRTGNSFEFTYSFEEVPFHCSFAPSQNLFKLANPLADQYLYRFVSTDTSGNIQFQKNLKARYANTYKNWFPGPMCRTQGWYTGSGTYNRSGVTNFATSNRMDLEGASYQVNPQPNGMTNTLQDSNKYALENTMISNSQNAEPGTTSLYRENNLLITSESETQPVNRVAYDTGGQMATNAQSTNLAPTVGTYNHQEMLPGSVWMDRDVYLQGPIWAKIPETGAHFHPSPAMGGFGLKHPPPMMLIKNTPVPSNVTAFSEIPVKSFITQYSTGQVTVEMEWELKKENSKRWNPEIQYTNNYNNPEFVDFAPDTSGEYRTTRAIGTRYLTRPL

SEQ ID NO: 53 - protein, 716 aa, Sus scrofa
  misc_feature (1)..(716): VP1 sequence (from pig (denoted as Bpo29))
MSFVDHPPDWLEEIGEGLKEFLGLEPGPPKPKPNQQKQDNARGLVLPGYNYLGPGNGLDRGEPVNRADEVAREHDISYNEQLQAGDNPYLKYNHADAEFQEKLADDTSFGGNLGKAVFQAKKRVLEPFGLVEEPAKTAAKGERIDDHYPKKKKARVEETEAGTSGGQQLQIPAQPASSLGADTMSAGGGSPLGDNNQGADGVGNASGDWHCDSTWMGDRVITKSTRTWVLPSYNNHLYKEIHSGSVDGSNANAYFGYSTPWGYFDFNRFHSHWSPRDWQRLVNNYWGFRPRSLKVKIFNIQVKEVTVQDATTTIANNLTSTVQVFTDDDYQLPYVIGNGTEGCLPAFPPQVFTLPQYGYATLNRNNTDDPTERSSFFCLEYFPSKMLRTGNSFEFTYSFEEVPFHCSFAPSQNLFKLANPLVDQYLYRFVSTDTSGNIQFQKNLKARYANTYKNWFPGPMCRTQGWYTGSGTYNRSGVTNFATSNRMDLEGASYQVNPQPNGMTNTLQDSNKYALENTMIFNSQNAEPGTTSLYQENNLLITSESGTQPVNRVAYDTGGQMATNAQSTNLAPTVGTYNHQEMLPGSVWMDRGVYLQGPIWAKIPETGAHFHPSPAMGGFGLKHPPPMMLIKNTPVPSNVTAFSEIPVKSFVTQYSTGQVTVEMEWELKKENSKRWNPEIQYTNNYNNPEFVDFAPDTSGEYRTTRAIGTRYLTRPL

SEQ ID NO: 54 - protein, 716 aa, Sus scrofa
  misc_feature (1)..(716): VP1 sequence (from pig (denoted as Bpo33))
MSFVDHPPDWLEEIGEGLKEFLGLEPGPPKPKPNQQKQDNARGLVLPGYNYLGPGNGLDRGEPVNRADEVAREHDISYNEQLQAGDNPYLKYNHADAEFQEKLADDTSFGGNLGKAVFQAKKRVLEPFGLVEEPAKTAAKGERIDDHYPKKKKARVEETEAGTSGGQQLQIPAQPASSLGADTMSAGGGSPLGDNNQGADGVGNASGDWHCDSTWMGDRVITKSTRTWVLPSYNNHLYKEIHSGSVDGSNAHAYFGYSTPWGYFDFNRFHSHWSPRDWQRLVNNYWGFRPRSLKVKIFNIQVKEVTVQDATTTIANNLTSTVQVFTDDDYQLPYVIGNGTEGCLPAFPPQVFTLPQYGYATLNRNNTDDPTERSSFFCLEYFPSKMLRTGNSFEFTYSFEEVPFHCSFAPSQNLFKLANPLVDQYLYRFVSTDTSGNIQFQKNLKARYANTYKNWFPGPMCRTQGWYTGSGTYNRSGVTNFATSNRMDLEGASYQVNPQPNGMTNTLQDSNKYALENTMIFNSQNAEPGTTSLYQENNLLITSESETQPVNRVAYDTGGQMATNAQSTNLAPTVGTYNHQEMLPGSVWMDRDVYLQGPIWAKIPETGAHFHPSPAMGGFGLKHPPPMMLIKNTPVPSNVTAFSEIPVKSFITQYSTGQVTVEMEWELKKENSKRWNPEIQYTNNYNNPEFVDFAPDTSGEYRTTRAIGTRYLTRPL

SEQ ID NO: 55 - protein, 716 aa, Sus scrofa
  misc_feature (1)..(716): VP1 sequence (from pig (denoted as Bpo35))
MSFVDHPPDWLEEIGEGLKEFLGLEPGPPKPKPNQQKRDNARGLVLPGYNYLGPGNGLDRGEPVNRADEVAREHDISYNEQLQAGDNPYLKYNHADAEFQEKLADDTSFGGNLGKAVFQAKKRVLEPFGLVEEPAKTAAKGERIDDHYPKKKKARVEETEAGTSGGQQLQIPAQPASSLGADTMSAGGGSPLGDNNQGADGVGNASGDWHCDSTWMGDRVITKSTRTWVLPSYNNHLYKEIHSGSVDGSNANAYFGYSTPWGYFDFNRFHSHWSPRDWQRLVNNYWGFRPRSLKVKIFNIQVKEVTVQDATTTIANNLTSTVQVFTDDDYQLPYVIGNGTEGCLPAFPPQVFTLPQYGYATLNRNNTDDPTERSSFFCLEYFPSKMLRTGNSFEFTYSFEEVPFHCSFAPSQNLFKLANPLVDQYLYRFVSTDTSGNIQFQKNLKARYANTYKNWFPGPMCRTQGWYTGSGTYNRSGVTNFATSNRMDLEGASYQVNPQPNGMTNTLQDSNKYALENTMIFNSQNAEPGTTSLYQENNLLITSESETQPVNRVAYDTGGQMATNAQSTNLAPTVGTYNHQEMLPGSVWMDRDVYLQGPIWAKIPETGAHFHPSPAMGGFGLKHPPPMMLIKNTPVPSNVTAFSEIPVKSFITQYSTGQVTVEMEWELKKENSKRWNPEIQYTNNYNNPEFVGFAPDTSGEYRTTRAIGTRYLTRPL

SEQ ID NO: 56 - protein, 716 aa, Sus scrofa
  misc_feature (1)..(716): VP1 sequence (from pig (denoted as Bpo36))
MSFVDHPPDWLEEIGEGLKEFLGLEPGPPKPKPNQQKQDNARGLVLPGYNYLGPGNGLDRGEPVNRADEVAREHDISYNEQLQAGDNPYLKYNHADAEFQEKLADDTSFGGNLGKAVFQAKKRVLEPFGLVEEPAKTAAKGERIGDHYPKKKKARVEETEAGTSGGQQPQIPAQPASSLGADTMSAGGGSPLGDNNQGADGVGNASGDWHCDSTWMGDRVITKSTRTWVLPSYNNHLYKEIHSGSVDGSNANAYFGYSTPWGYFDFNRFHSHWSPRDWQRLVNNYWGFRPRSLKVKIFNIQVKEVTVQDATTTIANNLTSTVQVFTDDDYQLPYVIGNGTEGCLPAFPPQVFTLPQYGYATLNRNNTDDPTERSSFFCLEYLPSKMLRTGNSFEFTYSFEEVPFHCSFAPSQNLFKLANPLVDQYLYRFVSTDTSGNIQFQKNLKARYANTYKNWFPGPMCRTQGWYTGSGTYNRSGVTNFATSNRMDLEGASYQVNPQPNGMTNTLQDSNKYALENTMIFNSQNAEPGTTSLYQENNLLITSESETQPVNRVAYNTGGQMATNAQSTNLAPTVGTYNHQEMLPGSVWMDRDVYLQGPIWAKIPETGAHFHPSPAMGGFGLKHPPPMMLIKNTPVPSNVTAFSEIPVKSFITQYSTGQVTVEMEWELKKENSKRWNPEIQYTNNYNNPEFVDFAPDTSGEYRTTRAIGTRYLTRPL

SEQ ID NO: 57 - protein, 716 aa, Sus scrofa
  misc_feature (1)..(716): VP1 sequence (from pig (denoted as Bpo37))
MSFVDHPPDWLEEIGEGLKEFLGLKPGPLKPKPNQQKQDNARGLVLPGYNYLGPGNGLDRGEPVNRADEVAREHDISYNEQLQAGDNPYLKYNHADAEFQEKLADDTSFGGNLGKAVFQAKKRVLEPFGLVEEPAKTAAKGERIDDHYPKKKKARVEETEAGTSGGQQLQIPAQPASSLGADTMSAGGGSPLGDNNQGADGVGNASGDWHCDSTWMGDRVITKSTRTWVLPSYNNHLYKEIHSGSVDGSNANAYFGYSTPWGYFDFNRFHSHWSPRDWQRLVNNYWGFRPRSLKVKIFNIQVKEVTVQDATTTIANNLTSTVQVFTDDDYQLPYVIGNGTEGCLPAFPPQVFTLPQYGYATLNRNNTDDPTERSSFFCLEYFPSKMLRTGNSFEFTYSFEEVPFHCSFAPSQNLFKLANPLVDQYLYRFVSTDTPGNIQFQKNLKARYANTYKNWFPGPMCRTQGWYTGSGTYNRSGVTNFATSNRMDLEGASYQANPQPNGMTNTLQDSNKYALENTMIFNSQNAEPGTTSLYQENNLLITSESETQPVNRVAYDTGGQMATNAQSTNLAPTVGTYNHQEMLPGSVWMDRDVYLQGPIWAKIPETGAHFHPSPAMGGFGLKHPPPMMLIKNTPVPSNVTAFSEIPVKSLITQYSTGQVTVEMEWELKKENSKRWNPEIQYTNNYNNPEFVDFAPDTSGEYRTTRAIGTRYLTRPL

SEQ ID NO: 58 - protein, 736 aa, Macaca mulatta
  misc_feature (1)..(736): VP1 sequence (from rhesus macaque (denoted as Brh26))
MAADGYLPDWLEDNLSEGIREWWDLKPGAPKPKANQQKQDDGRGLVLPGYKYLGPFNGLDKGEPANAADAAALEHDKAYDQQLKAGDNPYLRYNHADAEFQERLQEDTSFGGNLGRAIFQAKKRVLEPLGLVEEGAKTAPGKKGPVEQSPQEPDSSSGIGKTGQQPAKKRLNFGQTGDSESVPDPQPLGEPPAAPSGLGPNTMASGGGAPMADNNEGADGVGNSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTTNEGTKTIANNLTSTVQVFTDSEYQLPYVLGSAHQGCLPPFPADVFMVPQYGYLTLNNGSQALGRSSFYCLEYFPSQMLRTGNNFQFSYTFEDVPFHSSYAHSQSLDRLMNPLIDQYLYYLVRTQTTGTGGTQTLAFSQAGPSSMANQARNWVPGPCYRQQRVSTTTNQNNNSNFAWTGAAKFKLNGRDSLMNPGVAMASHKDDDDRFFPSSGVLIFGKQGTGNDGVDYSQVLITDEEEIKATNPVATEEYGAVAINNQAANTQAQTGLVHNQGVIPGMVWQNRDVYLQGPIWAKIPHTDGNFHPSPLMGGFGLKHPPPQILIKNTPVPADPPLTFNQAKLNSFITQYSTGQVSVEIEWELQKENSKRWNPEIQYTSNYYKSTNVDFAVNTEGVYSEPRPIGTRYLTRNL

SEQ ID NO: 59 - protein, 736 aa, Macaca mulatta
  misc_feature (1)..(736): VP1 sequence (from rhesus macaque (denoted as Brh27))
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAIFQAKKRVLEPLGLVEEGAKTAPGKKRPVEQSPQEPDSSSGIGKTGQQPAKKRLNFGQTGDSESVPDPQPLGEPPAAPSGLGPNTMASGGGAPMADNNEGADGVGNPSGNWHCDSTRLGDRVITTSTRTWALPTYNNHLYKQISNGTSGGSTNDNTYFGYSTPWGYFDFNRFHCHFSPRDWQRLINNNWGFRPKRLNFKLFNIQVKEVTTNEGTKTIANNLTSTVQVFTDSEYQLPYVLGSAHQGCLPPFPADVFMVPQYGYLTLNNGSQALGRSSFYCLEYFPSQMLRTSNNFQFSYTFEDVPFHSSYAHSQSLDRLMNPLIDQYLYYLVRTQTTGTGGTQTLAFSQAGPSSMANQARNWVPGPCYRQQRVSTTTNQNNNSNFAWTGAAKFKLNGRDSLMNPGVAMASHKDDDDRFFPSSGVLIFGKQGTGNDGVDYSQVLITDEEEIKATNPVATEEYGAVAINNQAANTQAQTGLVHNQGVIPGMVWQNRDVYLQGPIWAKIPHTDGNFHPSPLMGGFGLKLPPPQILIKNTPVPADPPLTFNQAKLNSFITQYSTGQVSVEIEWELQKENSKRWNPEIQYTSNYYKSTNVDFAVNTEGVYSEPRPIGTRYLTRNL

SEQ ID NO: 60 - protein, 736 aa, Macaca mulatta
  misc_feature (1)..(736): VP1 sequence (from rhesus macaque (denoted as Brh28))
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAIFQAKKRVLEPLGLVEEGAKTAPGKKRPVEQSPQEPDSPSGIGKTGQQPAKKRLNFGQTGDSESVPDPQPLGEPPAAPSGLGPNTMASGGGAPMADNNEGADGVGNSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTTNEGTKTIANNLTSTVQVFTDSEYQLPYVLGSAHQGCLPPFPADVFMVPQYGYLTLNNGSQALGRSSFYCLEYFPSQMLRTGNNFQFSYTFEDVPFHSSYAHSQSLDRLMNPLIDQYLYYLVRTQTTGTGGTQTLAFSQAGPSSMANQARNWVPGPCYRQQRVSTTTNQNNNSNFAWTGAAKFKLNGRDSLMNPGVAMASHKDDDDRFFPSSGVLIFGKQGTGNDGVDYSQVLITDEEEIKATNPVATEEYGAVAINNQAANTQAQTGLVHNQGVIPGMVWQNRDVYLQGPIWAKIPHTDGNFHPSPLMGGFGLKHPPPQILIKNTPVPADPPLTFNQAKLNSFITQYSTGQVSVEIEWELQKENSKRWNPEIQYTSNYYKSTNVDFAVNTEGVYSEPRPIGTRYLTRNL

SEQ ID NO: 61 - protein, 736 aa, Macaca mulatta
  misc_feature (1)..(736): VP1 sequence (from rhesus macaque (denoted as Brh29))
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAIFQAKRRVLEPLGLVEEGAKTAPGKKRPVEQSPQEPDSSSGIGKTGQQPAKKRLNFGQTGDSESVPDPQPLGEPPAAPSGLGPNTMASGGGAPMADNNEGADGVGNSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTTNEGTKTIANNLTSTVQVFTDSEYQLPYVLGSAHQGCLPPFPADVFMVPQYGYLTLNNGSQALGRSSFYCLEYFPSQMLRTSNNFQFSYTFEDVPFHSSCAHSQSLDRLMNPLIDQYLYYLVRTQTTGTGGTQTLAFSQAGPGSMANQARNWVPGPCYRQQRVSTTTNQNNNSNFAWTGAAKFKLNGRDSLMNPGVAMASHKDDDDRFFPSSGVLIFGKQGTGNDGVDYSQVLITDEEEIKATNPVATEEYGAVAINNQAANTQAQTGLVHNQGVIPGMVWQNRDVYLQGPIWAKIPHTDGNFHPPPLMGGFGLKHPPPQILIKNTPVPADPPLTFNQAKLNSFITQYSTGQVSVEIEWELQKENSKRWNPEIQYTSNYYKSTNVDFAVNTEGVYSEPRPIGTRYLTRNL

SEQ ID NO: 62 - protein, 736 aa, Macaca mulatta
  misc_feature (1)..(736): VP1 sequence (from rhesus macaque (denoted as Brh30))
MAADGYLPDWLEDNLSEGIREWWDLKPGAPKPKANQQKQDDGRGLVFPGYKYLGPFNGLDKGEPVNAADAAALEHDKAYDQQLKAGDNPYLRYNHADAEFQERLQEDTSFGGNLGRAIFQAKKRVLEPLGLVEEGAKTAPGKKRPVEQSPQEPDSSSGIGKTGRQPAKKRLNFGQTGDSESVPDPQPLGEPPAAPSGLGPNTMASGGGAPMADNNEGADGVGNSSGNWHCDSTWLGDRVITTSTRTWALPTYNNHLYKQISNGTPGGSTNDNTYFGYSAPWGYFDFNRFHCHFSPRDWQRLINNNWGFRPKRLNFKLFNIQVKEVTTNEGTKTIANNLTSTVQVFTDSEYQLPYVLGSAHQGCLPPFPADVFMVPQYGYLTLNNGSQALGRSSFYCLEYFPSQMLRTGNNFQFSYTFEDVPFHSSYAHSQSLDRLMNPLIDQYLYYLVRTQTTGTGGTQTLAFSQAGPSSMANQARNWVPGPCYRQQRVSTTTNQNNNSNFAWTGAAKFKLNGRDSLMNPGVAMASHKDDDDRFFPSSGVLIFGKQGTGNDGVDYSQVLITDEEEIKATNPVATEEYGAVAINNQAANTQAQTGLVHNQGVIPGMVWQNRDVYQQGPIWAKIPHTDGNFHPSPLMGGFGLKHPPPQILIKNTPVPADPPLTFNQAKLNSFITQYSTGQVSVEIEWELQKENSKRWNPEIQYTSNYYKSTNVDFAVNTEGVYSEPRPIGTRYLTRNL

SEQ ID NO: 63 - protein, 736 aa, Macaca mulatta
  misc_feature (1)..(736): VP1 sequence (from rhesus macaque (denoted as Brh31))
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAIFQAKKRVLEPLGLVEEGAKTAPGKKRPVEQSPQEPDSSSGIGKTGQQPAKKRLNSGQTGDSESVPDPQPLGEPPAAPSGLGPNTMASGGGAPMADNNEGADGVGNSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTTNEGTKTIANNLTSTVQVFTDSEYQLPYVLGSAYQGCLPPFPADVFMVPQYGYLTLNNGSQALGRSSFYCLEYFPSQMLRTGNNFQSSYTFEDVPFHSSYAHSQSLDRLMNPLIDQYLYYLVRTQTTGTGGTQTLAFSQAGPSSMANQARNWVPGPCYRQQRVSTTTNQNNNSNFAWTGAAKFKLNGRDSLMNPGVAMASHKDDDDRFFPSSGVLIFGKQGTGNDGVDYSQVLITDEEEIKATNPVATEEYGAVAINNQAANTQAQTGLVHNQGVIPGMVWQNRDVYLQGPIWAKIPHTDGNFHPSPLMGGFGLKHPPPQILIKNTPVPADPPLTFNQAKLNSFITQYSTGQVSVEIEWELQKENSKRWNPEIQYTSNYYKSTNVDFAVNTEGVYSEPRPIGTRYLTRNL

SEQ ID NO: 64 - protein, 736 aa, Macaca mulatta
  misc_feature (1)..(736): VP1 sequence (from rhesus macaque (denoted as Brh32))
MAADGYLPDWLEDNLSEGIREWWDLKPGASKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTVPGKKRPVEQSPQEPDSSSGIGKTGQQPAKKRLNFGQTGDSESVPDPQPLGEPPAAPSGLGPNTMASGGGAPMADNNEGADGVGNSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTTNEGTKTIANNLTSTVQVFTDSEYQLPYVLGSAYQGCLPPFPADVFMVPQYGYLTLNNGSQALGRSSFYCLEYFPSQMLRTGNNFQFSYTFEDVPFHSSYAHSQSLDRLMNPLIDQYLYYLVRTQTTGTGGTQTLAFSQAGPSSMANQARNWVPGPCYRQQRVSTTTNQNNNSNFAWTGAAKFKLNGRDSLMNPGVAMASHKDDDDRFFPSSGVLIFGKQGTGNDGVDYSQVLITDEEEIKATNPVATEEYGAVAINNQAANTQAQTGLVHNQGVIPGMVWQNRDVYLQGPIWAKIPHTDGNFHPSPLMGGFGLKHPPPQILIKNTPVPADPPLTFNQAKLNSFITQYSTGQVSVEIEWELQKENSKRWNPEIQYTSNYYKSTNVDFAVNTEGVYSEPRPIGTRYLTRNL

SEQ ID NO: 65 - protein, 736 aa, Macaca mulatta; identical to SEQ ID NO: 64
  misc_feature (1)..(736): VP1 sequence (from rhesus macaque (denoted as Brh33))

SEQ ID NO: 66 - protein, 737 aa, Macaca cyclopis
  misc_feature (1)..(737): VP1 sequence (from formosan macaque (denoted as Bfm17))
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEQSPQEPDSSSGIGKKGQQPAKKRLNFGQTGDSESVPDPQPLGEPPAAPSSLGLNTMAAGGGAPMADNNEGADGVGSSSGNWHCDSTRLGDRVITTSTRTWALPTYNNHLYKQISNGTSGGSTNDNTYFGYSTPWGYFDFNRFHCHFSPRDWQRLINNNWGFRPKKLSFKLFNIQVKEVTQNEGTKTIANNLTSTIQVFTDSEYQLSYVLGSAHQGCLPPFPADVFMIPQYGYLTLNNGSQAVGRSSFYCLEYFPSQMLRTGNNFSFSYTFEDVPFHSSYAHSQSLDRLMNPLIDQYLYYLSRTQSTGGTAGTQQLLFSQAGPNNMSAQAKNWLPGPMYRQQRVSTTLSQNNNSNFAWTGGTKYHLNGRDSLVNPGVAMATNKDDEDRFFPSSGVLMFGKQGAGKDNVDYSSVMLTSEEEIKTTNPVATEQYGVVADNLQQQNTAPIVGAVNSQEALPGMVWQNRDVYLQGPIWAKIPHTDGNFHPSPLMGGFGLKHPPPQILIKNTPVPADPPTAFNQAKLNSFITQYSTGQVSVEIEWELQKENSKRWNPEIQYTSNYYKSTNVDFAVDTEGVYSEHRPIGTRYLTRNL

SEQ ID NO: 67 - protein, 737 aa, Macaca cyclopis
  misc_feature (1)..(737): VP1 sequence (from formosan macaque (denoted as Bfm18))
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEQSPQEPDSSSGIGKKGQQPAKKRLNFGQTGDSESVPDPQPLGEPPAAPSSLGLNTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKKLSFKLFNIQVKEVTQNEGTKTIANNLTSTIQVFTDSEYQLPYVPGSAHQGCLPPFPADVFMIPQYGYLTLNNGSQAVGRSSFYCLEYFPSQMLRTGNNFSFSYTFEDVPFHSSYAHSQSLDRLMNPLIDQYLYYLSRTQSTGGTAGTQQLLFSQAGPNNMSAQAKNWLPGPMYRQQRVSTTLSQNNNSNFAWTGGTKYHLNGRDSLVNPGVAMATNKDDEDRFFPSSGVLMFGKQGAGKDNVDYSSVMLTSEEEIKTTNPVATEQYGVVADNLQQQNTAPIVGAVNSQGALPGMVWQNRDVYPQGPIWAKIPHTDGNFHPSPLMGGFGLKHPPPQILIKNTPVPADPPTAFNQAKLNSFITQYSTGQVSVEIEWELQKENSKRWNPEIQYTSNYYKSTNVDFAVNTEGVYSEPRPIGTRYLTRNL

SEQ ID NO: 68 - protein, 737 aa, Macaca cyclopis
  misc_feature (1)..(737): VP1 sequence (from formosan macaque (denoted as Bfm.20))
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPPGLVEEGAKTAPGKKRPVEQSPQEPDSSSGIGKKGQQPAKKRLNFGQTGDSESVPDPQPLGEPPAAPSSLGLNTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKKLSFKLFNIQVKEVTQNEGTKTIANNLTSTIQVFTDSEYQLPYVLGSAHQGCLPPFPADVFMIPQYGYLTLNNGSQAVGRSFFYCLEYFPSQMLRTGNNFSFSYTFEDVPFHSSYAHSQSLDRLMNPLIDQYLYYLSRTQSTGGTAGTQQLLFSQAGPNNMSAQAKNWLPGPMYRQQRVSTTLSQNNNSNFAWTGGTKYHLNGRDSLVNPGVAMATNKDDEDRFFPSSGVLMFGKQGAGKDNVDYSSVMLTSEEEIKTTNPVATEQYGVVADNLQQQNTAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIERELQKENSKRWNPEIQYTSNYYKSTNVDFAVNTEGVYSEPRPIGTRYLTRNL

SEQ ID NO: 69 - protein, 737 aa, Macaca cyclopis
  misc_feature (1)..(737): VP1 sequence (from formosan macaque (denoted as Bfm21))
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKERPVEQSPQEPDSSSGIGKKGQQPAKKRLNFGQTGDSESVPDPQPLGEPPAAPSSLGLNTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKKLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFSFSYTFEDVPFHSSYAHSQSLDRLMNPLIDQYLYYLSRTQSTGGTAGTQQLLFSQAGPNNMSAQAKNWLPGPMYRQQRVSTTLSQNNNSNFAWTGGTKYHLNGRDSLVNPGVAMATNKDDEDRFFPSSAVLMFGKQGAGKDNVDYSSVMLTSEEEIKTTNPVTTEQYGVVADNLQQQNTAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIEWELQKENSKRWNPEIQYTSNYYKSTNVDFAVNTESVYSEPRPIGTRYLTRNL

SEQ ID NO: 70 - protein, 737 aa, Macaca cyclopis
  misc_feature (1)..(737): VP1 sequence (from formosan macaque (denoted as Bfm24))
MAADGYLPDWLEDNLSEGIREWWALKPGAPKPKANQQKQDDGRGLVLPGYKYLGPFNGPDKGEPVNAADAAALEHDKAYDQQLQAGDNPYLRYNHADAEFQERLQEDTSFGGNLGRAVFQAKKRVLEPLGLVEEGAKTAPGKKRPVEQSPQEPDSSSGIGKKGQQPAKKRLNFGQTGDSESVPDPQPLGEPPAAPSSLGLNTMAAGGGAPMADNNEGADGVGSSSGNWHCDSTWLGDRVITTSTRTWALPTYNNHLYKQISNGTSGGSTNDNTYFGYSTPWGYFDFNRSHCHFSPRDWQRLINNNWGFRPKKLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFSFSYTFEDVPFHSSYAHSQSLDRLMNPLIDQYLYYLSRTQSTGGTAGTQQLLFSQAGPNNMSAQAKNWLPGPMYRQQRVSTTLSQNNNSNFAWTGGTKYHLNGRDSLVNPGVAMATNKDDEDRFFPSSGVLMFGKQGAGKDNVDYSSVMLTSEEEIRTTNPVATEQYGVVADNLQQQNTAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIEWELQKENSKRWNPEIQYTSNYYKSTNVDFAVNTEGVYSEPRPIGTRYLTRNL

SEQ ID NO: 71 - protein, 737 aa, Macaca cyclopis
  misc_feature (1)..(737): VP1 sequence (from formosan macaque (denoted as Bfm25))
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEQSPQEPDSSSGIGKKGQQPAKKRLNFGQTGDSESVPDPQPLGEPPAAPSSLGLNTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKKLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFSFSYTFEDVPFHSSYAHSQSLDRLMNPLIDQYLYYLSRTQSTGGTAGTQQLLFSQAGPNNMSAQAKNWLPGPMYRQQRVSTTLSQNNNSNFAWTGGTKYHLNGRDSLVNPGVAMATNKDDEDRLFPSSGVLMFGKQGAGKDNVDYSSVMLTSEEEVKTTNPVATEQYGVVADNLQQQNTAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIEWELQKENSKRWNPEIQYTSNYYKSTNVDFAVNTEGVYSEPRPIGTRYLTRNL

SEQ ID NO: 72 - protein, 737 aa, Macaca cyclopis
  misc_feature (1)..(737): VP1 sequence (from formosan macaque (denoted as Bfm27))
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKRRVLEPLGLVEEGAKTAPGKKRPVEQSPQEPDSSSGIGKKGQQPAKKRLNFGQTGDSESVPDPQPLGEPPAAPSSLGLNTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKKLSFKLFNIQVKEVTQNEGTKTIANNLTSTIQVFTDSEYQLPYVLGSARQGCLPPFPADVFMIPQYGYLTLNNGSQAVGRSSFYCLEYFPSQMLRTGNNFSFSYTFEDVPFHSSYAHSQSLDRLMNPLIDQYLYYLSRTQSTGGTAGTQQLLFSQAGPNNMSAQAKNWLPGPMYRQQRVSTTLSQNNNSNFAWTGGTKYHLNGRDSLVNPGVAMATNKDDEDRFFPSSGVLMFGKQGAGKDNVDYSSVMLTSEEEIKTTNPVATEQYGVVADNLQQQNTAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIEWELQKENSKRWNPEIQYTSNYYKSTNVDFAVDTEGVYSEPRPIGTRYLTRNL

SEQ ID NO: 73 - protein, 737 aa, Macaca cyclopis
  misc_feature (1)..(737): VP1 sequence (from formosan macaque (denoted as Bfm32))
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEQSPQEPDSSSGIGKKGQQPAKKRLNFGQTGDSESVPDPQPLGEPPAAPSSLGLNTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKKLSFKLFDIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFSFSYTFEDVPFHSSYAHSQSLDRLMNPLIDQYLYYLSRTQSTGGTAGTQQLLFSQAGPNNMSAQAKNWLPGPMYRQQRVSTTLSQNNNSNFAWTGGTKYHLNGRDSLVNPGVAMATNKDDEDRFFPSSGVLMFGKQGAGKDNVDYSSVMLTSEEEIKTTNPVATEQYGVVADNLQQQNTAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIEWELQKENSKRWNPEIQYTSNYYKSTNVDFAVNTEGVYSEPRPIGTRYLTRNL

SEQ ID NO: 74 - protein, 737 aa, Macaca cyclopis
  misc_feature (1)..(737): VP1 sequence (from formosan macaque (denoted as Bfm33))
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEQSPQEPDSSSGIGKKGQQPAKKRLNFGQTGDSESVPDPQPLGEPPAAPSSLGLNTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKKLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFSFSYTFEDVPFHSSYAHSQSLDRLMNPLIDQYLYYLSRTQSTGGTAGTQQLLFSQAGPNNMSAQAKNWLPGPMYRQQRVSTTLSQNNNSNFAWTGGTKYHLNGRDSLVNPGVAMATNKDDEDRFFPSSAVLMFGKQGAGKDNVDYSSVMLTSEEEIKTTNPVATEQYGVVADNLQQQNTAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIEWELQKENSKRWNPEIQYTSNYYKSTNVDFAVNTESVYSEPRPIGTRYLTRNL

SEQ ID NO: 75 - protein, 737 aa, Macaca cyclopis
  misc_feature (1)..(737): VP1 sequence (from formosan macaque (denoted as Bfm34))
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEQSPQEPDSSSGIGKKGQQPAKKRLNFGQTGDSESVPDPQPLGEPPAAPSSLGLNTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKKLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFSFSYTFEDVPFHSSYAHSQSLDRLMNPLIDQYLYYLSRTQSTGGTAGTQQLLFSQAGPNNMSAQAKNWLPGPMYRQQRVSTTLSQNNNSNFAWTGGTKYHLNGRDSLVYPGVAMATNKDDEDRFFPSSGVLMFGKQGAGKDNVDYSSVMLTSEEEIKTTNPVATEQYGVVADNLQQQNTAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIEWELQKENSKRWNPEIQYTSNYYKSTNVDFAVNTEGVYSEPRPIGTRYLTRNL

SEQ ID NO: 76 - protein, 737 aa, Macaca cyclopis
  misc_feature (1)..(737): VP1 sequence (from formosan macaque (denoted as Bfm35))
MAADGYLPDWLEDNLSEGIREWWALKPGAPKPKANQQKQDDGRGLVLPGYKYLGPFNGLDKGGPVNAADAAALEHDKAYDQQLQAGDNPYLRYNHADAEFRERLQEDTSFGGNLGRAVFQAKKRVLEPLGLVEEGAKTAPGKKRPVEQSPQEPDSSSGIGKKGQQPAKKRLNFGQTGDSESVPDPQPLGEPPAAPSSLGLNTMAAGGGAPMADNNEGADGVGSSSGNWHCDSTWLGDRVITTSTRTWALPTYNNHLYKQISNGTSGGSTNDSTYFGYSTPWGYFDFNRSHCHFSPRDWQRLINNNWGFRPKKLSFKLFNIQVKEVTQNEGTKTIANNLTSTIQVFTDSEYQLPYVLGSAHQGCLPPFPADVFMIPQYGYLTLNNGGQAVGRSSFYCLEYFPSQMLRTGNNFSFSYIFEDVPFHSSYAHSQSLDRLMNPLIDQYLYYLSRTQSTGGTAGTQQLLFSQAGPNNMSAQAKNWLPGPMYRQQRVSTTLSQNNNSNFAWTGGTKYHLNGRDSLVNPGVAMATNKDDEDRFFPSSGVLMFGKQGAGKDNVDYSSVMLTSEEEIKTTNPVATEQYGVVADNLQQQNTAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIEWELLKESSKRWNPEIQYTSNYYKSTNVDFAVNTEGVYSEPRPIGTRYLTRNL